SEQUENCE LISTING (118 sequences)

SEQ ID NO: 1 - protein, 11 aa, synthetic construct
  REGION 1..11: note = Synthetic
QASQSISSYLS

SEQ ID NO: 2 - protein, 7 aa, synthetic construct
  REGION 1..7: note = Synthetic
KASTLAS

SEQ ID NO: 3 - protein, 12 aa, synthetic construct
  REGION 1..12: note = Synthetic
QQGYTSSNIDNI

SEQ ID NO: 4 - protein, 5 aa, synthetic construct
  REGION 1..5: note = Synthetic
NYAMI

SEQ ID NO: 5 - protein, 7 aa, synthetic construct
  REGION 1..7: note = Synthetic
GFSLSNY

SEQ ID NO: 6 - protein, 16 aa, synthetic construct
  REGION 1..16: note = Synthetic
TISLGGYTYYANWAKG

SEQ ID NO: 7 - protein, 16 aa, synthetic construct
  REGION 1..16: note = Synthetic
ARWSTDSAIYTYAFDP

SEQ ID NO: 8 - protein, 121 aa, synthetic construct
  REGION 1..121: note = Synthetic
QSVEESGGRLVTPGTPLTLTCTVSGFSLSNYAMIWVRQAPGEGLEWVGTISLGGYTYYANWAKGRFTISKTSTTVDLKISSPTTEDTATYFCARARWSTDSAIYTYAFDPWGPGTLVTVSS

SEQ ID NO: 9 - protein, 110 aa, synthetic construct
  REGION 1..110: note = Synthetic
AYDMTQTPASVEVAVGGTVTIKCQASQSISSYLSWYQQKPGQRPELLIYKASTLASGVSSRFKGSGSGTQFTLTISDLECADAATYYCQQGYTSSNIDNIFGGGTEVVVK

SEQ ID NO: 10 - protein, 124 aa, synthetic construct
  REGION 1..124: note = Synthetic
EQQLLESGGGLVQPGGSLRLSCAVSGFSLSNYAMIWVRQAPGKGLEWVGTISLGGYTYYANWAKGRFTISKDSSKTTVYLQMNSLRAEDTAVYFCARARWSTDSAIYTYAFDPWGPGTLVTVSS

SEQ ID NO: 11 - protein, 124 aa, synthetic construct
  REGION 1..124: note = Synthetic
EVQLLESGGGLVQPGGSLRLSCAVSGFSLSNYAMIWVRQAPGKGLEWVGTISLGGYTYYANWAKGRFTISKDSSKTTVYLQMNSLRAEDTAVYFCARARWSTDSAIYTYAFDPWGPGTLVTVSS

SEQ ID NO: 12 - protein, 124 aa, synthetic construct
  REGION 1..124: note = Synthetic
EQQLLESGGGLVQPGGSLRLSCAASGFSLSNYAMIWVRQAPGKGLEWVGTISLGGYTYYANWAKGRFTISKDSSKTTVYLQMNSLRAEDTAVYFCARARWSTDSAIYTYAFDPWGPGTLVTVSS

SEQ ID NO: 13 - protein, 124 aa, synthetic construct
  REGION 1..124: note = Synthetic
EQQLLESGGGLVQPGGSLRLSCAVSGFSLSNYAMIWVRQAPGKGLEWVSTISLGGYTYYANWAKGRFTISKDSSKTTVYLQMNSLRAEDTAVYFCARARWSTDSAIYTYAFDPWGPGTLVTVSS

SEQ ID NO: 14 - protein, 124 aa, synthetic construct
  REGION 1..124: note = Synthetic
EQQLLESGGGLVQPGGSLRLSCAVSGFSLSNYAMIWVRQAPGKGLEWVGTISLGGYTYYANWAKGRFTISRDSSKTTVYLQMNSLRAEDTAVYFCARARWSTDSAIYTYAFDPWGPGTLVTVSS

SEQ ID NO: 15 - protein, 124 aa, synthetic construct
  REGION 1..124: note = Synthetic
EQQLLESGGGLVQPGGSLRLSCAVSGFSLSNYAMIWVRQAPGKGLEWVGTISLGGYTYYANWAKGRFTISKDNSKTTVYLQMNSLRAEDTAVYFCARARWSTDSAIYTYAFDPWGPGTLVTVSS

SEQ ID NO: 16 - protein, 124 aa, synthetic construct
  REGION 1..124: note = Synthetic
EQQLLESGGGLVQPGGSLRLSCAVSGFSLSNYAMIWVRQAPGKGLEWVGTISLGGYTYYANWAKGRFTISKDSSKNTVYLQMNSLRAEDTAVYFCARARWSTDSAIYTYAFDPWGPGTLVTVSS

SEQ ID NO: 17 - protein, 124 aa, synthetic construct
  REGION 1..124: note = Synthetic
EQQLLESGGGLVQPGGSLRLSCAVSGFSLSNYAMIWVRQAPGKGLEWVGTISLGGYTYYANWAKGRFTISKDSSKTTLYLQMNSLRAEDTAVYFCARARWSTDSAIYTYAFDPWGPGTLVTVSS

SEQ ID NO: 18 - protein, 124 aa, synthetic construct
  REGION 1..124: note = Synthetic
EQQLLESGGGLVQPGGSLRLSCAVSGFSLSNYAMIWVRQAPGKGLEWVGTISLGGYTYYANWAKGRFTISKDSSKTTVYLQMNSLRAEDTAVYYCARARWSTDSAIYTYAFDPWGPGTLVTVSS

SEQ ID NO: 19 - protein, 124 aa, synthetic construct
  REGION 1..124: note = Synthetic
EQQLLESGGGLVQPGGSLRLSCAVSGFSLSNYAMIWVRQAPGKGLEWVGTISLGGYTYYANWAKGRFTISKDSSKTTVYLQMNSLRAEDTAVYFCARARWSTDSAIYTYAFDPWGQGTLVTVSS

SEQ ID NO: 20 - protein, 124 aa, synthetic construct
  REGION 1..124: note = Synthetic
EVQLLESGGGLVQPGGSLRLSCAASGFSLSNYAMIWVRQAPGKGLEWVSTISLGGYTYYANWAKGRFTISRDNSKNTLYLQMNSLRAEDTAVYYCARARWSTDSAIYTYAFDPWGQGTLVTVSS

SEQ ID NO: 21 - protein, 124 aa, synthetic construct
  REGION 1..124: note = Synthetic
EVQLLESGGGLVQPGGSLRLSCAASGFSLSNYAMIWVRQAPGKGLEWVSTISLGGYTYYANWAKGRFTISRDSSKTTVYLQMNSLRAEDTAVYFCARARWSTDSAIYTYAFDPWGPGTLVTVSS

SEQ ID NO: 22 - protein, 110 aa, synthetic construct
  REGION 1..110: note = Synthetic
DYQMTQSPSSLSASVGDRVTITCQASQSISSYLSWYQQKPGKRPKLLIYKASTLASGVPSRFSGSGSGTDFTLTISSLQPEDFATYYCQQGYTSSNIDNIFGGGTKVEIK

SEQ ID NO: 23 - protein, 110 aa, synthetic construct
  REGION 1..110: note = Synthetic
DIQMTQSPSSLSASVGDRVTITCQASQSISSYLSWYQQKPGKRPKLLIYKASTLASGVPSRFSGSGSGTDFTLTISSLQPEDFATYYCQQGYTSSNIDNIFGGGTKVEIK

SEQ ID NO: 24 - protein, 110 aa, synthetic construct
  REGION 1..110: note = Synthetic
DYQMTQSPSSLSASVGDRVTITCQASQSISSYLSWYQQKPGKAPKLLIYKASTLASGVPSRFSGSGSGTDFTLTISSLQPEDFATYYCQQGYTSSNIDNIFGGGTKVEIK

SEQ ID NO: 25 - protein, 110 aa, synthetic construct
  REGION 1..110: note = Synthetic
DIQMTQSPSSLSASVGDRVTITCQASQSISSYLSWYQQKPGKAPKLLIYKASTLASGVPSRFSGSGSGTDFTLTISSLQPEDFATYYCQQGYTSSNIDNIFGGGTKVEIK

SEQ ID NO: 26 - protein, 11 aa, synthetic construct
  REGION 1..11: note = Synthetic
QASENIANALA

SEQ ID NO: 27 - protein, 7 aa, synthetic construct
  REGION 1..7: note = Synthetic
GASNLAS

SEQ ID NO: 28 - protein, 13 aa, synthetic construct
  REGION 1..13: note = Synthetic
QQAYYGNSFVEGT

SEQ ID NO: 29 - protein, 5 aa, synthetic construct
  REGION 1..5: note = Synthetic
TYAMG

SEQ ID NO: 30 - protein, 7 aa, synthetic construct
  REGION 1..7: note = Synthetic
GIDLSTY

SEQ ID NO: 31 - protein, 16 aa, synthetic construct
  REGION 1..16: note = Synthetic
LIHRSGRTYYATWAKG

SEQ ID NO: 32 - protein, 11 aa, synthetic construct
  REGION 1..11: note = Synthetic
SYPDYSATASI

SEQ ID NO: 33 - protein, 117 aa, synthetic construct
  REGION 1..117: note = Synthetic
QSVEESGGRLVSPGTPLTLTCTVSGIDLSTYAMGWVRQAPGKGLEWIGLIHRSGRTYYATWAKGRFTISKTSSTTVDLKITSPTTEDTATYFCTRSYPDYSATASIWGPGTLVTVSS

SEQ ID NO: 34 - protein, 111 aa, synthetic construct
  REGION 1..111: note = Synthetic
DIVVTQTPASVEAAVGGTVTIKCQASENIANALAWYQQKSGQPPMFLIYGASNLASGVSSRFKGSGSGTEFTLTISDLECADAAVYYCQCAYYGNSFVEGTFGGGTEVVVK

SEQ ID NO: 35 - protein, 119 aa, synthetic construct
  REGION 1..119: note = Synthetic
EQQLLESGGGLVQPGGSLRLSCAVSGIDLSTYAMGWVRQAPGKGLEWIGLIHRSGRTYYATWAKGRFTISKDSSKTTVYLQMNSLRAEDTAVYFCTRSYPDYSATASIWGPGTTVTVSS

SEQ ID NO: 36 - protein, 119 aa, synthetic construct
  REGION 1..119: note = Synthetic
EVQLLESGGGLVQPGGSLRLSCAVSGIDLSTYAMGWVRQAPGKGLEWIGLIHRSGRTYYATWAKGRFTISKDSSKTTVYLQMNSLRAEDTAVYFCTRSYPDYSATASIWGPGTTVTVSS

SEQ ID NO: 37 - protein, 119 aa, synthetic construct
  REGION 1..119: note = Synthetic
EQQLLESGGGLVQPGGSLRLSCAASGIDLSTYAMGWVRQAPGKGLEWIGLIHRSGRTYYATWAKGRFTISKDSSKTTVYLQMNSLRAEDTAVYFCTRSYPDYSATASIWGPGTTVTVSS

SEQ ID NO: 38 - protein, 119 aa, synthetic construct
  REGION 1..119: note = Synthetic
EQQLLESGGGLVQPGGSLRLSCAVSGIDLSTYAMGWVRQAPGKGLEWVGLIHRSGRTYYATWAKGRFTISKDSSKTTVYLQMNSLRAEDTAVYFCTRSYPDYSATASIWGPGTTVTVSS

SEQ ID NO: 39 - protein, 119 aa, synthetic construct
  REGION 1..119: note = Synthetic
EQQLLESGGGLVQPGGSLRLSCAVSGIDLSTYAMGWVRQAPGKGLEWISLIHRSGRTYYATWAKGRFTISKDSSKTTVYLQMNSLRAEDTAVYFCTRSYPDYSATASIWGPGTTVTVSS

SEQ ID NO: 40 - protein, 119 aa, synthetic construct
  REGION 1..119: note = Synthetic
EQQLLESGGGLVQPGGSLRLSCAVSGIDLSTYAMGWVRQAPGKGLEWIGLIHRSGRTYYATWAKGRFTISRDSSKTTVYLQMNSLRAEDTAVYFCTRSYPDYSATASIWGPGTTVTVSS

SEQ ID NO: 41 - protein, 119 aa, synthetic construct
  REGION 1..119: note = Synthetic
EQQLLESGGGLVQPGGSLRLSCAVSGIDLSTYAMGWVRQAPGKGLEWIGLIHRSGRTYYATWAKGRFTISKDNSKTTVYLQMNSLRAEDTAVYFCTRSYPDYSATASIWGPGTTVTVSS

SEQ ID NO: 42 - protein, 119 aa, synthetic construct
  REGION 1..119: note = Synthetic
EQQLLESGGGLVQPGGSLRLSCAVSGIDLSTYAMGWVRQAPGKGLEWIGLIHRSGRTYYATWAKGRFTISKDSSKNTVYLQMNSLRAEDTAVYFCTRSYPDYSATASIWGPGTTVTVSS

SEQ ID NO: 43 - protein, 119 aa, synthetic construct
  REGION 1..119: note = Synthetic
EQQLLESGGGLVQPGGSLRLSCAVSGIDLSTYAMGWVRQAPGKGLEWIGLIHRSGRTYYATWAKGRFTISKDSSKTTLYLQMNSLRAEDTAVYFCTRSYPDYSATASIWGPGTTVTVSS

SEQ ID NO: 44 - protein, 119 aa, synthetic construct
  REGION 1..119: note = Synthetic
EQQLLESGGGLVQPGGSLRLSCAVSGIDLSTYAMGWVRQAPGKGLEWIGLIHRSGRTYYATWAKGRFTISKDSSKTTVYLQMNSLRAEDTAVYYCTRSYPDYSATASIWGPGTTVTVSS

SEQ ID NO: 45 - protein, 119 aa, synthetic construct
  REGION 1..119: note = Synthetic
EQQLLESGGGLVQPGGSLRLSCAVSGIDLSTYAMGWVRQAPGKGLEWIGLIHRSGRTYYATWAKGRFTISKDSSKTTVYLQMNSLRAEDTAVYFCTRSYPDYSATASIWGQGTTVTVSS

SEQ ID NO: 46 - protein, 119 aa, synthetic construct
  REGION 1..119: note = Synthetic
EVQLLESGGGLVQPGGSLRLSCAASGIDLSTYAMGWVRQAPGKGLEWVSLIHRSGRTYYATWAKGRFTISRDNSKNTLYLQMNSLRAEDTAVYYCTRSYPDYSATASIWGQGTTVTVSS

SEQ ID NO: 47 - protein, 119 aa, synthetic construct
  REGION 1..119: note = Synthetic
EVQLLESGGGLVQPGGSLRLSCAASGIDLSTYAMGWVRQAPGKGLEWVGLIHRSGRTYYATWAKGRFTISKDSSKNTLYLQMNSLRAEDTAVYYCTRSYPDYSATASIWGQGTTVTVSS

SEQ ID NO: 48 - protein, 111 aa, synthetic construct
  REGION 1..111: note = Synthetic
DIQVTQSPSSLSASVGDRVTITCQASENIANALAWYQQKPGKPPKFLIYGASNLASGVPSRFSGSGSGTDFTFTISSLQPEDIATYYCQQAYYGNSFVEGTFGGGTKVEIK

SEQ ID NO: 49 - protein, 111 aa, synthetic construct
  REGION 1..111: note = Synthetic
DIQMTQSPSSLSASVGDRVTITCQASENIANALAWYQQKPGKPPKFLIYGASNLASGVPSRFSGSGSGTDFTFTISSLQPEDIATYYCQQAYYGNSFVEGTFGGGTKVEIK

SEQ ID NO: 50 - protein, 111 aa, synthetic construct
  REGION 1..111: note = Synthetic
DIQVTQSPSSLSASVGDRVTITCQASENIANALAWYQQKPGKAPKFLIYGASNLASGVPSRFSGSGSGTDFTFTISSLQPEDIATYYCQQAYYGNSFVEGTFGGGTKVEIK

SEQ ID NO: 51 - protein, 111 aa, synthetic construct
  REGION 1..111: note = Synthetic
DIQVTQSPSSLSASVGDRVTITCQASENIANALAWYQQKPGKPPKLLIYGASNLASGVPSRFSGSGSGTDFTFTISSLQPEDIATYYCQQAYYGNSFVEGTFGGGTKVEIK

SEQ ID NO: 52 - protein, 111 aa, synthetic construct
  REGION 1..111: note = Synthetic
DIQMTQSPSSLSASVGDRVTITCQASENIANALAWYQQKPGKAPKLLIYGASNLASGVPSRFSGSGSGTDFTFTISSLQPEDIATYYCQQAYYGNSFVEGTFGGGTKVEIK

SEQ ID NO: 53 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 107 aa, synthetic construct [sequence omitted]

SEQ ID NO: 55 - protein, 449 aa, synthetic construct
  REGION 1..449: note = Synthetic
EVQLLESGGGLVQPGGSLRLSCAASGIDLSTYAMGWVRQAPGKGLEWVGLIHRSGRTYYATWAKGRFTISKDSSKNTLYLQMNSLRAEDTAVYYCTRSYPDYSATASIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 218 aa, synthetic construct
  REGION 1..218: note = Synthetic
DIQVTQSPSSLSASVGDRVTITCQASENIANALAWYQQKPGKPPKFLIYGASNLASGVPSRFSGSGSGTDFTFTISSLQPEDIATYYCQQAYYGNSFVEGTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 454 aa, synthetic construct
  REGION 1..454: note = Synthetic
EVQLLESGGGLVQPGGSLRLSCAASGFSLSNYAMIWVRQAPGKGLEWVSTISLGGYTYYANWAKGRFTISRDSSKTTVYLQMNSLRAEDTAVYFCARARWSTDSAIYTYAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 217 aa, synthetic construct
  REGION 1..217: note = Synthetic
DYQMTQSPSSLSASVGDRVTITCQASQSISSYLSWYQQKPGKAPKLLIYKASTLASGVPSRFSGSGSGTDFTLTISSLQPEDFATYYCQQGYTSSNIDNIFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 449 aa, synthetic construct
  REGION 1..449: note = Synthetic
EVQLLESGGGLVQPGGSLRLSCAASGIDLSTYAMGWVRQAPGKGLEWVGLIHRSGRTYYATWAKGRFTISKDSSKNTLYLQMNSLRAEDTAVYYCTRSYPDYSATASIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 454 aa, synthetic construct
  REGION 1..454: note = Synthetic
EVQLLESGGGLVQPGGSLRLSCAASGFSLSNYAMIWVRQAPGKGLEWVSTISLGGYTYYANWAKGRFTISRDSSKTTVYLQMNSLRAEDTAVYFCARARWSTDSAIYTYAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 12 aa, synthetic construct
  REGION 1..12: note = Synthetic
RSSKSVYSNYLS

SEQ ID NO: 63 - protein, 7 aa, synthetic construct
  REGION 1..7: note = Synthetic
RASTLTP

SEQ ID NO: 64 - protein, 11 aa, synthetic construct
  REGION 1..11: note = Synthetic
AGGYSSGSDNT

SEQ ID NO: 65 - protein, 5 aa, synthetic construct
  REGION 1..5: note = Synthetic
EYSMA

SEQ ID NO: 66 - protein, 7 aa, synthetic construct
  REGION 1..7: note = Synthetic
RIDLNEY

SEQ ID NO: 67 - protein, 17 aa, synthetic construct
  REGION 1..17: note = Synthetic
YIDAGSGSAYYASWAKG

SEQ ID NO: 68 - protein, 14 aa, synthetic construct
  REGION 1..14: note = Synthetic
DVYPGYTTGTNLGL

SEQ ID NO: 69 - protein, 110 aa, synthetic construct
  REGION 1..110: note = Synthetic
AAVLTQTPASVSAAVGGTVSISCRSSKSVYSNYLSWYQQKPGQPPKLLIYRASTLTPGVPSRFKGSGSGTQFSLTIRDVQSADAGSYYCAGGYSSGSDNTFGGGTKLEIK

SEQ ID NO: 70 - protein, 121 aa, synthetic construct
  REGION 1..121: note = Synthetic
QSVKESGGRLVTPGGSLTLTCTVSRIDLNEYSMAWVRQAPGKGLEWIGYIDAGSGSAYYASWAKGRFTISKTSSTTVDLEMTTLTTEDTATYFCARDVYPGYTTGTNLGLWGPGTLVTVSS

SEQ ID NO: 71 - protein, 217 aa, synthetic construct
  REGION 1..217: note = Synthetic
AAVLTQTPASVSAAVGGTVSISCRSSKSVYSNYLSWYQQKPGQPPKLLIYRASTLTPGVPSRFKGSGSGTQFSLTIRDVQSADAGSYYCAGGYSSGSDNTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 72 - protein, 451 aa, synthetic construct
  REGION 1..451: note = Synthetic
QSVKESGGRLVTPGGSLTLTCTVSRIDLNEYSMAWVRQAPGKGLEWIGYIDAGSGSAYYASWAKGRFTISKTSSTTVDLEMTTLTTEDTATYFCARDVYPGYTTGTNLGLWGPGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 73 - protein, 11 aa, synthetic construct
  REGION 1..11: note = Synthetic
QASQRIGNALA

SEQ ID NO: 74 - protein, 7 aa, synthetic construct
  REGION 1..7: note = Synthetic
RASTLES

SEQ ID NO: 75 - protein, 12 aa, synthetic construct
  REGION 1..12: note = Synthetic
LGGYISIGNVYT

SEQ ID NO: 76 - protein, 12 aa, synthetic construct
  REGION 1..12: note = Synthetic
QSSKSVNNNNLA

SEQ ID NO: 77 - protein, 7 aa, synthetic construct
  REGION 1..7: note = Synthetic
FASALAS

SEQ ID NO: 78 - protein, 12 aa, synthetic construct
  REGION 1..12: note = Synthetic
QGAYSGGSDSYA

SEQ ID NO: 79 - protein, 11 aa, synthetic construct
  REGION 1..11: note = Synthetic
QASQSISNALA

SEQ ID NO: 80 - protein, 7 aa, synthetic construct
  REGION 1..7: note = Synthetic
EVSKVAS

SEQ ID NO: 81 - protein, 13 aa, synthetic construct
  REGION 1..13: note = Synthetic
QSAYYGNSYVWGA

SEQ ID NO: 82 - protein, 6 aa, synthetic construct
  REGION 1..6: note = Synthetic
SNYYMC

SEQ ID NO: 83 - protein, 7 aa, synthetic construct
  REGION 1..7: note = Synthetic
GFDFSSN

SEQ ID NO: 84 - protein, 18 aa, synthetic construct
  REGION 1..18: note = Synthetic
CIHISSVTNTWYASWATG

SEQ ID NO: 85 - protein, 11 aa, synthetic construct
  REGION 1..11: note = Synthetic
VGNSDYRYFNL

SEQ ID NO: 86 - protein, 6 aa, synthetic construct
  REGION 1..6: note = Synthetic
YGYDMC

SEQ ID NO: 87 - protein, 7 aa, synthetic construct
  REGION 1..7: note = Synthetic
GFSFSYG

SEQ ID NO: 88 - protein, 18 aa, synthetic construct
  REGION 1..18: note = Synthetic
CISAGSSDNTWYASWAKG

SEQ ID NO: 89 - protein, 4 aa, synthetic construct
  REGION 1..4: note = Synthetic
YLGL

SEQ ID NO: 90 - protein, 5 aa, synthetic construct
  REGION 1..5: note = Synthetic
SYAMS

SEQ ID NO: 91 - protein, 7 aa, synthetic construct
  REGION 1..7: note = Synthetic
GFSLSSY

SEQ ID NO: 92 - protein, 16 aa, synthetic construct
  REGION 1..16: note = Synthetic
VISASGRIIYATWAKG

SEQ ID NO: 93 - protein, 11 aa, synthetic construct
  REGION 1..11: note = Synthetic
GVPSYSLVMSD

SEQ ID NO: 94 - protein, 110 aa, synthetic construct
  REGION 1..110: note = Synthetic
DIQMTQSPSSLSASVGDRVTITCQASQRIGNALAWYQQKPGKPPKLLIYRASTLESGVPSRFSGSGSGTDFTLTISSLQPEDVATYYCLGGYISIGNVYTFGGGTKVEIK

SEQ ID NO: 95 - protein, 122 aa, synthetic construct
  REGION 1..122: note = Synthetic
EQQLVESAGGLVQPGGSLRLSCAASGFDFSSNYYMCWVRQAPGKGLEWIGCIHISSVTNTWYASWATGRFTISKDTSSTTVYLQMISLKTEDTAVYFCTRVGNSDYRYFNLWGPGTLVTVSS

SEQ ID NO: 96 - protein, 111 aa, synthetic construct
  REGION 1..111: note = Synthetic
EQVLTQSPGTLSLSPGERATLSCQSSKSVNNNNLAWYQQKPGQPPRLLIYFASALASGVPDRFSGSGSGTDFTLTISRLEPEDFAVYYCQGAYSGGSDSYAFGGGTKVEIK

SEQ ID NO: 97 - protein, 115 aa, synthetic construct
  REGION 1..115: note = Synthetic
EQQLVESGGGVVQPGRSLRLSCAASGFSFSYGYDMCWVRQAPGKGLEWIACISAGSSDNTWYASWAKGRFTISKDTSKTTVYLQMNSLRAEDTAVYFCSRYLGLWGPGTLVTVSS

SEQ ID NO: 98 - protein, 111 aa, synthetic construct
  REGION 1..111: note = Synthetic
EPVMTQSPATLSVSPGERATLSCQASQSISNALAWYQQKPGQPPRLLIYEVSKVASGVPARFSGSGSGTEFTLTISSLQSEDFAVYYCQSAYYGNSYVWGAFGQGTKVEIK

SEQ ID NO: 99 - protein, 119 aa, synthetic construct
  REGION 1..119: note = Synthetic
EQQLVESGGGLVQPGGSLRLSCAVSGFSLSSYAMSWVRQAPGKGLEWIGVISASGRIIYATWAKGRFTISKDSAKTSVYLQMNSLRDEDTAVYFCARGVPSYSLVMSDWGPGTTVTVSS

SEQ ID NO: 100 - protein, 217 aa, synthetic construct
  REGION 1..217: note = Synthetic
DIQMTQSPSSLSASVGDRVTITCQASQRIGNALAWYQQKPGKPPKLLIYRASTLESGVPSRFSGSGSGTDFTLTISSLQPEDVATYYCLGGYISIGNVYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 452 aa, synthetic construct
  REGION 1..452: note = Synthetic
EQQLVESAGGLVQPGGSLRLSCAASGFDFSSNYYMCWVRQAPGKGLEWIGCIHISSVTNTWYASWATGRFTISKDTSSTTVYLQMISLKTEDTAVYFCTRVGNSDYRYFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 218 aa, synthetic construct
  REGION 1..218: note = Synthetic
EQVLTQSPGTLSLSPGERATLSCQSSKSVNNNNLAWYQQKPGQPPRLLIYFASALASGVPDRFSGSGSGTDFTLTISRLEPEDFAVYYCQGAYSGGSDSYAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 445 aa, synthetic construct
  REGION 1..445: note = Synthetic
EQQLVESGGGVVQPGRSLRLSCAASGFSFSYGYDMCWVRQAPGKGLEWIACISAGSSDNTWYASWAKGRFTISKDTSKTTVYLQMNSLRAEDTAVYFCSRYLGLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 218 aa, synthetic construct
  REGION 1..218: note = Synthetic
EPVMTQSPATLSVSPGERATLSCQASQSISNALAWYQQKPGQPPRLLIYEVSKVASGVPARFSGSGSGTEFTLTISSLQSEDFAVYYCQSAYYGNSYVWGAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 449 aa, synthetic construct
  REGION 1..449: note = Synthetic
EQQLVESGGGLVQPGGSLRLSCAVSGFSLSSYAMSWVRQAPGKGLEWIGVISASGRIIYATWAKGRFTISKDSAKTSVYLQMNSLRDEDTAVYFCARGVPSYSLVMSDWGPGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 355 aa, Homo sapiens
  REGION 1..355: note = misc_feature - human CCR8
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 107 - protein, 355 aa, Macaca fascicularis
  REGION 1..355: note = misc_feature - cyno CCR8
MDYTLDPSMTTMTDYYYPDSLSSPCDGELIQRNDKLLLAVFYCLLFVFSLLGNSLVILVLVVCKKLRNITDIYLLNLALSDLLFVFSFPFQTYYQLDQWVFGTVMCKVVSGFYYIGFYSSMFFITLMSVDRYLAVVHAVYAIKVRTIRMGTTLSLVVWLTAIMATIPLLVFYQVASEDGVLQCYSFYNQQTLKWKIFTNFEMNILGLLIPFTIFMFCYIKILHQLKRCQNHNKTKAIRLVLIVVIASLLFWVPFNVVLFLTSLHSMHILDGCSISQQLNYATHVTEIISFTHCCVNPVIYAFVGEKFKKHLSEIFQKSCSHIFIYLGRQMPRESCEKSSSCQQHSFRSSSIDYIL

SEQ ID NO: 108 - protein, 353 aa, Mus musculus
  REGION 1..353: note = misc_feature - mouse CCR8
MDYTMEPNVTMTDYYPDFFTAPCDAEFLLRGSMLYLAILYCVLFVLGLLGNSLVILVLVGCKKLRSITDIYLLNLAASDLLFVLSIPFQTHNLLDQWVFGTAMCKVVSGLYYIGFFSSMFFITLMSVDRYLAIVHAVYAIKVRTASVGTALSLTVWLAAVTATIPLMVFYQVASEDGMLQCFQFYEEQSLRWKLFTHFEINALGLLLPFAILLFCYVRILQQLRGCLNHNRTRAIKLVLTVVIVSLLFWVPFNVALFLTSLHDLHILDGCATRQRLALAIHVTEVISFTHCCVNPVIYAFIGEKFKKHLMDVFQKSCSHIFLYLGRQMPVGALERQLSSNQRSSHSSTLDDIL

SEQ ID NO: 109 - protein, 13 aa, synthetic construct
  REGION 1..13: note = Synthetic
QCAYYGNSFVEGT

SEQ ID NO: 110 - protein, 24 aa, synthetic construct
  REGION 1..24: note = Synthetic
MDYTLDLSVTTVTDYYYPDIFSSP

SEQ ID NO: 111 - protein, 448 aa, synthetic construct
  REGION 1..448: note = Synthetic
EVQLLESGGGLVQPGGSLRLSCAASGIDLSTYAMGWVRQAPGKGLEWVGLIHRSGRTYYATWAKGRFTISKDSSKNTLYLQMNSLRAEDTAVYYCTRSYPDYSATASIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 112 - protein, 453 aa, synthetic construct
  REGION 1..453: note = Synthetic
EVQLLESGGGLVQPGGSLRLSCAASGFSLSNYAMIWVRQAPGKGLEWVSTISLGGYTYYANWAKGRFTISRDSSKTTVYLQMNSLRAEDTAVYFCARARWSTDSAIYTYAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 113 - protein, 448 aa, synthetic construct
  REGION 1..448: note = Synthetic
EVQLLESGGGLVQPGGSLRLSCAASGIDLSTYAMGWVRQAPGKGLEWVGLIHRSGRTYYATWAKGRFTISKDSSKNTLYLQMNSLRAEDTAVYYCTRSYPDYSATASIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 114 - protein, 453 aa, synthetic construct
  REGION 1..453: note = Synthetic
EVQLLESGGGLVQPGGSLRLSCAASGFSLSNYAMIWVRQAPGKGLEWVSTISLGGYTYYANWAKGRFTISRDSSKTTVYLQMNSLRAEDTAVYFCARARWSTDSAIYTYAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 115 - protein, 451 aa, synthetic construct
  REGION 1..451: note = Synthetic
EQQLVESAGGLVQPGGSLRLSCAASGFDFSSNYYMCWVRQAPGKGLEWIGCIHISSVTNTWYASWATGRFTISKDTSSTTVYLQMISLKTEDTAVYFCTRVGNSDYRYFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 116 - protein, 444 aa, synthetic construct
  REGION 1..444: note = Synthetic
EQQLVESGGGVVQPGRSLRLSCAASGFSFSYGYDMCWVRQAPGKGLEWIACISAGSSDNTWYASWAKGRFTISKDTSKTTVYLQMNSLRAEDTAVYFCSRYLGLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 117 - protein, 448 aa, synthetic construct
  REGION 1..448: note = Synthetic
EQQLVESGGGLVQPGGSLRLSCAVSGFSLSSYAMSWVRQAPGKGLEWIGVISASGRIIYATWAKGRFTISKDSAKTSVYLQMNSLRDEDTAVYFCARGVPSYSLVMSDWGPGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 118 - protein, 8 aa, synthetic construct
  REGION 1..8: note = Synthetic
DKYDDDDK

(19) United States
(12) Patent Application Publication
(10) Pub. No.: US 2025/0297019 A1
(43) Pub. Date: Sep. 25, 2025

(54) METHODS OF TREATING CANCER WITH ANTI-C-C MOTIF CHEMOKINE RECEPTOR 8 (CCR8) ANTIBODIES

(71) Applicant: Genentech, Inc., South San Francisco, CA (US)

(72) Inventors: Emel ESEN SERGIN, South San Francisco, CA (US); Gautham GAMPA, South San Francisco, CA (US); Iraj HOSSEINI, South San Francisco, CA (US); Robert Wenchen HSIEH, Millbrae, CA (US); Mahrukh HUSENI, South San Francisco, CA (US); Phillip SPINOSA, South San Francisco, CA (US); Maria Suhady ANDERSON, Camas, WA (US); Cassie Kai-Chi CHOU, Redwood City, CA (US)

(21) Appl. No.: 19/170,792

(22) Filed: Apr. 4, 2025

Related U.S. Application Data
(63) Continuation of application No. PCT/US2023/076241, filed on Oct. 6, 2023.
(60) Provisional application No. 63/378,826, filed on Oct. 7, 2022.
(60) Provisional application No. 63/487,487, filed on Feb. 28, 2023.

(51) Int. Cl. C07K 16/28; A61P 35/00

(52) U.S. Cl. CPC C07K 16/2866; A61P 35/00

(57) ABSTRACT

The present disclosure provides, inter alia, methods of treating a locally advanced, recurrent, or metastatic solid tumor malignancy in a subject in need thereof by administering an anti-CCR8 antibody to the subject, and related therapeutic uses.

### SEQUENCE LISTING

The instant application contains a Sequence Listing which has been submitted electronically in XML format and is hereby incorporated by reference in its entirety. Said XML copy, created on Apr. 1, 2025, is named 50474-310004_Sequence_Listing_4_1_25 and is 161,457 bytes in size.

### FIELD OF THE INVENTION

This invention relates to methods and compositions for use in treating cancer (e.g., a locally advanced, recurrent, or metastatic solid tumor malignancy) in a patient.

### BACKGROUND OF THE INVENTION

Regulatory T (Treg) cells expressing the transcription factor Foxp3 are important for maintaining peripheral immune tolerance and preventing autoimmunity. Treg cells also constitute a major component of the immune infiltrate of solid cancers, promoting tumor development and progression by establishing an immunosuppressive tumor microenvironment and dampening anti-tumor immune responses. Treg cells also hamper the efficacy of immunotherapies. An increased proportion of Treg cells among tumor-infiltrating lymphocytes is associated with poorer outcomes in several cancer indications.

Checkpoint blockade has led to improved overall survival for some patients; however, there remains a subset of patients whose cancer is either refractory to treatment (primary resistance) or progresses following treatment (secondary resistance).

Therefore, improved methods for treating cancer are needed.

### SUMMARY

The present disclosure provides, inter alia, methods of treating a locally advanced, recurrent, or metastatic solid tumor malignancy in a subject in need thereof, anti-CCR8 antibodies (e.g., monoclonal antibodies that bind to CCR8) for use in treating a locally advanced, recurrent, or metastatic solid tumor malignancy in a subject in need thereof, and related uses and kits.

In one aspect, the invention features a method of treating a locally advanced, recurrent, or metastatic solid tumor malignancy in a subject in need thereof, the method comprising administering to the subject a monoclonal antibody that binds to C-C motif chemokine receptor 8 (CCR8).

In another aspect, the invention features a monoclonal antibody that binds to CCR8 for use in treating a locally advanced, recurrent, or metastatic solid tumor malignancy in a subject in need thereof.

In some aspects, (i) the subject has progressed after at least one available standard therapy; and/or (ii) the subject is one for whom all available standard therapy has been proven to be ineffective or intolerable or is contraindicated.

In some aspects, the locally advanced, recurrent, or metastatic solid tumor is incurable.

In some aspects, the subject's age is 18 years or older.

In some aspects, the locally advanced, recurrent, or metastatic solid tumor malignancy is non-small cell lung cancer (NSCLC), head and neck squamous cell carcinoma (HNSCC), melanoma, triple-negative breast cancer (TNBC), urothelial carcinoma (UC), esophageal cancer, gastric cancer, cervical cancer, renal cell carcinoma (RCC), or hepatocellular carcinoma (HCC).

In some aspects, the RCC is clear cell RCC.

In some aspects, the HNSCC is HNSCC of the oral cavity, oropharynx, hypopharynx, or larynx.

In some aspects, the locally advanced, recurrent, or metastatic solid tumor malignancy is NSCLC.

In some aspects, the subject's tumor comprises a targetable somatic alteration, and the subject has experienced disease progression during or after treatment, or intolerance to treatment, with a targeted agent.

In some aspects, the targetable somatic alteration comprises a somatic alteration involving epidermal growth factor receptor (EGFR), anaplastic lymphoma kinase (ALK), ROS proto-oncogene 1 (ROS1), proto-oncogene B-Raf (BRAF) V600E, neurotrophic tyrosine receptor kinase (NTRK), MET proto-oncogene (MET), RET proto-oncogene (RET), or Kirsten rat sarcoma virus (KRAS).

In some aspects, the melanoma is cutaneous melanoma.

In some aspects, the subject's tumor comprises a BRAFV600 mutation, and the subject has experienced disease progression during or after treatment, or intolerance to treatment, with one or more serine/threonine-protein kinase B-Raf (BRAF) inhibitors and/or one or more mitogen-activated protein kinase kinase (MEK) inhibitors.

In some aspects, the locally advanced, recurrent, or metastatic solid tumor malignancy is UC.

In some aspects, the subject has: (i) histologically confirmed incurable advanced transitional cell carcinoma of the urothelium (including renal pelvis, ureters, urinary bladder, and urethra); and/or (ii) a mixed histology, wherein the subject's tumor has a dominant transitional cell pattern.

In some aspects, the locally advanced, recurrent, or metastatic solid tumor malignancy is TNBC.

In some aspects, TNBC is defined by the American Society of Clinical Oncology-College of American Pathologists guidelines: (i) <1% of tumor-cell nuclei immunoreactive for estrogen receptor and <1% of tumor-cell nuclei immunoreactive for progesterone receptor; and/or (ii) HER2-negative based on immunohistochemistry (IHC) and/or in situ hybridization.

In some aspects, the subject is checkpoint inhibitor (CPI)-naïve.

In some aspects, the subject's locally advanced, recurrent, or metastatic solid tumor malignancy is NSCLC or UC.

In some aspects, the subject's locally advanced, recurrent, or metastatic solid tumor malignancy is UC, wherein the subject is eligible for treatment with cisplatin, and the subject has experienced disease progression during or after treatment, or intolerance to treatment, with cisplatin.

In some aspects, the subject has had no prior treatment with a CPI, or wherein the subject has had adjuvant treatment with a CPI that was discontinued at least six months prior to first administration of the monoclonal antibody that binds to CCR8 to the subject.

In some aspects, the subject is CPI-experienced.

In some aspects, the subject's locally advanced, recurrent, or metastatic solid tumor malignancy is NSCLC, HNSCC, melanoma, UC, TNBC, esophageal cancer, gastric cancer, cervical cancer, clear cell RCC, or HCC.

In some aspects, the subject derived clinical benefit from treatment comprising a PD-1 axis binding antagonist prior to disease progression.

In some aspects, the PD-1 axis binding antagonist is an anti-PD-L1 antibody or an anti-PD-1 antibody.

In some aspects, the subject had a treatment duration with the treatment comprising the PD-1 axis binding antagonist of greater than or equal to 6 months and/or had a partial response or complete response as best objective response.

In some aspects, (i) the subject has not received treatment with a CPI, an immunomodulatory monoclonal antibody, or an immunomodulatory monoclonal antibody-derived therapy within 6 weeks prior to first administration of the monoclonal antibody that binds to CCR8 to the subject; or (ii) the subject was previously treated with a PD-1 axis binding antagonist, and the last administration of the PD-1 axis binding antagonist to the subject was at least 3 weeks prior to first administration of the monoclonal antibody that binds to CCR8 to the subject.

In some aspects, the CPI is a PD-1 axis binding antagonist or a CTLA4 antagonist.

In some aspects, the PD-1 axis binding antagonist is an anti-PD-L1 antibody or an anti-PD-1 antibody.

In some aspects, the monoclonal antibody that binds to CCR8 is administered to the subject in a dosing regimen comprising one or more dosing cycles.

In some aspects, the one or more dosing cycles comprise 21-day dosing cycles.

In some aspects, the monoclonal antibody that binds to CCR8 is administered to the subject on Day 1 of each 21-day dosing cycle.

In some aspects, the monoclonal antibody that binds to CCR8 is administered to the subject until disease progression or unacceptable toxicity.

In some aspects, the monoclonal antibody that binds to CCR8 is administered to the subject at a dose of 2 mg.

In some aspects, the monoclonal antibody that binds to CCR8 is administered to the subject intravenously.

In some aspects, the monoclonal antibody that binds to CCR8 is administered to the subject intravenously by infusion.

In some aspects, the monoclonal antibody that binds to CCR8 is administered to the subject as a monotherapy.

In some aspects, the monoclonal antibody that binds to CCR8 is administered to the subject in combination with one or more additional therapeutic agents.

In some aspects, the one or more additional therapeutic agents comprises atezolizumab.

In some aspects, the atezolizumab is administered to the subject in a dosing regimen comprising one or more dosing cycles.

In some aspects, the one or more dosing cycles comprise 21-day dosing cycles.

In some aspects, the atezolizumab is administered to the subject on Day 1 of each 21-day dosing cycle.

In some aspects, the atezolizumab is administered to the subject at a dose of 1200 mg.

In some aspects, the atezolizumab is administered to the subject intravenously.

In some aspects, the atezolizumab is administered to the subject intravenously by infusion.

In some aspects, a tumor sample from the subject has been determined to have a detectable level of PD-L1 expression.

In some aspects, the tumor sample from the subject has a Tumor Cell (TC), an Immune Cell (IC), a Combined Positive Score (CPS), or a Tumor Proportion Score (TPS) greater than or equal to 1%.

In some aspects, the subject has received at least two cycles of the monoclonal antibody that binds to CCR8 prior to administration of atezolizumab to the subject.

In some aspects, the monoclonal antibody that binds to CCR8 comprises a heavy chain variable domain (VH) comprising (a) CDR-H1 comprising the amino acid sequence of SEQ ID NO: 29 or SEQ ID NO: 30, (b) CDR-H2 comprising the amino acid sequence of SEQ ID NO: 31, and (c) CDR-H3 comprising the amino acid sequence of SEQ ID NO: 32, and a light chain variable domain (VL) comprising (d) CDR-L1 comprising the amino acid sequence of SEQ ID NO: 26, (e) CDR-L2 comprising the amino acid sequence of SEQ ID NO: 27, and (f) CDR-L3 comprising the amino acid sequence of SEQ ID NO: 28.

In some aspects, the monoclonal antibody that binds to CCR8 binds to CCR8 independent of sulfation of CCR8.

In some aspects, the monoclonal antibody that binds to CCR8 binds to an epitope comprised of one or more of amino acid residues 2-6 of SEQ ID NO: 106.

In some aspects, the monoclonal antibody that binds to CCR8 comprises a sequence selected from the group consisting of: (a) a VH sequence having at least about 95%, at least about 96%, at least about 97%, at least about 98%, or at least about 99% identity to an amino acid sequence selected from the group consisting of SEQ ID NOs: 35-47; (b) a VL sequence having at least about 95%, at least about 96%, at least about 97%, at least about 98%, or at least about 99% identity to an amino acid sequence selected from the group consisting of SEQ ID NOs: 48-52; and (c) a VH sequence as defined in (a) and a VL sequence as defined in (b).

In some aspects, the monoclonal antibody that binds to CCR8 comprises a VH sequence selected from the group consisting of SEQ ID NOs: 35-47 and a VL sequence selected from the group consisting of SEQ ID NOs: 48-52.

In some aspects, the monoclonal antibody that binds to CCR8 comprises a sequence selected from the group consisting of: (a) a VH sequence having at least about 95%, at least about 96%, at least about 97%, at least about 98%, or at least about 99% identity to an amino acid sequence of SEQ ID NO: 47; (b) a VL sequence having at least about 95%, at least about 96%, at least about 97%, at least about 98%, or at least about 99% identity to an amino acid sequence of SEQ ID NO: 48; and (c) a VH sequence as defined in (a) and a VL sequence as defined in (b).

In some aspects, the monoclonal antibody that binds to CCR8 comprises a VH sequence having at least about 95%, at least about 96%, at least about 97%, at least about 98%, or at least about 99% identity to the amino acid sequence of SEQ ID NO: 47 and a VL sequence having at least about 95%, at least about 96%, at least about 97%, at least about 98%, or at least about 99% identity to the amino acid sequence of SEQ ID NO: 48.

In some aspects, the VL comprises a V4M mutation, a P43A mutation, a F46L mutation, a C90Q mutation, or a combination thereof. In some embodiments, the V4M mutation, the P43A mutation, the F46L mutation, or the C90Q mutation is according to Kabat numbering.

In some aspects, the VH comprises a G49S mutation, a K71R mutation, a S73N mutation, or a combination thereof. In some embodiments, the G49S mutation, the K71R mutation, or the S73N mutation is according to Kabat numbering.

In some aspects, the monoclonal antibody that binds to CCR8 comprises the heavy chain amino acid sequence of SEQ ID NO: 55 and the light chain amino acid sequence of SEQ ID NO: 56.

In some aspects, the monoclonal antibody that binds to CCR8 comprises the heavy chain amino acid sequence of SEQ ID NO: 60 and the light chain amino acid sequence of SEQ ID NO: 56.

In some aspects, the monoclonal antibody that binds to CCR8 comprises the heavy chain amino acid sequence of SEQ ID NO: 111 and the light chain amino acid sequence of SEQ ID NO: 56.

In some aspects, the monoclonal antibody that binds to CCR8 comprises the heavy chain amino acid sequence of SEQ ID NO: 113 and the light chain amino acid sequence of SEQ ID NO: 56.

In some aspects, the monoclonal antibody that binds to CCR8 comprises a VH sequence selected from the group consisting of SEQ ID NOs: 35-47 and a VL sequence selected from the group consisting of SEQ ID NOs: 48-52.

In some aspects, the monoclonal antibody that binds to CCR8 comprises the VH sequence of SEQ ID NO: 47 and the VL sequence of SEQ ID NO: 48.

In some aspects, the monoclonal antibody that binds to CCR8 comprises a heavy chain variable domain (VH) comprising (a) CDR-H1 comprising the amino acid sequence of SEQ ID NO: 4 or SEQ ID NO: 5, (b) CDR-H2 comprising the amino acid sequence of SEQ ID NO: 6, and (c) CDR-H3 comprising the amino acid sequence of SEQ ID NO: 7, and a light chain variable domain (VL) comprising (d) CDR-L1 comprising the amino acid sequence of SEQ ID NO: 1, (e) CDR-L2 comprising the amino acid sequence of SEQ ID NO: 2, and (f) CDR-L3 comprising the amino acid sequence of SEQ ID NO: 3.

In some aspects, the monoclonal antibody that binds to CCR8 binds to CCR8 independent of sulfation of CCR8.

In some aspects, the monoclonal antibody that binds to CCR8 binds to an epitope comprised of one or more of amino acid residues 91-104 and 172-193 of SEQ ID NO: 106.

In some aspects, the monoclonal antibody that binds to CCR8 comprises a sequence selected from the group consisting of: (a) a VH sequence having at least about 95%, at least about 96%, at least about 97%, at least about 98%, or at least about 99% identity to an amino acid sequence selected from the group consisting of SEQ ID NOs: 10-21; (b) a VL sequence having at least about 95%, at least about 96%, at least about 97%, at least about 98%, or at least about 99% identity to an amino acid sequence selected from the group consisting of SEQ ID NOs: 22-25; and (c) a VH sequence as defined in (a) and a VL sequence as defined in (b).

In some aspects, the monoclonal antibody that binds to CCR8 comprises a VH sequence selected from the group consisting of SEQ ID NOs: 10-21 and a VL sequence selected from the group consisting of SEQ ID NOs: 22-25.

In some aspects, the monoclonal antibody that binds to CCR8 comprises a sequence selected from the group consisting of: (a) a VH sequence having at least about 95%, at least about 96%, at least about 97%, at least about 98%, or at least about 99% identity to an amino acid sequence of SEQ ID NO: 21; (b) a VL sequence having at least about 95%, at least about 96%, at least about 97%, at least about 98%, or at least about 99% identity to an amino acid sequence of SEQ ID NO: 24; and (c) a VH sequence as defined in (a) and a VL sequence as defined in (b).

In some aspects, the monoclonal antibody that binds to CCR8 comprises a VH sequence having at least about 95%, at least about 96%, at least about 97%, at least about 98%, or at least about 99% identity to an amino acid sequence of SEQ ID NO: 21 and a VL sequence having at least about 95%, at least about 96%, at least about 97%, at least about 98%, or at least about 99% identity to an amino acid sequence of SEQ ID NO: 24.

In some aspects, the VL comprises a Y21 mutation. In some embodiments, the Y21 mutation is according to Kabat numbering.

In some aspects, the VH comprises a S73N mutation, a V78L mutation, a T76N mutation, a F91Y mutation, and a P105Q mutation, or a combination thereof. In some embodiments, the S73N mutation, the V78L mutation, the T76N mutation, the F91Y mutation, or the P105Q mutation is according to Kabat numbering.

In some aspects, the monoclonal antibody that binds to CCR8 comprises the heavy chain amino acid sequence of SEQ ID NO: 57 and the light chain amino acid sequence of SEQ ID NO: 58.

In some aspects, the monoclonal antibody that binds to CCR8 comprises the heavy chain amino acid sequence of SEQ ID NO: 61 and the light chain amino acid sequence of SEQ ID NO: 58.

In some aspects, the monoclonal antibody that binds to CCR8 comprises the heavy chain amino acid sequence of SEQ ID NO: 112 and the light chain amino acid sequence of SEQ ID NO: 58.

In some aspects, the monoclonal antibody that binds to CCR8 comprises the heavy chain amino acid sequence of SEQ ID NO: 114 and the light chain amino acid sequence of SEQ ID NO: 58.

In some aspects, the monoclonal antibody that binds to CCR8 comprises the VH sequence of SEQ ID NO: 21 and the VL sequence of SEQ ID NO: 24.

In some aspects, the monoclonal antibody that binds to CCR8 comprises a heavy chain variable domain (VH) comprising (a) CDR-H1 comprising the amino acid sequence of SEQ ID NO: 82 or SEQ ID NO: 83, (b) CDR-H2 comprising the amino acid sequence of SEQ ID NO: 84, and (c) CDR-H3 comprising the amino acid sequence of SEQ ID NO: 85, and a light chain variable domain (VL) comprising (d) CDR-L1 comprising the amino acid sequence of SEQ ID NO: 73, (e) CDR-L2 comprising the amino acid sequence of SEQ ID NO: 74, and (f) CDR-L3 comprising the amino acid sequence of SEQ ID NO: 75.

In some aspects, the monoclonal antibody that binds to CCR8 comprises a sequence selected from the group consisting of: (a) a VH sequence having at least about 95%, at least about 96%, at least about 97%, at least about 98%, or at least about 99% identity to an amino acid sequence of SEQ ID NO: 95; (b) a VL sequence having at least about 95%, at least about 96%, at least about 97%, at least about 98%, or at least about 99% identity to an amino acid sequence of SEQ ID NO: 94; and (c) a VH sequence as defined in (a) and a VL sequence as defined in (b).

In some aspects, the monoclonal antibody that binds to CCR8 comprises the VH sequence of SEQ ID NO: 95 and the VL sequence of SEQ ID NO: 94.

In some aspects, the monoclonal antibody that binds to CCR8 comprises the heavy chain amino acid sequence of SEQ ID NO: 101 and the light chain amino acid sequence of SEQ ID NO: 100.

In some aspects, the monoclonal antibody that binds to CCR8 comprises the heavy chain amino acid sequence of SEQ ID NO: 115 and the light chain amino acid sequence of SEQ ID NO: 100.

In some aspects, the monoclonal antibody that binds to CCR8 comprises a heavy chain variable domain (VH) comprising (a) CDR-H1 comprising the amino acid sequence of SEQ ID NO: 86 or SEQ ID NO: 87, (b) CDR-H2 comprising the amino acid sequence of SEQ ID NO: 88, and (c) CDR-H3 comprising the amino acid sequence of SEQ ID NO: 89, and a light chain variable domain (VL) comprising (d) CDR-L1 comprising the amino acid sequence of SEQ ID NO: 76, (e) CDR-L2 comprising the amino acid sequence of SEQ ID NO: 77, and (f) CDR-L3 comprising the amino acid sequence of SEQ ID NO: 78.

In some aspects, the monoclonal antibody that binds to CCR8 comprises a sequence selected from the group consisting of: (a) a VH sequence having at least about 95%, at least about 96%, at least about 97%, at least about 98%, or at least about 99% identity to an amino acid sequence of SEQ ID NO: 97; (b) a VL sequence having at least about 95%, at least about 96%, at least about 97%, at least about 98%, or at least about 99% identity to an amino acid sequence of SEQ ID NO: 96; and (c) a VH sequence as defined in (a) and a VL sequence as defined in (b).

In some aspects, the monoclonal antibody that binds to CCR8 comprises the VH sequence of SEQ ID NO: 97 and the VL sequence of SEQ ID NO: 96.

In some aspects, the monoclonal antibody that binds to CCR8 comprises the heavy chain amino acid sequence of SEQ ID NO: 103 and the light chain amino acid sequence of SEQ ID NO: 102.

In some aspects, the monoclonal antibody that binds to CCR8 comprises the heavy chain amino acid sequence of SEQ ID NO: 116 and the light chain amino acid sequence of SEQ ID NO: 102.

In some aspects, the monoclonal antibody that binds to CCR8 comprises a heavy chain variable domain (VH) comprising (a) CDR-H1 comprising the amino acid sequence of SEQ ID NO: 90 or SEQ ID NO: 91, (b) CDR-H2 comprising the amino acid sequence of SEQ ID NO: 92, and (c) CDR-H3 comprising the amino acid sequence of SEQ ID NO: 93, and a light chain variable domain (VL) comprising (d) CDR-L1 comprising the amino acid sequence of SEQ ID NO: 79, (e) CDR-L2 comprising the amino acid sequence of SEQ ID NO: 80, and (f) CDR-L3 comprising the amino acid sequence of SEQ ID NO: 81.

In some aspects, the monoclonal antibody that binds to CCR8 comprises a sequence selected from the group consisting of: (a) a VH sequence having at least about 95%, at least about 96%, at least about 97%, at least about 98%, or at least about 99% identity to an amino acid sequence of SEQ ID NO: 99; (b) a VL sequence having at least about 95%, at least about 96%, at least about 97%, at least about 98%, or at least about 99% identity to an amino acid sequence of SEQ ID NO: 98; and (c) a VH sequence as defined in (a) and a VL sequence as defined in (b).

In some aspects, the monoclonal antibody that binds to CCR8 comprises the VH sequence of SEQ ID NO: 99 and the VL sequence of SEQ ID NO: 98.

In some aspects, the monoclonal antibody that binds to CCR8 comprises the heavy chain amino acid sequence of SEQ ID NO: 105 and the light chain amino acid sequence of SEQ ID NO: 104.

In some aspects, the monoclonal antibody that binds to CCR8 comprises the heavy chain amino acid sequence of SEQ ID NO: 117 and the light chain amino acid sequence of SEQ ID NO: 104.

In some aspects, the monoclonal antibody that binds to CCR8 binds to CCR8 independent of sulfation of CCR8.

In some aspects, the antibody binds to an epitope comprised of one or more of amino acid residues 2-6 of SEQ ID NO: 106.

In some aspects, the antibody binds to binds to an epitope comprised of one or more of amino acid residues 91-104 and 172-193 of SEQ ID NO: 106.

In some aspects, the monoclonal antibody that binds to CCR8 comprises a heavy chain variable domain (VH) comprising (a) CDR-H1 comprising the amino acid sequence of SEQ ID NO: 65 or SEQ ID NO: 66, (b) CDR-H2 comprising the amino acid sequence of SEQ ID NO: 67 and (c) CDR-H3 comprising the amino acid sequence of SEQ ID NO: 68, and a light chain variable domain (VL) comprising (d) CDR-L1 comprising the amino acid sequence of SEQ ID NO: 62, (e) CDR-L2 comprising the amino acid sequence of SEQ ID NO: 63, and (f) CDR-L3 comprising the amino acid sequence of SEQ ID NO: 64.

In some aspects, the monoclonal antibody that binds to CCR8 comprises a sequence selected from the group consisting of: (a) a VH sequence having at least about 95%, at least about 96%, at least about 97%, at least about 98%, or at least about 99% identity to an amino acid sequence of SEQ ID NO: 70; (b) a VL sequence having at least about 95%, at least about 96%, at least about 97%, at least about 98%, or at least about 99% identity to an amino acid sequence of SEQ ID NO: 69; and (c) a VH sequence as defined in (a) and a VL sequence as defined in (b).

In some aspects, the monoclonal antibody that binds to CCR8 comprises the VH sequence of SEQ ID NO: 70 and the VL sequence of SEQ ID NO: 69.

In some aspects, the monoclonal antibody that binds to CCR8 comprises the heavy chain amino acid sequence of SEQ ID NO: 72 and the light chain amino acid sequence of SEQ ID NO: 71.

In some aspects, the monoclonal antibody that binds to CCR8 is a human antibody.

In some aspects, the monoclonal antibody that binds to CCR8 is a humanized antibody.

In some aspects, the monoclonal antibody that binds to CCR8 is a chimeric antibody.

In some aspects, the monoclonal antibody that binds to CCR8 is an antibody fragment that binds to CCR8.

In some aspects, the monoclonal antibody that binds to CCR8 is a full-length antibody.

In some aspects, the monoclonal antibody that binds to CCR8 is a full-length IgG1 antibody.

In some aspects, the monoclonal antibody that binds to CCR8 comprises a IgG1 constant domain comprising the amino acid sequence of SEQ ID NO: 53 or SEQ ID NO: 59.

In some aspects, the monoclonal antibody that binds to CCR8 comprises a kappa constant domain comprising the amino acid sequence of SEQ ID NO: 54.

In some aspects, the monoclonal antibody that binds to CCR8 binds to CCR8 with a binding affinity (K_{d}) of from about 1×10⁻¹² M to about 1×10⁻¹¹ M.

In some aspects, the CCR8 is a human CCR8.

In some aspects, the monoclonal antibody that binds to CCR8 is afucosylated.

In some aspects, the proportion of afucosylation is between about 80% to about 95%.

In some aspects, the regulatory T cells present in the tumor microenvironment of the locally advanced, recurrent, or metastatic solid tumor malignancy are depleted.

In some aspects, the regulatory T cells outside of the tumor microenvironment of the locally advanced, recurrent, or metastatic solid tumor malignancy are depleted.

In some aspects, the subject is a human.

### BRIEF DESCRIPTION OF THE DRAWINGS

FIG. 1 depicts the results of a screening of anti-CCR8 monoclonal antibodies (mAbs) selectively binding to Treg cells (Tregs) from human colorectal cancer dissociated tumor cells (DTC) (obtained from Discovery Life Sciences). Shown are mean fluorescent intensity (MFI) values for CD8 T cells (defined as CD45+ CD14− CD3+ CD8+ CD4−) (circles, ∘), conventional CD4 T cells (defined as CD45+ CD14− CD3+ CD8− CD4+ FOXP3−) (squares, □), and Treg cells (defined as CD45+ CD14− CD3+ CD8− CD4+ FOXP3+) (triangles, ▴). Three of the five anti-CCR8 mAb clones Ab1-Ab5 specifically stained intratumoral Treg cells and not conventional CD4 or CD8 T cells, and were ranked based on CCR8 MFI: hu.Ab4.H1L1>hu.Ab5.H1L1>hu.Ab3.H1 L1.

FIGS. 2A and 2B depict the proposed mechanism of action of natural killer (NK) cell-mediated antibody-dependent cellular cytotoxicity (ADCC), resulting in depletion of tumor-infiltrating CCR8-expressing Tregs (FIG. 2A) and the ADCC activities of human/cyno cross-reactive anti-CCR8 mAbs brought forward for further study (FIG. 2B). EC₅₀ values were determined as 0.02 nM, 0.02 nM, and 0.08 nM for anti-CCR8 mAbs hu.Ab3.H1L1, hu.Ab5.H1L1, and hu.Ab4.H1L1, respectively.

FIGS. 3A-3D depict the agonist and antagonist activities of the human/cyno cross-reactive anti-CCR8 mAbs hu.Ab4.H1L1, hu.Ab5.H1L1, and hu.Ab3.H1L1, as well as comparator anti-CCR8 mAbs (the humanized anti-human Yoshida anti-CCR8 antibody, murine anti-human CCR8 mAb 433H (BD Biosciences), and murine anti-human CCR8 mAb L263G8 (Biolegend)). As shown in FIG. 3A, CCL1, a known ligand for CCR8, shows agonist activity, but none of the anti-CCR8 test mAbs show agonistic effects. The data in FIG. 3B shows anti-CCR8 mAb hu.Ab4.H1L1 demonstrates antagonistic (neutralizing/ligand blocking) activity against the CCR8 ligand CCL1 (20 nM of ligand), whereas anti-CCR8 mAbs hu.Ab5.H1L1 and hu.Ab3.H1L1 demonstrate no ligand blocking (non-neutralizing) activity at the concentration studied. The data in FIG. 3C shows that comparator anti-CCR8 mAbs (the humanized anti-human Yoshida anti-CCR8 antibody, murine anti-human CCR8 mAb 433H (BD Biosciences), and murine anti-human CCR8 mAb L263G8 (Biolegend)) do not show agonistic effects, whereas the CCR8 ligand CCL1 does. The data in FIG. 3D shows comparator anti-CCR8 mAbs (the humanized anti-human Yoshida anti-CCR8 antibody, murine anti-human CCR8 mAb 433H (BD Biosciences), and murine anti-human Biolegend L263G8 (Biolegend)) demonstrate antagonistic (neutralizing/ligand blocking) activity against the CCR8 ligand CCL1. The IC₅₀ values for the ligand blocking activity are provided in the Examples.

FIGS. 4A-4F depict the binding data of hu.Ab3.H1L1 (FIG. 4A), hu.Ab4.H1L1 (FIG. 4B), and hu.Ab5.H1L1 (FIG. 4C), as well as the commercial anti-CCR8 mAbs murine anti-human CCR8 mAb 433H (BD Biosciences) (FIG. 4D), and murine anti-human CCR8 mAb L263G8 (Biolegend) (FIG. 4E), and the humanized anti-human Yoshida anti-CCR8 mAb (FIG. 4F), to HEK293 cells that were transiently transfected with N-term FLAG® (DYKDDDDK, SEQ ID NO: 118)-tagged plasmids encoding for human GPCRs (CCR2, CCR3, CCR4, CCR5, CCR8, CXCR4, ACKR2, and ACKR4), hCCR8 constructs, or with a mock construct using TRANSIT-X2® (reagent:DNA=3:1). Cell surface expression of each GPCR was confirmed by staining with an anti-FLAG® antibody control (5 ug/mL). mAbs hu.Ab4.H1L1, and hu.Ab5.H1L1 only stained the hCCR8-containing cells, confirming their specificity to hCCR8. mAb hu.Ab3.H1L1 showed staining of multiple other GPCRs, indicating lack of specificity. The CCR8 selective hu.Ab4.H1L1 and hu.Ab5.H1L1 mAbs, which demonstrated the best ADCC activities (as noted in FIGS. 2A and 2B), were carried forward for further study.

FIGS. 5A-5D depict the light chain variable region (FIG. 5A) and heavy chain variable region (FIGS. 5B-5D) alignment of the sequences for rabbit (rb.Ab4) and humanized Ab4 (L1-L4 and H1-H12) CCR8 mAbs studied. Y2 on light chain (L3) and S73, T76, V78, F91 and P105 on heavy chain (H12) were determined to be the key rabbit Vernier residues based on binding evaluation of the variant antibodies. The CDRs, variable regions, constant regions, and Full-length sequences are provided in the Examples.

FIGS. 6A-6D depict the light chain variable region (FIG. 6A) and heavy chain variable region (FIGS. 6B-6D) alignment of the sequences for rabbit (rb.Ab5) and humanized Ab5 (L1-L5 and H1-H13) CCR8 mAbs studied. A C90Q mutation in CDR L3 was introduced to remove an unpaired cysteine that would be a liability during manufacturing. V4, P43 and F46 on light chain (L1), and G49, K71 and S73 (H13) on the heavy chain were determined to be the key rabbit Vernier residues based on binding evaluation of the variant antibodies. The CDRs, variable regions, constant regions, and Full-length sequences are provided in the Examples.

FIGS. 7A-7D depict the results of cell-based affinity measurements for hu.Ab5.H13L1 and hu.Ab4.H12L3 mAbs using radiolabeled IgGs and CHO cell lines stably expressing human CCR8 or cynomolgus monkey (“cyno”) CCR8. The data shows hu.Ab4.H12L3 and hu.Ab5.H13L1 mAbs have similar affinity for both human and cyno CCR8, indicating desirable cross-reactivity (Compare FIG. 7A to FIG. 7B, and compare FIG. 7C to FIG. 7D). Kd (nM) affinity data from these studies is provided in the Examples.

FIGS. 8A and 8B depict the binding data of hu.Ab4.H12L3 (FIG. 8A) and hu.Ab5.H13L1 (FIG. 8B) mAbs to a panel of sulfated GPCRs, and reconfirm that these Ab4 and Ab5 variants, similar to the Ab4 and Ab5 data provided in FIG. 4B and FIG. 4C, show selectivity for CCR8. Due to the weaker binding to the N-terminal FLAG® tag of hCCR8 (which impacts binding to the N-terminal epitope for Ab5; see FIGS. 16A and 16B) the CCR8 construct with the C-terminal FLAG® is also provided in FIG. 4C.

FIGS. 9A and 9B depict the effects of anti-CCR8 mAbs hu.Ab4.H12L3 and hu.Ab5.H13L1 on CCR8 activation as determined by Ca²⁺ influx assay (FIG. 9A) and CCR8 CCL1 ligand binding (FIG. 9B). Similar to FIG. 3A data, FIG. 9A reconfirms neither the Ab4 nor the Ab5 anti-CCR8 mAbs variants show agonistic effects in the absence of CCR8 ligand CCL1. Similar to FIG. 3B data, FIG. 9B reconfirms the Ab4 variants demonstrates antagonistic effects against the CCR8 ligand CCL1 (20 nM of ligand), whereas the Ab5 variant demonstrates no ligand blocking activity at the concentration studied. The IC₅₀ values for the ligand blocking activity are provided in the Examples.

FIGS. 10A-10E depict differences in staining of hu.Ab4.H12L3 and hu.Ab5.H13L1 compared to the humanized anti-human Yoshida CCR8 mAb and commercial antibodies murine anti-human CCR8 mAb 433H (BD Biosciences) and murine anti-human CCR8 mAb L263G8 (Biolegend) to CCR8+ HEK293 cells with (hCCR8.TPST1/2 NTC) and without tyrosyl protein sulfotransferase (TPST) 1 and tyrosyl protein sulfotransferase (TPST) 2 (hCCR8.TPST1/2 KO). hu.Ab4.H12L3 (FIG. 10A) and hu.Ab5.H13L1 (FIG. 10B) show similar binding/staining to both cell lines (hCCR8.TPST1/2 NTC and hCCR8.TPST1/2 KO), indicating they bind CCR8 independent of tyrosine sulfation (“sulfation independent”). In contrast, the humanized anti-human Yoshida CCR8 antibody (FIG. 10C) and commercial antibodies murine anti-human CCR8 mAb 433H (BD Biosciences) (FIG. 10D) and murine anti-human CCR8 mAb L263G8 (Biolegend) (FIG. 10E) failed to bind the TPST1/2 KO cells, indicating they require tyrosine sulfation of CCR8 for binding, and are thus considered “sulfation dependent.”

FIGS. 11A-11D depict that afucosylated CCR8 mAbs Afuc.hu.Ab5.H13L1 and Afuc.hu.Ab4.H12L3 show enhanced (>10-fold improved) ADCC activity compared to their fucosylated CCR8 counterparts hu.Ab5.H13L1 and hu.Ab4.H12L3 against CHO cells stably expressing hCCR8 using NK-92 F158 (FIG. 11A) and NK-92 V158 (FIG. 11B) as effector cells, and also show a 10-20 fold improvement in ADCC activity compared the humanized anti-human Yoshida anti-CCR8 antibody (FIG. 11C). Commercial anti-CCR8 mAbs murine anti-human CCR8 mAb 433H (BD Biosciences) and murine anti-human CCR8 mAb L263G8 (Biolegend) demonstrated (as expected) no ADCC activity as the assay used is primarily relevant for antibodies comprising human Fc regions (FIG. 11C). FIG. 11D shows murine anti-human CCR8 mAb 433H (BD Biosciences) and murine anti-human CCR8 mAb L263G8 (Biolegend) have ADCC activity using an assay specific for antibodies comprising murine Fc regions and human anti-CCR8 activity. Activity data is also provided in the Examples.

FIGS. 12A-12D depict the selective ADCC activity against human Treg cells compared to conventional human CD4 T cells from peripheral blood mononuclear cells (PBMC) that had been recovered after transfer into NOD.Cg-Prkdc^{scid} Il2rg^{tm1Wjl}/SzJ (NSG™) mice to induce CCR8 expression when incubated with afucosylated, fucosylated (hIgG1), and the afucosylated isotype control mAb (“gD.afuc”) and primary NK cells as effector cells. ADCC activity against Treg cells was measured by calculating the ratio of recovered Treg cells to recovered CD8 cells (Treg/CD8) or conventional CD4 T cells to recovered CD8 T cells (CD4conv/CD8). CCR8 mAbs Afuc.hu.Ab4.H12L3 and hu.Ab4.H12L3 selectively mediated ADCC activity against Treg cells (FIG. 12A) in comparison to conventional CD4 T cells (FIG. 12B), with the afucosylated variant demonstrating increased ADCC activity. Similarly, CCR8 mAbs Afuc.hu.Ab5.H13L1 and hu.Ab5.H13L1 selectively mediated ADCC activity against Treg cells (FIG. 12C) in comparison to conventional CD4 T cells (FIG. 12D), with the afucosylated variant demonstrating increased ADCC activity.

FIGS. 13A-13D depict the selective ADCC activity against Treg cells compared to conventional CD4 T cells when human dissociated renal cell carcinoma (RCC) cells were incubated with afucosylated, fucosylated (hIgG1), and the afucosylated isotype control mAb (“gD.afuc”) and primary NK cells as effector cells. ADCC activity against Treg cells was measured by calculating the ratio of recovered Treg cells to recovered CD8 cells (Treg/CD8) or conventional CD4 T cells to recovered CD8 T cells (CD4conv/CD8). CCR8 mAbs Afuc.hu.Ab4.H12L3 and hu.Ab4.H12L3 selectively mediated ADCC activity against Treg cells (FIG. 13A) in comparison to conventional CD4 T cells (FIG. 13B), with the afucosylated variant demonstrating increased ADCC activity. Similarly, CCR8 mAbs Afuc.hu.Ab5.H13L1 and hu.Ab5.H13L1 selectively mediated ADCC activity against Treg cells (FIG. 13C) in comparison to conventional CD4 T cells (FIG. 13D), with the afucosylated variant demonstrating increased ADCC activity.

FIGS. 14A-14E show that the afucosylated anti-CCR8 mAbs Afuc.hu.Ab5.H13L1 and Afuc.hu.Ab4.H12L3 exhibit enhanced ADCP activities compared to fucosylated mAbs hu.Ab5.H13L1 and hu.Ab4.H12L3 in CD14⁺ monocytes-derived macrophages from four different donors with FcgRIIa (H131R)/FcgRIIIa (V158F) genotypes of HR/FF (FIG. 14A), RR/FF (FIG. 14B), HR/VF (FIG. 14C), and RR/VF (FIG. 14D), and also show a 3-4 fold improvement in ADCP activity compared the humanized anti-human Yoshida anti-CCR8 antibody (FIG. 14E). Activity data is also provided in the Examples.

FIGS. 15A-15D show that the afucosylated anti-CCR8 mAb Afuc.hu.Ab5.H13L1 exhibits similar improved ADCP activities compared to the FcgRIIa-enhanced G236A.I332E variant Afuc.hu.Ab5.H13L1.G236A.I332E in CD14⁺ monocytes-derived macrophages from four different donors with FcgRIIa (H131R)/FcgRIIIa (V158F) genotypes of genotypes of HR/FF (FIG. 15A), RR/FF (FIG. 15B), HR/VF (FIG. 15C), and RR/VF (FIG. 15D).

FIGS. 16A and 16B depict the epitope maps for hu.Ab5.H13L1 (FIG. 16A) and hu.Ab4.H12L3 (FIG. 16B) mAbs. As shown in FIG. 16A, in which constructs encoding for individual alanine point mutations at positions 2-24 in hCCR8 with a C-terminal FLAG® tag were generated, hu.Ab5.H13L1 does not bind D2A, Y3A, L5A, and D6A, indicating that the epitope includes at least the DYTLD region of the human CCR8 N-terminus. As shown in FIG. 16B, in which constructs encoding for human CCR8.CCR5 chimeras (N-term1, N-term2, ECL1, ECL2, and ECL3) in which different extracellular regions of hCCR8 were replaced with the corresponding region from CCR5 with a C-terminal FLAG® tag were generated, hu.Ab4.H12L3 does not bind the ECL1 and ECL2 chimeras indicating that the epitope for this antibody includes at least the ECL1 and ECL2 regions of CCR8. huCCR8 N-terminus:

(SEQ ID NO: 110)
MDYTLDLSVTTVTDYYYPDIFSSP.

FIGS. 17A-17I depict the progressive depletion of Treg cells (measured as fraction of Treg cells with CD45+ leukocytes) in tumors (FIG. 17A) but not in spleen (FIG. 17B) or tumor-draining lymph nodes (FIG. 17C) in CT26 tumor-bearing mice three days after injection of a single dose of a mouse surrogate anti-CCR8 mAb of increasing concentration between 0.003-5 mg/kg. Anti-CCR8 mAb treatment did not result in CD4 conventional T cell (FIGS. 17D-17F) or CD8 T cell depletion (FIGS. 17G-17I). An isotype control antibody (anti-gp120) was used.

FIG. 17J depicts the depletion of Treg cells (measured as fraction of Treg cells with CD45+ leukocytes) in tumors but not in the spleen, draining lymph node (dLN), or blood in E0771 syngeneic tumor-bearing mice three days after injection of anti-CCR8 antibody of increasing concentration between 0.01 and 1 mg/kg.

FIGS. 17K-17O depict a minimal physiologically based pharmacokinetic-pharmacodynamic (PBPK-PD) model that was used to simulate the pharmacokinetic (PK)/receptor occupancy (RO) relationship as well as pharmacodynamic (PD) and efficacy of mouse surrogate anti-CCR8 antibody. The minimal PBPK model structure is shown in FIG. 17K. This model predicts PK (FIG. 17L), RO (FIG. 17M), Treg depletion (FIG. 17N), and anti-tumor efficacy (FIG. 17O) of anti-CCR8 antibody.

FIGS. 18A-18D depict tumor growth inhibition following treatment with a single dose (FIG. 18B) or twice weekly dosing (FIG. 18C) of a mouse surrogate anti-CCR8 mAb in mice with established CT26 syngeneic tumors compared to treatment with an anti-CD25 mAb (FIG. 18D) or an isotype control mAb (anti-gp120) (FIG. 18A). Treatment started when tumor reached a volume between 150-250 mm³. Tumor volume is measured over time. Grey lines represent individual mice, black lines represent the group fit.

FIGS. 19A-19E depict growth inhibition of CT26 tumors observed with an effector-competent mouse surrogate anti-CCR8 mAb administered at the time of tumor inoculation (FIG. 19B) or with tumors reached 150-250 mm³ (FIG. 19D). No tumor growth inhibition is observed with an effector-incompetent LALAPG variant of the same ligand-blocking anti-CCR8 mAb (FIGS. 19C and 19E). Tumor volume is measured over time. Grey lines represent individual mice, black lines represent the group fit. An isotype control mAb (anti-gp120) was used (FIG. 19A).

FIGS. 20A-20D show that the combination of a mouse surrogate anti-CCR8 mAb and an anti-PDL1 mAb (FIG. 20D) is unexpectedly more efficacious in growth inhibition of EMT6 tumors than anti-CCR8 mAb alone (FIG. 20B) or anti-PDL1 mAb alone (FIG. 20C). Treatment started when tumors reached 150-250 mm³. Tumor volume is measured over time. Grey lines represent individual mice, black lines represent the group fit. An isotype control mAb (anti-gp120) was used (FIG. 20A).

FIG. 21 depicts the serum pharmacokinetic profiles (mean±SD) of anti-gD (control)_and test anti-CCR8 mAbs Afuc.hu.Ab5.H13L1 and Afuc.hu.Ab4.H12L3 in cynomolgus monkey following a single dose 10 mg/kg IV bolus injection. Afuc.hu.Ab5.H13L1 exhibited desired sustained serum concentration levels over the 35-day post-dose period, which is expected to elicit a more sustained target engagement that may translate to better anti-cancer activity and less frequent dosing.

FIGS. 22A-22C depict the results of whole blood flow cytometry analysis for total Treg cell count of 9 male cynos dosed with 10 mg/kg afucosylated anti-gD (Control; Group 1, designated 1001, 1002, 1003; FIG. 22A), Afuc.hu.Ab5.H13L1 (Group 2, designated 2001, 2002, 2003; FIG. 22B), or Afuc.hu.Ab4.H12L3 (Group 3, designated 3001, 3002, 3003; FIG. 22C) via intravenous injection. Both test anti-CCR8 mAbs did not substantially reduce the total T-reg cell absolute counts in whole blood for up to 840 hours post dose.

FIGS. 23A-231 depict the results of whole blood flow cytometry analysis for reduction of CCR8+FoxP3+ Treg cells of 9 male cynos dosed with afucosylated anti-gD (Control; Group 1, designated 1001 (FIG. 23A), 1002 (FIG. 23B), 1003 (FIG. 23C)) Afuc.hu.Ab4.H12L3 (Group 3, designated 3001 (FIG. 23D), 3002 (FIG. 23E), 3003 (FIG. 23F)), or, Afuc.hu.Ab5.H13L1 (Group 2, designated 2001 (FIG. 23G), 2002 (FIG. 23H), 2003 (FIG. 231)). Blood was collected from each of the animals before dosing (“Pre-study”), as well as on Day 1 at 0 hours (“Pre-dose”). Each of the animals were then administered a single dose of 10 mg/kg afucosylated anti-gD (Control Group), Afuc.hu.Ab5.H13L1 (Group 2) or Afuc.hu.Ab4.H12L3 (Group 3) via intravenous injection. Blood was then collected from the animals and subjected to the following treatment prior to flow cytometry analysis: (i) blood sample not spiked with either test CCR8 mAb (“unspiked”), (ii) blood sample further spiked with a saturating concentration of Afuc.hu.Ab5.H13L1, and (iii) blood sample further spiked with a saturating concentration of Afuc.hu.Ab4.H12L3. Each of the unspiked and spiked samples were subsequently treated with a labeled goat anti-human IgG antibody and analyzed by flow cytometry. As can be seen in FIGS. 23A-23C, flow cytometry of blood initially treated with control (Group 1) but unspiked demonstrated no modulation of total CCR8+T-reg cells. Furthermore, flow cytometry of spiked blood also had very little effect on the total CCR8+T-reg cell count. With regard to Group 3, as can be seen in FIGS. 23D-23F, flow cytometry of blood analyzed in each of the three animals demonstrated a decrease in CCR8+T-reg cells up to 168 hours post dose. With regard to Group 2, as can be seen in FIGS. 23G-231, flow cytometry of blood analyzed demonstrated a decrease in CCR8+T-reg cells in Animals 2002 and 2003. Both Group 2 and 3 animals demonstrated little to no effect on the overall Treg cell count (FIGS. 22A-22C) but demonstrated reduced numbers of peripheral blood CCR8+T-reg cells following administration (FIGS. 23D-231), either spiked or unspiked, which is consistent with the proposed mechanism of action (see FIG. 2A).

FIG. 24 is a schematic diagram showing the study design for the Phase Ia portion (RO7502175 as a Single Agent) of the G043860 study. DL=dose level; DLT=dose-limiting toxicity; HCC=hepatocellular carcinoma; HNSCC=head and neck squamous cell carcinoma; MAD=maximum administered dose; MTD=maximum tolerated dose; NSCLC=non-small cell lung cancer; PD=pharmacodynamic; PK=pharmacokinetic; Q3W=every 3 weeks; RCC=renal cell carcinoma; TNBC=triple-negative breast cancer; UC=urothelial carcinoma. Actual patient counts may vary.

FIG. 25 is a schematic diagram showing the study design for Phase Ib portion (RO7502175 in Combination with Atezolizumab) of the G043860 study. TBD=to be determined.

FIG. 26 is a schematic diagram showing the conditions for continuing treatment beyond disease progression in the G043860 study. ECOG=Eastern Cooperative Oncology Group; RECIST=Response Evaluation Criteria in Solid Tumors.

FIGS. 27A and 27B are schematic diagrams showing the conditions for crossover from Phase Ia to Phase Ib for Phase Ia patients in the G043860 study. AE=adverse event; PD=progressive disease.

FIGS. 28A-28C depict in vitro pharmacology of RO7502175. (FIG. 28A) RO7502175 binds to CCR8 only from human and cynomolgus monkeys among the species assessed. Afucosylated RO7502175 shows enhanced binding to FcγRIIIA-F158 (FIG. 28B) and FcγRIIIA-V158 (FIG. 28C) compared to a trastuzumab control antibody.

FIG. 29 depicts in vitro ADCC activity of RO7502175 against human Treg cells from pre-activated PBMCs. The plots show ADCC activity of RO7502175, fucosylated (wild type) anti-CCR8 control, and afucosylated anti-gD (isotype) control against human Treg cells from pre-activated PBMCs isolated from three different donors.

FIGS. 30A and 30B depict in vitro ADCC activity of RO7502175 against dissociated tumor cells and CCR8-expressing CHO cells. (FIG. 30A) The plots show ADCC activity of RO7502175 against human regulatory, conventional CD4+ and CD8+ T cells from dissociated renal cell carcinoma tumors. (FIG. 30B) The plot shows percent cytotoxicity (mean±SD) of RO7502175 in an ADCC assay with CCR8-expressing CHO cells using PBMCs isolated from 8 different healthy donors. The geometric mean EC50 value is listed.

FIGS. 31A-31F depict in vitro cytokine release for RO7502175 in a PBMC assay with soluble and immobilized formats. Graphical depictions of (FIG. 31A) the soluble assay format and (FIG. 31B) the immobilized assay format combining test articles with PBMCs are shown. (FIGS. 31C-31F) Cytokine concentrations (pg/mL) following 18-hour incubation of indicated test articles in the soluble or immobilized formats with PBMCs (n=8), (FIG. 31C) IFNγ, (FIG. 31D) IL-2, (FIG. 31E) IL-6, and (FIG. 31F) TNFα are shown.

FIGS. 32A-32E depict in vivo activity of anti-murine CCR8 antibody in a syngeneic mouse tumor model. (FIG. 32A) Study design, frequencies of (FIG. 32B) Treg cells and (FIG. 32C) CD8+ T cells in tumor and blood, and (FIG. 32D) serum antibody concentrations (mean±SD) following single IV administration of anti-murine CCR8 antibody in C57BL/6 mice bearing E0771 tumors are shown. (FIG. 32E) Anti-tumor activity in C57BL/6 mice bearing E0771 tumors following single IV administration of anti-murine CCR8 antibody in an efficacy study is shown. dLN, draining lymph node; MQC, minimum quantifiable concentration.

FIGS. 33A and 33B depict in vivo activity of anti-murine CCR8 antibody in a syngeneic mouse tumor model (additional data from PD study described in FIGS. 32A-32D). Frequencies of (FIG. 33A) Treg cells and (FIG. 33B) CD8+ T cells in spleen and draining lymph nodes are shown.

FIG. 34 depicts anti-murine CCR8 antibody PK in C57BL/6 mice from the efficacy study described in FIG. 32E. Serum concentrations (mean±SD) of pharmacokinetic parameter estimates from non-compartmental analysis following single IV administration of anti-murine CCR8 antibody in C57BL/6 mice are shown.

FIGS. 35A-35D depict RO7502175 PK, PD, and safety profiles in cynomolgus monkeys. (FIG. 35A) Serum concentration-time profiles (mean±SD), and (FIG. 35B) plasma MCP-1 and IL-6 cytokine concentrations (mean±SD) following single IV administration of 10 mg/kg anti-gD (control) or RO7502175 to male cynomolgus monkeys are shown. (FIG. 35C) Serum concentration-time profiles (mean±SD), and (FIG. 35D) fold change from baseline of CCR8+ Treg cells in blood (mean±SD) of cynomolgus monkey following IV administration of vehicle control or RO7502175 in a repeat-dose study are shown.

FIGS. 36A and 36B depict RO7502175 PD profiles in single-dose study in cynomolgus monkeys. Relative percentages of CCR8+ Treg cells (mean±SEM; n=2 replicates) in blood of individual male cynomolgus monkeys following IV administration of 10 mg/kg (FIG. 36A) anti-gD (control) or (FIG. 36B) RO7502175 are shown.

FIGS. 37A-37H depict that a mPBPK-PD model reproduced preclinical PK, PD and anti-tumor efficacy data. (FIG. 37A) A schematic representation of the mPBPK-PD model structure is shown. (FIG. 37B) The model captured anti-murine CCR8 antibody PK in mice after a single dose administration of 0.01, 0.03, 0.1, and 1 mg/kg IV. (FIG. 37C) The model predicted the receptor occupancy (RO) over time of anti-murine CCR8 antibody. (FIG. 37D) The model is calibrated to tumor CCR8+ Treg cell counts. (FIG. 37E) The model captured CD8+ T cell increases and (FIG. 37F) average tumor killing. (FIG. 37G) The model is calibrated and validated against RO7502175 PK from the single-dose and repeat-dose studies in cynomolgus monkeys. (FIG. 37H) The RO predictions in cynomolgus monkeys at 10 mg/kg single dose, and 30 and 100 mg/kg qw dosing are shown. The symbols represent individual data points and the lines represent model fits or predictions.

FIGS. 38A-38D depict projected RO7502175 clinical PK profiles and receptor occupancy (RO) in plasma and tumor in patients. The clinical PK and RO predictions in (FIGS. 38A and 38B) blood and (FIGS. 38C and 38D) tumor compartments following administration of RO7502175 at dose levels of 0.2, 0.6, 2, 6, and 20 mg IV q3w are shown.

FIG. 39 depicts projected RO7502175 receptor occupancy (RO) in tumor for patients. The clinical average RO predictions in the tumor compartment over cycle 1 (21 days) following administration of RO7502175 at dose levels of 0.2, 0.6, 2, 6, and 20 mg IV q3w are shown. The ranges show the average RO in the tumor considering uncertainty in PK parameters (Vplasma=37.1 mL/kg+/−20%, CL=0.12 mL/h/kg+/−20%, sig_tumor=0.91-0.95 (for 5-10% tumor/plasma AUC), KD=0.032-0.060 nM). Vplasma, volume of plasma; CL, clearance; sig_tumor, tumor reflection coefficient; K_{D}, antibody binding affinity to CCR8.

FIGS. 40A and 40B depict First-in-Human dose selection for RO7502175. (FIG. 40A) MABEL-based approach, mPAD-based approach and an integrated approach based on the totality of preclinical data were considered for the selection of RO7502175 FiH dose. The workflows in the schematic show in vitro and in vivo datasets utilized as inputs and the criteria used for the generation of outcomes from each methodology assessed. (FIG. 40B) The plot shows the resulting FiH dose for RO7502175 based on each of the methods examined. RO, receptor occupancy; K_{D}, antibody binding affinity to CCR8; ADCC, antibody-dependent cell-mediated cytotoxicity; MABEL, minimum anticipated biological effect level; Cₘₐₓ, maximum observed concentration; mPAD, minimal pharmacologically active dose; C_{avg}, average concentration over a defined time period post-dose; NOAEL, no-observed-adverse-effect level; CRA, in vitro cytokine release assay.

### DETAILED DESCRIPTION OF THE INVENTION

### I. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some aspects, the number of amino acid changes are 10 or less, 9 or less, 8 or less, 7 or less, 6 or less, 5 or less, 4 or less, 3 or less, or 2 or less. In some aspects, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary methods for measuring binding affinity are also described herein.

An “affinity matured” antibody refers to an antibody with one or more alterations in one or more complementary determining regions (CDRs), compared to a parent antibody which does not possess such alterations, such alterations resulting in an improvement in the affinity of the antibody for antigen.

The terms “anti-CCR8 antibody” and “an antibody that binds to CCR8” refer to an antibody that is capable of binding CCR8 with sufficient affinity such that the antibody is useful as a diagnostic and/or therapeutic agent in targeting CCR8. In one aspect, the extent of binding of an anti-CCR8 antibody to an unrelated, non-CCR8 protein is less than about 10% of the binding of the antibody to CCR8 as measured, e.g., by surface plasmon resonance (SPR). In certain aspects, an antibody that binds to CCR8 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻¹³ M to 10⁻⁸ M, e.g., from 10⁻¹³ M to 10⁻⁹ M). In certain aspects, an antibody that binds to CCR8 has a K_{D} of from about 1×10⁻¹² M to about 1×10⁻¹⁰ M, from about 1×10⁻¹² M to about 1×10⁻¹¹ M, or from about 1×10⁻¹¹ M to about 5×10⁻¹¹ M. In certain aspects, an antibody that binds to CCR8 has a K_{D} of about 2×10⁻¹¹ M. In certain aspects, an antibody that binds to CCR8 has a K_{D} of about 5×10⁻¹² M. An antibody is said to “specifically bind” to CCR8 when the antibody has a K_{D} of 1 μM or less. In certain embodiments, an anti-CCR8 antibody binds to an epitope of CCR8 in at least two different species (e.g., human and cyno).

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology (2005) 23:1126-1136.

The term “epitope” denotes the site on an antigen, either proteinaceous or non-proteinaceous, to which an anti-CCR8 antibody binds. Epitopes can be formed both from contiguous amino acid stretches (linear epitope) or comprise non-contiguous amino acids (conformational epitope), e.g., coming in spatial proximity due to the folding of the antigen, i.e., by the tertiary folding of a proteinaceous antigen. Linear epitopes are typically still bound by an anti-CCR8 antibody after exposure of the proteinaceous antigen to denaturing agents, whereas conformational epitopes are typically destroyed upon treatment with denaturing agents. An epitope comprises at least 3, at least 4, at least 5, at least 6, at least 7, at least 8, at least 10, at least 15, at least 20, at least 30, or at least 35, or 3-25, 3-20, 3-15, 3-10, 3-5, 30-40, 35-40, or 5-10 amino acids in a unique spatial conformation.

Screening for antibodies binding to a particular epitope (i.e., those binding to the same epitope) can be done using methods routine in the art such as, e.g., without limitation, alanine scanning, peptide blots (see, e.g., Kobeissy et al., Meth. Mol. Biol. (2004) 248: 443-463), peptide cleavage analysis, epitope excision, epitope extraction, chemical modification of antigens (see Hochleitner et al., Prot. Sci. 9 (2000) 487-496), and cross-blocking (see “Antibodies”, Harlow and Lane (Cold Spring Harbor Press, Cold Spring Harb., NY).

Antigen Structure-based Antibody Profiling (ASAP), also known as Modification-Assisted Profiling (MAP), allows to bin a multitude of monoclonal antibodies specifically binding to CCR8 based on the binding profile of each of the antibodies from the multitude to chemically or enzymatically modified antigen surfaces (see, e.g., US 2004/0101920). The antibodies in each bin bind to the same epitope which may be a unique epitope either distinctly different from or partially overlapping with epitope represented by another bin.

Also, competitive binding can be used to easily determine whether an antibody binds to the same epitope of CCR8 as, or competes for binding with, a reference anti-CCR8 antibody. For example, an “antibody that binds to the same epitope” as a reference anti-CCR8 antibody refers to an antibody that blocks binding of the reference anti-CCR8 antibody to its antigen in a competition assay by 50% or more, and conversely, the reference antibody blocks binding of the antibody to its antigen in a competition assay by 50% or more. Also for example, to determine if an antibody binds to the same epitope as a reference anti-CCR8 antibody, the reference antibody is allowed to bind to CCR8 under saturating conditions. After removal of the excess of the reference anti-CCR8 antibody, the ability of an anti-CCR8 antibody in question to bind to CCR8 is assessed. If the anti-CCR8 antibody is able to bind to CCR8 after saturation binding of the reference anti-CCR8 antibody, it can be concluded that the anti-CCR8 antibody in question binds to a different epitope than the reference anti-CCR8 antibody. But, if the anti-CCR8 antibody in question is not able to bind to CCR8 after saturation binding of the reference anti-CCR8 antibody, then the anti-CCR8 antibody in question may bind to the same epitope as the epitope bound by the reference anti-CCR8 antibody. To confirm whether the antibody in question binds to the same epitope or is just hampered from binding by steric reasons routine experimentation can be used (e.g., peptide mutation and binding analyses using ELISA, RIA, surface plasmon resonance, flow cytometry or any other quantitative or qualitative antibody-binding assay available in the art). This assay should be carried out in two set-ups, i.e., with both of the antibodies being the saturating antibody. If, in both set-ups, only the first (saturating) antibody is capable of binding to CCR8, then it can be concluded that the anti-CCR8 antibody in question and the reference anti-CCR8 antibody compete for binding to CCR8.

In some aspects, two antibodies are deemed to bind to the same or an overlapping epitope if a 1-, 5-, 10-, 20- or 100-fold excess of one antibody inhibits binding of the other by at least 50%, at least 75%, at least 90% or even 99% or more as measured in a competitive binding assay (see, e.g., Junghans et al., Cancer Res. 50 (1990) 1495-1502).

In some aspects, two antibodies are deemed to bind to the same epitope if essentially all amino acid mutations in the antigen that reduce or eliminate binding of one antibody also reduce or eliminate binding of the other. Two antibodies are deemed to have “overlapping epitopes” if only a subset of the amino acid mutations that reduce or eliminate binding of one antibody reduce or eliminate binding of the other.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. In certain aspects, the antibody is of the IgG₁ isotype. In certain aspects, the antibody is of the IgG₁ isotype with the P329G, L234A and L235A mutation to reduce Fc-region effector function. In other aspects, the antibody is of the IgG₂ isotype. In certain aspects, the antibody is of the IgG₄ isotype with the S228P mutation in the hinge region to improve stability of IgG₄ antibody. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain.

The terms “constant region derived from human origin” or “human constant region” as used in the current application denotes a constant heavy chain region of a human antibody of the subclass IgG₁, IgG₂, IgG₃, or IgG₄ and/or a constant light chain kappa or lambda region. Such constant regions are well known in the state of the art and e.g., described by Kabat, E. A., et al., Sequences of Proteins of Immunological Interest, 5th ed., Public Health Service, National Institutes of Health, Bethesda, MD (1991) (see also e.g., Johnson, G., and Wu, T. T., Nucleic Acids Res. 28 (2000) 214-218; Kabat, E. A., et al., Proc. Natl. Acad. Sci. USA 72 (1975) 2785-2788). Unless otherwise specified herein, numbering of amino acid residues in the constant region is according to the EU numbering system, also called the EU index of Kabat, as described in Kabat, E. A. et al., Sequences of Proteins of Immunological Interest, 5th ed., Public Health Service, National Institutes of Health, Bethesda, MD (1991), NIH Publication 91-3242.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

An “effective amount” of an agent, e.g., in a pharmaceutical composition, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, EU numbering system). Therefore, the C-terminal lysine (Lys447), or the C-terminal glycine (Gly446) and lysine (Lys447), of the Fc region may or may not be present. In one aspect, a heavy chain including an Fc region as specified herein, comprised in an antibody according to the invention, comprises an additional C-terminal glycine-lysine dipeptide (G446 and K447, EU numbering system). In one aspect, a heavy chain including an Fc region as specified herein, comprised in an antibody according to the invention, comprises an additional C-terminal glycine residue (G446, numbering according to EU index). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2− CDR-H2(CDR-L2)-FR3-CDR-H3(CDR-L3)-FR4.

The terms “full-length antibody”, “intact antibody”, and “whole antibody” are used herein interchangeably to refer to an antibody having a structure substantially similar to a native antibody structure or having heavy chains that contain an Fc region as defined herein. It should be understood that the full-length antibody comprises a heavy chain variable domain and light chain variable domain, as defined herein, and an Fc region as defined herein.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one aspect, for the VL, the subgroup is subgroup kappa I as in Kabat et al., supra. In one aspect, for the VH, the subgroup is subgroup III as in Kabat et al., supra.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”).

In certain aspects, antibodies comprise six CDRs: three in the VH (CDR-H1, CDR-H2, CDR-H3), and three in the VL (CDR-L1, CDR-L2, CDR-L3). In certain aspects, the antibodies comprising six CDRs are full-length antibodies. In certain aspects, the antibodies comprising six CDRs are antibody fragments.

Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra and Chothia, supra. One of skill in the art will understand that the CDR designations can also be determined according to McCallum, supra, or any other scientifically accepted nomenclature system.

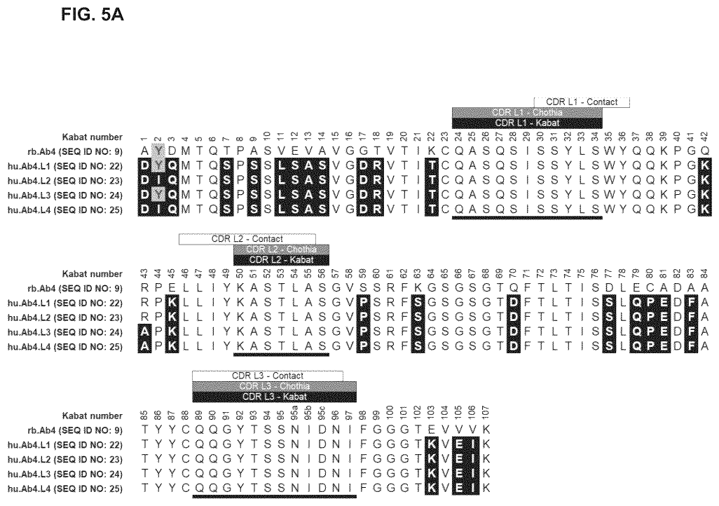

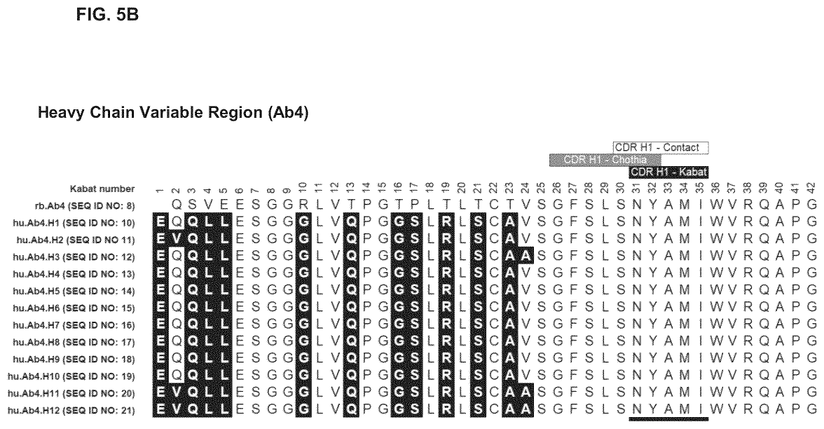

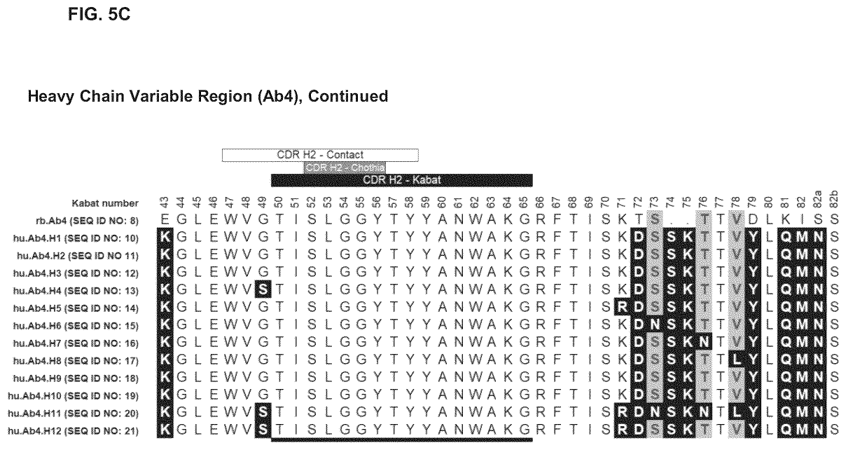

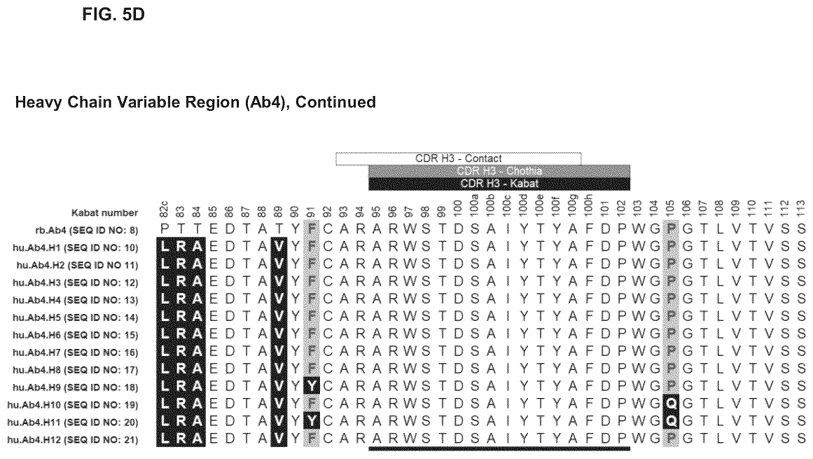

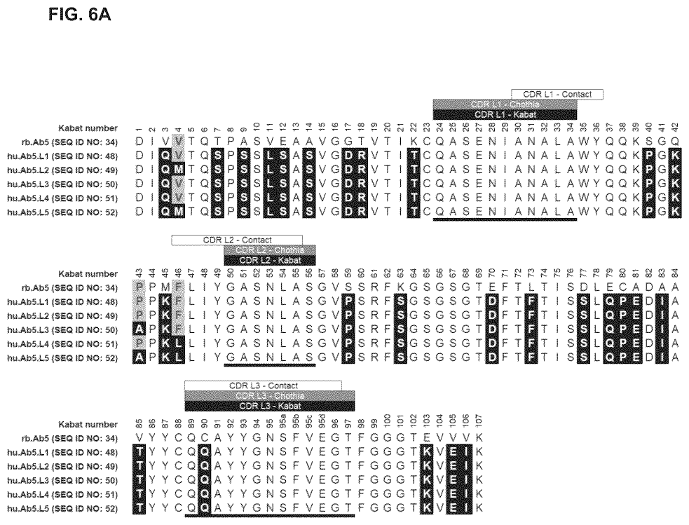

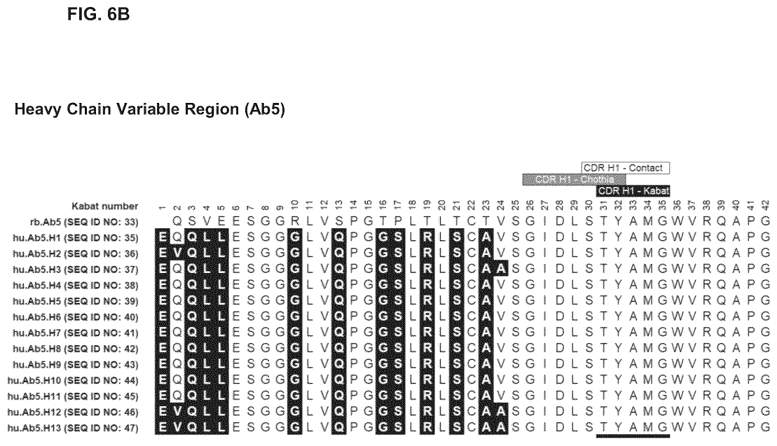

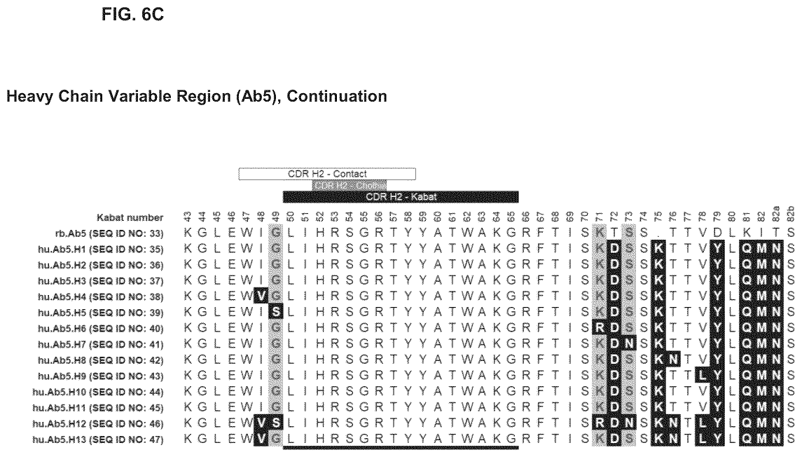

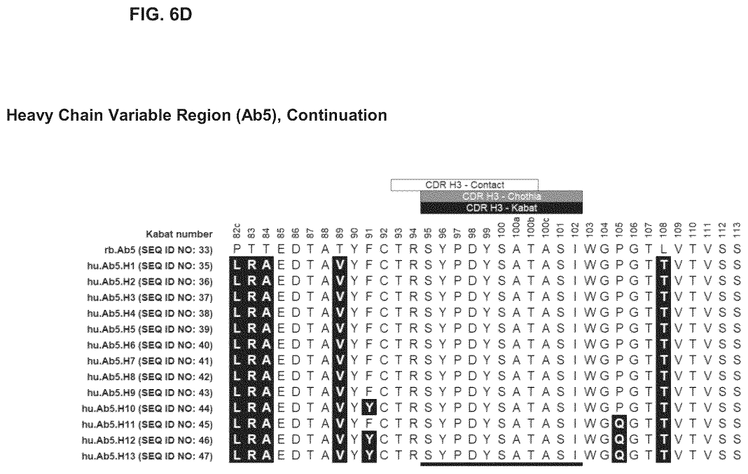

In one aspect, CDR residues comprise those identified in FIGS. 5A-5D and 6A-6D and Tables C1, C2, D1 and D2. In other aspects, CDR residues comprise those identified in Tables N1, N2, O1, and O2.

A “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the subject is a human. In some aspects, the subject is a patient.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

The term “nucleic acid molecule” or “polynucleotide” includes any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody as described herein in vitro and/or in vivo, e.g., in a host or subject. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler et al, Nature Medicine 2017, published online 12 Jun. 2017, doi:10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-CCR8 antibody” refers to one or more nucleic acid molecules encoding anti-CCR8 antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies in accordance with the present disclosure may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical composition.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N-to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full-length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36 and is publicly available from fasta.bioch.virginia.edu/fasta_www2/fasta_down.shtml or ebi.ac.uk/Tools/sss/fasta. Alternatively, a public server accessible at fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

The terms “pharmaceutical composition” and “pharmaceutical formulation” are used interchangeably herein and refer to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

The term “CCR8”, as used herein, refers to any native CCR8 from any vertebrate source, including mammals such as primates (e.g., humans, monkeys (cyno)), and rodents (e.g., mice and rats), unless otherwise indicated. The term encompasses “full-length”, unprocessed CCR8 as well as any form of CCR8 that results from processing in the cell. The term also encompasses naturally occurring variants of CCR8, e.g., splice variants or allelic variants. In certain aspects, the CCR8 is a human CCR8 (“hCCR8” or “huCCR8”). The amino acid sequence of an exemplary human CCR8 is set forth in SEQ ID NO: 106, as shown in the below Table. In certain aspects, the CCR8 is a cynomolgus monkey (“cyno”) CCR8. The amino acid sequence of an exemplary cyno CCR8 is set forth in SEQ ID NO: 107, as shown in the below Table. In certain aspects, the CCR8 is a mouse CCR8 (“mCCR8”). The amino acid sequence of an exemplary mouse CCR8 is set forth in SEQ ID NO: 108, as shown in the below Table.

TABLE 1
Exemplary CCR8 sequences

| Description | Sequence |
|---|---|
| human CCR8 | MDYTLDLSVTTVTDYYYPDIFSSPCDAELIQTNGKLLLAVFYCLLFVFSLLGNS |
| SEQ ID NO: 106 | LVILVLVVCKKLRSITDVYLLNLALSDLLFVFSFPFQTYYLLDQWVFGTVMCKV |
| | VSGFYYIGFYSSMFFITLMSVDRYLAVVHAVYALKVRTIRMGTTLCLAVWLTAI |
| | MATIPLLVFYQVASEDGVLQCYSFYNQQTLKWKIFTNFKMNILGLLIPFTIFMF |
| | CYIKILHQLKRCQNHNKTKAIRLVLIVVIASLLFWVPFNVVLFLTSLHSMHILDG |
| | CSISQQLTYATHVTEIISFTHCCVNPVIYAFVGEKFKKHLSEIFQKSCSQIFNYL |
| | GRQMPRESCEKSSSCQQHSSRSSSVDYIL |
| cyno CCR8 | MDYTLDPSMTTMTDYYYPDSLSSPCDGELIQRNDKLLLAVFYCLLFVFSLLGN |
| SEQ ID NO: 107 | SLVILVLVVCKKLRNITDIYLLNLALSDLLFVFSFPFQTYYQLDQWVFGTVMCK |
| | VVSGFYYIGFYSSMFFITLMSVDRYLAVVHAVYAIKVRTIRMGTTLSLVVWLTA |
| | IMATIPLLVFYQVASEDGVLQCYSFYNQQTLKWKIFTNFEMNILGLLIPFTIFMF |
| | CYIKILHQLKRCQNHNKTKAIRLVLIVVIASLLFWVPFNVVLFLTSLHSMHILDG |
| | CSISQQLNYATHVTEIISFTHCCVNPVIYAFVGEKFKKHLSEIFQKSCSHIFIYL |
| | GRQMPRESCEKSSSCQQHSFRSSSIDYIL |
| mouse CCR8 | MDYTMEPNVTMTDYYPDFFTAPCDAEFLLRGSMLYLAILYCVLFVLGLLGNSL |
| SEQ ID NO: 108 | VILVLVGCKKLRSITDIYLLNLAASDLLFVLSIPFQTHNLLDQWVFGTAMCKVV |
| | SGLYYIGFFSSMFFITLMSVDRYLAIVHAVYAIKVRTASVGTALSLTVWLAAVT |
| | ATIPLMVFYQVASEDGMLQCFQFYEEQSLRWKLFTHFEINALGLLLPFAILLFC |
| | YVRILQQLRGCLNHNRTRAIKLVLTVVIVSLLFWVPFNVALFLTSLHDLHILDG |
| | CATRQRLALAIHVTEVISFTHCCVNPVIYAFIGEKFKKHLMDVFQKSCSHIFLYL |
| | GRQMPVGALERQLSSNQRSSHSSTLDDIL |

The term “PD-1 axis binding antagonist” refers to a molecule that inhibits the interaction of a PD-1 axis binding partner with either one or more of its binding partners, so as to remove T-cell dysfunction resulting from signaling on the PD-1 signaling axis, with a result being to restore or enhance T-cell function (e.g., proliferation, cytokine production, and/or target cell killing). As used herein, a PD-1 axis binding antagonist includes a PD-L1 binding antagonist, a PD-1 binding antagonist, and a PD-L-2 binding antagonist. In some instances, the PD-1 axis binding antagonist includes a PD-L1 binding antagonist or a PD-1 binding antagonist. In a preferred aspect, the PD-1 axis binding antagonist is a PD-L1 binding antagonist.

The term “PD-L1 binding antagonist” refers to a molecule that decreases, blocks, inhibits, abrogates, or interferes with signal transduction resulting from the interaction of PD-L1 with either one or more of its binding partners, such as PD-1 and/or B7-1. In some instances, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some instances, the PD-L1 binding antagonists include anti-PD-L1 antibodies, antigen-binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one instance, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some instances, the PD-L1 binding antagonist binds to PD-L1. In some instances, a PD-L1 binding antagonist is an anti-PD-L1 antibody (e.g., an anti-PD-L1 antagonist antibody). Exemplary anti-PD-L1 antagonist antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. In some aspects, the anti-PD-L1 antibody is atezolizumab, MDX-1105, MEDI4736 (durvalumab), or MSB0010718C (avelumab). In one specific aspect, the PD-L1 binding antagonist is MDX-1105. In another specific aspect, the PD-L1 binding antagonist is MEDI4736 (durvalumab). In another specific aspect, the PD-L1 binding antagonist is MSB0010718C (avelumab). In other aspects, the PD-L1 binding antagonist may be a small molecule, e.g., GS-4224, INCB086550, MAX-10181, INCB090244, CA-170, or ABSK041, which in some instances may be administered orally. Other exemplary PD-L1 binding antagonists include AVA-004, MT-6035, VXM10, LYN192, GB7003, and JS-003. In one aspect, the PD-L1 binding antagonist is atezolizumab.

For the purposes herein, “atezolizumab” is an Fc-engineered, humanized, non-glycosylated IgG1 kappa immunoglobulin that binds PD-L1. Atezolizumab comprises a single amino acid substitution (asparagine to alanine) at position 297 on the heavy chain (N297A) using EU numbering of Fc region amino acid residues, which results in a non-glycosylated antibody that has minimal binding to Fc receptors. Atezolizumab is also described in WHO Drug Information (International Nonproprietary Names for Pharmaceutical Substances (proposed INN)) List 112, Vol. 28, No. 4, 2014, p. 488.

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. In some instances, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen-binding fragments thereof, immunoadhesins, fusion proteins, oligopeptides, and other molecules that decrease, block, inhibit, abrogate or interfere with signal transduction resulting from the interaction of PD-1 with PD-L1 and/or PD-L2. In one instance, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some instances, the PD-1 binding antagonist binds to PD-1. In some instances, the PD-1 binding antagonist is an anti-PD-1 antibody (e.g., an anti-PD-1 antagonist antibody). Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-I10A, zimberelimab, balstilimab, genolimzumab, BI 754091, cetrelimab, YBL-006, BAT1306, HX008, budigalimab, AMG 404, CX-88, JTX-4014, 609A, Sym021, LZM009, F520, SG001, AM0001, ENUM 244C8, ENUM 388D4, STI-1110, AK-103, and hAb21. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is a PD-L2 Fc fusion protein, e.g., AMP-224. In another specific aspect, a PD-1 binding antagonist is MED1-0680. In another specific aspect, a PD-1 binding antagonist is PDR001 (spartalizumab). In another specific aspect, a PD-1 binding antagonist is REGN2810 (cemiplimab). In another specific aspect, a PD-1 binding antagonist is BGB-108. In another specific aspect, a PD-1 binding antagonist is prolgolimab. In another specific aspect, a PD-1 binding antagonist is camrelizumab. In another specific aspect, a PD-1 binding antagonist is sintilimab. In another specific aspect, a PD-1 binding antagonist is tislelizumab. In another specific aspect, a PD-1 binding antagonist is toripalimab. Other exemplary PD-1 binding antagonists include BION-004, CB201, AUNP-012, ADG104, and LBL-006.

As used herein, “treatment” (and grammatical variations thereof such as “treat” or “treating”) refers to clinical intervention in an attempt to alter the natural course of a disease (e.g., cancer (e.g., a locally advanced, recurrent, or metastatic solid tumor malignancy)) in the subject being treated, and can be performed either for prophylaxis (“preventative treatment” or “prophylactically treating”) or during the course of clinical pathology (“therapeutic treatment” or “therapeutically treating”). Desirable effects of therapeutic treatment include, but are not limited to, alleviation of symptoms, diminishment of any direct or indirect pathological consequences of the disease, preventing metastasis of the cancer, decreasing the rate of disease progression, amelioration or palliation of the disease state, and remission or improved prognosis. Desirable effects of preventative treatment include, but are not limited to, preventing occurrence or recurrence of disease. In some aspects, antibodies as described herein are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6^{th} ed., W. H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

### II. Methods and Compositions

In one aspect, the present disclosure is based, in part, on the development of therapeutic methods and dosing regimens for treatment of locally advanced, recurrent, or metastatic solid tumor malignancies using anti-CCR8 antibodies, e.g., anti-CCR8 antibodies as disclosed herein.

### A. Therapeutic Methods and Compositions for Use

The present disclosure provides therapeutic methods and compositions for use in treatment of cancer (e.g., a locally advanced, recurrent, or metastatic solid tumor malignancy) in a subject in need thereof that may include administering an anti-CCR8 antibody as disclosed herein to the subject, either alone or in combination with one or more additional therapeutic agents (e.g., a PD-1 axis binding antagonist, e.g., an anti-PD-L1 antibody such as atezolizumab). Any of the anti-CCR8 antibodies (e.g., monoclonal antibodies that bind to CCR8) may be used.

In one aspect, provided herein is a method of treating a locally advanced, recurrent, or metastatic solid tumor malignancy in a subject in need thereof, the method comprising administering to the subject a monoclonal antibody that binds to CCR8.

In another aspect, provided herein is a monoclonal antibody that binds to CCR8 for use in treating a locally advanced, recurrent, or metastatic solid tumor malignancy in a subject in need thereof.

In another aspect, provided herein is the use of a monoclonal antibody that binds to CCR8 in the manufacture of a medicament for treating a locally advanced, recurrent, or metastatic solid tumor malignancy in a subject in need thereof.

In one aspect, provided herein is a method of depleting regulatory T cells (“Tregs”) in a tumor microenvironment of a locally advanced, recurrent, or metastatic solid tumor malignancy in a subject in need thereof, the method comprising administering to the subject a monoclonal antibody that binds to CCR8.

In another aspect, provided herein is a monoclonal antibody that binds to CCR8 for use in depleting Tregs in a tumor microenvironment of a locally advanced, recurrent, or metastatic solid tumor malignancy in a subject in need thereof.

In another aspect, provided herein is the use of a monoclonal antibody that binds to CCR8 in the manufacture of a medicament for depleting Tregs in a tumor microenvironment of a locally advanced, recurrent, or metastatic solid tumor malignancy in a subject in need thereof.

In some aspects, (i) the subject has progressed after at least one available standard therapy; and/or (ii) the subject is one for whom all available standard therapy has been proven to be ineffective or intolerable or is contraindicated.

In some aspects, the locally advanced, recurrent, or metastatic solid tumor is incurable.

In some aspects, the subject's age is 18 years or older. For example, the subject may be an adult.

Any suitable locally advanced, recurrent, or metastatic solid tumor malignancy may be treated.

For example, in some aspects, the locally advanced, recurrent, or metastatic solid tumor malignancy is non-small cell lung cancer (NSCLC), head and neck squamous cell carcinoma (HNSCC), melanoma, triple-negative breast cancer (TNBC), urothelial carcinoma (UC), esophageal cancer, gastric cancer, cervical cancer, renal cell carcinoma (RCC), or hepatocellular carcinoma (HCC).

In some aspects, the RCC is clear cell RCC.

In some aspects, the HNSCC is HNSCC of the oral cavity, oropharynx, hypopharynx, or larynx.

In some aspects, the locally advanced, recurrent, or metastatic solid tumor malignancy is NSCLC.

In some aspects, the subject's tumor comprises a targetable somatic alteration, and the subject has experienced disease progression during or after treatment, or intolerance to treatment, with a targeted agent.

In some aspects, the targetable somatic alteration comprises a somatic alteration involving epidermal growth factor receptor (EGFR), anaplastic lymphoma kinase (ALK), ROS proto-oncogene 1 (ROS1), proto-oncogene B-Raf (BRAF) V600E, neurotrophic tyrosine receptor kinase (NTRK), MET proto-oncogene (MET), RET proto-oncogene (RET), or Kirsten rat sarcoma virus (KRAS).

In some aspects, the melanoma is cutaneous melanoma.

In some aspects, the subject's tumor comprises a BRAFV600 mutation, and the subject has experienced disease progression during or after treatment, or intolerance to treatment, with one or more serine/threonine-protein kinase B-Raf (BRAF) inhibitors and/or one or more mitogen-activated protein kinase kinase (MEK) inhibitors.

In some aspects, the locally advanced, recurrent, or metastatic solid tumor malignancy is UC.

In some aspects, the subject has: (i) histologically confirmed incurable advanced transitional cell carcinoma of the urothelium (including renal pelvis, ureters, urinary bladder, and urethra); and/or (ii) a mixed histology, wherein the subject's tumor has a dominant transitional cell pattern.

In some aspects, the locally advanced, recurrent, or metastatic solid tumor malignancy is TNBC.

In some aspects, TNBC is defined by the American Society of Clinical Oncology-College of American Pathologists guidelines: (i) <1% of tumor-cell nuclei immunoreactive for estrogen receptor and <1% of tumor-cell nuclei immunoreactive for progesterone receptor; and/or (ii) HER2-negative based on immunohistochemistry (IHC) and/or in situ hybridization.

In some aspects, the subject is checkpoint inhibitor (CPI)-naïve.

In some aspects, the subject's locally advanced, recurrent, or metastatic solid tumor malignancy is NSCLC or UC.

In some aspects, the subject's locally advanced, recurrent, or metastatic solid tumor malignancy is UC, wherein the subject is eligible for treatment with cisplatin, and the subject has experienced disease progression during or after treatment, or intolerance to treatment, with cisplatin.

In some aspects, the subject has had no prior treatment with a CPI, or wherein the subject has had adjuvant treatment with a CPI that was discontinued at least six months prior to first administration of the monoclonal antibody that binds to CCR8 to the subject.

In some aspects, the subject is CPI-experienced.

In some aspects, the subject's locally advanced, recurrent, or metastatic solid tumor malignancy is NSCLC, HNSCC, melanoma, UC, TNBC, esophageal cancer, gastric cancer, cervical cancer, clear cell RCC, or HCC.

In some aspects, the subject derived clinical benefit from treatment comprising a PD-1 axis binding antagonist prior to disease progression.

In some aspects, the PD-1 axis binding antagonist is an anti-PD-L1 antibody or an anti-PD-1 antibody.

In some aspects, the subject had a treatment duration with the treatment comprising the PD-1 axis binding antagonist of greater than or equal to 6 months and/or had a partial response or complete response as best objective response.

In some aspects, (i) the subject has not received treatment with a CPI, an immunomodulatory monoclonal antibody, or an immunomodulatory monoclonal antibody-derived therapy within 6 weeks prior to first administration of the monoclonal antibody that binds to CCR8 to the subject; or (ii) the subject was previously treated with a PD-1 axis binding antagonist, and the last administration of the PD-1 axis binding antagonist to the subject was at least 3 weeks prior to first administration of the monoclonal antibody that binds to CCR8 to the subject.

In some aspects, the CPI is a PD-1 axis binding antagonist or a CTLA4 antagonist (e.g., an anti-CTLA4 antibody such as ipilimumab (YERVOY®).

In some aspects, the PD-1 axis binding antagonist is an anti-PD-L1 antibody (e.g., any anti-PD-L1 antibody disclosed herein, e.g., atezolizumab or avelumab) or an anti-PD-1 antibody (e.g., any anti-PD-1 antibody disclosed herein, e.g., pembrolizumab).

In some aspects, the monoclonal antibody that binds to CCR8 is administered to the subject in a dosing regimen comprising one or more dosing cycles.

In some aspects, the one or more dosing cycles comprise 21-day dosing cycles.

For example, in one aspect, provided herein is a method of treating a locally advanced, recurrent, or metastatic solid tumor malignancy in a subject in need thereof, the method comprising administering to the subject a monoclonal antibody that binds to CCR8 in a dosing regimen comprising one or more 21-day dosing cycles.

In another aspect, provided herein is a monoclonal antibody that binds to CCR8 for use in treating a locally advanced, recurrent, or metastatic solid tumor malignancy in a subject in need thereof in a dosing regimen comprising one or more 21-day dosing cycles.

In another aspect, provided herein is the use of a monoclonal antibody that binds to CCR8 in the manufacture of a medicament for treating a locally advanced, recurrent, or metastatic solid tumor malignancy in a subject in need thereof in a dosing regimen comprising one or more 21-day dosing cycles.

In some aspects, the monoclonal antibody that binds to CCR8 is administered to the subject on Day 1 of each 21-day dosing cycle.

In one aspect, provided herein is a method of treating a locally advanced, recurrent, or metastatic solid tumor malignancy in a subject in need thereof, the method comprising administering to the subject a monoclonal antibody that binds to CCR8 every three weeks (Q3W).

In another aspect, provided herein is a monoclonal antibody that binds to CCR8 for use in treating a locally advanced, recurrent, or metastatic solid tumor malignancy in a subject in need thereof every three weeks (Q3W).

In another aspect, provided herein is the use of a monoclonal antibody that binds to CCR8 in the manufacture of a medicament for treating a locally advanced, recurrent, or metastatic solid tumor malignancy in a subject in need thereof every three weeks (Q3W).

In one aspect, provided herein is a method of depleting Tregs in a tumor microenvironment of a locally advanced, recurrent, or metastatic solid tumor malignancy in a subject in need thereof, the method comprising administering to the subject a monoclonal antibody that binds to CCR8 every three weeks (Q3W).

In another aspect, provided herein is a monoclonal antibody that binds to CCR8 for use in depleting Tregs in a tumor microenvironment of a locally advanced, recurrent, or metastatic solid tumor malignancy in a subject in need thereof every three weeks (Q3W).

In one aspect, provided herein is a method of treating a locally advanced, recurrent, or metastatic solid tumor malignancy in a subject in need thereof, the method comprising administering to the subject a monoclonal antibody that binds to CCR8 at a dose of 2 mg.

In another aspect, provided herein is a monoclonal antibody that binds to CCR8 for use in treating a locally advanced, recurrent, or metastatic solid tumor malignancy in a subject in need thereof at a dose of 2 mg.

In another aspect, provided herein is the use of a monoclonal antibody that binds to CCR8 in the manufacture of a medicament for treating a locally advanced, recurrent, or metastatic solid tumor malignancy in a subject in need thereof at a dose of 2 mg.

In one aspect, provided herein is a method of depleting Tregs in a tumor microenvironment of a locally advanced, recurrent, or metastatic solid tumor malignancy in a subject in need thereof, the method comprising administering to the subject a monoclonal antibody that binds to CCR8 at a dose of 2 mg.

In another aspect, provided herein is a monoclonal antibody that binds to CCR8 for use in depleting Tregs in a tumor microenvironment of a locally advanced, recurrent, or metastatic solid tumor malignancy in a subject in need thereof at a dose of 2 mg.

In one aspect, provided herein is a method of treating a locally advanced, recurrent, or metastatic solid tumor malignancy in a subject in need thereof, the method comprising administering to the subject a monoclonal antibody that binds to CCR8 at a dose of 2 mg every three weeks (Q3W).

In another aspect, provided herein is a monoclonal antibody that binds to CCR8 for use in treating a locally advanced, recurrent, or metastatic solid tumor malignancy in a subject in need thereof at a dose of 2 mg every three weeks (Q3W).

In another aspect, provided herein is the use of a monoclonal antibody that binds to CCR8 in the manufacture of a medicament for treating a locally advanced, recurrent, or metastatic solid tumor malignancy in a subject in need thereof at a dose of 2 mg every three weeks (Q3W).

In one aspect, provided herein is a method of depleting Tregs in a tumor microenvironment of a locally advanced, recurrent, or metastatic solid tumor malignancy in a subject in need thereof, the method comprising administering to the subject a monoclonal antibody that binds to CCR8 at a dose of 2 mg every three weeks (Q3W).

In another aspect, provided herein is a monoclonal antibody that binds to CCR8 for use in depleting Tregs in a tumor microenvironment of a locally advanced, recurrent, or metastatic solid tumor malignancy in a subject in need thereof at a dose of 2 mg every three weeks (Q3W).

In some examples of any of the preceding aspects, the monoclonal antibody that binds to CCR8 may be administered to the subject until disease progression or unacceptable toxicity.

In some aspects, the monoclonal antibody that binds to CCR8 is administered to the subject at a dose of 2 mg.

Any suitable administration route may be used in the methods and compositions for use disclosed herein. In some aspects, the monoclonal antibody that binds to CCR8 is administered to the subject intravenously.

In some aspects, the monoclonal antibody that binds to CCR8 is administered to the subject intravenously by infusion.

In some aspects, the monoclonal antibody that binds to CCR8 is administered to the subject as a monotherapy.

In other aspects, the monoclonal antibody that binds to CCR8 is administered to the subject as a combination therapy. For instance, the combination therapy may include administering an antibody as described herein and administering at least one additional therapeutic agent (e.g., one, two, three, four, five, or six additional therapeutic agents).

The one or more additional therapeutic agents encompasses any agent that can be administered for treatment. In certain aspects, the additional therapeutic agent is an additional anti-cancer agent.

Exemplary anti-cancer agents include, but are not limited to, a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, antiangiogenic agent, an immunomodulatory agent, an inhibitor of cell adhesion, a cytotoxic or cytostatic agent, an activator of cell apoptosis, an agent that increases the sensitivity of cells to apoptotic inducers, a cytokine, an anti-cancer vaccine or oncolytic virus, a toll-like receptor (TLR) agent, a bispecific antibody, a cellular therapy, and immune cell engager. In certain aspects, the additional therapeutic agent is an immunomodulatory anti-cancer agent, e.g., a checkpoint inhibitor (CPI) such as an anti-CTLA4 antibody (e.g., ipilimumab) or a PD-1 axis binding antagonist (e.g., a PD-L1 binding antagonist (e.g., an anti-PD-L1 antibody (e.g., atezolizumab or avelumab), a PD-1 binding antagonist (e.g., pembrolizumab), or a PD-L2 binding antagonist).

In some aspects, the one or more additional therapeutic agents comprises an anti-PD-L1 antibody. For example, in some aspects, the one or more additional therapeutic agents comprises atezolizumab.

In some aspects, the atezolizumab is administered to the subject in a dosing regimen comprising one or more dosing cycles. In some aspects, the one or more dosing cycles comprise 14-day, 21-day, or 28-day dosing cycles.

In some aspects, the one or more dosing cycles comprise 21-day dosing cycles. In some aspects, the atezolizumab is administered to the subject on Day 1 of each 21-day dosing cycle.

Any suitable dose of atezolizumab may be administered to the subject. In some aspects, the atezolizumab is administered to the subject at a dose of 1200 mg. For example, in some aspects, the atezolizumab is administered to the subject at a dose of 1200 mg every three weeks (Q3W). In other examples, the atezolizumab is administered to the subject at a dose of 840 mg, e.g., every two weeks (Q2W). In yet other example, the atezolizumab is administered to the subject at a dose of 1680 mg, e.g., every four weeks (Q4W).

In some aspects, the atezolizumab is administered to the subject intravenously. In some aspects, the atezolizumab is administered to the subject intravenously by infusion.

In some aspects, a tumor sample from the subject has been determined to have a detectable level of PD-L1 expression. For example, in some aspects, the tumor sample from the subject has a Tumor Cell (TC), an Immune Cell (IC), a Combined Positive Score (CPS), or a Tumor Proportion Score (TPS) greater than or equal to 1%. The presence or expression level of PD-L1 may be determined using any suitable approach, e.g., immunohistochemistry with an anti-PD-L1 diagnostic antibody. Any suitable anti-PD-L1 diagnostic antibody may be used, e.g., SP142 (VENTANA), SP263 (VENTANA), 22C3 (Dako), 28-8 (Dako), E1 L3N, 4059, h5H1, 9A11, and the like.

In some aspects, the subject has received at least two cycles (e.g., at least two, three, four, five, six, seven, eight, nine, ten, or more cycles) of the monoclonal antibody that binds to CCR8 prior to administration of atezolizumab to the subject.

Such combination therapies noted above encompass combined administration (where two or more therapeutic agents are included in the same or separate pharmaceutical compositions), and separate administration, in which case, administration of the antibody as described herein can occur prior to, simultaneously, and/or following, administration of the additional therapeutic agent or agents. In one aspect, administration of the anti-CCR8 antibody and administration of an additional therapeutic agent occur within about one month, or within about one, two or three weeks, or within about one, two, three, four, five, or six days, of each other. In one aspect, the antibody and additional therapeutic agent are administered to the subject on Day 1 of the treatment. Antibodies as described herein can also be used in combination with radiation therapy.

In one aspect, an anti-CCR8 antibody for use as a medicament is provided. In further aspects, an anti-CCR8 antibody for use in treating cancer is provided. In certain aspects, an anti-CCR8 antibody for use in a method of treatment is provided. In certain aspects, the present disclosure provides an anti-CCR8 antibody for use in a method of treating a subject (e.g., a human subject) in need thereof comprising administering to the subject an effective amount of the anti-CCR8 antibody. In one such aspect, the method further comprises administering to the subject an effective amount of at least one additional therapeutic agent (e.g., one, two, three, four, five, or six additional therapeutic agents), e.g., as described below. In further aspects, the present disclosure provides an anti-CCR8 antibody for use in depleting Tregs in a tumor microenvironment. In certain aspects, the present disclosure provides an anti-CCR8 antibody for use in a method of depleting Tregs in a tumor microenvironment in a subject comprising administering to the subject an effective amount of the anti-CCR8 antibody in depletion of Tregs in the tumor microenvironment.

In a further aspect, the present disclosure provides for the use of an anti-CCR8 antibody in the manufacture or preparation of a medicament. In one aspect, the medicament is for treatment of cancer. In a further aspect, the medicament is for use in a method of treating cancer comprising administering to the subject (e.g., a human subject) in need thereof an effective amount of the medicament. In one such aspect, the method further comprises administering to the subject an effective amount of at least one additional therapeutic agent, e.g., as described below. In a further aspect, the medicament is for depleting Tregs in a tumor microenvironment. In a further aspect, the medicament is for use in a method of depleting Tregs in a tumor microenvironment in a subject comprising administering to the subject an effective amount of the medicament to deplete the Tregs in the tumor microenvironment.

In a further aspect, the present disclosure provides a method for treating cancer. In one aspect, the method comprises administering to a subject (e.g., a human subject) in need thereof an effective amount of an anti-CCR8 antibody in order to treat the cancer. In one such aspect, the method further comprises administering to the subject an effective amount of at least one additional therapeutic agent, as described below.

In a further aspect, the present disclosure provides an anti-CCR8 antibody for use in depleting Treg cells, e.g., outside or in a tumor microenvironment. For example, in certain embodiments, the present disclosure provides a method for depleting Treg cells in a tumor microenvironment in a subject (e.g., a human subject) in need thereof having cancer comprising administering to the subject an effective amount of an anti-CCR8 antibody sufficient to deplete the Treg cells in the tumor microenvironment, thereby treating the cancer. In certain aspects, the present disclosure provides a method for depleting Treg cells outside of a tumor microenvironment (e.g., in circulation) in a subject (e.g., a human subject) in need thereof having cancer comprising administering to the subject an effective amount of an anti-CCR8 antibody sufficient to deplete the Treg cells outside the tumor microenvironment, thereby treating the cancer. Without wishing to be bound by any particular theory, by reducing the number of Treg cells outside the tumor microenvironment, the cancer is treated as the number of Treg cells infiltrating into the tumor microenvironment is reduced, thereby reducing the number of Treg cells in the tumor microenvironment.

In one aspect, provided herein is a method of treating a cancer in a subject in need thereof, the method comprising administering to the subject a monoclonal antibody that binds to CCR8 every three weeks (Q3W).

In another aspect, provided herein is a monoclonal antibody that binds to CCR8 for use in treating a cancer in a subject in need thereof every three weeks (Q3W).

In another aspect, provided herein is the use of a monoclonal antibody that binds to CCR8 in the manufacture of a medicament for treating a cancer in a subject in need thereof every three weeks (Q3W).

In one aspect, provided herein is a method of depleting Tregs in a tumor microenvironment of a cancer in a subject in need thereof, the method comprising administering to the subject a monoclonal antibody that binds to CCR8 every three weeks (Q3W).

In another aspect, provided herein is a monoclonal antibody that binds to CCR8 for use in depleting Tregs in a tumor microenvironment of a cancer in a subject in need thereof every three weeks (Q3W).

In one aspect, provided herein is a method of treating a cancer in a subject in need thereof, the method comprising administering to the subject a monoclonal antibody that binds to CCR8 at a dose of 2 mg.

In another aspect, provided herein is a monoclonal antibody that binds to CCR8 for use in treating a cancer in a subject in need thereof at a dose of 2 mg.

In another aspect, provided herein is the use of a monoclonal antibody that binds to CCR8 in the manufacture of a medicament for treating a cancer in a subject in need thereof at a dose of 2 mg.

In one aspect, provided herein is a method of depleting Tregs in a tumor microenvironment of a cancer in a subject in need thereof, the method comprising administering to the subject a monoclonal antibody that binds to CCR8 at a dose of 2 mg.

In another aspect, provided herein is a monoclonal antibody that binds to CCR8 for use in depleting Tregs in a tumor microenvironment of a cancer in a subject in need thereof at a dose of 2 mg.

In one aspect, provided herein is a method of treating a cancer in a subject in need thereof, the method comprising administering to the subject a monoclonal antibody that binds to CCR8 at a dose of 2 mg every three weeks (Q3W).

In another aspect, provided herein is a monoclonal antibody that binds to CCR8 for use in treating a cancer in a subject in need thereof at a dose of 2 mg every three weeks (Q3W).

In another aspect, provided herein is the use of a monoclonal antibody that binds to CCR8 in the manufacture of a medicament for treating a cancer in a subject in need thereof at a dose of 2 mg every three weeks (Q3W).

In one aspect, provided herein is a method of depleting Tregs in a tumor microenvironment of a cancer in a subject in need thereof, the method comprising administering to the subject a monoclonal antibody that binds to CCR8 at a dose of 2 mg every three weeks (Q3W).

In another aspect, provided herein is a monoclonal antibody that binds to CCR8 for use in depleting Tregs in a tumor microenvironment of a cancer in a subject in need thereof at a dose of 2 mg every three weeks (Q3W).

Exemplary cancers includes, but is not limited to, bladder cancer (e.g., urothelial cancer), blastoma, blood cancer (e.g., lymphomas such as Non-Hodgkin's, leukemias), bone cancer, brain cancer, breast cancer (e.g., triple negative breast cancer), cervical cancer, colorectal cancer (e.g., colon cancer, rectal cancer), endometrial cancer, esophageal cancer, gastric cancer, head and neck cancer (e.g., squamous cell carcinoma of the head and neck), kidney cancer (e.g., renal cell carcinoma), liver cancer (e.g., hepatocellular carcinoma), lung cancer (e.g., non-small cell lung cancer, small cell lung carcinoma), ovarian cancer, pancreatic cancer, prostate cancer, sarcoma, skin cancer (e.g., melanoma, squamous cell carcinoma), testicular cancer, and uterine cancer.

In certain aspects, the cancer is bladder cancer, blood cancer, breast cancer, cervical cancer, colorectal cancer, esophageal cancer, gastric cancer, head and neck cancer, kidney cancer, liver cancer, lung cancer, and skin cancer.

In certain aspects, the cancer is bladder cancer, breast cancer, cervical cancer, colorectal cancer, esophageal cancer, head and neck cancer, liver cancer, lung cancer, or skin cancer.

In certain aspects, the cancer is a solid tumor, e.g., a locally advanced or metastatic solid tumor.

In certain aspects, the cancer (e.g., the locally advanced, recurrent, or metastatic solid tumor malignancy) expresses CCR8.

In certain aspects, the cancer (e.g., the locally advanced, recurrent, or metastatic solid tumor malignancy) is a T cell-inflamed tumor or comprises a T-cell-inflamed tumor microenvironment.

In certain aspects, the cancer (e.g., the locally advanced, recurrent, or metastatic solid tumor malignancy) comprises regulatory T cells in the tumor microenvironment, and for which exposure of the cancer to the CCR8 antibody, as described herein, results in depletion of the regulatory T cell in the tumor microenvironment. In a further aspect, the present disclosure provides pharmaceutical compositions comprising any of the anti-CCR8 antibodies described herein, e.g., for use in any of the above therapeutic methods. In one aspect, a pharmaceutical composition comprises any of the anti-CCR8 antibodies provided herein and a pharmaceutically acceptable carrier. In another aspect, a pharmaceutical composition comprises any of the anti-CCR8 antibodies provided herein and at least one additional therapeutic agent, e.g., as described below.

Any of the anti-CCR8 antibodies provided herein (e.g., in Section B below) may be used in therapeutic methods and compositions for use (e.g., anti-CCR8 antibodies for use (e.g., monoclonal antibodies that bind to CCR8 for use) as disclosed herein.

In some aspects, the monoclonal antibody that binds to CCR8 comprises a heavy chain variable domain (VH) comprising (a) CDR-H1 comprising the amino acid sequence of SEQ ID NO: 29 or SEQ ID NO: 30, (b) CDR-H2 comprising the amino acid sequence of SEQ ID NO: 31, and (c) CDR-H3 comprising the amino acid sequence of SEQ ID NO: 32, and a light chain variable domain (VL) comprising (d) CDR-L1 comprising the amino acid sequence of SEQ ID NO: 26, (e) CDR-L2 comprising the amino acid sequence of SEQ ID NO: 27, and (f) CDR-L3 comprising the amino acid sequence of SEQ ID NO: 28.

In some aspects, the monoclonal antibody that binds to CCR8 binds to CCR8 independent of sulfation of CCR8.

In some aspects, the monoclonal antibody that binds to CCR8 binds to an epitope comprised of one or more of amino acid residues 2-6 of SEQ ID NO: 106.

In some aspects, the monoclonal antibody that binds to CCR8 comprises a sequence selected from the group consisting of: (a) a VH sequence having at least about 95%, at least about 96%, at least about 97%, at least about 98%, or at least about 99% identity to an amino acid sequence selected from the group consisting of SEQ ID NOs: 35-47; (b) a VL sequence having at least about 95%, at least about 96%, at least about 97%, at least about 98%, or at least about 99% identity to an amino acid sequence selected from the group consisting of SEQ ID NOs: 48-52; and (c) a VH sequence as defined in (a) and a VL sequence as defined in (b).

In some aspects, the monoclonal antibody that binds to CCR8 comprises a VH sequence selected from the group consisting of SEQ ID NOs: 35-47 and a VL sequence selected from the group consisting of SEQ ID NOs: 48-52.

In some aspects, the monoclonal antibody that binds to CCR8 comprises a sequence selected from the group consisting of: (a) a VH sequence having at least about 95%, at least about 96%, at least about 97%, at least about 98%, or at least about 99% identity to an amino acid sequence of SEQ ID NO: 47; (b) a VL sequence having at least about 95%, at least about 96%, at least about 97%, at least about 98%, or at least about 99% identity to an amino acid sequence of SEQ ID NO: 48; and (c) a VH sequence as defined in (a) and a VL sequence as defined in (b).

In some aspects, the monoclonal antibody that binds to CCR8 comprises a VH sequence having at least about 95%, at least about 96%, at least about 97%, at least about 98%, or at least about 99% identity to the amino acid sequence of SEQ ID NO: 47 and a VL sequence having at least about 95%, at least about 96%, at least about 97%, at least about 98%, or at least about 99% identity to the amino acid sequence of SEQ ID NO: 48.

In some aspects, the VL comprises a V4M mutation, a P43A mutation, a F46L mutation, a C90Q mutation, or a combination thereof. In some instances, the V4M mutation, the P43A mutation, the F46L mutation, or the C90Q mutation is according to Kabat numbering.

In some aspects, the VH comprises a G49S mutation, a K71R mutation, a S73N mutation, or a combination thereof. In some instances, the G49S mutation, the K71R mutation, or the S73N mutation is according to Kabat numbering.

In some aspects, the monoclonal antibody that binds to CCR8 comprises the heavy chain amino acid sequence of SEQ ID NO: 55 and the light chain amino acid sequence of SEQ ID NO: 56.

In some aspects, the monoclonal antibody that binds to CCR8 comprises the heavy chain amino acid sequence of SEQ ID NO: 60 and the light chain amino acid sequence of SEQ ID NO: 56.

In some aspects, the monoclonal antibody that binds to CCR8 comprises the heavy chain amino acid sequence of SEQ ID NO: 111 and the light chain amino acid sequence of SEQ ID NO: 56.

In some aspects, the monoclonal antibody that binds to CCR8 comprises the heavy chain amino acid sequence of SEQ ID NO: 113 and the light chain amino acid sequence of SEQ ID NO: 56.

In some aspects, the monoclonal antibody that binds to CCR8 comprises a VH sequence selected from the group consisting of SEQ ID NOs: 35-47 and a VL sequence selected from the group consisting of SEQ ID NOs: 48-52.

In some aspects, the monoclonal antibody that binds to CCR8 comprises the VH sequence of SEQ ID NO: 47 and the VL sequence of SEQ ID NO: 48.

In some aspects, the monoclonal antibody that binds to CCR8 comprises a heavy chain variable domain (VH) comprising (a) CDR-H1 comprising the amino acid sequence of SEQ ID NO: 4 or SEQ ID NO: 5, (b) CDR-H2 comprising the amino acid sequence of SEQ ID NO: 6, and (c) CDR-H3 comprising the amino acid sequence of SEQ ID NO: 7, and a light chain variable domain (VL) comprising (d) CDR-L1 comprising the amino acid sequence of SEQ ID NO: 1, (e) CDR-L2 comprising the amino acid sequence of SEQ ID NO: 2, and (f) CDR-L3 comprising the amino acid sequence of SEQ ID NO: 3.

In some aspects, the monoclonal antibody that binds to CCR8 binds to CCR8 independent of sulfation of CCR8.

In some aspects, the monoclonal antibody that binds to CCR8 binds to an epitope comprised of one or more of amino acid residues 91-104 and 172-193 of SEQ ID NO: 106.

In some aspects, the monoclonal antibody that binds to CCR8 comprises a sequence selected from the group consisting of: (a) a VH sequence having at least about 95%, at least about 96%, at least about 97%, at least about 98%, or at least about 99% identity to an amino acid sequence selected from the group consisting of SEQ ID NOs: 10-21; (b) a VL sequence having at least about 95%, at least about 96%, at least about 97%, at least about 98%, or at least about 99% identity to an amino acid sequence selected from the group consisting of SEQ ID NOs: 22-25; and (c) a VH sequence as defined in (a) and a VL sequence as defined in (b).

In some aspects, the monoclonal antibody that binds to CCR8 comprises a VH sequence selected from the group consisting of SEQ ID NOs: 10-21 and a VL sequence selected from the group consisting of SEQ ID NOs: 22-25.

In some aspects, the monoclonal antibody that binds to CCR8 comprises a sequence selected from the group consisting of: (a) a VH sequence having at least about 95%, at least about 96%, at least about 97%, at least about 98%, or at least about 99% identity to an amino acid sequence of SEQ ID NO: 21; (b) a VL sequence having at least about 95%, at least about 96%, at least about 97%, at least about 98%, or at least about 99% identity to an amino acid sequence of SEQ ID NO: 24; and (c) a VH sequence as defined in (a) and a VL sequence as defined in (b).

In some aspects, the monoclonal antibody that binds to CCR8 comprises a VH sequence having at least about 95%, at least about 96%, at least about 97%, at least about 98%, or at least about 99% identity to an amino acid sequence of SEQ ID NO: 21 and a VL sequence having at least about 95%, at least about 96%, at least about 97%, at least about 98%, or at least about 99% identity to an amino acid sequence of SEQ ID NO: 24.

In some aspects, the VL comprises a Y21 mutation. In some instances, the Y21 mutation is according to Kabat numbering.

In some aspects, the VH comprises a S73N mutation, a V78L mutation, a T76N mutation, a F91Y mutation, and a P105Q mutation, or a combination thereof. In some instances, the S73N mutation, the V78L mutation, the T76N mutation, the F91 Y mutation, or the P105Q mutation is according to Kabat numbering.

In some aspects, the monoclonal antibody that binds to CCR8 comprises the heavy chain amino acid sequence of SEQ ID NO: 57 and the light chain amino acid sequence of SEQ ID NO: 58.

In some aspects, the monoclonal antibody that binds to CCR8 comprises the heavy chain amino acid sequence of SEQ ID NO: 61 and the light chain amino acid sequence of SEQ ID NO: 58.

In some aspects, the monoclonal antibody that binds to CCR8 comprises the heavy chain amino acid sequence of SEQ ID NO: 112 and the light chain amino acid sequence of SEQ ID NO: 58.

In some aspects, the monoclonal antibody that binds to CCR8 comprises the heavy chain amino acid sequence of SEQ ID NO: 114 and the light chain amino acid sequence of SEQ ID NO: 58.

In some aspects, the monoclonal antibody that binds to CCR8 comprises the VH sequence of SEQ ID NO: 21 and the VL sequence of SEQ ID NO: 24.

In some aspects, the monoclonal antibody that binds to CCR8 comprises a heavy chain variable domain (VH) comprising (a) CDR-H1 comprising the amino acid sequence of SEQ ID NO: 82 or SEQ ID NO: 83, (b) CDR-H2 comprising the amino acid sequence of SEQ ID NO: 84, and (c) CDR-H3 comprising the amino acid sequence of SEQ ID NO: 85, and a light chain variable domain (VL) comprising (d) CDR-L1 comprising the amino acid sequence of SEQ ID NO: 73, (e) CDR-L2 comprising the amino acid sequence of SEQ ID NO: 74, and (f) CDR-L3 comprising the amino acid sequence of SEQ ID NO: 75.

In some aspects, the monoclonal antibody that binds to CCR8 comprises a sequence selected from the group consisting of: (a) a VH sequence having at least about 95%, at least about 96%, at least about 97%, at least about 98%, or at least about 99% identity to an amino acid sequence of SEQ ID NO: 95; (b) a VL sequence having at least about 95%, at least about 96%, at least about 97%, at least about 98%, or at least about 99% identity to an amino acid sequence of SEQ ID NO: 94; and (c) a VH sequence as defined in (a) and a VL sequence as defined in (b).

In some aspects, the monoclonal antibody that binds to CCR8 comprises the VH sequence of SEQ ID NO: 95 and the VL sequence of SEQ ID NO: 94.

In some aspects, the monoclonal antibody that binds to CCR8 comprises the heavy chain amino acid sequence of SEQ ID NO: 101 and the light chain amino acid sequence of SEQ ID NO: 100.

In some aspects, the monoclonal antibody that binds to CCR8 comprises the heavy chain amino acid sequence of SEQ ID NO: 115 and the light chain amino acid sequence of SEQ ID NO: 100.

In some aspects, the monoclonal antibody that binds to CCR8 comprises a heavy chain variable domain (VH) comprising (a) CDR-H1 comprising the amino acid sequence of SEQ ID NO: 86 or SEQ ID NO: 87, (b) CDR-H2 comprising the amino acid sequence of SEQ ID NO: 88, and (c) CDR-H3 comprising the amino acid sequence of SEQ ID NO: 89, and a light chain variable domain (VL) comprising (d) CDR-L1 comprising the amino acid sequence of SEQ ID NO: 76, (e) CDR-L2 comprising the amino acid sequence of SEQ ID NO: 77, and (f) CDR-L3 comprising the amino acid sequence of SEQ ID NO: 78.

In some aspects, the monoclonal antibody that binds to CCR8 comprises a sequence selected from the group consisting of: (a) a VH sequence having at least about 95%, at least about 96%, at least about 97%, at least about 98%, or at least about 99% identity to an amino acid sequence of SEQ ID NO: 97; (b) a VL sequence having at least about 95%, at least about 96%, at least about 97%, at least about 98%, or at least about 99% identity to an amino acid sequence of SEQ ID NO: 96; and (c) a VH sequence as defined in (a) and a VL sequence as defined in (b).

In some aspects, the monoclonal antibody that binds to CCR8 comprises the VH sequence of SEQ ID NO: 97 and the VL sequence of SEQ ID NO: 96.

In some aspects, the monoclonal antibody that binds to CCR8 comprises the heavy chain amino acid sequence of SEQ ID NO: 103 and the light chain amino acid sequence of SEQ ID NO: 102.

In some aspects, the monoclonal antibody that binds to CCR8 comprises the heavy chain amino acid sequence of SEQ ID NO: 116 and the light chain amino acid sequence of SEQ ID NO: 102.

In some aspects, the monoclonal antibody that binds to CCR8 comprises a heavy chain variable domain (VH) comprising (a) CDR-H1 comprising the amino acid sequence of SEQ ID NO: 90 or SEQ ID NO: 91, (b) CDR-H2 comprising the amino acid sequence of SEQ ID NO: 92, and (c) CDR-H3 comprising the amino acid sequence of SEQ ID NO: 93, and a light chain variable domain (VL) comprising (d) CDR-L1 comprising the amino acid sequence of SEQ ID NO: 79, (e) CDR-L2 comprising the amino acid sequence of SEQ ID NO: 80, and (f) CDR-L3 comprising the amino acid sequence of SEQ ID NO: 81.

In some aspects, the monoclonal antibody that binds to CCR8 comprises a sequence selected from the group consisting of: (a) a VH sequence having at least about 95%, at least about 96%, at least about 97%, at least about 98%, or at least about 99% identity to an amino acid sequence of SEQ ID NO: 99; (b) a VL sequence having at least about 95%, at least about 96%, at least about 97%, at least about 98%, or at least about 99% identity to an amino acid sequence of SEQ ID NO: 98; and (c) a VH sequence as defined in (a) and a VL sequence as defined in (b).

In some aspects, the monoclonal antibody that binds to CCR8 comprises the VH sequence of SEQ ID NO: 99 and the VL sequence of SEQ ID NO: 98.

In some aspects, the monoclonal antibody that binds to CCR8 comprises the heavy chain amino acid sequence of SEQ ID NO: 105 and the light chain amino acid sequence of SEQ ID NO: 104.

In some aspects, the monoclonal antibody that binds to CCR8 comprises the heavy chain amino acid sequence of SEQ ID NO: 117 and the light chain amino acid sequence of SEQ ID NO: 104.

In some aspects, the monoclonal antibody that binds to CCR8 binds to CCR8 independent of sulfation of CCR8.

In some aspects, the antibody binds to an epitope comprised of one or more of amino acid residues 2-6 of SEQ ID NO: 106.

In some aspects, the antibody binds to binds to an epitope comprised of one or more of amino acid residues 91-104 and 172-193 of SEQ ID NO: 106.

In some aspects, the monoclonal antibody that binds to CCR8 comprises a heavy chain variable domain (VH) comprising (a) CDR-H1 comprising the amino acid sequence of SEQ ID NO: 65 or SEQ ID NO: 66, (b) CDR-H2 comprising the amino acid sequence of SEQ ID NO: 67 and (c) CDR-H3 comprising the amino acid sequence of SEQ ID NO: 68, and a light chain variable domain (VL) comprising (d) CDR-L1 comprising the amino acid sequence of SEQ ID NO: 62, (e) CDR-L2 comprising the amino acid sequence of SEQ ID NO: 63, and (f) CDR-L3 comprising the amino acid sequence of SEQ ID NO: 64.

In some aspects, the monoclonal antibody that binds to CCR8 comprises a sequence selected from the group consisting of: (a) a VH sequence having at least about 95%, at least about 96%, at least about 97%, at least about 98%, or at least about 99% identity to an amino acid sequence of SEQ ID NO: 70; (b) a VL sequence having at least about 95%, at least about 96%, at least about 97%, at least about 98%, or at least about 99% identity to an amino acid sequence of SEQ ID NO: 69; and (c) a VH sequence as defined in (a) and a VL sequence as defined in (b).

In some aspects, the monoclonal antibody that binds to CCR8 comprises the VH sequence of SEQ ID NO: 70 and the VL sequence of SEQ ID NO: 69.

In some aspects, the monoclonal antibody that binds to CCR8 comprises the heavy chain amino acid sequence of SEQ ID NO: 72 and the light chain amino acid sequence of SEQ ID NO: 71.

In some aspects, the monoclonal antibody that binds to CCR8 is a human antibody.

In some aspects, the monoclonal antibody that binds to CCR8 is a humanized antibody.

In some aspects, the monoclonal antibody that binds to CCR8 is a chimeric antibody.

In some aspects, the monoclonal antibody that binds to CCR8 is an antibody fragment that binds to CCR8.

In some aspects, the monoclonal antibody that binds to CCR8 is a full-length antibody.

In some aspects, the monoclonal antibody that binds to CCR8 is a full-length IgG1 antibody.

In some aspects, the monoclonal antibody that binds to CCR8 comprises a IgG1 constant domain comprising the amino acid sequence of SEQ ID NO: 53 or SEQ ID NO: 59.

In some aspects, the monoclonal antibody that binds to CCR8 comprises a kappa constant domain comprising the amino acid sequence of SEQ ID NO: 54.

In some aspects, the monoclonal antibody that binds to CCR8 binds to CCR8 with a binding affinity (K_{d}) of from about 1×10⁻¹² M to about 1×10⁻¹¹ M.

In some aspects, the CCR8 is a human CCR8.

In some aspects, the monoclonal antibody that binds to CCR8 is afucosylated.

In some aspects, the proportion of afucosylation is between about 80% to about 95%.

In some aspects, the regulatory T cells present in the tumor microenvironment of the locally advanced, recurrent, or metastatic solid tumor malignancy are depleted.

In some aspects, the regulatory T cells outside of the tumor microenvironment of the locally advanced, recurrent, or metastatic solid tumor malignancy are depleted.

In some aspects, the subject is a human.

An antibody as described herein (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies as described herein would be formulated, dosed, and administered in a fashion consistent with good medical practice. Factors for consideration in this context include the particular disorder being treated, the particular subject species being treated, the clinical condition of the subject, the cause of the disorder, the site of delivery of the agent, the method of administration, the scheduling of administration, and other factors known to medical practitioners. The antibody need not be, but is optionally formulated with, one or more agents currently used to treat the disorder in question. The effective amount of such other agents depends on the amount of antibody present in the pharmaceutical composition, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

The antibody is suitably administered to the subject at one time or over a series of treatments. For repeated administrations over several days or longer, depending on the condition, the treatment would generally be sustained until a desired suppression of disease symptoms occurs. However, other dosage regimens may be useful. The progress of this therapy can be monitored by conventional techniques and assays.

### B. Exemplary Anti-CCR8 Antibodies

Any of the anti-CCR8 antibodies may be used in any of the methods and compositions for use disclosed herein, e.g., as described above in Section A.

In one aspect, the present disclosure provides antibodies that bind to CCR8. In one aspect, the antibodies provided are isolated antibodies that bind to CCR8. In one aspect, the present disclosure provides antibodies that specifically bind to CCR8. In certain aspects, an anti-CCR8 antibody binds to an epitope comprised of one or more of amino acid residues 2-6 of SEQ ID NO: 106. In certain aspects, an anti-CCR8 antibody binds to an epitope comprised of one or more of the amino acid residues 91-104 and 172-193 of SEQ ID NO: 106. In certain aspects, the CCR8 is a human CCR8, a mouse CCR8 or a cyno CCR8. In certain aspects, the CCR8 is a human CCR8. In one aspect, the present disclosure provides antibodies that bind to CCR8 independent of tyrosine sulfation of CCR8 (“sulfation independent”).

Exemplary antibodies disclosed herein that are sulfation independent include Ab4 and Ab5, further described in more detail below.

In certain aspects, an antibody provided herein has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain aspects, the antibody that binds to CCR8 has a K_{D} of from about 1×10⁻¹² M to about 1×10⁻¹⁰ M, from about 1×10⁻¹² M to about 1×10⁻¹¹ M, or from about 1×10⁻¹¹ M to about 5×10⁻¹¹ M. In certain aspects, the antibody that binds to CCR8 has a K_{D} of about 2×10⁻¹¹ M. In certain aspects, the antibody that binds to CCR8 has a K_{D} of about 5×10⁻¹² M. In one aspect, K_{D} is measured using radiolabeled IgGs and CHO cell lines stably expressing antigen. Stable CHO cells expressing the antigen are seeded in cold binding buffer (Opti-MEM+2% fetal bovine serum (FBS)+50 mM HEPES, pH 7.2+0.1% Sodium Azide) at 50,000 cells per well. A fixed concentration of ¹²⁵I radiolabeled antigen of interest using the NEX244 IODOGEN® method (Perkin Elmer) is mixed with serially diluted antibodies of interest starting at 20 nM or 50 nM. The antibody mixture is added to the cells and incubated at room temperature for 12 hours under gentle agitation. The cells and antibodies are then transferred to Millipore multiscreen filter plates. The filter plates are washed 4 times with 250 μL of cold binding buffer and dried for at least 30 minutes and the filters are punched into 5 mL polystyrene tubes. The radioactivity is measured using a Perkin Elmer Wallac WIZARD®2470 Gamma Counter set at 1 count per minute with 0.8 counting efficiency. The data are fitted using the heterologous one site-fit Ki competitive binding model in GraphPad PRISM®.

In certain aspects, an antibody provided herein exhibits mean clearance after a single 10 mg/kg dose administered intravenously on day 1 of between about 3 to about 5 mL/day/kg over a 35-day period. For example, but not by way of limitation, such administration can comprise a single 10 mg/kg IV bolus of mAb. Blood samples for analysis can be collected, e.g., at 0.25, 2, and 6 hours, and 1, 2, 7, 14, 21, 28, and 35 days post-dose, and serum can be assayed for concentrations of mAb using a variety of means, e.g., a qualified ELISA analytical method. In certain aspects, the administration is to a mammal. In certain aspects the administration is to a primate. In certain aspects, the administration is to a non-human primate, e.g., a cyno. In certain aspects, the administration is to a human.

### (i) Embodiments of Ab5 and Fragments Thereof.

In one aspect, the present disclosure provides an anti-CCR8 antibody comprising at least one, at least two, at least three, at least four, at least five, or all six CDRs selected from the group consisting of (a) CDR-H1 comprising the amino acid sequence of SEQ ID NO: 29 or SEQ ID NO: 30, (b) CDR-H2 comprising the amino acid sequence of SEQ ID NO: 31, (c) CDR-H3 comprising the amino acid sequence of SEQ ID NO: 32, (d) CDR-L1 comprising the amino acid sequence of SEQ ID NO: 26, (e) CDR-L2 comprising the amino acid sequence of SEQ ID NO: 27, and (f) CDR-L3 comprising the amino acid sequence of SEQ ID NO: 28. In certain aspects, the anti-CCR8 antibody comprises all six of the aforementioned CDRs. In certain aspects, the anti-CCR8 antibody is a full-length antibody. In certain aspects, the anti-CCR8 antibody is a full-length antibody which binds to human CCR8. In certain aspects, the anti-CCR8 antibody is a full-length antibody which binds to both human CCR8 and cyno CCR8. In certain aspects, the anti-CCR8 antibody is a full-length antibody which binds to human CCR8 and is a human antibody. In certain aspects, the anti-CCR8 antibody is a full-length antibody which binds to human CCR8 and is a humanized antibody. In certain aspects, the anti-CCR8 antibody is a full-length antibody which binds to human CCR8 and is a chimeric antibody.

In one aspect, the present disclosure provides an antibody comprising at least one, at least two, or all three VH CDR sequences selected from the group consisting of (a) CDR-H1 comprising the amino acid sequence of SEQ ID NO: 29 or SEQ ID NO: 30, (b) CDR-H2 comprising the amino acid sequence of SEQ ID NO: 31, and (c) CDR-H3 comprising the amino acid sequence of SEQ ID NO: 32. In one aspect, the antibody comprises CDR-H3 comprising the amino acid sequence of SEQ ID NO: 32. In another aspect, the antibody comprises CDR-H3 comprising the amino acid sequence of SEQ ID NO: 32 and CDR-L3 comprising the amino acid sequence of SEQ ID NO: 28. In a further aspect, the antibody comprises CDR-H3 comprising the amino acid sequence of SEQ ID NO: 32, CDR-L3 comprising the amino acid sequence of SEQ ID NO: 28, and CDR-H2 comprising the amino acid sequence of SEQ ID NO: 31. In a further aspect, the antibody comprises (a) CDR-H1 comprising the amino acid sequence of SEQ ID NO: 29 or SEQ ID NO: 30, (b) CDR-H2 comprising the amino acid sequence of SEQ ID NO: 31, and (c) CDR-H3 comprising the amino acid sequence of SEQ ID NO: 32.

In another aspect, the present disclosure provides an antibody comprising at least one, at least two, or all three VL CDR sequences selected from the group consisting of (a) CDR-L1 comprising the amino acid sequence of SEQ ID NO: 26; (b) CDR-L2 comprising the amino acid sequence of SEQ ID NO: 27; and (c) CDR-L3 comprising the amino acid sequence of SEQ ID NO: 28. In one aspect, the antibody comprises (a) CDR-L1 comprising the amino acid sequence of SEQ ID NO: 26; (b) CDR-L2 comprising the amino acid sequence of SEQ ID NO: 27; and (c) CDR-L3 comprising the amino acid sequence of SEQ ID NO: 28.

In another aspect, an antibody as described herein comprises (a) a VH domain comprising at least one, at least two, or all three VH CDR sequences selected from the group consisting of (i) CDR-H1 comprising the amino acid sequence of SEQ ID NO: 29 or SEQ ID NO: 30, (ii) CDR-H2 comprising the amino acid sequence of SEQ ID NO: 31, and (iii) CDR-H3 comprising the amino acid sequence of SEQ ID NO: 32; and (b) a VL domain comprising at least one, at least two, or all three VL CDR sequences selected from the group consisting of (i) CDR-L1 comprising the amino acid sequence of SEQ ID NO: 26; (ii) CDR-L2 comprising the amino acid sequence of SEQ ID NO: 27; and (iii) CDR-L3 comprising the amino acid sequence of SEQ ID NO: 28. In certain aspects, the anti-CCR8 antibody comprises all six of the aforementioned CDRs. In certain aspects, the anti-CCR8 antibody is a full-length antibody. In certain aspects, the anti-CCR8 antibody is a full-length antibody which binds to human CCR8. In certain aspects, the anti-CCR8 antibody is a full-length antibody which binds to both human CCR8 and cyno CCR8. In certain aspects, the anti-CCR8 antibody is a full-length antibody which binds to human CCR8 and is a human antibody. In certain aspects, the anti-CCR8 antibody is a full-length antibody which binds to human CCR8 and is a humanized antibody. In certain aspects, the anti-CCR8 antibody is a full-length antibody which binds to human CCR8 and is a chimeric antibody.

In another aspect, the present disclosure provides an antibody comprising (a) CDR-H1 comprising the amino acid sequence of SEQ ID NO: 29 or SEQ ID NO: 30, (b) CDR-H2 comprising the amino acid sequence of SEQ ID NO: 31, and (c) CDR-H3 comprising the amino acid sequence of SEQ ID NO: 32, and a light chain variable domain (VL) comprising (d) CDR-L1 comprising the amino acid sequence of SEQ ID NO: 26, (e) CDR-L2 comprising the amino acid sequence of SEQ ID NO: 27, and (f) CDR-L3 comprising the amino acid sequence of SEQ ID NO: 28.

In another aspect, an anti-CCR8 antibody comprises one or more of the CDR sequences of the VH sequence selected from the group consisting of SEQ ID NOs: 35-47. In another embodiment, an anti-CCR8 antibody comprises one or more of the CDR sequences of the VL sequence selected from the group consisting of SEQ ID NOs: 48-52. In another embodiment, an anti-CCR8 antibody comprises the CDR sequences of the VH sequence selected from the group consisting of SEQ ID NOs: 35-47 and the CDR sequences of the VL sequence selected from the group consisting of SEQ ID NOs: 48-52.

In another aspect, an anti-CCR8 antibody comprises one or more of the CDR sequences of the VH sequence of SEQ ID NO: 47. In another embodiment, an anti-CCR8 antibody comprises one or more of the CDR sequences of the VL sequence of SEQ ID NO: 48. In another embodiment, an anti-CCR8 antibody comprises the CDR sequences of the VH sequence of SEQ ID NO: 47. In another embodiment, an anti-CCR8 antibody comprises one or more of the CDR sequences of the VL sequence of SEQ ID NO: 48.

In a further aspect, an anti-CCR8 antibody comprises the CDR-H1, CDR-H2 and CDR-H3 amino acid sequences of the VH domain selected from the group consisting of SEQ ID NOs: 35-47 and the CDR-L1, CDR-L2 and CDR-L3 amino acid sequences of the VL domain selected from the group consisting of SEQ ID NOs: 48-52.

In a further aspect, an anti-CCR8 antibody comprises the CDR-H1, CDR-H2 and CDR-H3 amino acid sequences of the VH domain of SEQ ID NO: 47 and the CDR-L1, CDR-L2 and CDR-L3 amino acid sequences of the VL domain of SEQ ID NO: 48.

In one aspect, an anti-CCR8 antibody comprises one or more of the heavy chain CDR amino acid sequences of the VH domain selected from the group consisting of SEQ ID NOs: 35-47 and a framework of at least 85%, 86%, 87%, 88%, 89%, 90%, 91%, 92%, 93%, 94%, 95%, 96%, 97%, 98%, 99% sequence identity to the framework amino acid sequence of the VH domain selected from the group consisting of SEQ ID NOs: 35-47. In one aspect, the anti-CCR8 antibody comprises the three heavy chain CDR amino acid sequences of the VH domain selected from the group consisting of SEQ ID NOs: 35-47 and a framework of at least 85%, 86%, 87%, 88%, 89%, 90%, 91%, 92%, 93%, 94%, 95%, 96%, 97%, 98%, 99% sequence identity to the framework amino acid sequence of the VH domain selected from the group consisting of SEQ ID NOs: 35-47. In one aspect, the anti-CCR8 antibody comprises the three heavy chain CDR amino acid sequences of the VH domain of selected from the group consisting of SEQ ID NOs: 35-47 and a framework of at least 95% sequence identity to the framework amino acid sequence of the VH domain selected from the group consisting of SEQ ID NOs: 35-47. In another aspect, the anti-CCR8 antibody comprises the three heavy chain CDR amino acid sequences of the VH domain of selected from the group consisting of SEQ ID NOs: 35-47 and a framework of at least of at least 98% sequence identity to the framework amino acid sequence of the VH domain of selected from the group consisting of SEQ ID NOs: 35-47.

In one aspect, an anti-CCR8 antibody comprises one or more of the heavy chain CDR amino acid sequences of the VH domain of SEQ ID NO: 47 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 47. In one aspect, the anti-CCR8 antibody comprises the three heavy chain CDR amino acid sequences of the VH domain of SEQ ID NO: 47 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 47. In one aspect, the anti-CCR8 antibody comprises the three heavy chain CDR amino acid sequences of the VH domain of SEQ ID NO: 47 and a framework of at least 95% sequence identity to the framework amino acid sequence of the VH domain of SEQ ID NO: 47. In another aspect, the anti-CCR8 antibody comprises the three heavy chain CDR amino acid sequences of the VH domain of SEQ ID NO: 47 and a framework of at least of at least 98% sequence identity to the framework amino acid sequence of the VH domain of SEQ ID NO: 47.

In one aspect, an anti-CCR8 antibody comprises one or more of the light chain CDR amino acid sequences of the VL domain selected from the group consisting of SEQ ID NOs: 48-52 and a framework of at least 85%, 86%, 87%, 88%, 89%, 90%, 91%, 92%, 93%, 94%, 95%, 96%, 97%, 98%, 99% sequence identity to the framework amino acid sequence of the VL domain selected from the group consisting of SEQ ID NOs: 48-52. In one aspect, the anti-CCR8 antibody comprises the three light chain CDR amino acid sequences of the VL domain selected from the group consisting of SEQ ID NOs: 48-52 and a framework of at least 85%, 86%, 87%, 88%, 89%, 90%, 91%, 92%, 93%, 94%, 95%, 96%, 97%, 98%, 99% sequence identity to the framework amino acid sequence of the VL domain selected from the group consisting of SEQ ID NOs: 48-52. In one aspect, the anti-CCR8 antibody comprises the three light chain CDR amino acid sequences of the VL domain selected from the group consisting of SEQ ID NOs: 48-52 and a framework of at least 95% sequence identity to the framework amino acid sequence of the VL domain selected from the group consisting of SEQ ID NOs: 48-52. In another aspect, the anti-CCR8 antibody comprises the three light chain CDR amino acid sequences of the VL domain selected from the group consisting of SEQ ID NOs: 48-52 and a framework of at least particularly of at least 98% sequence identity to the framework amino acid sequence of the VL domain selected from the group consisting of SEQ ID NOs: 48-52.

In one aspect, an anti-CCR8 antibody comprises one or more of the light chain CDR amino acid sequences of the VL domain of SEQ ID NO: 48 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 48. In one aspect, the anti-CCR8 antibody comprises the three light chain CDR amino acid sequences of the VL domain of SEQ ID NO: 48 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 48. In one aspect, the anti-CCR8 antibody comprises the three light chain CDR amino acid sequences of the VL domain of SEQ ID NO: 48 and a framework of at least 95% sequence identity to the framework amino acid sequence of the VL domain of SEQ ID NO: 48. In another aspect, the anti-CCR8 antibody comprises the three light chain CDR amino acid sequences of the VL domain of SEQ ID NO: 48 and a framework of at least particularly of at least 98% sequence identity to the framework amino acid sequence of the VL domain of SEQ ID NO: 48.

In one aspect, the anti-CCR8 antibody comprises (a) CDR-H1 comprising the amino acid sequence of SEQ ID NO: 29 or SEQ ID NO: 30, (b) CDR-H2 comprising the amino acid sequence of SEQ ID NO: 31, (c) CDR-H3 comprising the amino acid sequence of SEQ ID NO: 32, (d) CDR-L1 comprising the amino acid sequence of SEQ ID NO: 26, (e) CDR-L2 comprising the amino acid sequence of SEQ ID NO: 27, and (f) CDR-L3 comprising the amino acid sequence of SEQ ID NO: 28, and a VH domain having at least 90%, 91%, 92%, 93%, 94%, 95%, 96%, 97%, 98%, 99%, or 100% sequence identity to an amino acid sequence selected from the group consisting of SEQ ID NOs: 35-47, and a VL domain having at least 90%, 91%, 92%, 93%, 94%, 95%, 96%, 97%, 98%, 99%, or 100% sequence identity to an amino acid sequence selected from the group consisting of SEQ ID NOs: 48-52. In one aspect, the VH domain has at least 95% sequence identity to an amino acid sequence selected from the group consisting of SEQ ID NOs: 35-47. In one aspect, the VL domain has at least 95% sequence identity to an amino acid sequence selected from the group consisting of SEQ ID NOs: 48-52. In one aspect, the antibody binds to CCR8 having a dissociation constant (K_{D}) that is up to 10-fold reduced or up to 10-fold increased when compared to the dissociation constant (K_{D}) of an antibody comprising a VH sequence selected from the group consisting of SEQ ID NOs: 35-47 and a VL sequence selected from the group consisting of SEQ ID NOs: 48-52.

In one aspect, the anti-CCR8 antibody comprises (a) CDR-H1 comprising the amino acid sequence of SEQ ID NO: 29 or SEQ ID NO: 30, (b) CDR-H2 comprising the amino acid sequence of SEQ ID NO: 31, (c) CDR-H3 comprising the amino acid sequence of SEQ ID NO: 32, (d) CDR-L1 comprising the amino acid sequence of SEQ ID NO: 26, (e) CDR-L2 comprising the amino acid sequence of SEQ ID NO: 27, and (f) CDR-L3 comprising the amino acid sequence of SEQ ID NO: 28, and a VH domain having at least 90%, 91%, 92%, 93%, 94%, 95%, 96%, 97%, 98%, 99%, or 100% sequence identity to the amino acid sequence of SEQ ID NO: 47, and a VL domain having at least 90%, 91%, 92%, 93%, 94%, 95%, 96%, 97%, 98%, 99%, or 100% sequence identity to the amino acid sequence of SEQ ID NO: 48. In one aspect, the VH domain has at least 95% sequence identity to the amino acid sequence of SEQ ID NO: 47. In one aspect, the VL domain has at least 95% sequence identity to the amino acid sequence of SEQ ID NO: 48. In one aspect, the antibody binds to CCR8 having a dissociation constant (K_{D}) that is up to 10-fold reduced or up to 10-fold increased when compared to the dissociation constant (K_{D}) of an antibody comprising a VH sequence of SEQ ID NO: 47 and a VL sequence of SEQ ID NO: 48.

In another aspect, an anti-CCR8 antibody comprises a heavy chain variable domain (VH) sequence having at least 90%, 91%, 92%, 93%, 94%, 95%, 96%, 97%, 98%, 99%, or 100% sequence identity to an amino acid sequence selected from the group consisting of SEQ ID NOs: 35-47. In one aspect, an anti-CCR8 antibody comprises a heavy chain variable domain (VH) sequence having at least 95%, sequence identity to an amino acid sequence selected from the group consisting of SEQ ID NOs: 35-47. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-CCR8 antibody comprising that sequence retains the ability to bind to CCR8. In certain aspects, a total of 1 to 10 amino acids have been substituted, inserted and/or deleted in an amino acid sequence selected from the group consisting of SEQ ID NOs: 35-47. In certain aspects, substitutions, insertions, or deletions occur in regions outside the CDRs (i.e., in the FRs). Optionally, the anti-CCR8 antibody comprises the VH sequence selected from the group consisting of SEQ ID NOs: 35-47, including post-translational modifications of that sequence. In a particular aspect, the VH comprises one, two or three CDRs selected from: (a) CDR-H1 comprising the amino acid sequence of SEQ ID NO: 29 or SEQ ID NO: 30, (b) CDR-H2 comprising the amino acid sequence of SEQ ID NO: 31, (c) CDR-H3 comprising the amino acid sequence of SEQ ID NO: 32. In another aspect, an anti-CCR8 antibody is provided, wherein the antibody comprises a light chain variable domain (VL) sequence having at least 90%, 91%, 92%, 93%, 94%, 95%, 96%, 97%, 98%, 99%, or 100% sequence identity to an amino acid sequence selected from the group consisting of SEQ ID NOs: 48-52. In one aspect, an anti-CCR8 antibody comprises a light chain variable domain (VL) sequence having at least 95% sequence identity to an amino acid sequence selected from the group consisting of SEQ ID NOs: 48-52. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-CCR8 antibody comprising that sequence retains the ability to bind to CCR8. In certain aspects, a total of 1 to 10 amino acids have been substituted, inserted and/or deleted in the amino acid sequence selected from the group consisting of SEQ ID NOs: 48-52. In certain aspects, the substitutions, insertions, or deletions occur in regions outside the CDRs (i.e., in the FRs). Optionally, the anti-CCR8 antibody comprises the VL sequence selected from the group consisting of SEQ ID NOs: 48-52, including post-translational modifications of that sequence. In a particular aspect, the VL comprises one, two or three CDRs selected from: (a) CDR-L1 comprising the amino acid sequence of SEQ ID NO: 26, (b) CDR-L2 comprising the amino acid sequence of SEQ ID NO: 27, and (c) CDR-L3 comprising the amino acid sequence of SEQ ID NO: 28.

In another aspect, an anti-CCR8 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 47. In one aspect, an anti-CCR8 antibody comprises a heavy chain variable domain (VH) sequence having at least 95%, sequence identity to the amino acid sequence of SEQ ID NO: 47. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-CCR8 antibody comprising that sequence retains the ability to bind to CCR8. In certain aspects, a total of 1 to 10 amino acids have been substituted, inserted and/or deleted in the amino acid sequence of SEQ ID NO: 47. In certain aspects, substitutions, insertions, or deletions occur in regions outside the CDRs (i.e., in the FRs). Optionally, the anti-CCR8 antibody comprises the VH sequence of SEQ ID NO: 47, including post-translational modifications of that sequence. In a particular aspect, the VH comprises one, two or three CDRs selected from: (a) CDR-H1 comprising the amino acid sequence of SEQ ID NO: 29 or SEQ ID NO: 30, (b) CDR-H2 comprising the amino acid sequence of SEQ ID NO: 31, (c) CDR-H3 comprising the amino acid sequence of SEQ ID NO: 32. In another aspect, an anti-CCR8 antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO: 48. In one aspect, an anti-CCR8 antibody comprises a light chain variable domain (VL) sequence having at least 95% sequence identity to the amino acid sequence of SEQ ID NO: 48. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-CCR8 antibody comprising that sequence retains the ability to bind to CCR8. In certain aspects, a total of 1 to 10 amino acids have been substituted, inserted and/or deleted in the amino acid sequence of SEQ ID NO: 48. In certain aspects, the substitutions, insertions, or deletions occur in regions outside the CDRs (i.e., in the FRs). Optionally, the anti-CCR8 antibody comprises the VL sequence of SEQ ID NO: 48, including post-translational modifications of that sequence. In a particular aspect, the VL comprises one, two or three CDRs selected from: (a) CDR-L1 comprising the amino acid sequence of SEQ ID NO: 26, (b) CDR-L2 comprising the amino acid sequence of SEQ ID NO: 27, and (c) CDR-L3 comprising the amino acid sequence of SEQ ID NO: 28.

In another aspect, an anti-CCR8 antibody is provided, wherein the antibody comprises a VH sequence as in any of the aspects provided above, and a VL sequence as in any of the aspects provided above. In one aspect, the antibody comprises the VH sequence selected from the group consisting of SEQ ID NOs: 35-47 and the VL sequence selected from the group consisting of SEQ ID NOs: 48-52, including post-translational modifications of those sequences. In one aspect, the antibody comprises the VH sequence of SEQ ID NO: 47 and the VL sequence of SEQ ID NO: 48, including post-translational modifications of those sequences.

In one aspect, the VL sequence comprises a V4M mutation, a P43A mutation, a F46L mutation, a C90Q mutation, or a combination thereof (e.g., according to Kabat numbering). In one aspect, the VH comprises a G49S mutation, a K71R mutation, a S73N mutation, or a combination thereof (e.g., according to Kabat numbering).

In another aspect, an anti-CCR8 antibody is provided, wherein the antibody comprises a IgG1 constant domain comprising the amino acid sequence of SEQ ID NO: 53 or SEQ ID NO: 59. In one aspect, the antibody comprises a kappa constant domain comprising the amino acid sequence of SEQ ID NO: 54. In another aspect, an anti-CCR8 antibody is provided, wherein the antibody comprises (a) a IgG1 constant domain comprising the amino acid sequence of SEQ ID NO: 53 or SEQ ID NO: 59, and (b) a kappa constant domain comprising the amino acid sequence of SEQ ID NO: 54.

In another aspect, an anti-CCR8 antibody is provided, wherein the antibody comprises a heavy chain variable domain (VH) comprising (a) CDR-H1 comprising the amino acid sequence of SEQ ID NO: 29 or SEQ ID NO: 30, (b) CDR-H2 comprising the amino acid sequence of SEQ ID NO: 31, and (c) CDR-H3 comprising the amino acid sequence of SEQ ID NO: 32, and a light chain variable domain (VL) comprising (d) CDR-L1 comprising the amino acid sequence of SEQ ID NO: 26, (e) CDR-L2 comprising the amino acid sequence of SEQ ID NO: 27, and (f) CDR-L3 comprising the amino acid sequence of SEQ ID NO: 28. In one aspect, the anti-CCR8 antibody comprises a VH sequence of SEQ ID NO: 47 and a VL sequence of SEQ ID NO: 48.

In one aspect, the anti-CCR8 antibody comprises a heavy chain of SEQ ID NO: 55 and a light chain of SEQ ID NO: 56.

In one aspect, the anti-CCR8 antibody comprises a heavy chain of SEQ ID NO: 60 and a light chain of SEQ ID NO: 56.

In another aspect of any of the above-described embodiments, an anti-CCR8 antibody is provided, wherein the heavy chain of the antibody comprises a shortened C-terminus in which one or two of the C terminal amino acid residues have been removed. In one aspect, the C-terminus of the heavy chain is a shortened C-terminus ending PG. In one aspect, the anti-CCR8 antibody comprises a heavy chain of SEQ ID NO: 111 and a light chain of SEQ ID NO: 56. In one aspect, the anti-CCR8 antibody comprises a heavy chain of SEQ ID NO: 113 and a light chain of SEQ ID NO: 56.

In another aspect of any of the above-described embodiments, an anti-CCR8 antibody is provided, wherein the antibody does not bind to CCR8 ligands. In one aspect, the anti-CCR8 antibody has no CCR8 ligand blocking activity. In one aspect, the anti-CCR8 antibody is a non-neutralizing antibody. In one aspect, the CCR8 ligand is CCL1.

In another aspect of any of the above-described embodiments, an anti-CCR8 antibody is provided, wherein the antibody binds to CCR8 independent of tyrosine sulfation of CCR8 for binding (i.e., sulfation independent).

In another aspect of any of the above-described embodiments, an anti-CCR8 antibody is provided, wherein the antibody is an afucosylated antibody variant. In one aspect, the afucosylated antibody variant has enhanced FcγRIIIa receptor binding. In one aspect, the afucosylated anti-CCR8 antibody variant has enhanced antibody-dependent cellular cytotoxicity (ADCC). In one aspect, the anti-CCR8 afucosylated antibody variant has antibody-dependent cellular phagocytosis (ADCP) activities.

In another aspect of any of the above-described embodiments, an anti-CCR8 antibody is provided, wherein the antibody has improved antibody stability. In one aspect, the anti-CCR8 antibody has low aggregation, good solubility, and/or low viscosity. In certain aspects of any of the above-described embodiments, an anti-CCR8 antibody is provided, wherein the antibody has a K_{D} of from about from about 1×10⁻¹² M to about 1×10⁻¹¹ M. In certain aspects, the antibody that binds to CCR8 has a K_{D} of about 5×10⁻¹² M. In certain aspects, the antibody that binds to CCR8 has a K_{D} of about 4×10⁻¹² M. In certain aspects, the antibody that binds to CCR8 has a K_{D} of about 3×10⁻¹² M.

In one aspect, the anti-CCR8 antibody is named as “hu.Ab5.H13L1” in the present disclosure, which can be fucosylated or afucosylated, which optionally contains one or more heavy chain mutations at G236A and I331E, and which optionally comprises a shortened C-terminus of the heavy chain in which one or two of the C terminal amino acid residues have been removed. In some embodiments, the heavy chain mutations are numbered according to the EU index.

In a further aspect, an anti-CCR8 antibody according to any of the above aspects is a monoclonal antibody, including a chimeric, humanized or human antibody. In one aspect, an anti-CCR8 antibody is an antibody fragment, e.g., a Fv, Fab, Fab′, scFv, diabody, or F(ab′)₂ fragment.

### (ii) Embodiments of Ab4 and Fragments Thereof.

In one aspect, the present disclosure provides an anti-CCR8 antibody comprising at least one, at least two, at least three, at least four, at least five, or all six CDRs selected from the group consisting of (a) CDR-H1 comprising the amino acid sequence of SEQ ID NO: 4 or SEQ ID NO: 5, (b) CDR-H2 comprising the amino acid sequence of SEQ ID NO: 6, (c) CDR-H3 comprising the amino acid sequence of SEQ ID NO: 7, (d) CDR-L1 comprising the amino acid sequence of SEQ ID NO: 1, (e) CDR-L2 comprising the amino acid sequence of SEQ ID NO: 2, and (f) CDR-L3 comprising the amino acid sequence of SEQ ID NO: 3. In certain aspects, the anti-CCR8 antibody comprises all six of the aforementioned CDRs. In certain aspects, the anti-CCR8 antibody is a full-length antibody. In certain aspects, the anti-CCR8 antibody is a full-length antibody which binds to human CCR8. In certain aspects, the anti-CCR8 antibody is a full-length antibody which binds to both human CCR8 and cyno CCR8. In certain aspects, the anti-CCR8 antibody is a full-length antibody which binds to human CCR8 and is a human antibody. In certain aspects, the anti-CCR8 antibody is a full-length antibody which binds to human CCR8 and is a humanized antibody. In certain aspects, the anti-CCR8 antibody is a full-length antibody which binds to human CCR8 and is a chimeric antibody.

In one aspect, the present disclosure provides an antibody comprising at least one, at least two, or all three VH CDR sequences selected from the group consisting of (a) CDR-H1 comprising the amino acid sequence of SEQ ID NO: 4 or SEQ ID NO: 5, (b) CDR-H2 comprising the amino acid sequence of SEQ ID NO: 6, and (c) CDR-H3 comprising the amino acid sequence of SEQ ID NO: 7. In one aspect, the antibody comprises CDR-H3 comprising the amino acid sequence of SEQ ID NO: 7. In another aspect, the antibody comprises CDR-H3 comprising the amino acid sequence of SEQ ID NO: 7 and CDR-L3 comprising the amino acid sequence of SEQ ID NO: 3. In a further aspect, the antibody comprises CDR-H3 comprising the amino acid sequence of SEQ ID NO: 7, CDR-L3 comprising the amino acid sequence of SEQ ID NO: 3, and CDR-H2 comprising the amino acid sequence of SEQ ID NO: 6. In a further aspect, the antibody comprises (a) CDR-H1 comprising the amino acid sequence of SEQ ID NO: 4 or SEQ ID NO: 5, (b) CDR-H2 comprising the amino acid sequence of SEQ ID NO: 6, and (c) CDR-H3 comprising the amino acid sequence of SEQ ID NO: 7.

In another aspect, the present disclosure provides an antibody comprising at least one, at least two, or all three VL CDR sequences selected from the group consisting of (a) CDR-L1 comprising the amino acid sequence of SEQ ID NO: 1; (b) CDR-L2 comprising the amino acid sequence of SEQ ID NO: 2; and (c) CDR-L3 comprising the amino acid sequence of SEQ ID NO: 3. In one aspect, the antibody comprises (a) CDR-L1 comprising the amino acid sequence of SEQ ID NO: 1; (b) CDR-L2 comprising the amino acid sequence of SEQ ID NO: 2; and (c) CDR-L3 comprising the amino acid sequence of SEQ ID NO: 3.

In another aspect, an antibody as described herein comprises (a) a VH domain comprising at least one, at least two, or all three VH CDR sequences selected from the group consisting of (i) CDR-H1 comprising the amino acid sequence of SEQ ID NO: 4 or SEQ ID NO: 5, (ii) CDR-H2 comprising the amino acid sequence of SEQ ID NO: 6, and (iii) CDR-H3 comprising the amino acid sequence of SEQ ID NO: 7; and (b) a VL domain comprising at least one, at least two, or all three VL CDR sequences selected from the group consisting of (i) CDR-L1 comprising the amino acid sequence of SEQ ID NO: 1; (ii) CDR-L2 comprising the amino acid sequence of SEQ ID NO: 2; and (iii) CDR-L3 comprising the amino acid sequence of SEQ ID NO: 3. In certain aspects, the anti-CCR8 antibody comprises all six of the aforementioned CDRs. In certain aspects, the anti-CCR8 antibody is a full-length antibody. In certain aspects, the anti-CCR8 antibody is a full-length antibody which binds to human CCR8. In certain aspects, the anti-CCR8 antibody is a full-length antibody which binds to both human CCR8 and cyno CCR8. In certain aspects, the anti-CCR8 antibody is a full-length antibody which binds to human CCR8 and is a human antibody. In certain aspects, the anti-CCR8 antibody is a full-length antibody which binds to human CCR8 and is a humanized antibody. In certain aspects, the anti-CCR8 antibody is a full-length antibody which binds to human CCR8 and is a chimeric antibody.

In another aspect, the present disclosure provides an antibody comprising (a) CDR-H1 comprising the amino acid sequence of SEQ ID NO: 4 or SEQ ID NO: 5, (b) CDR-H2 comprising the amino acid sequence of SEQ ID NO: 6, and (c) CDR-H3 comprising the amino acid sequence of SEQ ID NO: 7, and a light chain variable domain (VL) comprising (d) CDR-L1 comprising the amino acid sequence of SEQ ID NO: 1, (e) CDR-L2 comprising the amino acid sequence of SEQ ID NO: 2, and (f) CDR-L3 comprising the amino acid sequence of SEQ ID NO: 3.

In another aspect, an anti-CCR8 antibody comprises one or more of the CDR sequences of the VH sequence selected from the group consisting of SEQ ID NOs: 10-21. In another aspect, an anti-CCR8 antibody comprises one or more of the CDR sequences of the VL sequence selected from the group consisting of SEQ ID NOs: 22-25. In another aspect, an anti-CCR8 antibody comprises the CDR sequences of the VH sequence selected from the group consisting of SEQ ID NOs: 10-21 and the CDR sequences of the VL sequence selected from the group consisting of SEQ ID NOs: 22-25.

In another aspect, an anti-CCR8 antibody comprises one or more of the CDR sequences of the VH sequence of SEQ ID NO: 21. In another aspect, an anti-CCR8 antibody comprises one or more of the CDR sequences of the VL sequence of SEQ ID NO: 24. In another aspect, an anti-CCR8 antibody comprises the CDR sequences of the VH sequence of SEQ ID NO: 21. In another aspect, an anti-CCR8 antibody comprises one or more of the CDR sequences of the VL sequence of SEQ ID NO: 24.

In a further aspect, an anti-CCR8 antibody comprises the CDR-H1, CDR-H2 and CDR-H3 amino acid sequences of the VH domain selected from the group consisting of SEQ ID NOs: 10-21 and the CDR-L1, CDR-L2 and CDR-L3 amino acid sequences of the VL domain selected from the group consisting of SEQ ID NOs: 22-25.

In a further aspect, an anti-CCR8 antibody comprises the CDR-H1, CDR-H2 and CDR-H3 amino acid sequences of the VH domain of SEQ ID NO: 21 and the CDR-L1, CDR-L2 and CDR-L3 amino acid sequences of the VL domain of SEQ ID NO: 24.

In one aspect, an anti-CCR8 antibody comprises one or more of the heavy chain CDR amino acid sequences of the VH domain selected from the group consisting of SEQ ID NOs: 10-21 and a framework of at least 85%, 86%, 87%, 88%, 89%, 90%, 91%, 92%, 93%, 94%, 95%, 96%, 97%, 98%, 99% sequence identity to the framework amino acid sequence of the VH domain selected from the group consisting of SEQ ID NOs: 10-21. In one aspect, the anti-CCR8 antibody comprises the three heavy chain CDR amino acid sequences of the VH domain selected from the group consisting of SEQ ID NOs: 10-21 and a framework of at least 85%, 86%, 87%, 88%, 89%, 90%, 91%, 92%, 93%, 94%, 95%, 96%, 97%, 98%, 99% sequence identity to the framework amino acid sequence of the VH domain selected from the group consisting of SEQ ID NOs: 10-21. In one aspect, the anti-CCR8 antibody comprises the three heavy chain CDR amino acid sequences of the VH domain of selected from the group consisting of SEQ ID NOs: 10-21 and a framework of at least 95% sequence identity to the framework amino acid sequence of the VH domain selected from the group consisting of SEQ ID NOs: 10-21. In another aspect, the anti-CCR8 antibody comprises the three heavy chain CDR amino acid sequences of the VH domain of selected from the group consisting of SEQ ID NOs: 10-21 and a framework of at least of at least 98% sequence identity to the framework amino acid sequence of the VH domain of selected from the group consisting of SEQ ID NOs: 10-21.

In one aspect, an anti-CCR8 antibody comprises one or more of the heavy chain CDR amino acid sequences of the VH domain of SEQ ID NO: 21 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 21. In one aspect, the anti-CCR8 antibody comprises the three heavy chain CDR amino acid sequences of the VH domain of SEQ ID NO: 21 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 21. In one aspect, the anti-CCR8 antibody comprises the three heavy chain CDR amino acid sequences of the VH domain of SEQ ID NO: 21 and a framework of at least 95% sequence identity to the framework amino acid sequence of the VH domain of SEQ ID NO: 21. In another aspect, the anti-CCR8 antibody comprises the three heavy chain CDR amino acid sequences of the VH domain of SEQ ID NO: 21 and a framework of at least of at least 98% sequence identity to the framework amino acid sequence of the VH domain of SEQ ID NO: 21.

In one aspect, an anti-CCR8 antibody comprises one or more of the light chain CDR amino acid sequences of the VL domain selected from the group consisting of SEQ ID NOs: 22-25 and a framework of at least 85%, 86%, 87%, 88%, 89%, 90%, 91%, 92%, 93%, 94%, 95%, 96%, 97%, 98%, 99% sequence identity to the framework amino acid sequence of the VL domain selected from the group consisting of SEQ ID NOs: 22-25. In one aspect, the anti-CCR8 antibody comprises the three light chain CDR amino acid sequences of the VL domain selected from the group consisting of SEQ ID NOs: 22-25 and a framework of at least 85%, 86%, 87%, 88%, 89%, 90%, 91%, 92%, 93%, 94%, 95%, 96%, 97%, 98%, 99% sequence identity to the framework amino acid sequence of the VL domain selected from the group consisting of SEQ ID NOs: 22-25. In one aspect, the anti-CCR8 antibody comprises the three light chain CDR amino acid sequences of the VL domain selected from the group consisting of SEQ ID NOs: 22-25 and a framework of at least 95% sequence identity to the framework amino acid sequence of the VL domain selected from the group consisting of SEQ ID NOs: 22-25. In another aspect, the anti-CCR8 antibody comprises the three light chain CDR amino acid sequences of the VL domain selected from the group consisting of SEQ ID NOs: 22-25 and a framework of at least particularly of at least 98% sequence identity to the framework amino acid sequence of the VL domain selected from the group consisting of SEQ ID NOs: 22-25.

In one aspect, an anti-CCR8 antibody comprises one or more of the light chain CDR amino acid sequences of the VL domain of SEQ ID NO: 24 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 24. In one aspect, the anti-CCR8 antibody comprises the three light chain CDR amino acid sequences of the VL domain of SEQ ID NO: 24 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 24. In one aspect, the anti-CCR8 antibody comprises the three light chain CDR amino acid sequences of the VL domain of SEQ ID NO: 24 and a framework of at least 95% sequence identity to the framework amino acid sequence of the VL domain of SEQ ID NO: 24. In another aspect, the anti-CCR8 antibody comprises the three light chain CDR amino acid sequences of the VL domain of SEQ ID NO: 24 and a framework of at least particularly of at least 98% sequence identity to the framework amino acid sequence of the VL domain of SEQ ID NO: 24.

In one aspect, the anti-CCR8 antibody comprises (a) CDR-H1 comprising the amino acid sequence of SEQ ID NO: 4 or SEQ ID NO: 5, (b) CDR-H2 comprising the amino acid sequence of SEQ ID NO: 6, (c) CDR-H3 comprising the amino acid sequence of SEQ ID NO: 7, (d) CDR-L1 comprising the amino acid sequence of SEQ ID NO: 1, (e) CDR-L2 comprising the amino acid sequence of SEQ ID NO: 2, and (f) CDR-L3 comprising the amino acid sequence of SEQ ID NO: 3, and a VH domain having at least 90%, 91%, 92%, 93%, 94%, 95%, 96%, 97%, 98%, 99%, or 100% sequence identity to an amino acid sequence selected from the group consisting of SEQ ID NOs: 10-21, and a VL domain having at least 90%, 91%, 92%, 93%, 94%, 95%, 96%, 97%, 98%, 99%, or 100% sequence identity to an amino acid sequence selected from the group consisting of SEQ ID NOs: 22-25. In one aspect, the VH domain has at least 95% sequence identity to an amino acid sequence selected from the group consisting of SEQ ID NOs: 10-21. In one aspect, the VL domain has at least 95% sequence identity to an amino acid sequence selected from the group consisting of SEQ ID NOs: 22-25. In one aspect, the antibody binds to CCR8 having a dissociation constant (K_{D}) that is up to 10-fold reduced or up to 10-fold increased when compared to the dissociation constant (K_{D}) of an antibody comprising a VH sequence selected from the group consisting of SEQ ID NOs: 10-21 and a VL sequence selected from the group consisting of SEQ ID NOs: 22-25.

In one aspect, the anti-CCR8 antibody comprises (a) CDR-H1 comprising the amino acid sequence of SEQ ID NO: 4 or SEQ ID NO: 5, (b) CDR-H2 comprising the amino acid sequence of SEQ ID NO: 6, (c) CDR-H3 comprising the amino acid sequence of SEQ ID NO: 7, (d) CDR-L1 comprising the amino acid sequence of SEQ ID NO: 1, (e) CDR-L2 comprising the amino acid sequence of SEQ ID NO: 2, and (f) CDR-L3 comprising the amino acid sequence of SEQ ID NO: 3, and a VH domain having at least 90%, 91%, 92%, 93%, 94%, 95%, 96%, 97%, 98%, 99%, or 100% sequence identity to the amino acid sequence of SEQ ID NO: 21, and a VL domain having at least 90%, 91%, 92%, 93%, 94%, 95%, 96%, 97%, 98%, 99%, or 100% sequence identity to the amino acid sequence of SEQ ID NO: 24. In one aspect, the VH domain has at least 95% sequence identity to the amino acid sequence of SEQ ID NO: 21. In one aspect, the VL domain has at least 95% sequence identity to the amino acid sequence of SEQ ID NO: 24. In one aspect, the antibody binds to CCR8 having a dissociation constant (K_{D}) that is up to 10-fold reduced or up to 10-fold increased when compared to the dissociation constant (K_{D}) of an antibody comprising a VH sequence of SEQ ID NO: 21 and a VL sequence of SEQ ID NO: 24.

In another aspect, an anti-CCR8 antibody comprises a heavy chain variable domain (VH) sequence having at least 90%, 91%, 92%, 93%, 94%, 95%, 96%, 97%, 98%, 99%, or 100% sequence identity to an amino acid sequence selected from the group consisting of SEQ ID NOs: 10-21. In one aspect, an anti-CCR8 antibody comprises a heavy chain variable domain (VH) sequence having at least 95%, sequence identity to an amino acid sequence selected from the group consisting of SEQ ID NOs: 10-21. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-CCR8 antibody comprising that sequence retains the ability to bind to CCR8. In certain aspects, a total of 1 to 10 amino acids have been substituted, inserted and/or deleted in an amino acid sequence selected from the group consisting of SEQ ID NOs: 10-21. In certain aspects, substitutions, insertions, or deletions occur in regions outside the CDRs (i.e., in the FRs). Optionally, the anti-CCR8 antibody comprises the VH sequence selected from the group consisting of SEQ ID NOs: 10-21, including post-translational modifications of that sequence. In a particular aspect, the VH comprises one, two or three CDRs selected from: (a) CDR-H1 comprising the amino acid sequence of SEQ ID NO: 4 or SEQ ID NO: 5, (b) CDR-H2 comprising the amino acid sequence of SEQ ID NO: 6, and (c) CDR-H3 comprising the amino acid sequence of SEQ ID NO: 7. In another aspect, an anti-CCR8 antibody is provided, wherein the antibody comprises a light chain variable domain (VL) sequence having at least 90%, 91%, 92%, 93%, 94%, 95%, 96%, 97%, 98%, 99%, or 100% sequence identity to an amino acid sequence selected from the group consisting of SEQ ID NOs: 22-25. In one aspect, an anti-CCR8 antibody comprises a light chain variable domain (VL) sequence having at least 95% sequence identity to an amino acid sequence selected from the group consisting of SEQ ID NOs: 22-25. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-CCR8 antibody comprising that sequence retains the ability to bind to CCR8. In certain aspects, a total of 1 to 10 amino acids have been substituted, inserted and/or deleted in the amino acid sequence selected from the group consisting of SEQ ID NOs: 22-25. In certain aspects, the substitutions, insertions, or deletions occur in regions outside the CDRs (i.e., in the FRs). Optionally, the anti-CCR8 antibody comprises the VL sequence selected from the group consisting of SEQ ID NOs: 22-25, including post-translational modifications of that sequence. In a particular aspect, the VL comprises one, two or three CDRs selected from: (a) CDR-L1 comprising the amino acid sequence of SEQ ID NO: 1, (b) CDR-L2 comprising the amino acid sequence of SEQ ID NO: 2, and (c) CDR-L3 comprising the amino acid sequence of SEQ ID NO: 3.

In another aspect, an anti-CCR8 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 21. In one aspect, an anti-CCR8 antibody comprises a heavy chain variable domain (VH) sequence having at least 95%, sequence identity to the amino acid sequence of SEQ ID NO: 21. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-CCR8 antibody comprising that sequence retains the ability to bind to CCR8. In certain aspects, a total of 1 to 10 amino acids have been substituted, inserted and/or deleted in the amino acid sequence of SEQ ID NO: 21. In certain aspects, substitutions, insertions, or deletions occur in regions outside the CDRs (i.e., in the FRs). Optionally, the anti-CCR8 antibody comprises the VH sequence of SEQ ID NO: 21, including post-translational modifications of that sequence. In a particular aspect, the VH comprises one, two or three CDRs selected from: (a) CDR-H1 comprising the amino acid sequence of SEQ ID NO: 4 or SEQ ID NO: 5, (b) CDR-H2 comprising the amino acid sequence of SEQ ID NO: 6, (c) CDR-H3 comprising the amino acid sequence of SEQ ID NO: 7. In another aspect, an anti-CCR8 antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO: 24. In one aspect, an anti-CCR8 antibody comprises a light chain variable domain (VL) sequence having at least 95% sequence identity to the amino acid sequence of SEQ ID NO: 24. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-CCR8 antibody comprising that sequence retains the ability to bind to CCR8. In certain aspects, a total of 1 to 10 amino acids have been substituted, inserted and/or deleted in the amino acid sequence of SEQ ID NO: 24. In certain aspects, the substitutions, insertions, or deletions occur in regions outside the CDRs (i.e., in the FRs). Optionally, the anti-CCR8 antibody comprises the VL sequence of SEQ ID NO: 24, including post-translational modifications of that sequence. In a particular aspect, the VL comprises one, two or three CDRs selected from: (a) CDR-L1 comprising the amino acid sequence of SEQ ID NO: 1, (b) CDR-L2 comprising the amino acid sequence of SEQ ID NO: 2, and (c) CDR-L3 comprising the amino acid sequence of SEQ ID NO: 3.

In another aspect, an anti-CCR8 antibody is provided, wherein the antibody comprises a VH sequence as in any of the aspects provided above, and a VL sequence as in any of the aspects provided above. In one aspect, the antibody comprises the VH sequence selected from the group consisting of SEQ ID NOs: 10-21 and the VL sequence selected from the group consisting of SEQ ID NOs: 22-25, including post-translational modifications of those sequences. In one aspect, the antibody comprises the VH sequence of SEQ ID NO: 21 and the VL sequence of SEQ ID NO: 24, including post-translational modifications of those sequences.

In one aspect, the VL sequence comprises a Y21 mutation. In one aspect, the VH sequence comprises a S73N mutation, a V78L mutation, a T76N mutation, a F91 Y mutation, and a P105Q mutation, or a combination thereof (e.g., according to Kabat numbering).

In another aspect, an anti-CCR8 antibody is provided, wherein the antibody comprises a IgG1 constant domain comprising the amino acid sequence of SEQ ID NO: 53 or SEQ ID NO: 59. In one aspect, the antibody comprises a kappa constant domain comprising the amino acid sequence of SEQ ID NO: 54. In another aspect, an anti-CCR8 antibody is provided, wherein the antibody comprises (a) a IgG1 constant domain comprising the amino acid sequence of SEQ ID NO: 53 or SEQ ID NO: 59, and (b) a kappa constant domain comprising the amino acid sequence of SEQ ID NO: 54.

In another aspect, an anti-CCR8 antibody is provided, wherein the antibody comprises a heavy chain variable domain (VH) comprising (a) CDR-H1 comprising the amino acid sequence of SEQ ID NO: 4 or SEQ ID NO: 5, (b) CDR-H2 comprising the amino acid sequence of SEQ ID NO: 6, and (c) CDR-H3 comprising the amino acid sequence of SEQ ID NO: 7, and a light chain variable domain (VL) comprising (d) CDR-L1 comprising the amino acid sequence of SEQ ID NO: 1, (e) CDR-L2 comprising the amino acid sequence of SEQ ID NO: 2, and (f) CDR-L3 comprising the amino acid sequence of SEQ ID NO: 3.

In one aspect, the anti-CCR8 antibody comprises a VH sequence of SEQ ID NO: 21 and a VL sequence of SEQ ID NO: 24.

In one aspect, the anti-CCR8 antibody comprises a heavy chain of SEQ ID NO: 57, and a light chain of SEQ ID NO: 58.

In one aspect, the anti-CCR8 antibody comprises a heavy chain of SEQ ID NO: 61, and a light chain of SEQ ID NO: 58.

In another aspect of any of the above-described embodiments, an anti-CCR8 antibody is provided, wherein the heavy chain of the antibody comprises a shortened C-terminus in which one or two of the C terminal amino acid residues have been removed. In one aspect, the C-terminus of the heavy chain is a shortened C-terminus ending PG. In one aspect, the anti-CCR8 antibody comprises a heavy chain of SEQ ID NO: 112, and a light chain of SEQ ID NO: 58. In one aspect, the anti-CCR8 antibody comprises a heavy chain of SEQ ID NO: 114, and a light chain of SEQ ID NO: 58.

In another aspect of any of the above-described embodiments, an anti-CCR8 antibody is provided, wherein the antibody binds to CCR8 ligands. In one aspect, the anti-CCR8 antibody has antagonistic effects against the CCR8 ligand. In one aspect, the anti-CCR8 antibody has CCR8 ligand blocking activity. In one aspect, the anti-CCR8 antibody is a neutralizing antibody. In one aspect, the CCR8 ligand is CCL1.

In another aspect of any of the above-described embodiments, an anti-CCR8 antibody is provided, wherein the antibody binds to CCR8 independent of tyrosine sulfation of CCR8 for binding (i.e., sulfation independent).

In another aspect of any of the above-described embodiments, an anti-CCR8 antibody is provided, wherein the antibody is an afucosylated antibody variant. In one aspect, the afucosylated antibody variant has enhanced FcγRIIIa receptor binding. In one aspect, the afucosylated anti-CCR8 antibody variant has enhanced antibody-dependent cellular cytotoxicity (ADCC). In one aspect, the anti-CCR8 afucosylated antibody variant has antibody-dependent cellular phagocytosis (ADCP) activities.

In another aspect of any of the above-described embodiments, an anti-CCR8 antibody is provided, wherein the antibody has improved antibody stability. In one aspect, the anti-CCR8 antibody has low aggregation, good solubility, and/or low viscosity. In certain aspects of any of the above-described embodiments, an anti-CCR8 antibody is provided, wherein the antibody has a K_{D} of from about from about 1×10⁻¹¹ M to about 5×10⁻¹¹ M. In certain aspects, the antibody that binds to CCR8 has a K_{D} of about 2×10⁻¹¹ M.

In one aspect, the anti-CCR8 antibody is named as “hu.Ab4.H112L3” in the present disclosure, which can be fucosylated or afucosylated, which optionally contains one or more heavy chain mutations at G236A and I331E, and which optionally comprises a shortened C-terminus of the heavy chain in which one or two of the C terminal amino acid residues have been removed. In some instances, the heavy chain mutations are numbered according to the EU index.

In a further aspect, an anti-CCR8 antibody according to any of the above aspects is a monoclonal antibody, including a chimeric, humanized or human antibody. In one aspect, an anti-CCR8 antibody is an antibody fragment, e.g., a Fv, Fab, Fab′, scFv, diabody, or F(ab′)₂ fragment.

(iii) Embodiments of Ab1 and Fragments Thereof.

In one aspect, the present disclosure provides an anti-CCR8 antibody comprising at least one, at least two, at least three, at least four, at least five, or all six CDRs selected from the group consisting of (a) CDR-H1 comprising the amino acid sequence of SEQ ID NO: 82 or SEQ ID NO: 83, (b) CDR-H2 comprising the amino acid sequence of SEQ ID NO: 84, (c) CDR-H3 comprising the amino acid sequence of SEQ ID NO: 85, (d) CDR-L1 comprising the amino acid sequence of SEQ ID NO: 73, (e) CDR-L2 comprising the amino acid sequence of SEQ ID NO: 74, and (f) CDR-L3 comprising the amino acid sequence of SEQ ID NO: 75. In certain aspects, the anti-CCR8 antibody comprises all six of the aforementioned CDRs. In certain aspects, the anti-CCR8 antibody is a full-length antibody. In certain aspects, the anti-CCR8 antibody is a full-length antibody which binds to human CCR8. In certain aspects, the anti-CCR8 antibody is a full-length antibody which binds to human CCR8 and is a human antibody. In certain aspects, the anti-CCR8 antibody is a full-length antibody which binds to human CCR8 and is a humanized antibody. In certain aspects, the anti-CCR8 antibody is a full-length antibody which binds to human CCR8 and is a chimeric antibody.

In one aspect, the present disclosure provides an antibody comprising at least one, at least two, or all three VH CDR sequences selected from the group consisting of (a) CDR-H1 comprising the amino acid sequence of SEQ ID NO: 82 or SEQ ID NO: 83, (b) CDR-H2 comprising the amino acid sequence of SEQ ID NO: 84, and (c) CDR-H3 comprising the amino acid sequence of SEQ ID NO: 85. In one aspect, the antibody comprises CDR-H3 comprising the amino acid sequence of SEQ ID NO: 85. In another aspect, the antibody comprises CDR-H3 comprising the amino acid sequence of SEQ ID NO: 85 and CDR-L3 comprising the amino acid sequence of SEQ ID NO: 75. In a further aspect, the antibody comprises CDR-H3 comprising the amino acid sequence of SEQ ID NO: 85, CDR-L3 comprising the amino acid sequence of SEQ ID NO: 75, and CDR-H2 comprising the amino acid sequence of SEQ ID NO: 84. In a further aspect, the antibody comprises (a) CDR-H1 comprising the amino acid sequence of SEQ ID NO: 82 or SEQ ID NO: 83, (b) CDR-H2 comprising the amino acid sequence of SEQ ID NO: 84, and (c) CDR-H3 comprising the amino acid sequence of SEQ ID NO: 85.

In another aspect, the present disclosure provides an antibody comprising at least one, at least two, or all three VL CDR sequences selected from the group consisting of (a) CDR-L1 comprising the amino acid sequence of SEQ ID NO: 73; (b) CDR-L2 comprising the amino acid sequence of SEQ ID NO: 74; and (c) CDR-L3 comprising the amino acid sequence of SEQ ID NO: 75. In one aspect, the antibody comprises (a) CDR-L1 comprising the amino acid sequence of SEQ ID NO: 73; (b) CDR-L2 comprising the amino acid sequence of SEQ ID NO: 74; and (c) CDR-L3 comprising the amino acid sequence of SEQ ID NO: 75.

In another aspect, an antibody as described herein comprises (a) a VH domain comprising at least one, at least two, or all three VH CDR sequences selected from the group consisting of (i) CDR-H1 comprising the amino acid sequence of SEQ ID NO: 82 or SEQ ID NO: 83, (ii) CDR-H2 comprising the amino acid sequence of SEQ ID NO: 84, and (iii) CDR-H3 comprising the amino acid sequence of SEQ ID NO: 85; and (b) a VL domain comprising at least one, at least two, or all three VL CDR sequences selected from the group consisting of (i) CDR-L1 comprising the amino acid sequence of SEQ ID NO: 73; (ii) CDR-L2 comprising the amino acid sequence of SEQ ID NO: 74; and (iii) CDR-L3 comprising the amino acid sequence of SEQ ID NO: 75. In certain aspects, the anti-CCR8 antibody comprises all six of the aforementioned CDRs. In certain aspects, the anti-CCR8 antibody is a full-length antibody. In certain aspects, the anti-CCR8 antibody is a full-length antibody which binds to human CCR8. In certain aspects, the anti-CCR8 antibody is a full-length antibody which binds to human CCR8 and is a human antibody. In certain aspects, the anti-CCR8 antibody is a full-length antibody which binds to human CCR8 and is a humanized antibody. In certain aspects, the anti-CCR8 antibody is a full-length antibody which binds to human CCR8 and is a chimeric antibody.

In another aspect, the present disclosure provides an antibody comprising (a) CDR-H1 comprising the amino acid sequence of SEQ ID NO: 82 or SEQ ID NO: 83, (b) CDR-H2 comprising the amino acid sequence of SEQ ID NO: 84, and (c) CDR-H3 comprising the amino acid sequence of SEQ ID NO: 85, and a light chain variable domain (VL) comprising (d) CDR-L1 comprising the amino acid sequence of SEQ ID NO: 73, (e) CDR-L2 comprising the amino acid sequence of SEQ ID NO: 74, and (f) CDR-L3 comprising the amino acid sequence of SEQ ID NO: 75.

In another aspect, an anti-CCR8 antibody comprises one or more of the CDR sequences of the VH sequence of SEQ ID NO: 95. In another aspect, an anti-CCR8 antibody comprises one or more of the CDR sequences of the VL sequence of SEQ ID NO: 94. In another aspect, an anti-CCR8 antibody comprises the CDR sequences of the VH sequence of SEQ ID NO: 95. In another aspect, an anti-CCR8 antibody comprises one or more of the CDR sequences of the VL sequence of SEQ ID NO: 94.

In a further aspect, an anti-CCR8 antibody comprises the CDR-H1, CDR-H2 and CDR-H3 amino acid sequences of the VH domain of SEQ ID NO: 95 and the CDR-L1, CDR-L2 and CDR-L3 amino acid sequences of the VL domain of SEQ ID NO: 94.

In one aspect, an anti-CCR8 antibody comprises one or more of the heavy chain CDR amino acid sequences of the VH domain of SEQ ID NO: 95 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 95. In one aspect, the anti-CCR8 antibody comprises the three heavy chain CDR amino acid sequences of the VH domain of SEQ ID NO: 95 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 95. In one aspect, the anti-CCR8 antibody comprises the three heavy chain CDR amino acid sequences of the VH domain of SEQ ID NO: 95 and a framework of at least 95% sequence identity to the framework amino acid sequence of the VH domain of SEQ ID NO: 95. In another aspect, the anti-CCR8 antibody comprises the three heavy chain CDR amino acid sequences of the VH domain of SEQ ID NO: 95 and a framework of at least of at least 98% sequence identity to the framework amino acid sequence of the VH domain of SEQ ID NO: 95.

In one aspect, an anti-CCR8 antibody comprises one or more of the light chain CDR amino acid sequences of the VL domain of SEQ ID NO: 94 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 94. In one aspect, the anti-CCR8 antibody comprises the three light chain CDR amino acid sequences of the VL domain of SEQ ID NO: 94 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 94. In one aspect, the anti-CCR8 antibody comprises the three light chain CDR amino acid sequences of the VL domain of SEQ ID NO: 94 and a framework of at least 95% sequence identity to the framework amino acid sequence of the VL domain of SEQ ID NO: 94. In another aspect, the anti-CCR8 antibody comprises the three light chain CDR amino acid sequences of the VL domain of SEQ ID NO: 94 and a framework of at least particularly of at least 98% sequence identity to the framework amino acid sequence of the VL domain of SEQ ID NO: 94.

In one aspect, the anti-CCR8 antibody comprises (a) CDR-H1 comprising the amino acid sequence of SEQ ID NO: 82 or SEQ ID NO: 83, (b) CDR-H2 comprising the amino acid sequence of SEQ ID NO: 84, (c) CDR-H3 comprising the amino acid sequence of SEQ ID NO: 85, (d) CDR-L1 comprising the amino acid sequence of SEQ ID NO: 73, (e) CDR-L2 comprising the amino acid sequence of SEQ ID NO: 74, and (f) CDR-L3 comprising the amino acid sequence of SEQ ID NO: 75, and a VH domain having at least 90%, 91%, 92%, 93%, 94%, 95%, 96%, 97%, 98%, 99%, or 100% sequence identity to the amino acid sequence of SEQ ID NO: 95, and a VL domain having at least 90%, 91%, 92%, 93%, 94%, 95%, 96%, 97%, 98%, 99%, or 100% sequence identity to the amino acid sequence of SEQ ID NO: 94. In one aspect, the VH domain has at least 95% sequence identity to the amino acid sequence of SEQ ID NO: 95. In one aspect, the VL domain has at least 95% sequence identity to the amino acid sequence of SEQ ID NO: 94. In one aspect, the antibody binds to CCR8 having a dissociation constant (K_{D}) that is up to 10-fold reduced or up to 10-fold increased when compared to the dissociation constant (K_{D}) of an antibody comprising a VH sequence of SEQ ID NO: 95 and a VL sequence of SEQ ID NO: 94.

In another aspect, an anti-CCR8 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 95. In one aspect, an anti-CCR8 antibody comprises a heavy chain variable domain (VH) sequence having at least 95%, sequence identity to the amino acid sequence of SEQ ID NO: 95. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-CCR8 antibody comprising that sequence retains the ability to bind to CCR8. In certain aspects, a total of 1 to 10 amino acids have been substituted, inserted and/or deleted in the amino acid sequence of SEQ ID NO: 95. In certain aspects, substitutions, insertions, or deletions occur in regions outside the CDRs (i.e., in the FRs). Optionally, the anti-CCR8 antibody comprises the VH sequence of SEQ ID NO: 95, including post-translational modifications of that sequence. In a particular aspect, the VH comprises one, two or three CDRs selected from: (a) CDR-H1 comprising the amino acid sequence of SEQ ID NO: 82 or SEQ ID NO: 83, (b) CDR-H2 comprising the amino acid sequence of SEQ ID NO: 84, (c) CDR-H3 comprising the amino acid sequence of SEQ ID NO: 85. In another aspect, an anti-CCR8 antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO: 94. In one aspect, an anti-CCR8 antibody comprises a light chain variable domain (VL) sequence having at least 95% sequence identity to the amino acid sequence of SEQ ID NO: 94. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-CCR8 antibody comprising that sequence retains the ability to bind to CCR8. In certain aspects, a total of 1 to 10 amino acids have been substituted, inserted and/or deleted in the amino acid sequence of SEQ ID NO: 94. In certain aspects, the substitutions, insertions, or deletions occur in regions outside the CDRs (i.e., in the FRs). Optionally, the anti-CCR8 antibody comprises the VL sequence of SEQ ID NO: 94, including post-translational modifications of that sequence. In a particular aspect, the VL comprises one, two or three CDRs selected from: (a) CDR-L1 comprising the amino acid sequence of SEQ ID NO: 73, (b) CDR-L2 comprising the amino acid sequence of SEQ ID NO: 74, and (c) CDR-L3 comprising the amino acid sequence of SEQ ID NO: 75.

In another aspect, an anti-CCR8 antibody is provided, wherein the antibody comprises a VH sequence as in any of the aspects provided above, and a VL sequence as in any of the aspects provided above. In one aspect, the antibody comprises the VH sequence of SEQ ID NO: 95 and the VL sequence of SEQ ID NO: 94, including post-translational modifications of those sequences.

In another aspect, an anti-CCR8 antibody is provided, wherein the antibody comprises a IgG1 constant domain comprising the amino acid sequence of SEQ ID NO: 53 or SEQ ID NO: 59. In one aspect, the antibody comprises a kappa constant domain comprising the amino acid sequence of SEQ ID NO: 54. In another aspect, an anti-CCR8 antibody is provided, wherein the antibody comprises (a) a IgG1 constant domain comprising the amino acid sequence of SEQ ID NO: 53 or SEQ ID NO: 59, and (b) a kappa constant domain comprising the amino acid sequence of SEQ ID NO: 54.

In another aspect, an anti-CCR8 antibody is provided, wherein the antibody comprises a heavy chain variable domain (VH) comprising (a) CDR-H1 comprising the amino acid sequence of SEQ ID NO: 82 or SEQ ID NO: 83, (b) CDR-H2 comprising the amino acid sequence of SEQ ID NO: 84, and (c) CDR-H3 comprising the amino acid sequence of SEQ ID NO: 85, and a light chain variable domain (VL) comprising (d) CDR-L1 comprising the amino acid sequence of SEQ ID NO: 73, (e) CDR-L2 comprising the amino acid sequence of SEQ ID NO: 74, and (f) CDR-L3 comprising the amino acid sequence of SEQ ID NO: 75. In one aspect, the anti-CCR8 antibody comprises a VH sequence of SEQ ID NO: 95 and a VL sequence of SEQ ID NO: 94.

In one aspect, the anti-CCR8 antibody comprises a heavy chain of SEQ ID NO: 101, and a light chain of SEQ ID NO: 100.

In another aspect of any of the above-described embodiments, an anti-CCR8 antibody is provided, wherein the heavy chain of the antibody comprises a shortened C-terminus in which one or two of the C terminal amino acid residues have been removed. In one aspect, the C-terminus of the heavy chain is a shortened C-terminus ending PG. In one aspect, the anti-CCR8 antibody comprises a heavy chain of SEQ ID NO: 115, and a light chain of SEQ ID NO: 100.

In one aspect, the anti-CCR8 antibody is named as “hu.Ab1.H1 L1” in the present disclosure, which can be fucosylated or afucosylated, which optionally contains one or more heavy chain mutations at G236A and I331E, and which optionally comprises a shortened C-terminus of the heavy chain in which one or two of the C terminal amino acid residues have been removed. In some instances, the heavy chain mutations are numbered according to the EU index.

In a further aspect, an anti-CCR8 antibody according to any of the above aspects is a monoclonal antibody, including a chimeric, humanized or human antibody. In one aspect, an anti-CCR8 antibody is an antibody fragment, e.g., a Fv, Fab, Fab′, scFv, diabody, or F(ab′)₂ fragment.

### (iv) Embodiments of Ab2 and Fragments Thereof.

In one aspect, the present disclosure provides an anti-CCR8 antibody comprising at least one, at least two, at least three, at least four, at least five, or all six CDRs selected from the group consisting of (a) CDR-H1 comprising the amino acid sequence of SEQ ID NO: 86 or SEQ ID NO: 87, (b) CDR-H2 comprising the amino acid sequence of SEQ ID NO: 88, (c) CDR-H3 comprising the amino acid sequence of SEQ ID NO: 89, (d) CDR-L1 comprising the amino acid sequence of SEQ ID NO: 76, (e) CDR-L2 comprising the amino acid sequence of SEQ ID NO: 77, and (f) CDR-L3 comprising the amino acid sequence of SEQ ID NO: 78. In certain aspects, the anti-CCR8 antibody comprises all six of the aforementioned CDRs. In certain aspects, the anti-CCR8 antibody is a full-length antibody. In certain aspects, the anti-CCR8 antibody is a full-length antibody which binds to human CCR8. In certain aspects, the anti-CCR8 antibody is a full-length antibody which binds to human CCR8 and is a human antibody. In certain aspects, the anti-CCR8 antibody is a full-length antibody which binds to human CCR8 and is a humanized antibody. In certain aspects, the anti-CCR8 antibody is a full-length antibody which binds to human CCR8 and is a chimeric antibody.

In one aspect, the present disclosure provides an antibody comprising at least one, at least two, or all three VH CDR sequences selected from the group consisting of (a) CDR-H1 comprising the amino acid sequence of SEQ ID NO: 86 or SEQ ID NO: 87, (b) CDR-H2 comprising the amino acid sequence of SEQ ID NO: 88, and (c) CDR-H3 comprising the amino acid sequence of SEQ ID NO: 89. In one aspect, the antibody comprises CDR-H3 comprising the amino acid sequence of SEQ ID NO: 89. In another aspect, the antibody comprises CDR-H3 comprising the amino acid sequence of SEQ ID NO: 89 and CDR-L3 comprising the amino acid sequence of SEQ ID NO: 78. In a further aspect, the antibody comprises CDR-H3 comprising the amino acid sequence of SEQ ID NO: 89, CDR-L3 comprising the amino acid sequence of SEQ ID NO: 78, and CDR-H2 comprising the amino acid sequence of SEQ ID NO: 88. In a further aspect, the antibody comprises (a) CDR-H1 comprising the amino acid sequence of SEQ ID NO: 86 or SEQ ID NO: 87, (b) CDR-H2 comprising the amino acid sequence of SEQ ID NO: 88, and (c) CDR-H3 comprising the amino acid sequence of SEQ ID NO: 89.

In another aspect, the present disclosure provides an antibody comprising at least one, at least two, or all three VL CDR sequences selected from the group consisting of (a) CDR-L1 comprising the amino acid sequence of SEQ ID NO: 76; (b) CDR-L2 comprising the amino acid sequence of SEQ ID NO: 77; and (c) CDR-L3 comprising the amino acid sequence of SEQ ID NO: 78. In one aspect, the antibody comprises (a) CDR-L1 comprising the amino acid sequence of SEQ ID NO: 76; (b) CDR-L2 comprising the amino acid sequence of SEQ ID NO: 77; and (c) CDR-L3 comprising the amino acid sequence of SEQ ID NO: 78.

In another aspect, an antibody as described herein comprises (a) a VH domain comprising at least one, at least two, or all three VH CDR sequences selected from the group consisting of (i) CDR-H1 comprising the amino acid sequence of SEQ ID NO: 86 or SEQ ID NO: 87, (ii) CDR-H2 comprising the amino acid sequence of SEQ ID NO: 88, and (iii) CDR-H3 comprising the amino acid sequence of SEQ ID NO: 89; and (b) a VL domain comprising at least one, at least two, or all three VL CDR sequences selected from the group consisting of (i) CDR-L1 comprising the amino acid sequence of SEQ ID NO: 76; (ii) CDR-L2 comprising the amino acid sequence of SEQ ID NO: 77; and (iii) CDR-L3 comprising the amino acid sequence of SEQ ID NO: 78. In certain aspects, the anti-CCR8 antibody comprises all six of the aforementioned CDRs. In certain aspects, the anti-CCR8 antibody is a full-length antibody. In certain aspects, the anti-CCR8 antibody is a full-length antibody which binds to human CCR8. In certain aspects, the anti-CCR8 antibody is a full-length antibody which binds to human CCR8 and is a human antibody. In certain aspects, the anti-CCR8 antibody is a full-length antibody which binds to human CCR8 and is a humanized antibody. In certain aspects, the anti-CCR8 antibody is a full-length antibody which binds to human CCR8 and is a chimeric antibody.

In another aspect, the present disclosure provides an antibody comprising (a) CDR-H1 comprising the amino acid sequence of SEQ ID NO: 86 or SEQ ID NO: 87, (b) CDR-H2 comprising the amino acid sequence of SEQ ID NO: 88, and (c) CDR-H3 comprising the amino acid sequence of SEQ ID NO: 89, and a light chain variable domain (VL) comprising (d) CDR-L1 comprising the amino acid sequence of SEQ ID NO: 76, (e) CDR-L2 comprising the amino acid sequence of SEQ ID NO: 77, and (f) CDR-L3 comprising the amino acid sequence of SEQ ID NO: 78.

In another aspect, an anti-CCR8 antibody comprises one or more of the CDR sequences of the VH sequence of SEQ ID NO: 97. In another aspect, an anti-CCR8 antibody comprises one or more of the CDR sequences of the VL sequence of SEQ ID NO: 96. In another aspect, an anti-CCR8 antibody comprises the CDR sequences of the VH sequence of SEQ ID NO: 97. In another aspect, an anti-CCR8 antibody comprises one or more of the CDR sequences of the VL sequence of SEQ ID NO: 96.

In a further aspect, an anti-CCR8 antibody comprises the CDR-H1, CDR-H2 and CDR-H3 amino acid sequences of the VH domain of SEQ ID NO: 97 and the CDR-L1, CDR-L2 and CDR-L3 amino acid sequences of the VL domain of SEQ ID NO: 96.

In one aspect, an anti-CCR8 antibody comprises one or more of the heavy chain CDR amino acid sequences of the VH domain of SEQ ID NO: 97 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 97. In one aspect, the anti-CCR8 antibody comprises the three heavy chain CDR amino acid sequences of the VH domain of SEQ ID NO: 97 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 97. In one aspect, the anti-CCR8 antibody comprises the three heavy chain CDR amino acid sequences of the VH domain of SEQ ID NO: 97 and a framework of at least 95% sequence identity to the framework amino acid sequence of the VH domain of SEQ ID NO: 97. In another aspect, the anti-CCR8 antibody comprises the three heavy chain CDR amino acid sequences of the VH domain of SEQ ID NO: 97 and a framework of at least of at least 98% sequence identity to the framework amino acid sequence of the VH domain of SEQ ID NO: 97.

In one aspect, an anti-CCR8 antibody comprises one or more of the light chain CDR amino acid sequences of the VL domain of SEQ ID NO: 96 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 96. In one aspect, the anti-CCR8 antibody comprises the three light chain CDR amino acid sequences of the VL domain of SEQ ID NO: 96 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 96. In one aspect, the anti-CCR8 antibody comprises the three light chain CDR amino acid sequences of the VL domain of SEQ ID NO: 96 and a framework of at least 95% sequence identity to the framework amino acid sequence of the VL domain of SEQ ID NO: 96. In another aspect, the anti-CCR8 antibody comprises the three light chain CDR amino acid sequences of the VL domain of SEQ ID NO: 96 and a framework of at least particularly of at least 98% sequence identity to the framework amino acid sequence of the VL domain of SEQ ID NO: 96.

In one aspect, the anti-CCR8 antibody comprises (a) CDR-H1 comprising the amino acid sequence of SEQ ID NO: 86 or SEQ ID NO: 87, (b) CDR-H2 comprising the amino acid sequence of SEQ ID NO: 88, (c) CDR-H3 comprising the amino acid sequence of SEQ ID NO: 89, (d) CDR-L1 comprising the amino acid sequence of SEQ ID NO: 76, (e) CDR-L2 comprising the amino acid sequence of SEQ ID NO: 77, and (f) CDR-L3 comprising the amino acid sequence of SEQ ID NO: 78, and a VH domain having at least 90%, 91%, 92%, 93%, 94%, 95%, 96%, 97%, 98%, 99%, or 100% sequence identity to the amino acid sequence of SEQ ID NO: 97, and a VL domain having at least 90%, 91%, 92%, 93%, 94%, 95%, 96%, 97%, 98%, 99%, or 100% sequence identity to the amino acid sequence of SEQ ID NO: 96. In one aspect, the VH domain has at least 95% sequence identity to the amino acid sequence of SEQ ID NO: 97. In one aspect, the VL domain has at least 95% sequence identity to the amino acid sequence of SEQ ID NO: 96. In one aspect, the antibody binds to CCR8 having a dissociation constant (K_{D}) that is up to 10-fold reduced or up to 10-fold increased when compared to the dissociation constant (K_{D}) of an antibody comprising a VH sequence of SEQ ID NO: 97 and a VL sequence of SEQ ID NO: 96.

In another aspect, an anti-CCR8 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 97. In one aspect, an anti-CCR8 antibody comprises a heavy chain variable domain (VH) sequence having at least 95%, sequence identity to the amino acid sequence of SEQ ID NO: 97. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-CCR8 antibody comprising that sequence retains the ability to bind to CCR8. In certain aspects, a total of 1 to 10 amino acids have been substituted, inserted and/or deleted in the amino acid sequence of SEQ ID NO: 97. In certain aspects, substitutions, insertions, or deletions occur in regions outside the CDRs (i.e., in the FRs). Optionally, the anti-CCR8 antibody comprises the VH sequence of SEQ ID NO: 97, including post-translational modifications of that sequence. In a particular aspect, the VH comprises one, two or three CDRs selected from: (a) CDR-H1 comprising the amino acid sequence of SEQ ID NO: 86 or SEQ ID NO: 87, (b) CDR-H2 comprising the amino acid sequence of SEQ ID NO: 88, (c) CDR-H3 comprising the amino acid sequence of SEQ ID NO: 89. In another aspect, an anti-CCR8 antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO: 96. In one aspect, an anti-CCR8 antibody comprises a light chain variable domain (VL) sequence having at least 95% sequence identity to the amino acid sequence of SEQ ID NO: 96. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-CCR8 antibody comprising that sequence retains the ability to bind to CCR8. In certain aspects, a total of 1 to 10 amino acids have been substituted, inserted and/or deleted in the amino acid sequence of SEQ ID NO: 96. In certain aspects, the substitutions, insertions, or deletions occur in regions outside the CDRs (i.e., in the FRs). Optionally, the anti-CCR8 antibody comprises the VL sequence of SEQ ID NO: 96, including post-translational modifications of that sequence. In a particular aspect, the VL comprises one, two or three CDRs selected from: (a) CDR-L1 comprising the amino acid sequence of SEQ ID NO: 76, (b) CDR-L2 comprising the amino acid sequence of SEQ ID NO: 75, and (c) CDR-L3 comprising the amino acid sequence of SEQ ID NO: 78.

In another aspect, an anti-CCR8 antibody is provided, wherein the antibody comprises a VH sequence as in any of the aspects provided above, and a VL sequence as in any of the aspects provided above. In one aspect, the antibody comprises the VH sequence of SEQ ID NO: 97 and the VL sequence of SEQ ID NO: 96, including post-translational modifications of those sequences.

In another aspect, an anti-CCR8 antibody is provided, wherein the antibody comprises a IgG1 constant domain comprising the amino acid sequence of SEQ ID NO: 53 or SEQ ID NO: 59. In one aspect, the antibody comprises a kappa constant domain comprising the amino acid sequence of SEQ ID NO: 54. In another aspect, an anti-CCR8 antibody is provided, wherein the antibody comprises (a) a IgG1 constant domain comprising the amino acid sequence of SEQ ID NO: 53 or SEQ ID NO: 59, and (b) a kappa constant domain comprising the amino acid sequence of SEQ ID NO: 54.

In another aspect, an anti-CCR8 antibody is provided, wherein the antibody comprises a heavy chain variable domain (VH) comprising (a) CDR-H1 comprising the amino acid sequence of SEQ ID NO: 86 or SEQ ID NO: 87, (b) CDR-H2 comprising the amino acid sequence of SEQ ID NO: 88, and (c) CDR-H3 comprising the amino acid sequence of SEQ ID NO: 89, and a light chain variable domain (VL) comprising (d) CDR-L1 comprising the amino acid sequence of SEQ ID NO: 76, (e) CDR-L2 comprising the amino acid sequence of SEQ ID NO: 77, and (f) CDR-L3 comprising the amino acid sequence of SEQ ID NO: 78.

In one aspect, the anti-CCR8 antibody comprises a VH sequence of SEQ ID NO: 97 and a VL sequence of SEQ ID NO: 96.

In one aspect, the anti-CCR8 antibody comprises a heavy chain of SEQ ID NO: 103, and a light chain of SEQ ID NO: 102.

In another aspect of any of the above-described embodiments, an anti-CCR8 antibody is provided, wherein the heavy chain of the antibody comprises a shortened C-terminus in which one or two of the C terminal amino acid residues have been removed. In one aspect, the C-terminus of the heavy chain is a shortened C-terminus ending PG. In one aspect, the anti-CCR8 antibody comprises a heavy chain of SEQ ID NO: 116, and a light chain of SEQ ID NO: 102.

In one aspect, the anti-CCR8 antibody is named as “hu.Ab2.H1 L1” in the present disclosure, which can be fucosylated or afucosylated, which optionally contains one or more heavy chain mutations at G236A and I331E, and which optionally comprises a shortened C-terminus of the heavy chain in which one or two of the C terminal amino acid residues have been removed. In some instances, the heavy chain mutations are numbered according to the EU index.

In a further aspect, an anti-CCR8 antibody according to any of the above aspects is a monoclonal antibody, including a chimeric, humanized or human antibody. In one aspect, an anti-CCR8 antibody is an antibody fragment, e.g., a Fv, Fab, Fab′, scFv, diabody, or F(ab′)₂ fragment.

### (v) Embodiments of Ab3 and Fragments Thereof.

In one aspect, the present disclosure provides an anti-CCR8 antibody comprising at least one, at least two, at least three, at least four, at least five, or all six CDRs selected from the group consisting of (a) CDR-H1 comprising the amino acid sequence of SEQ ID NO: 90 or SEQ ID NO: 91, (b) CDR-H2 comprising the amino acid sequence of SEQ ID NO: 92, (c) CDR-H3 comprising the amino acid sequence of SEQ ID NO: 93, (d) CDR-L1 comprising the amino acid sequence of SEQ ID NO: 79, (e) CDR-L2 comprising the amino acid sequence of SEQ ID NO: 80, and (f) CDR-L3 comprising the amino acid sequence of SEQ ID NO: 81. In certain aspects, the anti-CCR8 antibody comprises all six of the aforementioned CDRs. In certain aspects, the anti-CCR8 antibody is a full-length antibody. In certain aspects, the anti-CCR8 antibody is a full-length antibody which binds to human CCR8. In certain aspects, the anti-CCR8 antibody is a full-length antibody which binds to human CCR8 and is a human antibody. In certain aspects, the anti-CCR8 antibody is a full-length antibody which binds to human CCR8 and is a humanized antibody. In certain aspects, the anti-CCR8 antibody is a full-length antibody which binds to human CCR8 and is a chimeric antibody.

In one aspect, the present disclosure provides an antibody comprising at least one, at least two, or all three VH CDR sequences selected from the group consisting of (a) CDR-H1 comprising the amino acid sequence of SEQ ID NO: 90 or SEQ ID NO: 91, (b) CDR-H2 comprising the amino acid sequence of SEQ ID NO: 92, and (c) CDR-H3 comprising the amino acid sequence of SEQ ID NO: 93. In one aspect, the antibody comprises CDR-H3 comprising the amino acid sequence of SEQ ID NO: 93. In another aspect, the antibody comprises CDR-H3 comprising the amino acid sequence of SEQ ID NO: 93 and CDR-L3 comprising the amino acid sequence of SEQ ID NO: 81. In a further aspect, the antibody comprises CDR-H3 comprising the amino acid sequence of SEQ ID NO: 93, CDR-L3 comprising the amino acid sequence of SEQ ID NO: 81, and CDR-H2 comprising the amino acid sequence of SEQ ID NO: 92. In a further aspect, the antibody comprises (a) CDR-H1 comprising the amino acid sequence of SEQ ID NO: 90 or SEQ ID NO: 91, (b) CDR-H2 comprising the amino acid sequence of SEQ ID NO: 92, and (c) CDR-H3 comprising the amino acid sequence of SEQ ID NO: 93.

In another aspect, the present disclosure provides an antibody comprising at least one, at least two, or all three VL CDR sequences selected from the group consisting of (a) CDR-L1 comprising the amino acid sequence of SEQ ID NO: 79; (b) CDR-L2 comprising the amino acid sequence of SEQ ID NO: 80; and (c) CDR-L3 comprising the amino acid sequence of SEQ ID NO: 81. In one aspect, the antibody comprises (a) CDR-L1 comprising the amino acid sequence of SEQ ID NO: 79; (b) CDR-L2 comprising the amino acid sequence of SEQ ID NO: 80; and (c) CDR-L3 comprising the amino acid sequence of SEQ ID NO: 81.

In another aspect, an antibody as described herein comprises (a) a VH domain comprising at least one, at least two, or all three VH CDR sequences selected from the group consisting of (i) CDR-H1 comprising the amino acid sequence of SEQ ID NO: 90 or SEQ ID NO: 91, (ii) CDR-H2 comprising the amino acid sequence of SEQ ID NO: 92, and (iii) CDR-H3 comprising the amino acid sequence of SEQ ID NO: 93; and (b) a VL domain comprising at least one, at least two, or all three VL CDR sequences selected from the group consisting of (i) CDR-L1 comprising the amino acid sequence of SEQ ID NO: 79; (ii) CDR-L2 comprising the amino acid sequence of SEQ ID NO: 80; and (iii) CDR-L3 comprising the amino acid sequence of SEQ ID NO: 81. In certain aspects, the anti-CCR8 antibody comprises all six of the aforementioned CDRs. In certain aspects, the anti-CCR8 antibody is a full-length antibody. In certain aspects, the anti-CCR8 antibody is a full-length antibody which binds to human CCR8. In certain aspects, the anti-CCR8 antibody is a full-length antibody which binds to human CCR8 and is a human antibody. In certain aspects, the anti-CCR8 antibody is a full-length antibody which binds to human CCR8 and is a humanized antibody. In certain aspects, the anti-CCR8 antibody is a full-length antibody which binds to human CCR8 and is a chimeric antibody.

In another aspect, the present disclosure provides an antibody comprising (a) CDR-H1 comprising the amino acid sequence of SEQ ID NO: 90 or SEQ ID NO: 91, (b) CDR-H2 comprising the amino acid sequence of SEQ ID NO: 92, and (c) CDR-H3 comprising the amino acid sequence of SEQ ID NO: 93, and a light chain variable domain (VL) comprising (d) CDR-L1 comprising the amino acid sequence of SEQ ID NO: 79, (e) CDR-L2 comprising the amino acid sequence of SEQ ID NO: 80, and (f) CDR-L3 comprising the amino acid sequence of SEQ ID NO: 81.

In another aspect, an anti-CCR8 antibody comprises one or more of the CDR sequences of the VH sequence of SEQ ID NO: 99. In another aspect, an anti-CCR8 antibody comprises one or more of the CDR sequences of the VL sequence of SEQ ID NO: 98. In another aspect, an anti-CCR8 antibody comprises the CDR sequences of the VH sequence of SEQ ID NO: 99. In another aspect, an anti-CCR8 antibody comprises one or more of the CDR sequences of the VL sequence of SEQ ID NO: 98.

In a further aspect, an anti-CCR8 antibody comprises the CDR-H1, CDR-H2 and CDR-H3 amino acid sequences of the VH domain of SEQ ID NO: 99 and the CDR-L1, CDR-L2 and CDR-L3 amino acid sequences of the VL domain of SEQ ID NO: 98.

In one aspect, an anti-CCR8 antibody comprises one or more of the heavy chain CDR amino acid sequences of the VH domain of SEQ ID NO: 99 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 99. In one aspect, the anti-CCR8 antibody comprises the three heavy chain CDR amino acid sequences of the VH domain of SEQ ID NO: 99 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 99. In one aspect, the anti-CCR8 antibody comprises the three heavy chain CDR amino acid sequences of the VH domain of SEQ ID NO: 99 and a framework of at least 95% sequence identity to the framework amino acid sequence of the VH domain of SEQ ID NO: 99. In another aspect, the anti-CCR8 antibody comprises the three heavy chain CDR amino acid sequences of the VH domain of SEQ ID NO: 99 and a framework of at least of at least 98% sequence identity to the framework amino acid sequence of the VH domain of SEQ ID NO: 99.

In one aspect, an anti-CCR8 antibody comprises one or more of the light chain CDR amino acid sequences of the VL domain of SEQ ID NO: 98 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 98. In one aspect, the anti-CCR8 antibody comprises the three light chain CDR amino acid sequences of the VL domain of SEQ ID NO: 98 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 98. In one aspect, the anti-CCR8 antibody comprises the three light chain CDR amino acid sequences of the VL domain of SEQ ID NO: 98 and a framework of at least 95% sequence identity to the framework amino acid sequence of the VL domain of SEQ ID NO: 98. In another aspect, the anti-CCR8 antibody comprises the three light chain CDR amino acid sequences of the VL domain of SEQ ID NO: 98 and a framework of at least particularly of at least 98% sequence identity to the framework amino acid sequence of the VL domain of SEQ ID NO: 98.

In one aspect, the anti-CCR8 antibody comprises (a) CDR-H1 comprising the amino acid sequence of SEQ ID NO: 90 or SEQ ID NO: 91, (b) CDR-H2 comprising the amino acid sequence of SEQ ID NO: 92, (c) CDR-H3 comprising the amino acid sequence of SEQ ID NO: 93, (d) CDR-L1 comprising the amino acid sequence of SEQ ID NO: 79, (e) CDR-L2 comprising the amino acid sequence of SEQ ID NO: 80, and (f) CDR-L3 comprising the amino acid sequence of SEQ ID NO: 81, and a VH domain having at least 90%, 91%, 92%, 93%, 94%, 95%, 96%, 97%, 98%, 99%, or 100% sequence identity to the amino acid sequence of SEQ ID NO: 99, and a VL domain having at least 90%, 91%, 92%, 93%, 94%, 95%, 96%, 97%, 98%, 99%, or 100% sequence identity to the amino acid sequence of SEQ ID NO: 98. In one aspect, the VH domain has at least 95% sequence identity to the amino acid sequence of SEQ ID NO: 99. In one aspect, the VL domain has at least 95% sequence identity to the amino acid sequence of SEQ ID NO: 98. In one aspect, the antibody binds to CCR8 having a dissociation constant (K_{D}) that is up to 10-fold reduced or up to 10-fold increased when compared to the dissociation constant (K_{D}) of an antibody comprising a VH sequence of SEQ ID NO: 99 and a VL sequence of SEQ ID NO: 98.

In another aspect, an anti-CCR8 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 99. In one aspect, an anti-CCR8 antibody comprises a heavy chain variable domain (VH) sequence having at least 95%, sequence identity to the amino acid sequence of SEQ ID NO: 99. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-CCR8 antibody comprising that sequence retains the ability to bind to CCR8. In certain aspects, a total of 1 to 10 amino acids have been substituted, inserted and/or deleted in the amino acid sequence of SEQ ID NO: 99. In certain aspects, substitutions, insertions, or deletions occur in regions outside the CDRs (i.e., in the FRs). Optionally, the anti-CCR8 antibody comprises the VH sequence of SEQ ID NO: 99, including post-translational modifications of that sequence. In a particular aspect, the VH comprises one, two or three CDRs selected from: SEQ ID NO: 90 or SEQ ID NO: 91, (b) CDR-H2 comprising the amino acid sequence of SEQ ID NO: 92, (c) CDR-H3 comprising the amino acid sequence of SEQ ID NO: 93. In another aspect, an anti-CCR8 antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO: 98. In one aspect, an anti-CCR8 antibody comprises a light chain variable domain (VL) sequence having at least 95% sequence identity to the amino acid sequence of SEQ ID NO: 98. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-CCR8 antibody comprising that sequence retains the ability to bind to CCR8. In certain aspects, a total of 1 to 10 amino acids have been substituted, inserted and/or deleted in the amino acid sequence of SEQ ID NO: 98. In certain aspects, the substitutions, insertions, or deletions occur in regions outside the CDRs (i.e., in the FRs). Optionally, the anti-CCR8 antibody comprises the VL sequence of SEQ ID NO: 98, including post-translational modifications of that sequence. In a particular aspect, the VL comprises one, two or three CDRs selected from: (a) CDR-L1 comprising the amino acid sequence of SEQ ID NO: 79, (b) CDR-L2 comprising the amino acid sequence of SEQ ID NO: 80, and (c) CDR-L3 comprising the amino acid sequence of SEQ ID NO: 81.

In another aspect, an anti-CCR8 antibody is provided, wherein the antibody comprises a VH sequence as in any of the aspects provided above, and a VL sequence as in any of the aspects provided above. In one aspect, the antibody comprises the VH sequence of SEQ ID NO: 99 and the VL sequence of SEQ ID NO: 98, including post-translational modifications of those sequences.

In another aspect, an anti-CCR8 antibody is provided, wherein the antibody comprises a IgG1 constant domain comprising the amino acid sequence of SEQ ID NO: 53 or SEQ ID NO: 59. In one aspect, the antibody comprises a kappa constant domain comprising the amino acid sequence of SEQ ID NO: 54. In another aspect, an anti-CCR8 antibody is provided, wherein the antibody comprises (a) a IgG1 constant domain comprising the amino acid sequence of SEQ ID NO: 53 or SEQ ID NO: 59, and (b) a kappa constant domain comprising the amino acid sequence of SEQ ID NO: 54.

In another aspect, an anti-CCR8 antibody is provided, wherein the antibody comprises a heavy chain variable domain (VH) comprising (a) CDR-H1 comprising the amino acid sequence of SEQ ID NO: 90 or SEQ ID NO: 91, (b) CDR-H2 comprising the amino acid sequence of SEQ ID NO: 92, and (c) CDR-H3 comprising the amino acid sequence of SEQ ID NO: 93, and a light chain variable domain (VL) comprising (d) CDR-L1 comprising the amino acid sequence of SEQ ID NO: 79, (e) CDR-L2 comprising the amino acid sequence of SEQ ID NO: 80, and (f) CDR-L3 comprising the amino acid sequence of SEQ ID NO: 81. In one aspect, the anti-CCR8 antibody comprises a VH sequence of SEQ ID NO: 99 and a VL sequence of SEQ ID NO: 98.

In one aspect, the anti-CCR8 antibody comprises a heavy chain of SEQ ID NO: 105, and a light chain of SEQ ID NO: 104.

In another aspect of any of the above-described embodiments, an anti-CCR8 antibody is provided, wherein the heavy chain of the antibody comprises a shortened C-terminus in which one or two of the C terminal amino acid residues have been removed. In one aspect, the C-terminus of the heavy chain is a shortened C-terminus ending PG. In one aspect, the anti-CCR8 antibody comprises a heavy chain of SEQ ID NO: 117, and a light chain of SEQ ID NO: 104.

In one aspect, the anti-CCR8 antibody is named as “hu.Ab3.H1 L1” in the present disclosure, which can be fucosylated or afucosylated, which optionally contains one or more heavy chain mutations at G236A and I331E, and which optionally comprises a shortened C-terminus of the heavy chain in which one or two of the C terminal amino acid residues have been removed. In some instances, the heavy chain mutations are numbered according to the EU index.

In a further aspect, an anti-CCR8 antibody according to any of the above aspects is a monoclonal antibody, including a chimeric, humanized or human antibody. In one aspect, an anti-CCR8 antibody is an antibody fragment, e.g., a Fv, Fab, Fab′, scFv, diabody, or F(ab′)₂ fragment.

### (vi) Embodiments of a Mouse Surrogate

In one aspect, the present disclosure provides an anti-CCR8 antibody which binds to mouse CCR8, and comprises at least one, at least two, at least three, at least four, at least five, or all six CDRs selected from the group consisting of (a) CDR-H1 comprising the amino acid sequence of SEQ ID NO: 65 or SEQ ID NO: 66, (b) CDR-H2 comprising the amino acid sequence of SEQ ID NO: 67, (c) CDR-H3 comprising the amino acid sequence of SEQ ID NO: 68, (d) CDR-L1 comprising the amino acid sequence of SEQ ID NO: 62, (e) CDR-L2 comprising the amino acid sequence of SEQ ID NO: 63, and (f) CDR-L3 comprising the amino acid sequence of SEQ ID NO: 64. In certain aspects, the anti-CCR8 antibody comprises all six of the aforementioned CDRs. In certain aspects, the anti-CCR8 antibody is a full-length antibody. In certain aspects, the anti-CCR8 antibody is a full-length antibody which binds to mouse CCR8. In certain aspects, the anti-CCR8 antibody is a full-length antibody which binds to mouse CCR8 and is a chimeric antibody (e.g., a rabbit and mouse chimera).

In one aspect, the present disclosure provides an antibody comprising at least one, at least two, or all three VH CDR sequences selected from the group consisting of (a) CDR-H1 comprising the amino acid sequence of SEQ ID NO: 65 or SEQ ID NO: 66, (b) CDR-H2 comprising the amino acid sequence of SEQ ID NO: 67, and (c) CDR-H3 comprising the amino acid sequence of SEQ ID NO: 68. In one aspect, the antibody comprises CDR-H3 comprising the amino acid sequence of SEQ ID NO: 68. In another aspect, the antibody comprises CDR-H3 comprising the amino acid sequence of SEQ ID NO: 68 and CDR-L3 comprising the amino acid sequence of SEQ ID NO: 64. In a further aspect, the antibody comprises CDR-H3 comprising the amino acid sequence of SEQ ID NO: 68, CDR-L3 comprising the amino acid sequence of SEQ ID NO: 64, and CDR-H2 comprising the amino acid sequence of SEQ ID NO: 6. In a further aspect, the antibody comprises (a) CDR-H1 comprising the amino acid sequence of SEQ ID NO: 65 or SEQ ID NO: 66, (b) CDR-H2 comprising the amino acid sequence of SEQ ID NO: 67, and (c) CDR-H3 comprising the amino acid sequence of SEQ ID NO: 68.

In another aspect, the present disclosure provides an antibody comprising at least one, at least two, or all three VL CDR sequences selected from the group consisting of (a) CDR-L1 comprising the amino acid sequence of SEQ ID NO: 62; (b) CDR-L2 comprising the amino acid sequence of SEQ ID NO: 63; and (c) CDR-L3 comprising the amino acid sequence of SEQ ID NO: 64. In one aspect, the antibody comprises (a) CDR-L1 comprising the amino acid sequence of SEQ ID NO: 62; (b) CDR-L2 comprising the amino acid sequence of SEQ ID NO: 63; and (c) CDR-L3 comprising the amino acid sequence of SEQ ID NO: 64.

In another aspect, an antibody as described herein comprises (a) a VH domain comprising at least one, at least two, or all three VH CDR sequences selected from the group consisting of (i) CDR-H1 comprising the amino acid sequence of SEQ ID NO: 65 or SEQ ID NO: 66, (ii) CDR-H2 comprising the amino acid sequence of SEQ ID NO: 67, and (iii) CDR-H3 comprising the amino acid sequence of SEQ ID NO: 68; and (b) a VL domain comprising at least one, at least two, or all three VL CDR sequences selected from the group consisting of (i) CDR-L1 comprising the amino acid sequence of SEQ ID NO: 62; (ii) CDR-L2 comprising the amino acid sequence of SEQ ID NO: 63; and (iii) CDR-L3 comprising the amino acid sequence of SEQ ID NO: 64.

In another aspect, the present disclosure provides an antibody comprising (a) CDR-H1 comprising the amino acid sequence of SEQ ID NO: 65 or SEQ ID NO: 66, (b) CDR-H2 comprising the amino acid sequence of SEQ ID NO: 67, and (c) CDR-H3 comprising the amino acid sequence of SEQ ID NO: 68, and a light chain variable domain (VL) comprising (d) CDR-L1 comprising the amino acid sequence of SEQ ID NO: 62, (e) CDR-L2 comprising the amino acid sequence of SEQ ID NO: 63, and (f) CDR-L3 comprising the amino acid sequence of SEQ ID NO: 64.

In another aspect, an anti-CCR8 antibody comprises one or more of the CDR sequences of the VH sequence of SEQ ID NO: 70. In another aspect, an anti-CCR8 antibody comprises one or more of the CDR sequences of the VL sequence of SEQ ID NO: 69. In another aspect, an anti-CCR8 antibody comprises the CDR sequences of the VH sequence of SEQ ID NO: 70. In another aspect, an anti-CCR8 antibody comprises one or more of the CDR sequences of the VL sequence of SEQ ID NO: 69.

In a further aspect, an anti-CCR8 antibody comprises the CDR-H1, CDR-H2 and CDR-H3 amino acid sequences of the VH domain of SEQ ID NO: 70 and the CDR-L1, CDR-L2 and CDR-L3 amino acid sequences of the VL domain of SEQ ID NO: 69.

In one aspect, an anti-CCR8 antibody comprises one or more of the heavy chain CDR amino acid sequences of the VH domain of SEQ ID NO: 70 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 70. In one aspect, the anti-CCR8 antibody comprises the three heavy chain CDR amino acid sequences of the VH domain of SEQ ID NO: 70 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 70. In one aspect, the anti-CCR8 antibody comprises the three heavy chain CDR amino acid sequences of the VH domain of SEQ ID NO: 70 and a framework of at least 95% sequence identity to the framework amino acid sequence of the VH domain of SEQ ID NO: 70. In another aspect, the anti-CCR8 antibody comprises the three heavy chain CDR amino acid sequences of the VH domain of SEQ ID NO: 70 and a framework of at least of at least 98% sequence identity to the framework amino acid sequence of the VH domain of SEQ ID NO: 70.

In one aspect, an anti-CCR8 antibody comprises one or more of the light chain CDR amino acid sequences of the VL domain of SEQ ID NO: 69 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 69. In one aspect, the anti-CCR8 antibody comprises the three light chain CDR amino acid sequences of the VL domain of SEQ ID NO: 69 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 69. In one aspect, the anti-CCR8 antibody comprises the three light chain CDR amino acid sequences of the VL domain of SEQ ID NO: 69 and a framework of at least 95% sequence identity to the framework amino acid sequence of the VL domain of SEQ ID NO: 69. In another aspect, the anti-CCR8 antibody comprises the three light chain CDR amino acid sequences of the VL domain of SEQ ID NO: 69 and a framework of at least particularly of at least 98% sequence identity to the framework amino acid sequence of the VL domain of SEQ ID NO: 69.

In one aspect, the anti-CCR8 antibody comprises (a) CDR-H1 comprising the amino acid sequence of SEQ ID NO: 65 or SEQ ID NO: 66, (b) CDR-H2 comprising the amino acid sequence of SEQ ID NO: 67, (c) CDR-H3 comprising the amino acid sequence of SEQ ID NO: 68, (d) CDR-L1 comprising the amino acid sequence of SEQ ID NO: 62, (e) CDR-L2 comprising the amino acid sequence of SEQ ID NO: 63, and (f) CDR-L3 comprising the amino acid sequence of SEQ ID NO: 64, and a VH domain having at least 90%, 91%, 92%, 93%, 94%, 95%, 96%, 97%, 98%, 99%, or 100% sequence identity to the amino acid sequence of SEQ ID NO: 70, and a VL domain having at least 90%, 91%, 92%, 93%, 94%, 95%, 96%, 97%, 98%, 99%, or 100% sequence identity to the amino acid sequence of SEQ ID NO: 69. In one aspect, the VH domain has at least 95% sequence identity to the amino acid sequence of SEQ ID NO: 70. In one aspect, the VL domain has at least 95% sequence identity to the amino acid sequence of SEQ ID NO: 69. In one aspect, the antibody binds to mouse CCR8 having a dissociation constant (K_{D}) that is up to 10-fold reduced or up to 10-fold increased when compared to the dissociation constant (K_{D}) of an antibody comprising a VH sequence of SEQ ID NO: 70 and a VL sequence of SEQ ID NO: 69.

In another aspect, an anti-CCR8 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 70. In one aspect, an anti-CCR8 antibody comprises a heavy chain variable domain (VH) sequence having at least 95%, sequence identity to the amino acid sequence of SEQ ID NO: 70. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-CCR8 antibody comprising that sequence retains the ability to bind to mouse CCR8. In certain aspects, a total of 1 to 10 amino acids have been substituted, inserted and/or deleted in the amino acid sequence of SEQ ID NO: 70. In certain aspects, substitutions, insertions, or deletions occur in regions outside the CDRs (i.e., in the FRs). Optionally, the anti-CCR8 antibody comprises the VH sequence of SEQ ID NO: 70, including post-translational modifications of that sequence. In a particular aspect, the VH comprises one, two or three CDRs selected from: SEQ ID NO: 65 or SEQ ID NO: 66, (b) CDR-H2 comprising the amino acid sequence of SEQ ID NO: 67, (c) CDR-H3 comprising the amino acid sequence of SEQ ID NO: 68. In another aspect, an anti-CCR8 antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO: 69. In one aspect, an anti-CCR8 antibody comprises a light chain variable domain (VL) sequence having at least 95% sequence identity to the amino acid sequence of SEQ ID NO: 69. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-CCR8 antibody comprising that sequence retains the ability to bind to CCR8. In certain aspects, a total of 1 to 10 amino acids have been substituted, inserted and/or deleted in the amino acid sequence of SEQ ID NO: 69. In certain aspects, the substitutions, insertions, or deletions occur in regions outside the CDRs (i.e., in the FRs). Optionally, the anti-CCR8 antibody comprises the VL sequence of SEQ ID NO: 69, including post-translational modifications of that sequence. In a particular aspect, the VL comprises one, two or three CDRs selected from: (a) CDR-L1 comprising the amino acid sequence of SEQ ID NO: 62, (b) CDR-L2 comprising the amino acid sequence of SEQ ID NO: 63, and (c) CDR-L3 comprising the amino acid sequence of SEQ ID NO: 64.

In another aspect, an anti-CCR8 antibody is provided, wherein the antibody comprises a VH sequence as in any of the aspects provided above, and a VL sequence as in any of the aspects provided above. In one aspect, the antibody comprises the VH sequence of SEQ ID NO: 70 and the VL sequence of SEQ ID NO: 69, including post-translational modifications of those sequences.

In another aspect, an anti-CCR8 antibody is provided which binds to mouse CCR8, wherein the antibody comprises a heavy chain variable domain (VH) comprising (a) CDR-H1 comprising the amino acid sequence of SEQ ID NO: 65 or SEQ ID NO: 66, (b) CDR-H2 comprising the amino acid sequence of SEQ ID NO: 67 and (c) CDR-H3 comprising the amino acid sequence of SEQ ID NO: 68, and a light chain variable domain (VL) comprising (d) CDR-L1 comprising the amino acid sequence of SEQ ID NO: 62, (e) CDR-L2 comprising the amino acid sequence of SEQ ID NO: 63, and (f) CDR-L3 comprising the amino acid sequence of SEQ ID NO: 64. In one aspect, the anti-CCR8 antibody comprises a VH sequence of SEQ ID NO: 70 and a VL sequence of SEQ ID NO: 69.

In one aspect, the anti-CCR8 antibody comprises a heavy chain of SEQ ID NO: 72, and a light chain of SEQ ID NO: 71.

In a further aspect, an anti-CCR8 antibody according to any of the above aspects is a monoclonal antibody, including a chimeric antibody. In one aspect, an anti-CCR8 antibody is an antibody fragment, e.g., a Fv, Fab, Fab′, scFv, diabody, or F(ab′)₂ fragment.

(vii) Other Embodiments

In a further aspect, an anti-CCR8 antibody according to any of the above aspects may incorporate any of the features, singly or in combination, as described in Sections 1-5 below:

### 1. Antibody Fragments

In certain aspects, an antibody provided herein is an antibody fragment.

In one aspect, the antibody fragment is a Fab, Fab′, Fab′-SH, or F(ab′)₂ fragment, in particular a Fab fragment. Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain. “Fab′ fragments” differ from Fab fragments by the addition of residues at the carboxy terminus of the CH1 domain including one or more cysteines from the antibody hinge region. Fab‘-SH are Fab′ fragments in which the cysteine residue(s) of the constant domains bear a free thiol group. Pepsin treatment yields an F(ab′)₂ fragment that has two antigen-binding sites (two Fab fragments) and a part of the Fc region. For discussion of Fab and F(ab′)₂ fragments comprising salvage receptor binding epitope residues and having increased in vivo half-life, see U.S. Pat. No. 5,869,046.

In another aspect, the antibody fragment is a diabody, a triabody or a tetrabody. “Diabodies” are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

In a further aspect, the antibody fragment is a single chain Fab fragment. A “single chain Fab fragment” or “scFab” is a polypeptide consisting of an antibody heavy chain variable domain (VH), an antibody heavy chain constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. In particular, said linker is a polypeptide of at least 30 amino acids, preferably between 32 and 50 amino acids. Said single chain Fab fragments are stabilized via the natural disulfide bond between the CL domain and the CH1 domain. In addition, these single chain Fab fragments might be further stabilized by generation of interchain disulfide bonds via insertion of cysteine residues (e.g., position 44 in the variable heavy chain and position 100 in the variable light chain according to Kabat numbering).

In another aspect, the antibody fragment is single-chain variable fragment (scFv). A “single-chain variable fragment” or “scFv” is a fusion protein of the variable domains of the heavy (VH) and light chains (VL) of an antibody, connected by a linker. In particular, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Plückthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

In another aspect, the antibody fragment is a single-domain antibody. “Single-domain antibodies” are antibody fragments comprising all or a portion of the heavy chain variable domain or all or a portion of the light chain variable domain of an antibody. In certain aspects, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as recombinant production by recombinant host cells (e.g., E. coli), as described herein.

### 2. Chimeric and Humanized Antibodies

In certain aspects, an antibody provided herein is a chimeric antibody. Certain chimeric antibodies are described, e.g., in U.S. Pat. No. 4,816,567; and Morrison et al., Proc. Nat. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In some aspects, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Nat. Acad. Sci. USA, 89:4285 (1992); and Presta et al. J. Immunol., 151:2623 (1993)); human mature (somatically mutated) framework regions or human germline framework regions (see, e.g., Almagro and Fransson, Front.

Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 3. Human Antibodies

In certain aspects, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating variable domain sequences selected from human-derived phage display libraries. Such variable domain sequences may then be combined with a desired human constant domain. Techniques for selecting human antibodies from antibody libraries are described below.

### 4. Multispecific Antibodies

In certain aspects, an antibody provided herein is a multispecific antibody, e.g., a bispecific antibody. “Multispecific antibodies” are monoclonal antibodies that have binding specificities for at least two different sites, i.e., different epitopes on different antigens or different epitopes on the same antigen. In certain aspects, the multispecific antibody has three or more binding specificities. In certain aspects, one of the binding specificities is for CCR8 and the other specificity is for any other antigen. In certain aspects, bispecific antibodies may bind to two (or more) different epitopes of CCR8. Multispecific (e.g., bispecific) antibodies may also be used to localize cytotoxic agents or cells to cells which express CCR8. Multispecific antibodies may be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (see, e.g., U.S. Pat. No. 5,731,168, and Atwell et al., J. Mol. Biol. 270:26 (1997)). Multi-specific antibodies may also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

### 5. Antibody Variants

In certain aspects, amino acid sequence variants of the antibodies provided herein are contemplated. For example, it may be desirable to alter the binding affinity and/or other biological properties of the antibody. Amino acid sequence variants of an antibody may be prepared by introducing appropriate modifications into the nucleotide sequence encoding the antibody, or by peptide synthesis.

Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, and Deletion Variants

In certain aspects, antibody variants having one or more amino acid substitutions are provided. Sites of interest for substitutional mutagenesis include the CDRs and FRs.

In one aspect, the VL sequence of the antibody disclosed herein comprises a V4M mutation, a P43A mutation, a F46L mutation, a C90Q mutation, or a combination thereof. In one aspect, the VH sequence of the antibodies disclosed herein comprises a G49S mutation, a K71R mutation, a S73N mutation, or a combination thereof. In one aspect, the VL sequence of the antibodies disclosed herein comprises a Y21 mutation. In one aspect, the VH sequence of the antibodies disclosed herein comprises a S73N mutation, a V78L mutation, a T76N mutation, a F91Y mutation, and a P105Q mutation, or a combination thereof. In some instances, any of the foregoing mutations are numbered according to Kabat.

Conservative substitutions are shown in Table 2 under the heading of “conservative substitutions”. More substantial changes are provided in Table 2 under the heading of “exemplary substitutions”, and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 2
Contemplated amino acid substitutions

| Original | Exemplary | Conservative |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for a member of another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more. CDR residues are mutated and the variant antibodies displayed on phage and screened for a particular biological activity (e.g., binding affinity).

Alterations (e.g., substitutions) may be made in CDRs, e.g., to improve antibody affinity. Such alterations may be made in CDR “hotspots”, i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain aspects, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in the CDRs. Such alterations may, for example, be outside of antigen contacting residues in the CDRs. In certain variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT (antibody directed enzyme prodrug therapy)) or a polypeptide which increases the serum half-life of the antibody.

### b) Glycosylation Variants

In certain aspects, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the oligosaccharide attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody as described herein may be made in order to create antibody variants with certain improved properties.

In one aspect, antibody variants are provided having a non-fucosylated oligosaccharide, i.e., an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure, and such antibodies are further referred to herein as an “afucosylated antibodies.” In one aspect, antibody variants are provided having an increased proportion of non-fucosylated oligosaccharides in the Fc region as compared to a native or parent antibody. For example, the proportion of non-fucosylated oligosaccharides may be at least about 20%, at least about 40%, at least about 60%, at least about 80%, or even about 100% (i.e., no fucosylated oligosaccharides are present). In certain embodiments, the proportion of afucosylation is between about 65% to about 100%, between about 80% to about 100%, or between about 80% to about 95%. The percentage of non-fucosylated oligosaccharides is the (average) amount of oligosaccharides lacking fucose residues, relative to the sum of all oligosaccharides attached to Asn 297 (e. g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies, e.g., Asn 299. Such antibodies having an increased proportion of non-fucosylated oligosaccharides in the Fc region may have improved FcγRIIIa receptor binding and/or improved effector function, in particular improved ADCC function. See, e.g., US 2003/0157108; US 2004/0093621.

In one aspect, the present disclosure provides afucosylated antibody variants that have enhanced FcγRIIIa receptor binding. In one aspect, the present disclosure provides afucosylated antibody variants that have enhanced antibody-dependent cellular cytotoxicity (ADCC). In one aspect, the present disclosure provides afucosylated antibody variants that have antibody-dependent cellular phagocytosis (ADCP) activities.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUTB, knockout CHO cells (see, e.g., Yamane-Ohnuki et al. Biotech. Bioeng. 87:614-622 (2004); Kanda, Y. et al., Biotechnol. Bioeng., 94(4):680-688 (2006); and WO 2003/085107), or cells with reduced or abolished activity of a GDP-fucose synthesis or transporter protein (see, e.g., US2004259150, US2005031613, US2004132140, US2004110282). See also Pereira et al., MABS (2018) 693-711.

In a further aspect, antibody variants are provided with bisected oligosaccharides, e.g., in which a biantennary oligosaccharide attached to the Fc region of the antibody is bisected by GlcNAc. Such antibody variants may have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

### c) Fc Region Variants

In certain aspects, one or more amino acid modifications may be introduced into the Fc region of an antibody provided herein, thereby generating an Fc region variant. The Fc region variant may comprise a human Fc region sequence (e.g., a human IgG₁, IgG₂, IgG₃ or IgG₄ Fc region) comprising an amino acid modification (e.g., a substitution) at one or more amino acid positions.

In certain aspects, the invention contemplates an antibody variant that possesses some but not all effector functions, which make it a desirable candidate for applications in which the half-life of the antibody in vivo is important yet certain effector functions (such as complement-dependent cytotoxicity (CDC) and antibody-dependent cell-mediated cytotoxicity (ADCC)) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Natl Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1 q binding assays may also be carried out to confirm that the antibody is unable to bind C1 q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006); WO 2013/120929 A1).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).) In certain aspects, an antibody variant comprises an Fc region with one or more amino acid substitutions which improve ADCC, e.g., substitutions at positions 298, 333, and/or 334 of the Fc region (EU numbering of residues).

In certain aspects, an antibody variant comprises an Fc region with one or more amino acid substitutions which diminish FcγR binding, e.g., substitutions at positions 234 and 235 of the Fc region (EU numbering of residues). In one aspect, the substitutions are L234A and L235A (LALA). In certain aspects, the antibody variant further comprises D265A and/or P329G in an Fc region derived from a human IgG₁ Fc region. In one aspect, the substitutions are L234A, L235A and P329G (LALA-PG) in an Fc region derived from a human IgG₁ Fc region. (See, e.g., WO 2012/130831). In another aspect, the substitutions are L234A, L235A and D265A (LALA-DA) in an Fc region derived from a human IgG₁ Fc region.

In certain aspects, an antibody variant comprises an Fc region with one or more amino acid substitutions which improve FcγR binding (and thereby improve effector function), e.g., substitutions at positions. In certain aspects, the antibody variant comprises an Fc region with at least one amino acid substitutions of G236A, 1332E, S298A, E333A, K334A, S239D, A330L, F243L, R292P, Y300L, V3051, P396L, L235V, L234Y, L235Q, G236W, S239M, H268D, D270E, K326D, A330M, K334E (See, e.g., Liu et al., Antibodies (Basel) (2020); 9(4):64).

In some aspects, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (See, e.g., U.S. Pat. No. 7,371,826; Dall'Acqua, W. F., et al. J. Biol. Chem. 281 (2006) 23514-23524).

Fc region residues critical to the mouse Fc-mouse FcRn interaction have been identified by site-directed mutagenesis (see e.g., Dall'Acqua, W. F., et al. J. Immuno/169 (2002) 5171-5180). Residues 1253, H310, H433, N434, and H435 (EU numbering of residues) are involved in the interaction (Medesan, C., et al., Eur. J. Immunol. 26 (1996) 2533; Firan, M., et al., Int. Immunol. 13 (2001) 993; Kim, J. K., et al., Eur. J. Immunol. 24 (1994) 542). Residues 1253, H310, and H435 were found to be critical for the interaction of human Fc with murine FcRn (Kim, J. K., et al., Eur. J. Immunol. 29 (1999) 2819). Studies of the human Fc-human FcRn complex have shown that residues 1253, S254, H435, and Y436 are crucial for the interaction (Firan, M., et al., Int. Immunol. 13 (2001) 993; Shields, R. L., et al., J. Biol. Chem. 276 (2001) 6591-6604). In Yeung, Y. A., et al. (J. Immunol. 182 (2009) 7667-7671) various mutants of residues 248 to 259 and 301 to 317 and 376 to 382 and 424 to 437 have been reported and examined.

In certain aspects, an antibody variant comprises an Fc region with one or more amino acid substitutions, which reduce FcRn binding, e.g., substitutions at positions 253, and/or 310, and/or 435 of the Fc-region (EU numbering of residues). In certain aspects, the antibody variant comprises an Fc region with the amino acid substitutions at positions 253, 310 and 435. In one aspect, the substitutions are 1253A, H310A and H435A in an Fc region derived from a human IgG1 Fc-region. See, e.g., Grevys, A., et al., J. Immunol. 194 (2015) 5497-5508.

In certain aspects, an antibody variant comprises an Fc region with one or more amino acid substitutions, which reduce FcRn binding, e.g., substitutions at positions 310, and/or 433, and/or 436 of the Fc region (EU numbering of residues). In certain aspects, the antibody variant comprises an Fc region with the amino acid substitutions at positions 310, 433 and 436. In one aspect, the substitutions are H310A, H433A and Y436A in an Fc region derived from a human IgG1 Fc-region. (See, e.g., WO 2014/177460 A1).

In certain aspects, an antibody variant comprises an Fc region with one or more amino acid substitutions which increase FcRn binding, e.g., substitutions at positions 252, and/or 254, and/or 256 of the Fc region (EU numbering of residues). In certain aspects, the antibody variant comprises an Fc region with amino acid substitutions at positions 252, 254, and 256. In one aspect, the substitutions are M252Y, S254T and T256E in an Fc region derived from a human IgG₁ Fc-region. See also Duncan & Winter, Nature 322:738-40 (1988); U.S. Pat. Nos. 5,648,260; 5,624,821; and WO 94/29351 concerning other examples of Fc region variants.

The C-terminus of the heavy chain of the antibody as reported herein can be a complete C-terminus ending with the amino acid residues PGK. The C-terminus of the heavy chain can be a shortened C-terminus in which one or two of the C terminal amino acid residues have been removed. In one aspect, the C-terminus of the heavy chain is a shortened C-terminus ending PG. In one aspect of all aspects as reported herein, an antibody comprising a heavy chain including a C-terminal CH3 domain as specified herein, comprises the C-terminal glycine-lysine dipeptide (G446 and K447, EU index numbering of amino acid positions). In one aspect of all aspects as reported herein, an antibody comprising a heavy chain including a C-terminal CH3 domain, as specified herein, comprises a C-terminal glycine residue (G446, EU index numbering of amino acid positions). In one aspect of all aspects as reported herein, an antibody comprising a heavy chain including a C-terminal CH3 domain, as specified herein, comprises a C-terminal proline residue (P445, EU index numbering of amino acid positions).

### d) Cysteine Engineered Antibody Variants

In certain aspects, it may be desirable to create cysteine engineered antibodies, e.g., THIOMAB™ antibodies, in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. Cysteine engineered antibodies may be generated as described, e.g., in U.S. Pat. No. 7,521,541, 8,30,930, 7,855,275, 9,000,130, or WO 2016040856.

### e) Antibody Derivatives

In certain aspects, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### C. Recombinant Methods and Compositions

Antibodies as disclosed herein may be produced using recombinant methods and compositions, e.g., as described in U.S. Pat. No. 4,816,567. For these methods one or more isolated nucleic acid(s) encoding an antibody are provided.

In case of a native antibody or native antibody fragment two nucleic acids are required, one for the light chain or a fragment thereof and one for the heavy chain or a fragment thereof. Such nucleic acid(s) encode an amino acid sequence comprising the VL and/or an amino acid sequence comprising the VH of the antibody (e.g., the light and/or heavy chain(s) of the antibody). These nucleic acids can be on the same expression vector or on different expression vectors.

In case of a bispecific antibody with heterodimeric heavy chains four nucleic acids are required, one for the first light chain, one for the first heavy chain comprising the first heteromonomeric Fc-region polypeptide, one for the second light chain, and one for the second heavy chain comprising the second heteromonomeric Fc-region polypeptide. The four nucleic acids can be comprised of one or more nucleic acid molecules or expression vectors. Such nucleic acid(s) encode an amino acid sequence comprising the first VL and/or an amino acid sequence comprising the first VH including the first heteromonomeric Fc-region and/or an amino acid sequence comprising the second VL and/or an amino acid sequence comprising the second VH including the second heteromonomeric Fc-region of the antibody (e.g., the first and/or second light and/or the first and/or second heavy chains of the antibody). These nucleic acids can be on the same expression vector or on different expression vectors, normally these nucleic acids are located on two or three expression vectors, i.e., one vector can comprise more than one of these nucleic acids. Examples of these bispecific antibodies are CROSSMAB® (see, e.g., Schaefer, W. et al, PNAS, 108 (2011) 11187-1191). For example, one of the heteromonomeric heavy chain comprises the so-called “knob mutations” (T366W and optionally one of S354C or Y349C) and the other comprises the so-called “hole mutations” (T366S, L368A and Y407V and optionally Y349C or S354C) (see, e.g., Carter, P. et al., Immunotechnol. 2 (1996) 73) according to EU index numbering.

In one aspect, isolated nucleic acids encoding an antibody as used in the methods as reported herein are provided.

In one aspect, a method of making an anti-CCR8 antibody is provided, wherein the method comprises culturing a host cell comprising nucleic acid(s) encoding the antibody, as provided above, under conditions suitable for expression of the antibody, and optionally recovering the antibody from the host cell (or host cell culture medium).

For recombinant production of an anti-CCR8 antibody, nucleic acids encoding the antibody, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, NJ (2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized”, resulting in the production of an antibody with a partially or fully human glycosylation pattern. See Gerngross, T. U., Nat. Biotech. 22 (2004) 1409-1414; and Li, H. et al., Nat. Biotech. 24 (2006) 210-215.

Suitable host cells for the expression of (glycosylated) antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

In one aspect, the host cell is eukaryotic, e.g., a Chinese Hamster Ovary (CHO) cell or lymphoid cell (e.g., Y0, NS0, Sp20 cell).

### D. Assays

Anti-CCR8 antibodies provided herein may be identified, screened for, or characterized for their physical/chemical properties and/or biological activities by various assays known in the art.

### 1. Binding Assays and Other Assays

In one aspect, an antibody as described herein is tested for its antigen binding activity, e.g., by known methods such as ELISA, Western blot, etc.

In another aspect, competition assays may be used to identify an antibody that competes with an anti-CCR8 antibody of the presently disclosed subject matter, e.g., Ab1, Ab2, Ab3, Ab4, and Ab5, for binding to CCR8. In certain aspects, such a competing antibody binds to the same epitope (e.g., a linear or a conformational epitope) that is bound by an anti-CCR8 antibody of the presently disclosed subject matter, e.g., Ab1, Ab2, Ab3, Ab4, and Ab5. Detailed exemplary methods for mapping an epitope to which an antibody binds are provided in Morris (1996) “Epitope Mapping Protocols”, in Methods in Molecular Biology vol. 66 (Humana Press, Totowa, NJ).

In an exemplary competition assay, immobilized CCR8 is incubated in a solution comprising a first labeled antibody that binds to CCR8 (e.g., an anti-CCR8 antibody of the presently disclosed subject matter, e.g., Ab1, Ab2, Ab3, Ab4, and Ab5) and a second unlabeled antibody that is being tested for its ability to compete with the first antibody for binding to CCR8. The second antibody may be present in a hybridoma supernatant. As a control, immobilized CCR8 is incubated in a solution comprising the first labeled antibody but not the second unlabeled antibody. After incubation under conditions permissive for binding of the first antibody to CCR8, excess unbound antibody is removed, and the amount of label associated with immobilized CCR8 is measured. If the amount of label associated with immobilized CCR8 is substantially reduced in the test sample relative to the control sample, then that indicates that the second antibody is competing with the first antibody for binding to CCR8. See Harlow and Lane (1988) Antibodies: A Laboratory Manual ch. 14 (Cold Spring Harbor Laboratory, Cold Spring Harbor, NY).

### 2. Activity Assays

In one aspect, assays are provided for identifying anti-CCR8 antibodies thereof having biological activity. Biological activity may include, e.g., antibody-dependent cellular cytotoxicity (ADCC), ADCC against Tregs, antibody-dependent cellular phagocytosis (ADCP), depletion of Tregs. Antibodies having such biological activity in vivo and/or in vitro are also provided.

In certain aspects, an anti-CCR8 antibody as described herein is tested for measuring ADCC of the antibody. ADCC assays are performed as previously reported in Kamen, L., et al., Development of a kinetic antibody-dependent cellular cytotoxicity assay. J Immunol Methods, 2019. 468: p. 49-54, and Schnueriger, A., et al., Development of a quantitative, cell-line based assay to measure ADCC activity mediated by therapeutic antibodies. Mol Immunol, 2011. 48(12-13): p. 1512-17, with some modifications, using CD16 engineered NK-92_F158 as effector cells and CHO cells that stably express human CCR8 and Ga 15 subunit (CHO/hCCR8.Gna15) as target cells. Briefly, lysis of target cells by ADCC is measured by the calcein release method. The target cells are labeled with Calcein-AM, then washed and plated onto 384-well plates at a density of 3000 cells/well. Anti-CCR8 antibody is added at various concentrations from 0.004 to 1 μg/mL, followed by the addition of NK-92_F158 cells at an effector:target (E:T) ratio of 10:1. The plates are then incubated for 2.5 hours at 37° C. After incubation, the plates are centrifuged at 200×g for 3 minutes, the supernatants are transferred to a white opaque 384-well microplate, and fluorescent signals are measured in relative fluorescence units (RFU). Signals from the wells containing only the target cells represent spontaneous release of the calcein from labeled cells (spontaneous release), whereas wells containing target cells lysed with TRITON™ X-100 provide the maximal signal available (maximal release). Antibody-independent cell-mediated cytotoxicity (AICC) are measured in wells containing target and effector cells without the addition of the antibody. Samples and controls are tested at least in duplicate in the same plates. The extent of specific ADCC activity is calculated as follows:

$$\% ADCC = 100 \times ( mean experimental release - mean AICC ) / ( mean maximum release - mean spontaneous release )$$

The ADCC activity is plotted as a function of antibody concentrations and the data are fitted to an asymmetric sigmoidal four-parameter logistic (4PL) model.

In certain aspects, an anti-CCR8 antibody as described herein is tested for measuring ADCC against Treg cells. To induce CCR8 expression on T cells from human peripheral blood mononuclear cells (PBMC), 107 human PBMC are intraperitoneally transferred to NOD.Cg-Prkdc^{scid} Il2rg^{tm1Wjl}/SzJ (NSG™) mice (JAX) and spleens are collected 2-3 weeks post-transfer. Human T cells are enriched from single cell suspensions of NSG™ splenocytes and primary NK cells are enriched from human PBMC. Human T cells are incubated with 0.001-1 μg/mL anti-CCR8 antibody for 30 minutes at room temperature prior to the addition of primary NK cells at an effector:target ratio of 2:1. After overnight incubation at 37° C., cells are collected, surface stained, and intracellularly stained. Antibodies used to define T cell populations are CD45 (H130), CD3 (SK7), CD8 (RPA-T8), and CD14 (63D3), CD4 (RPA-T4), and FOXP3 (236A/E7). COUNTBRIGHT™ Absolute Counting Beads is added to each sample prior to acquisition. Flow cytometry is performed. Absolute cell counts are calculated. ADCC activity against Treg cells is measured by calculating the ratio of recovered Treg cells to recovered CD8 cells (Treg/CD8) or conventional CD4 T cells to recovered CD8 T cells (CD4conv/CD8).

In certain aspects, an anti-CCR8 antibody as described herein is tested for measuring its binding to regulatory T cells (Treg cells or Tregs) by Fluorescence-Activated Cell Sorting (FACS) flow cytometry. Human colorectal dissociated tumor cells (DTC) are thawed. Cells are surface stained with EFLuOR™ 780-conjugated Fixable Viability Dye and 2 μg/mL mAb specific for CCR8, OX40 (positive control), Herceptin (negative control), or anti-hIgG (negative control) for 20 min at 4° C. followed by secondary detection with AF647-conjugated AffiniPure F(ab′)₂ Fragment Goat anti-Human IgG, Fcg fragment-specific for 10 min at 4° C. Cells are then intracellularly stained. Antibodies used to define T cell populations are CD45 (H130), CD3 (SK7), CD8 (RPA-T8), and CD14 (63D3) from BD Biosciences, CD4 (RPA-T4), and FOXP3 (236A/E7). Flow cytometry is performed and analyzed.

In certain aspects, an anti-CCR8 antibody as described herein is tested for measuring ADCP of the antibody. Human CD14₊ monocytes are first isolated from blood of donors with known FcgRIIa and FcgRIIIa genotype information. The purified CD14⁺ monocytes are differentiated into macrophages. Then 50 ng/mL of hIL-10 are added to polarize the macrophages for 24 hours prior to ADCP assay. NUCLIGHT™ Red transfected CHO/hCCR8.Gna15 target cells are pre-incubated with anti-CCR8 antibodies for 20 minutes in the presence of 20 mg/mL of non-specific human IgG. Then the above cell mixtures are added to the macrophage (effector cell) plate at an E:T ratio of 1:1. Cell images are obtained with bright field and red laser settings every one hour for a period of 6 hours. The red cell count in each well (remaining target cells) is normalized by the macrophage numbers. The ADCP activity is calculated as the percentage of decrease of the normalized red cell count in each sample compared to the negative control where isotype control antibody is present. Then the ADCP activity is plotted as a function of antibody concentrations and the data are fitted to an asymmetric sigmoidal four-parameter logistic (4PL) model. The EC₅₀ value for each antibody is determined as the concentration reaching 50% target cell killing.

In certain aspects, an anti-CCR8 antibody (e.g., a mouse surrogate antibody) as described herein is tested for measuring depletion of Treg cells in vivo, mice with established tumors are treated with an anti-CCR8 antibody (e.g., a mouse surrogate antibody disclosed herein) and the proportion of Treg cells, conventional CD4 T cells and CD8 T cells among leukocytes in tumors, spleen and tumor-draining lymph nodes are analyzed. To this end, tumor cells are harvested in log-phase growth and resuspended in Hanks' Balanced Salt Solution (HBSS) containing MATRIGEL® at a 1:1 ratio. Mice are inoculated subcutaneously in the flank with 0.1 million tumor cells in 100 microliters of HBSS+MATRIGEL®. Tumors are monitored until they become established and reached a mean tumor volume 130-230 mm³. Mice are then randomized into treatment groups. Treatment with an anti-CCR8 or an anti-gp120 isotype control Ab is administered intravenously. Three days later mice are sacrificed, and tumors, spleens and tumor-draining lymph nodes obtained for analysis. To generate single cell suspensions, tumors are minced and digested. Single cell suspensions are surface stained with fluorescently labelled anti-CD45, anti-CD4 and anti-CD8 antibodies and intracellularly stained with fluorescently labelled anti-Foxp3 antibody. Flow cytometry may be performed on a FORTESSA™ X-20 or FACSYMPHONY™ and analyzed with FLOWJO™ software.

In certain aspects, an anti-CCR8 antibody (e.g., a mouse surrogate antibody) as described herein is tested for tumor growth inhibition following anti-CCR8-mediated depletion of tumor-infiltrating Treg cells in vivo. Mice with established tumors are treated with a mouse surrogate anti-CCR8 antibody and are monitored for tumor growth over time.

### E. Methods and Compositions for Diagnostics and Detection

In certain aspects, any of the anti-CCR8 antibodies provided herein is useful for detecting the presence of CCR8 in a biological sample. The term “detecting” as used herein encompasses quantitative or qualitative detection. In certain aspects, a biological sample comprises a cell or tissue, such as tumor.

In one aspect, an anti-CCR8 antibody for use in a method of diagnosis or detection is provided. In a further aspect, a method of detecting the presence of CCR8 in a biological sample is provided. In certain aspects, the method comprises contacting the biological sample with an anti-CCR8 antibody as described herein under conditions permissive for binding of the anti-CCR8 antibody to CCR8, and detecting whether a complex is formed between the anti-CCR8 antibody and CCR8. Such method may be an in vitro or in vivo method. In one aspect, an anti-CCR8 antibody is used to select subjects eligible for therapy with an anti-CCR8 antibody, e.g., where CCR8 is a biomarker for selection of subjects.

In certain aspects, labeled anti-CCR8 antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luciferases, e.g., firefly luciferase and bacterial luciferase (U.S. Pat. No. 4,737,456), luciferin, 2,3-dihydrophthalazinediones, horseradish peroxidase (HRP), alkaline phosphatase, p-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### F. Pharmaceutical Compositions

In a further aspect, provided are pharmaceutical compositions comprising any of the antibodies provided herein, e.g., for use in any of the methods and compositions for use described herein. In one aspect, a pharmaceutical composition comprises any of the antibodies provided herein and a pharmaceutically acceptable carrier. In another aspect, a pharmaceutical composition comprises any of the antibodies provided herein and at least one additional therapeutic agent, e.g., as described below.

Pharmaceutical compositions (formulations) of an anti-CCR8 antibody as described herein can be prepared by combining the antibody with pharmaceutically acceptable carriers or excipients known to the skilled person. See, for example Remington's Pharmaceutical Sciences 16th edition, Osol, A. Ed. (1980), Shire S., Monoclonal Antibodies: Meeting the Challenges in Manufacturing, Formulation, Delivery and Stability of Final Drug Product, 1^{st} Ed., Woodhead Publishing (2015), § 4 and Falconer R. J., Biotechnology Advances (2019), 37, 107412. Exemplary pharmaceutical compositions of an anti-CCR8 antibody as described herein are lyophilized, aqueous, frozen, etc.

Pharmaceutically acceptable carriers are generally nontoxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

The pharmaceutical composition herein may also contain more than one active ingredient as necessary for the particular indication being treated, preferably those with complementary activities that do not adversely affect each other. For example, it may be desirable to further provide an additional therapeutic agent useful for treatment of the same disease. Such active ingredients are suitably present in combination in amounts that are effective for the purpose intended.

The pharmaceutical compositions to be used for in vivo administration are generally sterile. Sterility may be readily accomplished, e.g., by filtration through sterile filtration membranes.

### G. Articles of Manufacture

In another aspect, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody as disclosed herein. The label or package insert indicates that the composition is used for treating the condition of choice. Moreover, the article of manufacture may comprise (a) a first container with a composition contained therein, wherein the composition comprises an antibody as disclosed herein; and (b) a second container with a composition contained therein, wherein the composition comprises a further cytotoxic or otherwise therapeutic agent. The article of manufacture in this aspect as described herein may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

For example, provided herein is an article of manufacture or a kit for carrying out any of the methods disclosed herein. In some instances, the article of manufacture or kit includes an anti-CCR8 antibody as disclosed herein. In some instances, the article of manufacture or kit includes instructions to administer the anti-CCR8 antibody to a subject in accordance with any one of the methods disclosed herein. In some instances, the article of manufacture or kit further includes one or more additional therapeutic agents. In some instances, the one or more additional therapeutic agents comprise atezolizumab. In some instances, the article of manufacture or kit includes instructions to administer the anti-CCR8 antibody and the atezolizumab to a subject in accordance with any one of the methods disclosed herein.

### EXAMPLES

The following are further non-limiting examples of antibodies, methods and compositions as described herein. It is understood that various other embodiments may be practiced, given the general description provided above.

### Example 1. Discovery and Engineering of Anti-CCR8 Monoclonal Antibodies

New Zealand White rabbits were immunized with recombinant huCCR8, a huCCR8+ rabbit cell line, extracellular vesicles containing huCCR8, and sulfated and unsulfated peptides derived from N-terminal regions of huCCR8. Single B cells were isolated following the protocol set forth in Lin et al., PLoS ONE 15(12), 2020. The B cell culture supernatants were then assayed by direct Flow Activated Cell Sorting (FACS; flow cytometry) of IgG+ B cells into single wells for binding to human and cyno CCR8+ CHO cells and control CHO cells. CCR8 specific B cells were lysed and immediately frozen in −80° C. for storage until molecular cloning. Variable regions (VH and VL) of each monoclonal antibody from rabbit B cells were cloned into expression vectors from extracted mRNA as described in Lin et al., PLoS ONE 15(12), 2020. Individual recombinant rabbit antibodies were expressed in Expi293 cells and subsequently purified with protein A.

Over 480 anti-CCR8 antibodies were obtained that bound to either human or cyno CCR8 CHO cells. Antibodies were further selected based on their relative mean fluorescent intensities (MFIs) on the human and cyno CCR8 CHO cell lines and sequence diversity. From the antibodies that showed MFI differences less than 5-fold on human and cyno CCR8 CHO cells, five unique groups of antibodies were identified (designated Ab1-Ab5). One representative sequence from each group was selected for humanization.

Variants constructed during the humanization of the rabbit monoclonal antibodies were assessed in the form of human IgG1. Hypervariable regions from each of the rabbit antibodies (namely positions 24-34 (L1), 50-56 (L2) and 89-97 (L3) in VL domain, and 26-35 (H1), 50-65 (H2) and 95-102 (H3) in VH domain) were grafted into various acceptor frameworks. Residue numbers are according to Kabat et al., Sequences of proteins of immunological interest, 5th Ed., Public Health Service, National Institutes of Health, Bethesda, Md. (1991). All VL and VH Vernier positions from rabbit antibodies were also grafted into their respective human germline frameworks. The grafts with all rabbit amino acids in Vernier positions are referred to as H1L1. The binding ability of humanized CCR8 antibodies to CHO-huCCR8.Gna15 stable cell line was compared to their chimeric parental clones. Rabbit Vernier positions of version H1 L1 antibodies were converted back to human residues to evaluate the contribution of each rabbit Vernier position to binding to huCCR8.

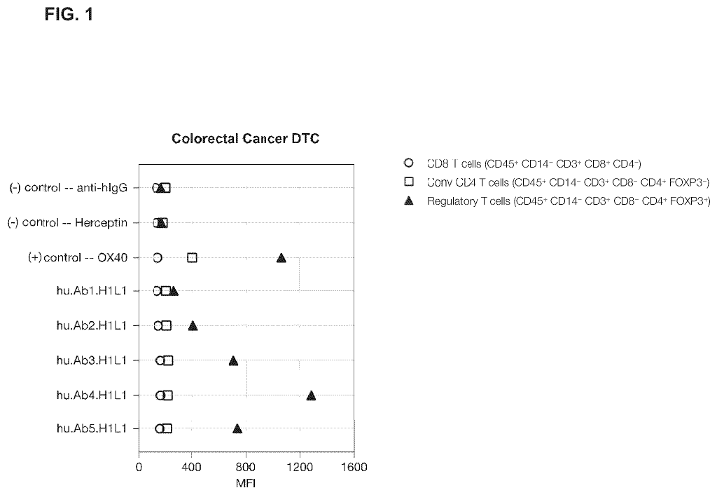

The mAbs were evaluated for binding to regulatory T cells (Treg cells or Tregs) by Fluorescence-Activated Cell Sorting (FACS) flow cytometry. Human colorectal dissociated tumor cells (DTC) (Discovery Life Sciences) were thawed according to vendor's protocol. Cells were surface stained with EFLUOR™ 780-conjugated Fixable Viability Dye (ThermoFisher Scientific) and 2 μg/mL mAb specific for CCR8, OX40 (positive control), Herceptin (negative control), or anti-hIgG (negative control) for 20 min at 4° C. followed by secondary detection with AF647-conjugated AffiniPure F(ab′)₂ Fragment Goat anti-Human IgG, Fcg fragment-specific (Jackson ImmunoResearch) for 10 min at 4° C. Cells were then intracellularly stained using the EBIOSCIENCE™ Foxp3/Transcription Factor Staining Buffer Set (ThermoFisher Scientific) according to the manufacturer's protocol. Antibodies used to define T cell populations were CD45 (H130), CD3 (SK7), CD8 (RPA-T8), and CD14 (63D3) from BD Biosciences, CD4 (RPA-T4) from BioLegend, and FOXP3 (236A/E7) from ThermoFisher Scientific. Flow cytometry was performed on a FORTESSA™ X-20 (BD Biosciences) and analyzed with FLOWJO™ software (BD Biosciences, Version 10.5.3). Shown in FIG. 1 are mean fluorescent intensity (MFI) values for CD8 T cells (defined as CD45+ CD14− CD3+ CD8+ CD4−) (circles, ◯), conventional CD4 T cells (defined as CD45+ CD14− CD3+ CD8− CD4+ FOXP3−) (squares, □), and Treg cells (defined as CD45+ CD14− CD3+ CD8− CD4+ FOXP3+) (triangles, ▴). Three of the five CCR8 mAb clones specifically stained Treg cells and not conventional CD4 or CD8 T cells and were ranked according to CCR8 MFI, greater than 500 MFI: hu.Ab4.H1L1>hu.Ab5.H1 L1>hu.Ab3.H1L1. Upon confirmation that these three CCR8 mAb clones, namely, hu.Ab3.H1L1, hu.Ab4.H1L1, and hu.Ab5.H1L1, also retained human-cyno cross-reactivity (differences less than 5-fold on human and cyno CCR8 CHO cells), these antibodies carried forward for further exploration.

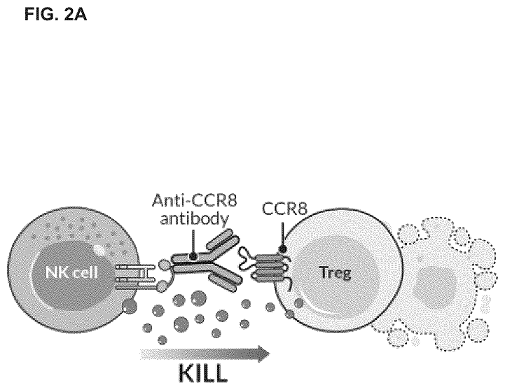

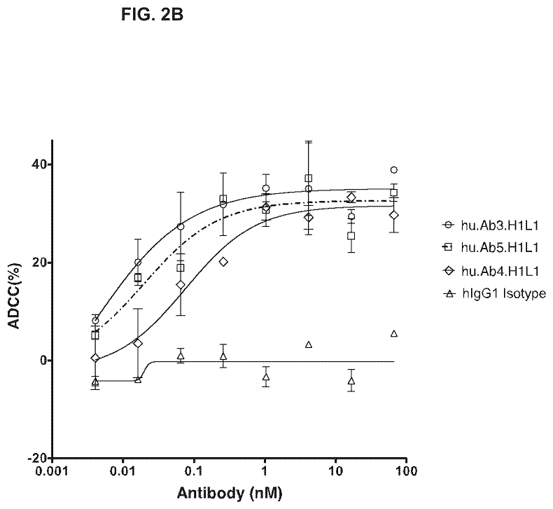

For example, hu.Ab3.H1L1, hu.Ab4.H1L1, and hu.Ab5.H1L1 were further studied for antibody-dependent cellular cytotoxicity (ADCC). hIgG1 isotype was used as a negative control. See FIG. 2. ADCC assays were performed as previously reported in Kamen, L., et al., Development of a kinetic antibody-dependent cellular cytotoxicity assay. J Immunol Methods, 2019. 468: p. 49-54, and Schnueriger, A., et al., Development of a quantitative, cell-line based assay to measure ADCC activity mediated by therapeutic antibodies. Mol Immunol, 2011. 48(12-13): p. 1512-17, with some modifications, using CD16 engineered NK-92_F158 as effector cells and CHO cells that stably express human CCR8 and G-alpha 15 subunit (CHO/hCCR8.Gna15) as target cells. Briefly, lysis of target cells by ADCC was measured by the calcein release method. The target cells were labeled with Calcein-AM (C3100MP, ThermoFisher Scientific) according to the manufacturer's protocol, then washed and plated onto 384-well plates at a density of 3000 cells/well. Anti-CCR8 antibody was added at various concentrations from 0.004 to 1 μg/mL, followed by the addition of NK-92_F158 cells at an effector:target (E:T) ratio of 10:1. The plates were then incubated for 2.5 hours at 37° C. After incubation, the plates were centrifuged at 200×g for 3 minutes, the supernatants were transferred to a white opaque 384-well microplate (OptiPlate-384, PerkinElmer, Waltham, MA), and fluorescent signals were measured in relative fluorescence units (RFU) using an ENSIGHT® Multimode Plate Reader (PerkinElmer) with excitation/emission at 485/520 nm. Signals from the wells containing only the target cells represented spontaneous release of the calcein from labeled cells (spontaneous release), whereas wells containing target cells lysed with TRITON™ X-100 (Sigma-Aldrich, St. Louis, MO) provided the maximal signal available (maximal release). Antibody-independent cell-mediated cytotoxicity (AICC) was measured in wells containing target and effector cells without the addition of the antibody. Samples and controls were tested at least in duplicate in the same plates. The extent of specific ADCC activity was calculated as follows:

$$\% ADCC = 100 \times ( mean experimental release - mean AICC ) / ( mean maximum release - mean spontaneous release )$$

The ADCC activity was plotted as a function of antibody concentrations and the data were fitted to an asymmetric sigmoidal four-parameter logistic (4PL) model using PRISM® (Graphpad; La Jolla, CA).

See FIG. 2. The EC₅₀ value was determined as the concentration reaching 50% maximum ADCC activity of each individual antibody. EC₅₀ values are also tabulated below.

TABLE A
ADCC Activity

| Antibody | EC₅₀ (nM) |
|---|---|
| hu.Ab3.H1L1 | 0.02 |
| hu.Ab5.H1L1 | 0.02 |
| hu.Ab4.H1L1 | 0.08 |

hu.Ab3.H1L1, hu.Ab4.H1L1, and hu.Ab5.H1L1 were further analyzed for their agonist (CCR8 activation) and antagonist (inhibition of CCL1; neutralizing) activity. hIgG1 isotype was used as a negative control. CCR8 activation was monitored by Ca²⁺ influx using Fluorescent Imaging Plate Reader (FLIPR) FDSS/iCell (Hamamatsu, Japan). Briefly, the CHO/hCCR8.Gna 15 cells were loaded with fluorescence Ca²⁺ dye Fluo-8 NW (Cat #36307, AAT Bioquest) and incubated 30 minutes at 37° C., and then at room temperature for another 30 minutes. Serial diluted test anti-CCR8 antibodies were prepared in HHBS buffer in a clear 384-well plate and hCCL1 in HHBS buffer was also aliquoted in a clear 384-well plate. Set-up FLIPR assay on FDSS/μCell with antibody addition at 10 second and hCCL1 addition at 300 second and monitoring total 500 seconds. Set excitation and emission wavelength at 485 nm and 525 nm respectively. After the run, negative control correction is applied and data were normalized against hCCL1 signal (100%) and plotted as a function of antibody concentrations using PRISM®.

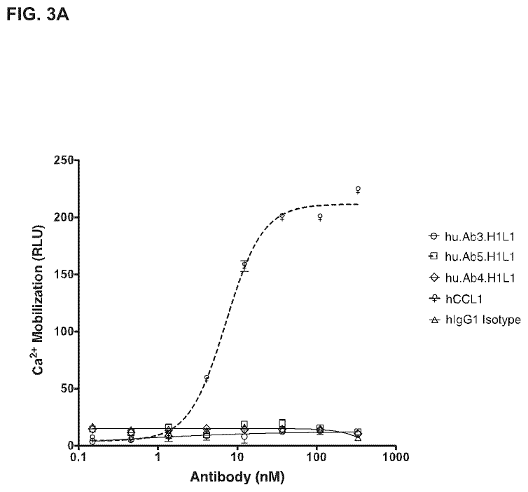

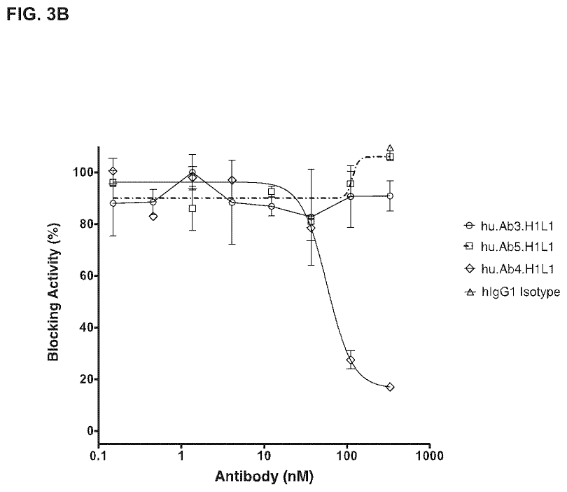

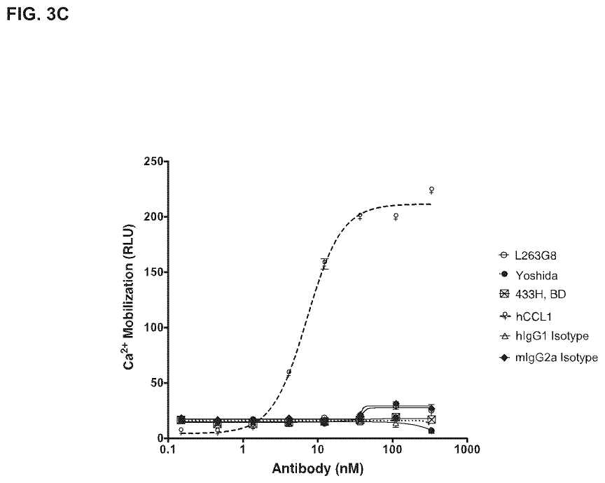

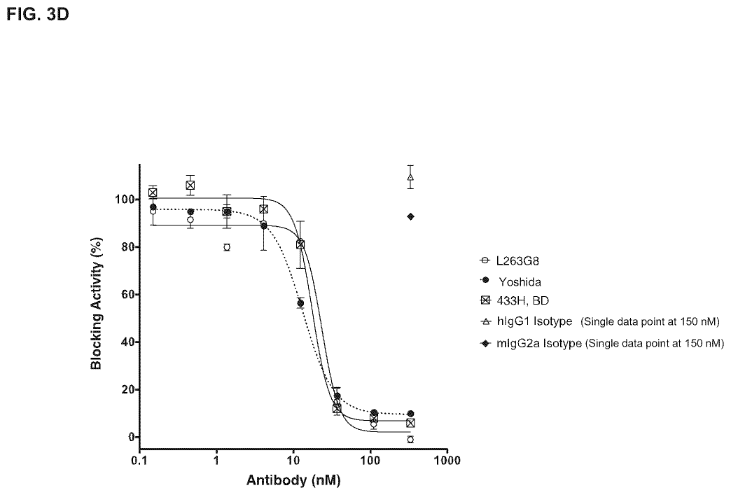

As shown in FIG. 3A, CCL1, a known ligand for CCR8, shows agonist activity, but none of the anti-CCR8 test antibodies show agonistic effects. The data in FIG. 3B indicates anti-CCR8 antibody hu.Ab4.H1L1 has antagonistic (neutralizing) activity against the CCR8 ligand CCL1 (20 nM of ligand), whereas anti-CCR8 antibody hu.Ab5.H1L1 and hu.Ab3.H1L1 demonstrates no ligand blocking (non-neutralizing) activity at the concentration studied. The data in FIG. 3C further demonstrates comparator anti-CCR8 antibodies (the Yoshida humanized anti-human CCR8 antibody, murine anti-human CCR8 mAb 433H (BD Biosciences), and murine anti-human CCR8 mAb L263G8 (Biolegend)) also show antagonistic (neutralizing) activity by blocking the activation of CCR8 by the CCR8 ligand CCL1. The IC₅₀ values for the ligand blocking activity are provided in Table B. As noted in Van Damme et al., J. Immunother. Cancer (2021), 9:e001749, ligand blocking alone is not sufficient for Treg cell depletion in mouse tumors. Thus, even though hu.Ab5.H1L1 and hu.Ab3.H1L1 demonstrated no ligand blocking, these two antibodies were considered still promising candidates, as the goal was to find a selective anti-CCR8 antibody which binds to CCR8 and depletes Treg cells.

TABLE B
CCL1 blocking potencies of anti-CCR8 antibodies

| Antibody | IC₅₀ (nM) |
|---|---|
| hu.Ab5.H1L1 | No inhibition |
| hu.Ab4.H1L1 | 57.9 |
| hu.Ab3.H1L1 | No inhibition |
| murine anti-human CCR8 Ab L263G8 (Biolegend, | 23.6 |
| Commercial Ab) | |
| murine anti-human CCR8 Ab 433H (BD | 17.6 |
| Biosciences, Commercial Ab) | |
| Yoshida humanized anti-human CCR8 antibody | 13.3 |
| (Comparator) | |

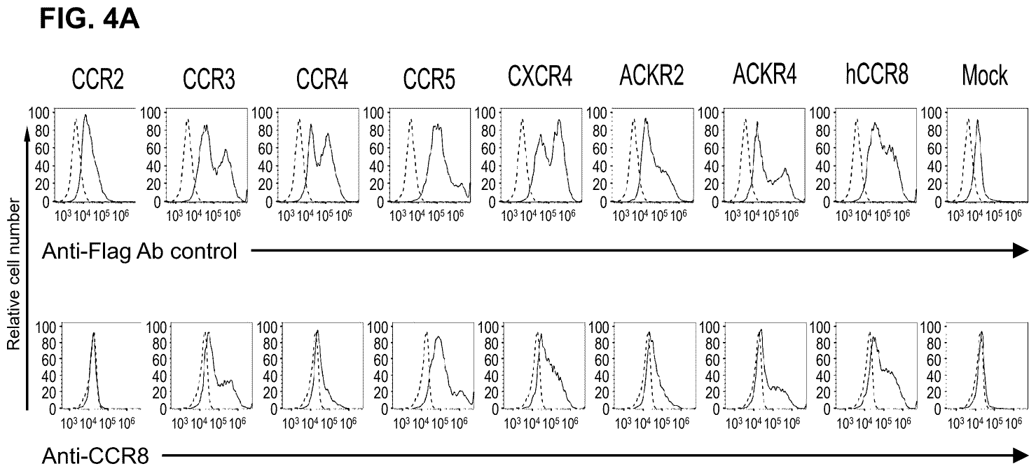

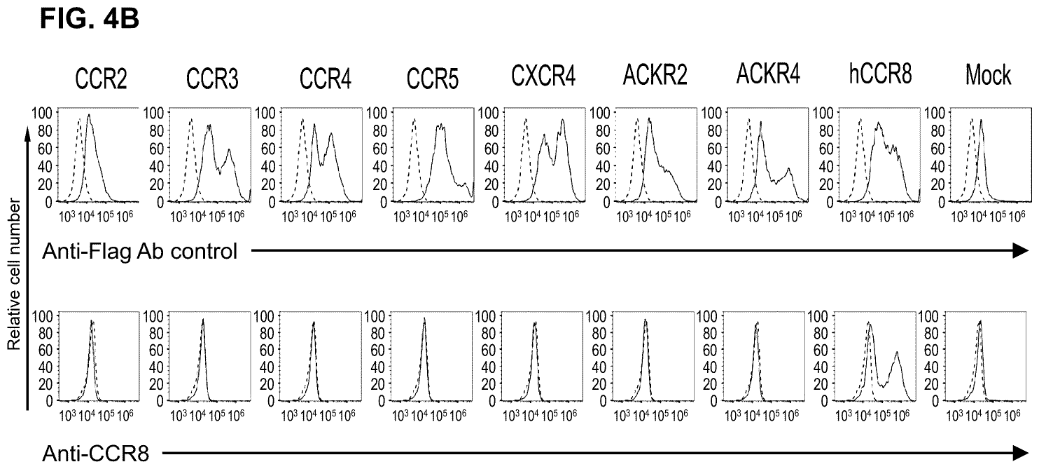

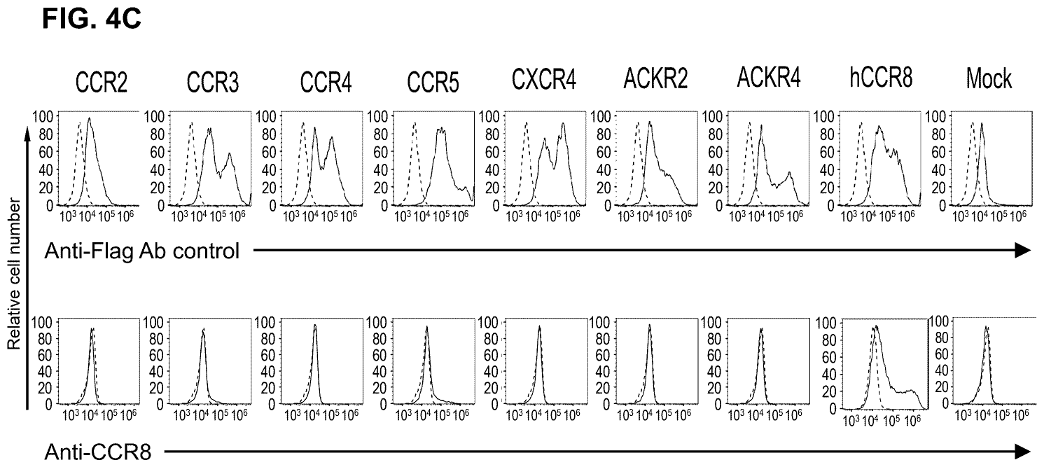

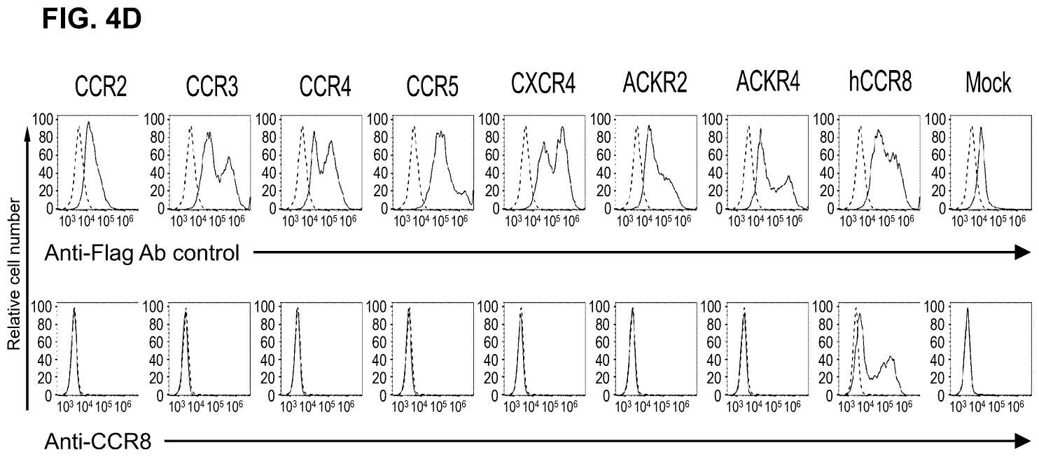

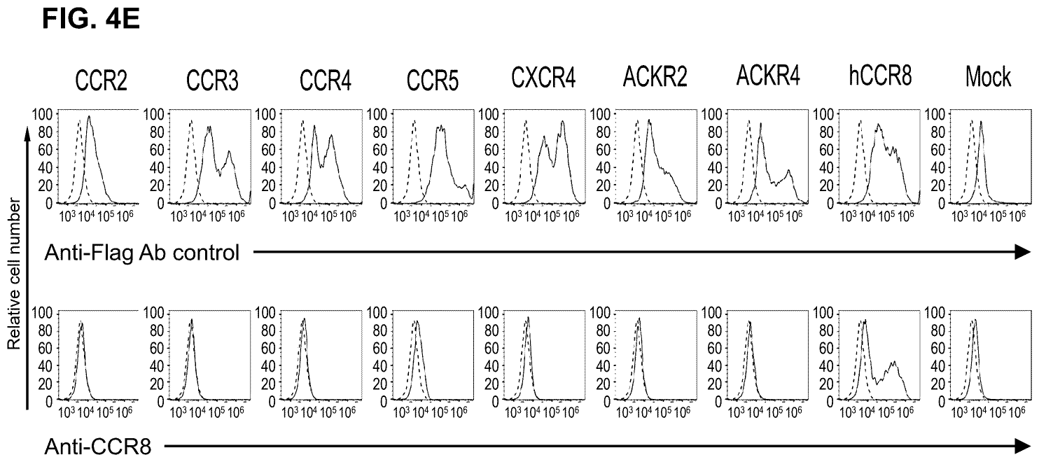

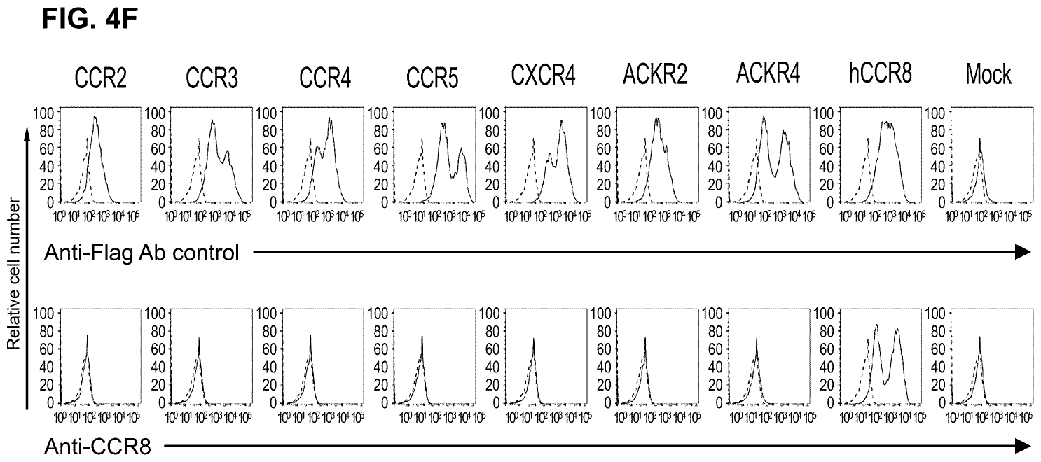

To confirm the selectivity to CCR8, the binding of hu.Ab3.H1L1, hu.Ab4.H1L1, and hu.Ab5.H1L1, as well as the Yoshida humanized anti-human CCR8, murine anti-human CCR8 mAb L263G8 (Biolegend, Commercial Ab) and murine anti-human CCR8 mAb 433H (BD Biosciences, Commercial Ab), were characterized by flow cytometry on HEK293 cells that were transiently transfected with plasmids encoding for FLAG®-tagged other related human GPCRs (CCR2-5, CXCR4, ACKR2, and ACKR4). Cell surface expression of each GPCR was confirmed by staining with an anti-FLAG® antibody control. See FIG. 4A-4F. In particular, HEK293 cells were transfected with N-term FLAG®-tagged human CCR2, CCR3, CCR4, CCR5, CXCR4, ACKR2, ACKR4, hCCR8 constructs, or with a Mock construct using TRANSIT-X2® (reagent:DNA=3:1) for 24 hours, and surface stained with various anti-hCCR8 monoclonal antibodies at 5 μg/ml, or rabbit anti-FLAG® pAb (Sigma) followed by AF647-anti-hIgG or AF647-anti-RbIgG respectively. Antibodies hu.Ab4.H1L1, and hu.Ab5.H1L1, only stained the hCCR8-containing cells, confirming their specificity to hCCR8. Antibody hu.Ab3.H1L1 showed staining of multiple other GPCRs, indicating lack of specificity. Thus, the CCR8 selective hu.Ab4.H1L1 and hu.Ab5.H1L1 antibodies with the best ADCC activities were carried forward.

### Example 2. Mutational Analysis of Ab4 and Ab5 Anti-CCR8 Antibodies

Variants of hu.Ab4.H1L1 and hu.Ab5.H1L1 anti-CCR8 antibodies were further explored and characterized. FIGS. 5A-5D depict the alignment of light chain variable region (FIG. 5A) and heavy chain variable region (FIGS. 5B-5D) of the sequences for rabbit (rb.Ab4) and humanized Ab4 (L1-L4 and H1-H12) CCR8 antibodies studied. FIGS. 6A-6D depict the alignment of light chain variable region (FIG. 6A) and heavy chain variable region (FIGS. 6B-6D) of the sequences for rabbit (rb.Ab5) and humanized Ab5 (L1-L5 and H1-H13) CCR8 antibodies studied. See also Tables 01-03 and D1-D3, below. Table E provides the heavy and light constant domains.

TABLE C1
Light Chain CDR Regions for Ab4 Variants

| | CDR L1 | CDR L2 | CDR L3 |
|---|---|---|---|
| Description | (Kabat and Chothia) | (Kabat and Chothia) | (Kabat and Chothia) |
| rb.Ab4 | QASQSISSYLS | KASTLAS | QQGYTSSNIDNI |
| | (SEQ ID NO: 1) | (SEQ ID NO: 2) | (SEQ ID NO: 3) |
| hu.Ab4.L1 | QASQSISSYLS | KASTLAS | QQGYTSSNIDNI |
| | (SEQ ID NO: 1) | (SEQ ID NO: 2) | (SEQ ID NO: 3) |
| hu.Ab4.L2 | QASQSISSYLS | KASTLAS | QQGYTSSNIDNI |
| | (SEQ ID NO: 1) | (SEQ ID NO: 2) | (SEQ ID NO: 3) |
| hu.Ab4.L3 | QASQSISSYLS | KASTLAS | QQGYTSSNIDNI |
| | (SEQ ID NO: 1) | (SEQ ID NO: 2) | (SEQ ID NO: 3) |
| hu.Ab4.L4 | QASQSISSYLS | KASTLAS | QQGYTSSNIDNI |
| | (SEQ ID NO: 1) | (SEQ ID NO: 2) | (SEQ ID NO: 3) |

TABLE C2
Heavy Chain CDR Regions for Ab4 Variants

| | | | CDR H2 | CDR H3 |
|---|---|---|---|---|
| | | | (Kabat and | (Kabat and |
| Description | CDR H1 (Kabat) | CDRH1 (Chothia) | Chothia) | Chothia) |
| rb.Ab4 | NYAMI | GFSLSNY | TISLGGYTYYAN | ARWSTDSAIYTY |
| | (SEQ ID NO: 4) | (SEQ ID NO: 5) | WAKG | AFDP |
| | | | (SEQ ID NO: 6) | (SEQ ID NO: 7) |
| hu.Ab4.H1 | NYAMI | GFSLSNY | TISLGGYTYYAN | ARWSTDSAIYTY |
| | (SEQ ID NO: 4) | (SEQ ID NO: 5) | WAKG | AFDP |
| | | | (SEQ ID NO: 6) | (SEQ ID NO: 7) |
| hu.Ab4.H2 | NYAMI | GFSLSNY | TISLGGYTYYAN | ARWSTDSAIYTY |
| | (SEQ ID NO: 4) | (SEQ ID NO: 5) | WAKG | AFDP |
| | | | (SEQ ID NO: 6) | (SEQ ID NO: 7) |
| hu.Ab4.H3 | NYAMI | GFSLSNY | TISLGGYTYYAN | ARWSTDSAIYTY |
| | (SEQ ID NO: 4) | (SEQ ID NO: 5) | WAKG | AFDP |
| | | | (SEQ ID NO: 6) | (SEQ ID NO: 7) |
| hu.Ab4.H4 | NYAMI | GFSLSNY | TISLGGYTYYAN | ARWSTDSAIYTY |
| | (SEQ ID NO: 4) | (SEQ ID NO: 5) | WAKG | AFDP |
| | | | (SEQ ID NO: 6) | (SEQ ID NO: 7) |
| hu.Ab4.H5 | NYAMI | GFSLSNY | TISLGGYTYYAN | ARWSTDSAIYTY |
| | (SEQ ID NO: 4) | (SEQ ID NO: 5) | WAKG | AFDP |
| | | | (SEQ ID NO: 6) | (SEQ ID NO: 7) |
| hu.Ab4.H6 | NYAMI | GFSLSNY | TISLGGYTYYAN | ARWSTDSAIYTY |
| | (SEQ ID NO: 4) | (SEQ ID NO: 5) | WAKG | AFDP |
| | | | (SEQ ID NO: 6) | (SEQ ID NO: 7) |
| hu.Ab4.H7 | NYAMI | GFSLSNY | TISLGGYTYYAN | ARWSTDSAIYTY |
| | (SEQ ID NO: 4) | (SEQ ID NO: 5) | WAKG | AFDP |
| | | | (SEQ ID NO: 6) | (SEQ ID NO: 7) |
| hu.Ab4.H8 | NYAMI | GFSLSNY | TISLGGYTYYAN | ARWSTDSAIYTY |
| | (SEQ ID NO: 4) | (SEQ ID NO: 5) | WAKG | AFDP |
| | | | (SEQ ID NO: 6) | (SEQ ID NO: 7) |
| hu.Ab4.H9 | NYAMI | GFSLSNY | TISLGGYTYYAN | ARWSTDSAIYTY |
| | (SEQ ID NO: 4) | (SEQ ID NO: 5) | WAKG | AFDP |
| | | | (SEQ ID NO: 6) | (SEQ ID NO: 7) |
| hu.Ab4.H10 | NYAMI | GFSLSNY | TISLGGYTYYAN | ARWSTDSAIYTY |
| | (SEQ ID NO: 4) | (SEQ ID NO: 5) | WAKG | AFDP |
| | | | (SEQ ID NO: 6) | (SEQ ID NO: 7) |
| hu.Ab4.H11 | NYAMI | GFSLSNY | TISLGGYTYYAN | ARWSTDSAIYTY |
| | (SEQ ID NO: 4) | (SEQ ID NO: 5) | WAKG | AFDP |
| | | | (SEQ ID NO: 6) | (SEQ ID NO: 7) |
| hu.Ab4.H12 | NYAMI | GFSLSNY | TISLGGYTYYAN | ARWSTDSAIYTY |
| | (SEQ ID NO: 4) | (SEQ ID NO: 5) | WAKG | AFDP |
| | | | (SEQ ID NO: 6) | (SEQ ID NO: 7) |

TABLE C3
Heavy Chain and Light Chain Variable Regions for Ab4 Variants

| Description | Sequence |
|---|---|
| rb.Ab4 | QSVEESGGRLVTPGTPLTLTCTVSGFSLSNYAMIWVRQAPGEGLEWV |
| Heavy Chain Variable | GTISLGGYTYYANWAKGRFTISKTSTTVDLKISSPTTEDTATYFCARAR |
| Region | WSTDSAIYTYAFDPWGPGTLVTVSS |
| (SEQ ID NO: 8) | |
| rb.Ab4 | AYDMTQTPASVEVAVGGTVTIKCQASQSISSYLSWYQQKPGQRPELLIY |
| Light Chain Variable | KASTLASGVSSRFKGSGSGTQFTLTISDLECADAATYYCQQGYTSSNID |
| Region | NIFGGGTEVVVK |
| (SEQ ID NO: 9) | |
| hu.Ab4.H1 | EQQLLESGGGLVQPGGSLRLSCAVSGFSLSNYAMIWVRQAPGKGLEW |
| Heavy Chain Variable | VGTISLGGYTYYANWAKGRFTISKDSSKTTVYLQMNSLRAEDTAVYFCA |
| Region | RARWSTDSAIYTYAFDPWGPGTLVTVSS |
| (SEQ ID NO: 10) | |
| hu.Ab4.H2 | EVQLLESGGGLVQPGGSLRLSCAVSGFSLSNYAMIWVRQAPGKGLEW |
| Heavy Chain Variable | VGTISLGGYTYYANWAKGRFTISKDSSKTTVYLQMNSLRAEDTAVYFCA |
| Region | RARWSTDSAIYTYAFDPWGPGTLVTVSS |
| (SEQ ID NO: 11) | |
| hu.Ab4.H3 | EQQLLESGGGLVQPGGSLRLSCAASGFSLSNYAMIWVRQAPGKGLEW |
| Heavy Chain Variable | VGTISLGGYTYYANWAKGRFTISKDSSKTTVYLQMNSLRAEDTAVYFCA |
| Region | RARWSTDSAIYTYAFDPWGPGTLVTVSS |
| (SEQ ID NO: 12) | |
| hu.Ab4.H4 | EQQLLESGGGLVQPGGSLRLSCAVSGFSLSNYAMIWVRQAPGKGLEW |
| Heavy Chain Variable | VSTISLGGYTYYANWAKGRFTISKDSSKTTVYLQMNSLRAEDTAVYFCA |
| Region | RARWSTDSAIYTYAFDPWGPGTLVTVSS |
| (SEQ ID NO: 13) | |
| hu.Ab4.H5 | EQQLLESGGGLVQPGGSLRLSCAVSGFSLSNYAMIWVRQAPGKGLEW |
| Heavy Chain Variable | VGTISLGGYTYYANWAKGRFTISRDSSKTTVYLQMNSLRAEDTAVYFCA |
| Region | RARWSTDSAIYTYAFDPWGPGTLVTVSS |
| (SEQ ID NO: 14) | |
| hu.Ab4.H6 | EQQLLESGGGLVQPGGSLRLSCAVSGFSLSNYAMIWVRQAPGKGLEW |
| Heavy Chain Variable | VGTISLGGYTYYANWAKGRFTISKDNSKTTVYLQMNSLRAEDTAVYFCA |
| Region | RARWSTDSAIYTYAFDPWGPGTLVTVSS |
| (SEQ ID NO: 15) | |
| hu.Ab4.H7 | EQQLLESGGGLVQPGGSLRLSCAVSGFSLSNYAMIWVRQAPGKGLEW |
| Heavy Chain Variable | VGTISLGGYTYYANWAKGRFTISKDSSKNTVYLQMNSLRAEDTAVYFCA |
| Region | RARWSTDSAIYTYAFDPWGPGTLVTVSS |
| (SEQ ID NO: 16) | |
| hu.Ab4.H8 | EQQLLESGGGLVQPGGSLRLSCAVSGFSLSNYAMIWVRQAPGKGLEW |
| Heavy Chain Variable | VGTISLGGYTYYANWAKGRFTISKDSSKTTLYLQMNSLRAEDTAVYFCA |
| Region | RARWSTDSAIYTYAFDPWGPGTLVTVSS |
| (SEQ ID NO: 17) | |
| hu.Ab4.H9 | EQQLLESGGGLVQPGGSLRLSCAVSGFSLSNYAMIWVRQAPGKGLEW |
| Heavy Chain Variable | VGTISLGGYTYYANWAKGRFTISKDSSKTTVYLQMNSLRAEDTAVYYCA |
| Region | RARWSTDSAIYTYAFDPWGPGTLVTVSS |
| (SEQ ID NO: 18) | |
| hu.Ab4.H10 | EQQLLESGGGLVQPGGSLRLSCAVSGFSLSNYAMIWVRQAPGKGLEW |
| Heavy Chain Variable | VGTISLGGYTYYANWAKGRFTISKDSSKTTVYLQMNSLRAEDTAVYFCA |
| Region | RARWSTDSAIYTYAFDPWGQGTLVTVSS |
| (SEQ ID NO: 19) | |
| hu.Ab4.H11 | EVQLLESGGGLVQPGGSLRLSCAASGFSLSNYAMIWVRQAPGKGLEW |
| Heavy Chain Variable | VSTISLGGYTYYANWAKGRFTISRDNSKNTLYLQMNSLRAEDTAVYYCA |
| Region | RARWSTDSAIYTYAFDPWGQGTLVTVSS |
| (SEQ ID NO: 20) | |
| hu.Ab4.H12 | EVQLLESGGGLVQPGGSLRLSCAASGFSLSNYAMIWVRQAPGKGLEW |
| Heavy Chain Variable | VSTISLGGYTYYANWAKGRFTISRDSSKTTVYLQMNSLRAEDTAVYFCA |
| Region | RARWSTDSAIYTYAFDPWGPGTLVTVSS |
| (SEQ ID NO: 21) | |
| hu.Ab4.L1 | DYQMTQSPSSLSASVGDRVTITCQASQSISSYLSWYQQKPGKRPKLLIY |
| Light Chain Variable | KASTLASGVPSRFSGSGSGTDFTLTISSLQPEDFATYYCQQGYTSSNID |
| Region | NIFGGGTKVEIK |
| (SEQ ID NO: 22) | |
| hu.Ab4.L2 | DIQMTQSPSSLSASVGDRVTITCQASQSISSYLSWYQQKPGKRPKLLIY |
| Light Chain Variable | KASTLASGVPSRFSGSGSGTDFTLTISSLQPEDFATYYCQQGYTSSNID |
| Region | NIFGGGTKVEIK |
| (SEQ ID NO: 23) | |
| hu.Ab4.L3 | DYQMTQSPSSLSASVGDRVTITCQASQSISSYLSWYQQKPGKAPKLLIY |
| Light Chain Variable | KASTLASGVPSRFSGSGSGTDFTLTISSLQPEDFATYYCQQGYTSSNID |
| Region | NIFGGGTKVEIK |
| (SEQ ID NO: 24) | |
| hu.Ab4.L4 | DIQMTQSPSSLSASVGDRVTITCQASQSISSYLSWYQQKPGKAPKLLIY |
| Light Chain Variable | KASTLASGVPSRFSGSGSGTDFTLTISSLQPEDFATYYCQQGYTSSNID |
| Region | NIFGGGTKVEIK |
| (SEQ ID NO: 25) | |

TABLE D1
Light Chain CDR Regions for Ab5 Variants

| | CDR L1 | CDR L2 | CDR L3 |
|---|---|---|---|
| | (Kabat and | (Kabat and | (Kabat and |
| Description | Chothia) | Chothia) | Chothia) |
| rb.Ab5 | QASENIANALA | GASNLAS | QCAYYGNSFVEGT |
| | (SEQ ID NO: 26) | (SEQ ID NO: 27) | (SEQ ID NO: 109) |
| hu.Ab5.L1 | QASENIANALA | GASNLAS | QQAYYGNSFVEGT |
| | (SEQ ID NO: 26) | (SEQ ID NO: 27) | (SEQ ID NO: 28) |
| hu.Ab5.L2 | QASENIANALA | GASNLAS | QQAYYGNSFVEGT |
| | (SEQ ID NO: 26) | (SEQ ID NO: 27) | (SEQ ID NO: 28) |
| hu.Ab5.L3 | QASENIANALA | GASNLAS | QQAYYGNSFVEGT |
| | (SEQ ID NO: 26) | (SEQ ID NO: 27) | (SEQ ID NO: 28) |
| hu.Ab5.L4 | QASENIANALA | GASNLAS | QQAYYGNSFVEGT |
| | (SEQ ID NO: 26) | (SEQ ID NO: 27) | (SEQ ID NO: 28) |
| hu.Ab5.L5 | QASENIANALA | GASNLAS | QQAYYGNSFVEGT |
| | (SEQ ID NO: 26) | (SEQ ID NO: 27) | (SEQ ID NO: 28) |

TABLE D2
Heavy Chain CDR Regions for Ab5 Variants

| | | | CDR H2 | CDR H3 |
|---|---|---|---|---|
| Descrip- | CDR H1 | CDR H1 | (Kabat and | (Kabat and |
| tion | (Kabat) | (Chothia) | Chothia) | Chothia) |
| rb.Ab5 | TYAMG | GIDLSTY | LIHRSGRTYYA | SYPDYSATASI |
| | (SEQ ID | (SEQ ID | TWAKG (SEQ | (SEQ ID NO: |
| | NO: 29) | NO: 30) | ID NO: 31) | 32) |
| hu.Ab5. | TYAMG | GIDLSTY | LIHRSGRTYYA | SYPDYSATASI |
| H1 | (SEQ ID | (SEQ ID | TWAKG (SEQ | (SEQ ID NO: |
| | NO: 29) | NO: 30) | ID NO: 31) | 32) |
| hu.Ab5. | TYAMG | GIDLSTY | LIHRSGRTYYA | SYPDYSATASI |
| H2 | (SEQ ID | (SEQ ID | TWAKG (SEQ | (SEQ ID NO: |
| | NO: 29) | NO: 30) | ID NO: 31) | 32) |
| hu.Ab5. | TYAMG | GIDLSTY | LIHRSGRTYYA | SYPDYSATASI |
| H3 | (SEQ ID | (SEQ ID | TWAKG (SEQ | (SEQ ID NO: |
| | NO: 29) | NO: 30) | ID NO: 31) | 32) |
| hu.Ab5. | TYAMG | GIDLSTY | LIHRSGRTYYA | SYPDYSATASI |
| H4 | (SEQ ID | (SEQ ID | TWAKG (SEQ | (SEQ ID NO: |
| | NO: 29) | NO: 30) | ID NO: 31) | 32) |
| hu.Ab5. | TYAMG | GIDLSTY | LIHRSGRTYYA | SYPDYSATASI |
| H5 | (SEQ ID | (SEQ ID | TWAKG (SEQ | (SEQ ID NO: |
| | NO: 29) | NO: 30) | ID NO: 31) | 32) |
| hu.Ab5. | TYAMG | GIDLSTY | LIHRSGRTYYA | SYPDYSATASI |
| H6 | (SEQ ID | (SEQ ID | TWAKG (SEQ | (SEQ ID NO: |
| | NO: 29) | NO: 30) | ID NO: 31) | 32) |
| hu.Ab5. | TYAMG | GIDLSTY | LIHRSGRTYYA | SYPDYSATASI |
| H7 | (SEQ ID | (SEQ ID | TWAKG (SEQ | (SEQ ID NO: |
| | NO: 29) | NO: 30) | ID NO: 31) | 32) |
| hu.Ab5. | TYAMG | GIDLSTY | LIHRSGRTYYA | SYPDYSATASI |
| H8 | (SEQ ID | (SEQ ID | TWAKG (SEQ | (SEQ ID NO: |
| | NO: 29) | NO: 30) | ID NO: 31) | 32) |
| hu.Ab5. | TYAMG | GIDLSTY | LIHRSGRTYYA | SYPDYSATASI |
| H9 | (SEQ ID | (SEQ ID | TWAKG (SEQ | (SEQ ID NO: |
| | NO: 29) | NO: 30) | ID NO: 31) | 32) |
| hu.Ab5. | TYAMG | GIDLSTY | LIHRSGRTYYA | SYPDYSATASI |
| H10 | (SEQ ID | (SEQ ID | TWAKG (SEQ | (SEQ ID NO: |
| | NO: 29) | NO: 30) | ID NO: 31) | 32) |
| hu.Ab5. | TYAMG | GIDLSTY | LIHRSGRTYYA | SYPDYSATASI |
| H11 | (SEQ ID | (SEQ ID | TWAKG (SEQ | (SEQ ID NO: |
| | NO: 29) | NO: 30) | ID NO: 31) | 32) |
| hu.Ab5. | TYAMG | GIDLSTY | LIHRSGRTYYA | SYPDYSATASI |
| H12 | (SEQ ID | (SEQ ID | TWAKG (SEQ | (SEQ ID NO: |
| | NO: 29) | NO: 30) | ID NO: 31) | 32) |
| hu.Ab5. | TYAMG | GIDLSTY | LIHRSGRTYYA | SYPDYSATASI |
| H13 | (SEQ ID | (SEQ ID | TWAKG (SEQ | (SEQ ID NO: |
| | NO: 29) | NO: 30) | ID NO: 31) | 32) |

TABLE D3
Heavy Chain and Light Chain Variable Regions for Ab5 Variants

| Description | Sequence |
|---|---|
| rb.Ab5 | QSVEESGGRLVSPGTPLTLTCTVSGIDLSTYAMGWVRQAPGKGLEWIG |
| Heavy Chain Variable | LIHRSGRTYYATWAKGRFTISKTSSTTVDLKITSPTTEDTATYFCTRS |
| Region | YPDYSATASIWGPGTLVTVSS |
| (SEQ ID NO: 33) | |
| rb.Ab5 | DIVVTQTPASVEAAVGGTVTIKCQASENIANALAWYQQKSGQPPMFLI |
| Light Chain Variable | YGASNLASGVSSRFKGSGSGTEFTLTISDLECADAAVYYCQCAYYGNS |
| Region | FVEGTFGGGTEVVVK |
| (SEQ ID NO: 34) | |
| hu.Ab5.H1 | EQQLLESGGGLVQPGGSLRLSCAVSGIDLSTYAMGWVRQAPGKGLEWI |
| Heavy Chain Variable | GLIHRSGRTYYATWAKGRFTISKDSSKTTVYLQMNSLRAEDTAVYFCT |
| Region | RSYPDYSATASIWGPGTTVTVSS |
| (SEQ ID NO: 35) | |
| hu.Ab5.H2 | EVQLLESGGGLVQPGGSLRLSCAVSGIDLSTYAMGWVRQAPGKGLEWI |
| Heavy Chain Variable | GLIHRSGRTYYATWAKGRFTISKDSSKTTVYLQMNSLRAEDTAVYFCT |
| Region | RSYPDYSATASIWGPGTTVTVSS |
| (SEQ ID NO: 36) | |
| hu.Ab5.H3 | EQQLLESGGGLVQPGGSLRLSCAASGIDLSTYAMGWVRQAPGKGLEWI |
| Heavy Chain Variable | GLIHRSGRTYYATWAKGRFTISKDSSKTTVYLQMNSLRAEDTAVYFCT |
| Region | RSYPDYSATASIWGPGTTVTVSS |
| (SEQ ID NO: 37) | |
| hu.Ab5.H4 | EQQLLESGGGLVQPGGSLRLSCAVSGIDLSTYAMGWVRQAPGKGLEWV |
| Heavy Chain Variable | GLIHRSGRTYYATWAKGRFTISKDSSKTTVYLQMNSLRAEDTAVYFCT |
| Region | RSYPDYSATASIWGPGTTVTVSS |
| (SEQ ID NO: 38) | |
| hu.Ab5.H5 | EQQLLESGGGLVQPGGSLRLSCAVSGIDLSTYAMGWVRQAPGKGLEWI |
| Heavy Chain Variable | SLIHRSGRTYYATWAKGRFTISKDSSKTTVYLQMNSLRAEDTAVYFCT |
| Region | RSYPDYSATASIWGPGTTVTVSS |
| (SEQ ID NO: 39) | |
| hu.Ab5.H6 | EQQLLESGGGLVQPGGSLRLSCAVSGIDLSTYAMGWVRQAPGKGLEWI |
| Heavy Chain Variable | GLIHRSGRTYYATWAKGRFTISRDSSKTTVYLQMNSLRAEDTAVYFCT |
| Region | RSYPDYSATASIWGPGTTVTVSS |
| (SEQ ID NO: 40) | |
| hu.Ab5.H7 | EQQLLESGGGLVQPGGSLRLSCAVSGIDLSTYAMGWVRQAPGKGLEWI |
| Heavy Chain Variable | GLIHRSGRTYYATWAKGRFTISKDNSKTTVYLQMNSLRAEDTAVYFCT |
| Region | RSYPDYSATASIWGPGTTVTVSS |
| (SEQ ID NO: 41) | |
| hu.Ab5.H8 | EQQLLESGGGLVQPGGSLRLSCAVSGIDLSTYAMGWVRQAPGKGLEWI |
| Heavy Chain Variable | GLIHRSGRTYYATWAKGRFTISKDSSKNTVYLQMNSLRAEDTAVYFCT |
| Region | RSYPDYSATASIWGPGTTVTVSS |
| (SEQ ID NO: 42) | |
| hu.Ab5.H9 | EQQLLESGGGLVQPGGSLRLSCAVSGIDLSTYAMGWVRQAPGKGLEWI |
| Heavy Chain Variable | GLIHRSGRTYYATWAKGRFTISKDSSKTTLYLQMNSLRAEDTAVYFCT |
| Region | RSYPDYSATASIWGPGTTVTVSS |
| (SEQ ID NO: 43) | |
| hu.Ab5.H10 | EQQLLESGGGLVQPGGSLRLSCAVSGIDLSTYAMGWVRQAPGKGLEWI |
| Heavy Chain Variable | GLIHRSGRTYYATWAKGRFTISKDSSKTTVYLQMNSLRAEDTAVYYCT |
| Region | RSYPDYSATASIWGPGTTVTVSS |
| (SEQ ID NO: 44) | |
| hu.Ab5.H11 | EQQLLESGGGLVQPGGSLRLSCAVSGIDLSTYAMGWVRQAPGKGLEWI |
| Heavy Chain Variable | GLIHRSGRTYYATWAKGRFTISKDSSKTTVYLQMNSLRAEDTAVYFCT |
| Region | RSYPDYSATASIWGQGTTVTVSS |
| (SEQ ID NO: 45) | |
| hu.Ab5.H12 | EVQLLESGGGLVQPGGSLRLSCAASGIDLSTYAMGWVRQAPGKGLEWV |
| Heavy Chain Variable | SLIHRSGRTYYATWAKGRFTISRDNSKNTLYLQMNSLRAEDTAVYYCT |
| Region | RSYPDYSATASIWGQGTTVTVSS |
| (SEQ ID NO: 46) | |
| hu.Ab5.H13 | EVQLLESGGGLVQPGGSLRLSCAASGIDLSTYAMGWVRQAPGKGLEWV |
| Heavy Chain Variable | GLIHRSGRTYYATWAKGRFTISKDSSKNTLYLQMNSLRAEDTAVYYCT |
| Region | RSYPDYSATASIWGQGTTVTVSS |
| (SEQ ID NO: 47) | |
| hu.Ab5.L1 | DIQVTQSPSSLSASVGDRVTITCQASENIANALAWYQQKPGKPPKFLI |
| Light Chain Variable | YGASNLASGVPSRFSGSGSGTDFTFTISSLQPEDIATYYCQQAYYGNS |
| Region | FVEGTFGGGTKVEIK |
| (SEQ ID NO: 48) | |
| hu.Ab5.L2 | DIQMTQSPSSLSASVGDRVTITCQASENIANALAWYQQKPGKPPKFLI |
| Light Chain Variable | YGASNLASGVPSRFSGSGSGTDFTFTISSLQPEDIATYYCQQAYYGNS |
| Region | FVEGTFGGGTKVEIK |
| (SEQ ID NO: 49) | |
| hu.Ab5.L3 | DIQVTQSPSSLSASVGDRVTITCQASENIANALAWYQQKPGKAPKFLI |
| Light Chain Variable | YGASNLASGVPSRFSGSGSGTDFTFTISSLQPEDIATYYCQQAYYGNS |
| Region | FVEGTFGGGTKVEIK |
| (SEQ ID NO: 50) | |
| hu.Ab5.L4 | DIQVTQSPSSLSASVGDRVTITCQASENIANALAWYQQKPGKPPKLLI |
| Light Chain Variable | YGASNLASGVPSRFSGSGSGTDFTFTISSLQPEDIATYYCQQAYYGNS |
| Region | FVEGTFGGGTKVEIK |
| (SEQ ID NO: 51) | |
| hu.Ab5.L5 | DIQMTQSPSSLSASVGDRVTITCQASENIANALAWYQQKPGKAPKLLI |
| Light Chain Variable | YGASNLASGVPSRFSGSGSGTDFTFTISSLQPEDIATYYCQQAYYGNS |
| Region | FVEGTFGGGTKVEIK |
| (SEQ ID NO: 52) | |

TABLE E
Constant Domains

| Description | Sequence |
|---|---|
| hIgG1 constant | ASTKGPSVFPLAPSSKSTSGGTAALGCLVK |
| domain | DYFPEPVTVSWNSGALTSGVHTFPAVLQSS |
| (Heavy Chain) | GLYSLSSVVTVPSSSLGTQTYICNVNHKPS |
| (SEQ ID NO: 53) | NTKVDKKVEPKSCDKTHTCPPCPAPELLGG |
| | PSVFLFPPKPKDTLMISRTPEVTCVVVDVS |
| | HEDPEVKFNWYVDGVEVHNAKTKPREEQYN |
| | STYRVVSVLTVLHQDWLNGKEYKCKVSNKA |
| | LPAPIEKTISKAKGQPREPQVYTLPPSREE |
| | MTKNQVSLTCLVKGFYPSDIAVEWESNGQP |
| | ENNYKTTPPVLDSDGSFFLYSKLTVDKSRW |
| | QQGNVFSCSVMHEALHNHYTQKSLSLSPGK |
| Kappa constant | RTVAAPSVFIFPPSDEQLKSGTASVVCLLN |
| domain | NFYPREAKVQWKVDNALQSGNSQESVTEQD |
| (Light Chain) | SKDSTYSLSSTLTLSKADYEKHKVYACEVT |
| (SEQ ID NO: 54) | HQGLSSPVTKSFNRGEC |

Evaluation of CCR8 binding of the humanized variants with a hIgG1 Fc involved screening by flow cytometry and comparing the relative EC₅₀ and MFI on human CCR8 CHO cells to the parental rabbit antibodies. Specifically, stable CHO-huCCR8.Gna15 cells were stained with various concentrations (starting from 10 ug/ml or 66.66 nM, 1:4 serial dilution for total 8 concentration points) of Ab4 and Ab5 variants at 4° C. for 30 minutes, then washed twice with FACS buffer (PBS with 0.5% BSA and 0.2 mM EDTA) and followed by staining with AF647-anti-hIgG at 4° C. for 15 min. Cells were washed twice with FACS buffer, and re-suspended in FACS buffer with propidium iodide (0.5 ug/ml) and analyzed with IQUE® 3 (Sartorius).

For the Ab4 LC variants L1-L4, as provided in Table F1, variants L2 and L4, containing a Y21 mutation, showed significant changes in either EC₅₀ or MFI. It was thus determined that Y2 on light chain is a key rabbit Vernier residue. Variants L1 and L3 contained this Y2 residue, and variant L3 was selected for further analysis.

TABLE F1
Relative EC₅₀ and MFIs for Ab4 LC variants

| Antibody | Relative EC₅₀ | Relative MFI |
|---|---|---|
| rb.Ab4 | 1.0 | 1.0 |
| hu.Ab4.H1L1 | 0.88 | 0.95 |
| hu.Ab4.H1L2 | 1.06 | 0.73 |
| hu.Ab4.H1L3 | 1.23 | 1.00 |
| hu.Ab4.H1L4 | 1.04 | 0.80 |

For the Ab4 HC variants H2-H11, as provided in Table F2, variant H6 (with a S73N mutation), variant H7 (with a T76N mutation), variant H8 (with a V78L mutation), variant H9 (with a F91 Y mutation), variant H10 (with a P105Q mutation), and variant H11 (with a S73N, V78L, F91 Y, and P105Q mutations) showed significant changes in either EC₅₀ or MFI. It was thus determined that S73, T76, V78, F91 and P105 on the heavy chain were the key rabbit Vernier residues. These five residues were combined to construct variant H12 (hu.Ab4.H12).

TABLE F2
Relative EC₅₀ and MFIs for Ab4 HC variants

| Antibody | Relative EC₅₀ | Relative MFI |
|---|---|---|
| rb.Ab4 | 1.0 | 1.0 |
| hu.Ab4.H2L1 | 0.99 | 1.07 |
| hu.Ab4.H3L1 | 1.00 | 1.02 |
| hu.Ab4.H4L1 | 0.96 | 1.06 |
| hu.Ab4.H5L1 | 1.02 | 0.90 |
| hu.Ab4.H6L1 | 1.06 | 0.68 |
| hu.Ab4.H7L1 | 1.05 | 0.44 |
| hu.Ab4.H8L1 | 1.03 | 0.72 |
| hu.Ab4.H9L1 | 1.04 | 0.87 |
| hu.Ab4.H10L1 | 1.07 | 0.77 |
| hu.Ab4.H11L1 | 0.88 | 0.66 |

For the Ab5 LC variants L2-L5, as provided in Table F3, variant L2 (with a V4M mutation), variant L3 (with a P43A mutation), variant L4 (with a F46L mutation), and variant L5 (with a V4M, P43A, and F46L mutation) showed significant changes in either EC₅₀ or MFI. It was thus determined that V4, P43 and F46 on the light chain were the key rabbit Vernier residues. All variants contained a C90Q mutation in CDR L3, which was introduced to remove an unpaired cysteine that would be a liability during manufacturing. Variant L1, which contains all three V4, P43 and F46 residues, was selected for further study.

TABLE F3
Relative EC₅₀ and MFIs for Ab5 LC variants

| Antibody | Relative EC₅₀ | Relative MFI |
|---|---|---|
| rb.Ab5 | 1.0 | 1.0 |
| hu.Ab5.H1L2 | 1.4 | 0.79 |
| hu.Ab5.H1L3 | 1.5 | 0.81 |
| hu.Ab5.H1L4 | 1.7 | 0.51 |
| hu.Ab5.H1L5 | 0.3 | 0.31 |

For the Ab5 HC variants H2-H12, as provided in Table F4, variant H5 (with a G49S mutation), variant H6 (with a K71R mutation), variant H7 (with a S73N mutation), and H12 (with a G49S, K71R, and S73N mutation) showed significant changes in either EC₅₀ or MFI. Thus, it was determined that G49, K71 and S73 on the heavy chain were the key rabbit Vernier residues. These three residues were combined to construct variant H13.

TABLE F4
Relative EC₅₀ and MFIs for Ab5 HC variants

| Antibody | Relative EC₅₀ | Relative MFI |
|---|---|---|
| rb.Ab5 | 1 | 1 |
| hu.Ab5.H2L1 | 1.1 | 0.93 |
| hu.Ab5.H3L1 | 1.5 | 1.0 |
| hu.Ab5.H4L1 | 1.5 | 0.95 |
| hu.Ab5.H5L1 | 7.6 | 0.35 |
| hu.Ab5.H6L1 | 2.4 | 0.97 |
| hu.Ab5.H7L1 | 2.2 | 0.96 |
| hu.Ab5.H8L1 | 1.4 | 0.94 |
| hu.Ab5.H9L1 | 1.3 | 0.92 |
| hu.Ab5.H10L1 | 1.2 | 0.87 |
| hu.Ab5.H11L1 | 1.3 | 0.87 |
| hu.Ab5.H12L1 | 2.3 | 0.26 |

### Example 3. Characterization of hu.Ab4.H12L3 and hu.Ab5.H13L1 Variants

### (a) Human-Cyno Cross-Reactivity

Cell-based affinity measurements were performed using radiolabeled IgGs and CHO cell lines stably expressing human or cyno CCR8 for hu.Ab5.H13L1 and hu.Ab4.H12L3.

TABLE G1
hu.Ab4.H12L3 Full-length Sequence

| Description | Sequence |
|---|---|
| hu.Ab4.H12 | EVQLLESGGGLVQPGGSLRLSCAASGFSLSNYAMIWVRQAPGKGLEWVSTISL |
| Full-length | GGYTYYANWAKGRFTISRDSSKTTVYLQMNSLRAEDTAVYFCARARWSTDSAI |
| Heavy Chain | YTYAFDPWGPGTLVTVSSASTKGPSVFPLAPSSKSTSGGTAALGCLVKDYFPE |
| (SEQ ID NO: | PVTVSWNSGALTSGVHTFPAVLQSSGLYSLSSVVTVPSSSLGTQTYICNVNHK |
| 57) | PSNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQVS |
| | LTCLVKGFYPSDIAVEWESNGQPENNYKTTPPVLDSDGSFFLYSKLTVDKSRW |
| | QQGNVFSCSVMHEALHNHYTQKSLSLSPGK |
| hu.Ab4.L3 | DYQMTQSPSSLSASVGDRVTITCQASQSISSYLSWYQQKPGKAPKLLIYKAST |
| Full-length | LASGVPSRFSGSGSGTDFTLTISSLQPEDFATYYCQQGYTSSNIDNIFGGGTK |
| Light Chain | VEIKRTVAAPSVFIFPPSDEQLKSGTASVVCLLNNFYPREAKVQWKVDNALQS |
| (SEQ ID NO: | GNSQESVTEQDSKDSTYSLSSTLTLSKADYEKHKVYACEVTHQGLSSPVTKSF |
| 58) | NRGEC |

TABLE G2
hu.Ab5.H13L1 Full-length Sequence

| Description | Sequence |
|---|---|
| hu.Ab5.H13 | EVQLLESGGGLVQPGGSLRLSCAASGIDLSTYAMGWVRQAPGKGLEWVGLIH |
| Full-length | RSGRTYYATWAKGRFTISKDSSKNTLYLQMNSLRAEDTAVYYCTRSYPDYSA |
| Heavy Chain | TASIWGQGTTVTVSSASTKGPSVFPLAPSSKSTSGGTAALGCLVKDYFPEPV |
| (SEQ ID NO: | TVSWNSGALTSGVHTFPAVLQSSGLYSLSSVVTVPSSSLGTQTYICNVNHKP |
| 55) | SNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLTVLH |
| | QDWLNGKEYKCKVSNKALPAPIEKTISKAKGQPREPQVYTLPPSREEMTKNQ |
| | VSLTCLVKGFYPSDIAVEWESNGQPENNYKTTPPVLDSDGSFFLYSKLTVDK |
| | SRWQQGNVFSCSVMHEALHNHYTQKSLSLSPGK |
| hu.Ab5.L1 | DIQVTQSPSSLSASVGDRVTITCQASENIANALAWYQQKPGKPPKFLIYGAS |
| Full-length | NLASGVPSRFSGSGSGTDFTFTISSLQPEDIATYYCQQAYYGNSFVEGTFGG |
| Light Chain | GTKVEIKRTVAAPSVFIFPPSDEQLKSGTASVVCLLNNFYPREAKVQWKVDN |
| (SEQ ID NO: | ALQSGNSQESVTEQDSKDSTYSLSSTLTLSKADYEKHKVYACEVTHQGLSSP |
| 56) | VTKSFNRGEC |

Briefly, stable CHO cells expressing human or cyno CCR8 were seeded in cold binding buffer (Opti-MEM+2% FBS+50 mM HEPES, pH 7.2+0.1% Sodium Azide) at 50,000 cells per well. A fixed concentration of ¹²⁵I-anti-CCR8 radiolabeled using the NEX244 IODOGEN® method (Perkin Elmer) was mixed with serially diluted anti-CCR8 antibodies starting at 20 nM or 50 nM. The antibody mixture was added to the cells and incubated at room temperature for 12 hours under gentle agitation. The cells and antibodies were then transferred to Millipore multiscreen filter plates. The filter plates were washed 4 times with 250 μL of cold binding buffer and dried for at least 30 minutes and the filters were punched into 5 mL polystyrene tubes. The radioactivity was measured using a Perkin Elmer Wallac Wizard 2470 Gamma Counter set at 1 count per minute with 0.8 counting efficiency. The data were fitted using the heterologous one site-fit Ki competitive binding model in GraphPad PRISM®.

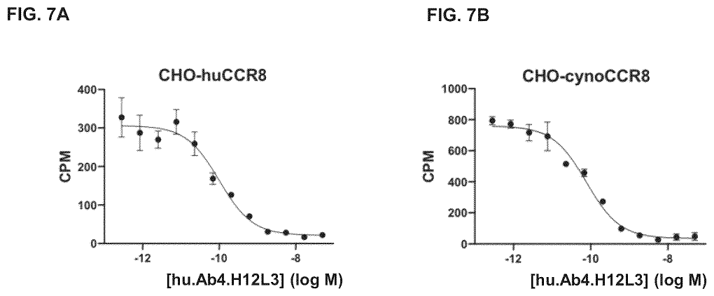

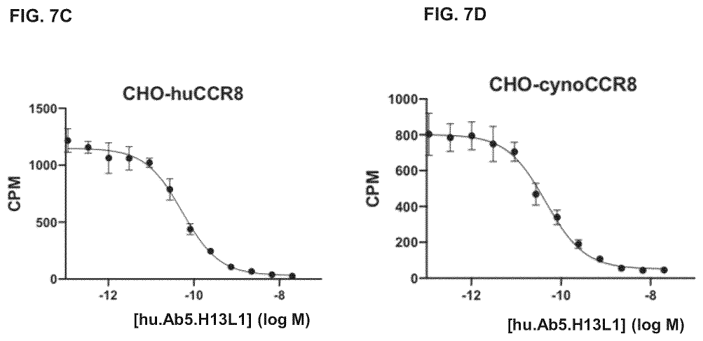

As shown in FIGS. 7A-7D, both hu.Ab4.H12L3 and hu.Ab5.H13L1 have similar affinity for both human and cyno CCR8, indicating desirable cross-reactivity. Tabulated affinity K_{d} (nM) data from these studies is provided below.

TABLE G3
CCR8 human and cyno affinity

| Antibody | K_{D} (nM) CCR8 human | K_{D} (nM) CCR8 cyno |
|---|---|---|
| hu.Ab4.H12L3 | 0.0284 | 0.0243 |
| hu.Ab5.H13L1 | 0.0040 | 0.0034 |

### (b) CCR8 Selectivity

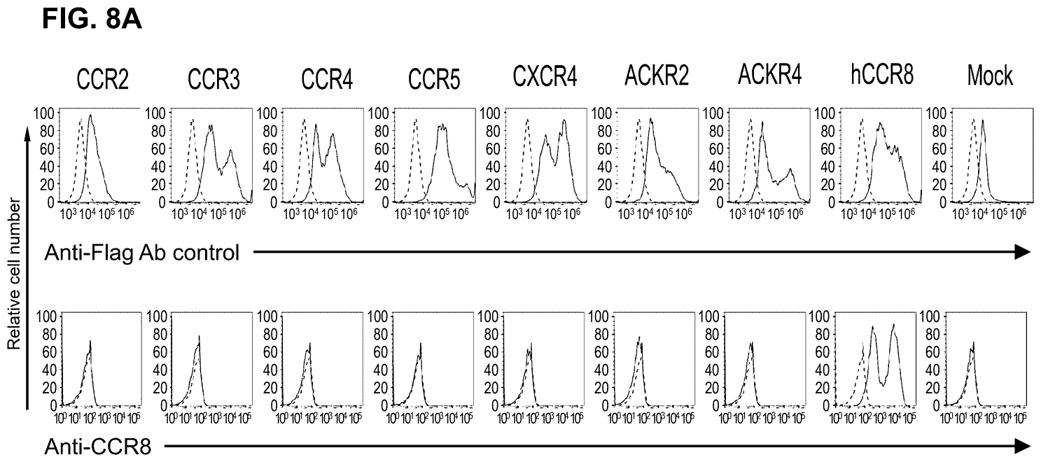

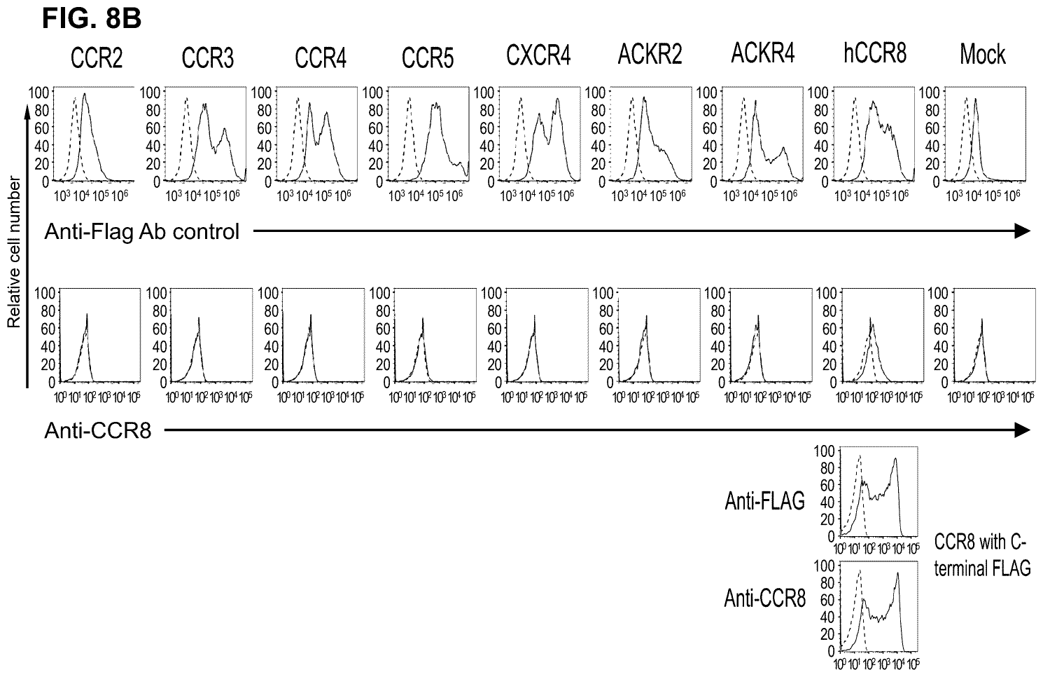

To reconfirm that the Ab4 and Ab5 variants remained selective to CCR8 compared to the corresponding H1 L1 variants, binding was analyzed by flow cytometry following the procedure as described for FIG. 4A and FIG. 4B. As before, both hu.Ab4.H12L3 (FIG. 8A) and hu.Ab5.H13L1 (FIG. 8B) bound selectively to CCR8-expressing cells.

### (c) CCR8 Activation and Ligand Blocking

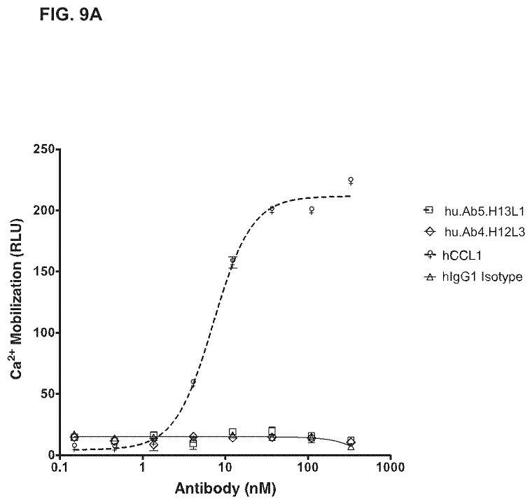

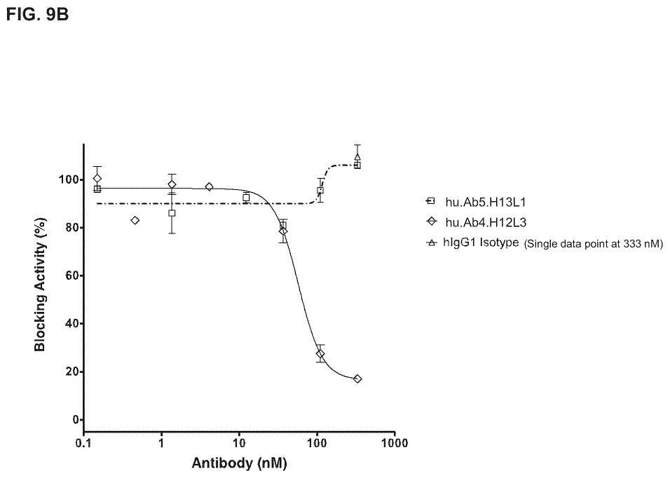

To reconfirm that the Ab4 and Ab5 variants retained their properties regarding CCR8 activation and ligand blocking ability, experiments were conducted with hu.Ab4.H12L3 and hu.Ab5.H13L1 antibodies as previously described in Example 1 and FIG. 3A-3C. See FIGS. 9A-9B. Similar to FIG. 3A, the data in FIG. 9A reconfirms neither the Ab4 nor the Ab5 anti-CCR8 antibody variants show agonistic effects in the absence of CCR8 ligand CCL1. Similar to FIG. 3B data, the data in FIG. 9B reconfirms the Ab4 variant demonstrates antagonistic effects by blocking the activation of CCR8 by the CCR8 ligand CCL1 (20 nM of ligand), whereas the Ab5 variant demonstrates no ligand blocking activity at the concentration studied. The IC₅₀ values for the ligand blocking activity are provided in the below Table.

TABLE H1
Ligand Blocking Activity

| Antibody | IC₅₀ (nM) |
|---|---|
| hu.Ab5.H13L1 | No inhibition |
| hu.Ab4.H12L3 | 57.9 |

### (d) Sulfation Independence

Human CCR8 contains four potential sites of tyrosine sulfation within the N-terminus and existing evidence indicates that modification at these sites exhibits some heterogeneity (Gutierrez et al. JBC 2004; Jen, et al. Biochemistry 2010). As such, antibodies that recognize these sulfated tyrosines in CCR8 may exhibit variability in CCR8 binding, and thus mediate variable Treg cell depletion. Human CCR8+ HEK293 cells were generated that lack tyrosyl protein sulfotransferase (TPST) 1 and 2, which are the enzymes that catalyze tyrosine sulfation. Binding was then analyzed of various anti-CCR8 mAbs to wild type (293T) and TPST1/2 NTC and KO cells.

In particular, HEK293, HEK293-hCCR8.TPST1/2 NTC and HEK293-hCCR8.TPST1/2 KO stable cell lines were stained with test and comparator anti-CCR8 antibodies (1 ug/ml) at 4° C. for 30 minutes, then washed twice with FACS buffer (PBS with 0.5% BSA and 0.2 mM EDTA) and followed by staining with AF647-anti-hIgG at 4° C. for 15 minutes. Cells were washed twice with FACS buffer, and re-suspended in FACS buffer with propidium iodide (0.5 ug/ml) and analyzed with BD FACSCELESTA™ Flow Cytometer or IQUE® 3 (Sartorius).

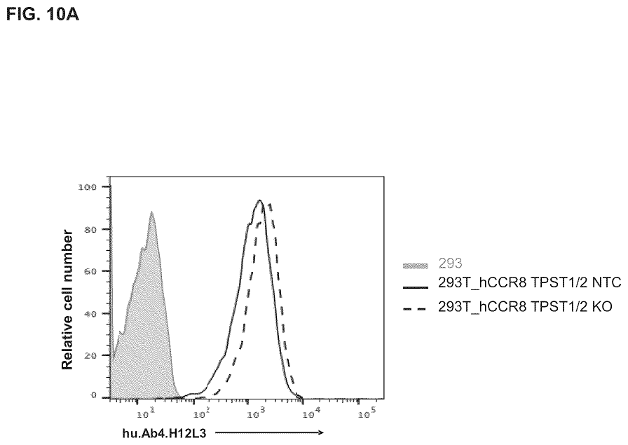

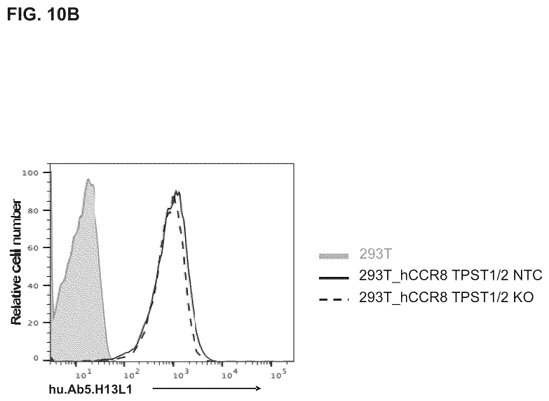

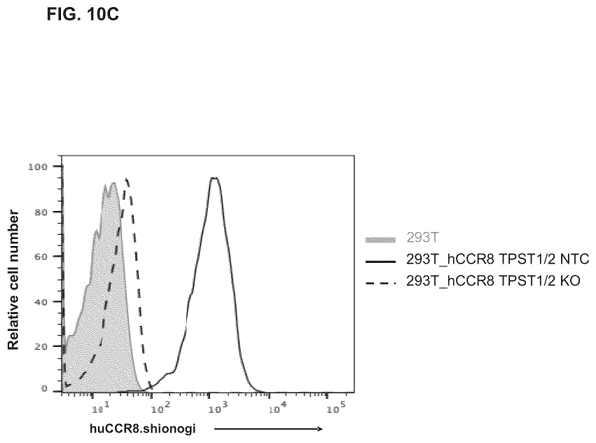

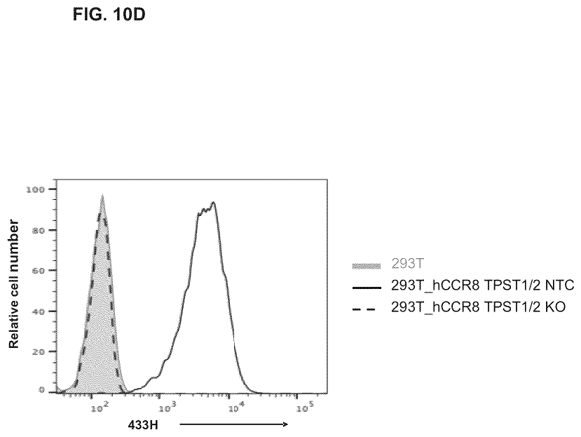

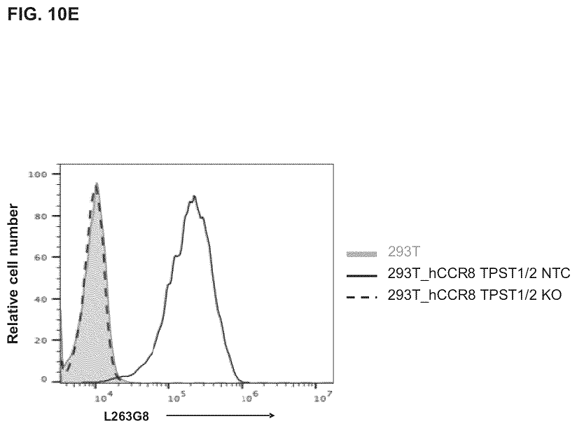

FIGS. 10A-10E depicts differences in staining of hu.Ab4.H12L3 and hu.Ab5.H13L1 compared to the Yoshida humanized anti-human CCR8 antibody and commercial antibodies murine anti-human CCR8 mAb 433H (BD Biosciences) and murine anti-human CCR8 mAb L263G8 (Biolegend) to CCR8+ HEK293 cells with (hCCR8.TPST1/2 NTC) and without tyrosyl protein sulfotransferase (TPST) 1 and tyrosyl protein sulfotransferase (TPST) 2 (hCCR8.TPST1/2 KO). hu.Ab4.H12L3 (FIG. 10A) and hu.Ab5.H13L1 (FIG. 10B) show similar binding/staining to both cell lines (hCCR8.TPST1/2 NTC and hCCR8.TPST1/2 KO), indicating they bind CCR8 independent of tyrosine sulfation (“sulfation independent”). In contrast, the Yoshida humanized anti-human CCR8 antibody (FIG. 10C) and commercial antibodies murine anti-human CCR8 mAb 433H (BD Biosciences) (FIG. 10D) and murine anti-human CCR8 mAb L263G8 (Biolegend) (FIG. 10E) failed to bind the TPST1/2 KO cells, indicating they require tyrosine sulfation of CCR8 for binding, and are thus considered “sulfation dependent.”

### Example 4. hu.Ab4.H12L3 and hu.Ab5.H13L1 Afucosylated Variants

Afucosylated hu.Ab5.H13L1 and hu.Ab4.H12L3 variants (at Fc N-glycan position Asn 299), and the afucosylated anti-gD control, were prepared by expression and purification from FUT8 knock-out (KO) CHO cells as described in Wong et al., Biotechnology and Bioengineering (2010) 106:751-763.

### (a) Percent Afucosylation

Titration of fucose in media of CHO FUT8KO yielded a panel of hu.Ab5.H13L1 with varying levels of afucosylation, e.g., between about 14% to about 93% afucosylated hu.Ab5.H13L1.

As noted in the below table, increasing the afucosylation level from 14% to 49% produced a greater than 4-fold increase in ADCC activity, and a greater than 3-fold increase in ADCP activity.

Afucosylated hu.Ab5.H13L1 and hu.Ab4.H12L3 studied in the in vitro and in vivo experiments to follow contained levels of between about 80% to about 95% afucosylation.

TABLE H2
Percent Afucosylation Effect on ADCC and ADCP activity

| % afucosylation | ADCC EC₅₀ (pM) | ADCP EC₅₀ (pM) |
|---|---|---|
| 93 | 3.53 | 0.049 |
| 89 | 4.63 | 0.052 |
| 73 | 5.42 | 0.047 |
| 62 | 6.61 | 0.068 |
| 53 | 8.45 | 0.093 |
| 49 | 9.24 | 0.075 |
| 14 | 39.7 | 0.25 |

### (b) Enhanced Fcgamma RIIIa Binding of Afucosylated Variants

The binding of fucosylated and afucosylated variants of hu.Ab5.H13L1 and hu.Ab4.H12L3 to both FcgR3a proteins by ELISA was studied. Briefly, an anti-GST antibody was coated on Nunc MAXISORP™ plates. GST-FcgR3a.V158 and GST-FcgR3a.F158 were captured at 500 ng/mL. Plates were then washed and then serially diluted anti-CCR8 antibodies starting at 100 μg/mL were incubated on the plate for 1 hour at room temperature. Plates were washed and bound antibody was detected by an HRP-conjugated anti-human IgG secondary antibody. Absorbance at 450 nm was measured by a plate reader. The data were fitted using a 4-parameter logistic curve in SOFTM^{AX}® Pro. As shown in the Table below, afucosylated IgG1 anti-CCR8 antibodies Afuc.hu.Ab5.H13L1 and Afuc.hu.Ab4.H12L3 exhibited enhanced Fcgamma Rilla binding activities (approximately a 10-fold increase in binding potency) compared to their fucosylated counterparts hu.Ab5.H13L1 and hu.Ab4.H12L3.

TABLE I
Fcgamma RIIIa Binding Activity

| | FcgR3a.F158 protein | FcgR3a.V158 protein |
|---|---|---|
| Antibody | EC₅₀ (ug/mL) | EC₅₀ (ug/mL) |
| hu.Ab5.H13L1 | 4.107 □ 1.036 | 0.39 □ 0.049 |
| Afuc.hu.Ab5.H13L1 | 0.101 □ 0.057 | 0.035 □ 0.006 |
| hu.Ab4.H12L3 | 3.74 □ 2.03 | 0.388 □ 0.178 |
| Afuc.hu.Ab4.H12L3 | 0.08 □ 0.025 | 0.033 □ 0.006 |

### (C) Enhanced ADCC Activity of Afucosylated Variants

Afuc.hu.Ab4.H12L3, hu.Ab4.H12L3, Afuc.hu.Ab5.H13L1, and hu.Ab5.H13L1 were analyzed for antibody-dependent cellular cytotoxicity (ADCC). ADCC assays were performed as previously reported in Kamen et al., Development of a kinetic antibody-dependent cellular cytotoxicity assay. J Immunol Methods (2019) 468:49-54, and Schnueriger et al., Development of a quantitative, cell-line based assay to measure ADCC activity mediated by therapeutic antibodies. Mol Immunol (2011) 48:1512-17, with some modifications, using CD16 engineered NK-92_F158 as effector cells and CHO cells that stably express human CCR8 and Ga 15 subunit (CHO/hCCR8.Gna15) as target cells. Briefly, lysis of target cells by ADCC was measured by the calcein release method. The target cells were labeled with Calcein-AM (C3100MP, ThermoFisher Scientific) according to the manufacturer's protocol, then washed and plated onto 384-well plates at a density of 3000 cells/well. Anti-CCR8 antibody was added at various concentrations from 0.004 to 1 μg/mL, followed by the addition of NK-92_F158 cells at an effector:target (E:T) ratio of 10:1. The plates were then incubated for 2.5 hours at 37° C. After incubation, the plates were centrifuged at 200×g for 3 minutes, the supernatants were transferred to a white opaque 384-well microplate (OptiPlate-384, PerkinElmer, Waltham, MA), and fluorescent signals were measured in relative fluorescence units (RFU) using an ENSIGHT® Multimode Plate Reader (PerkinElmer) with excitation/emission at 485/520 nm. Signals from the wells containing only the target cells represented spontaneous release of the calcein from labeled cells (spontaneous release), whereas wells containing target cells lysed with TRITON™ X-100 (Sigma-Aldrich, St. Louis, MO) provided the maximal signal available (maximal release). Antibody-independent cell-mediated cytotoxicity (AICC) was measured in wells containing target and effector cells without the addition of the antibody. Samples and controls were tested at least in duplicate in the same plates. The extent of specific ADCC activity was calculated as follows:

% ADCC=100×(mean experimental release-mean AICC)/(mean maximum release-mean spontaneous release)

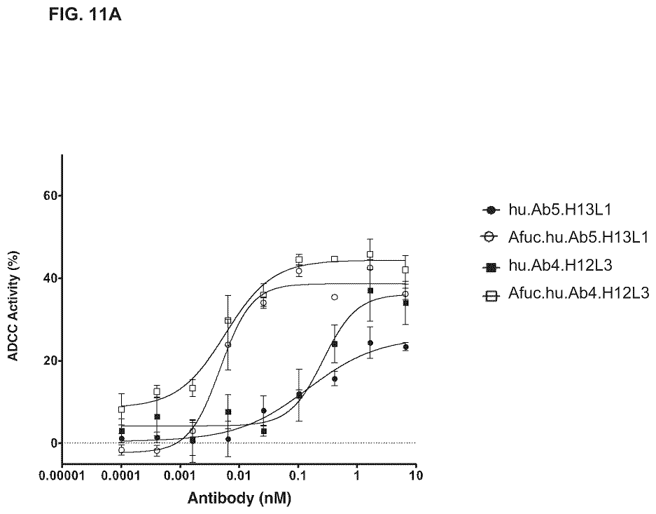

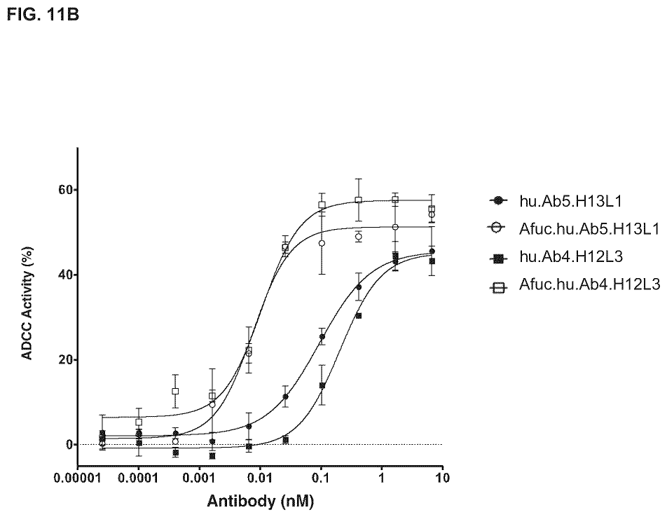

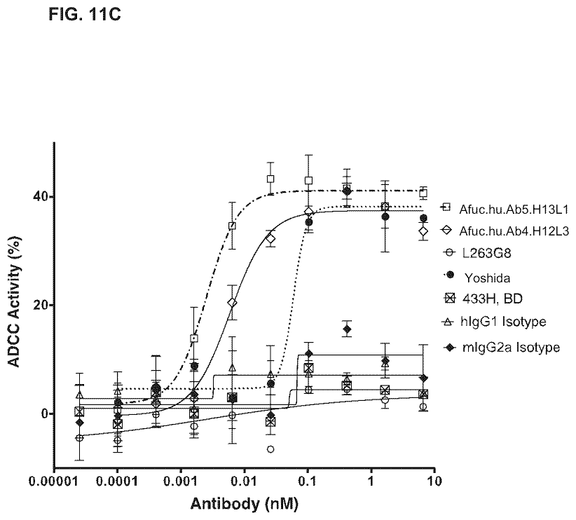

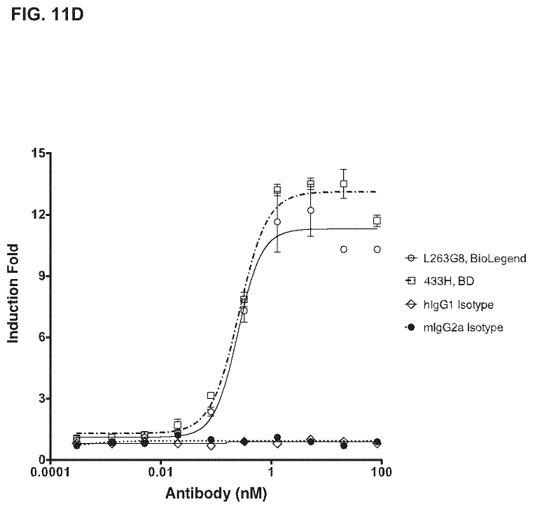

The ADCC activity was plotted as a function of antibody concentrations and the data were fitted to an asymmetric sigmoidal four-parameter logistic (4PL) model using PRISM® (Graphpad; La Jolla, CA). FIGS. 11A-11B show afucosylated CCR8 antibodies Afuc.hu.Ab5.H13L1 and Afuc.hu.Ab4.H12L3 have enhanced (>10-fold improved) ADCC activity compared to their fucosylated counterparts hu.Ab5.H13L1 and hu.Ab4.H12L3 against CHO cells stably expressing hCCR8 using NK-92 F158 (FIG. 11A) and NK-92 V158 (FIG. 11B) as effector cells.

The ADCC activity for Afuc.hu.Ab5.H13L1 and Afuc.hu.Ab4.H12L3 was also measured against the Yoshida humanized anti-human CCR8 antibody and commercial antibodies murine anti-human CCR8 mAb 433H (BD Biosciences) and murine anti-human CCR8 mAb L263G8 (Biolegend). See FIG. 11C. The data demonstrates the Yoshida humanized anti-human CCR8 antibody exhibits weaker ADCC activity (less than 10- to 20-fold less ADCC activity) than the anti-CCR8 antibodies Afuc.Ab5.H13L1, Afuc.Ab4.H12L3. Commercially available antibodies murine anti-human CCR8 mAb 433H (BD Biosciences) and murine anti-human CCR8 mAb L263G8 (Biolegend), which comprise murine Fc domains, as expected demonstrated no ADCC activity as the assay employed in this instance is primarily relevant for antibodies comprising human Fc domains.

Murine anti-human CCR8 mAb 433H (BD Biosciences) and murine anti-human CCR8 mAb L263G8 (Biolegend) were tested for ADCC activity in an assay relevant for antibodies comprising murine Fc regions but anti-human CCR8 activity, i.e., using Jurkat/mFcgR4 stable line as the effector cells and CHO/hCCR8 as the target cells. Human CCR8 (hCCR8) was used to mimic a human clinical setting. Specifically, the assay consists of a genetically engineered Jurkat T cell line that expresses mouse FcgRIV receptor and a luciferase reporter driven by an NFAT-response element (NFAT-RE). When co-cultured with a target cell and relevant antibody, the mFcgRIV Effector Cells bind the Fc domain of the antibody, resulting in mFcgRIV signaling and NFAT-RE-mediated luciferase activity. Materials and Reagents: Assay buffer: RPMI1640 without phenol red supplemented with 4% low IgG; 96-well White Flat Bottom Polystyrene TC-treated Microplates, Corning #3601; BIO-GLO™ reagent. Assay Procedures: Add 25 μL/well diluted antibody in assay buffer (Prepared 3×, staring at 30 ug/mL serial diluted at 1:4 for 10 points). Resuspend the target cell in assay buffer, adjust final density to 1×10⁶/ml; dispense 25 μL cells to each well for target cell density of 25,000/well; incubate the plate for 20 minutes at room temperature. Add 25 μL/well Jurkat/mFcgRIV cell (at 5×10⁶ cells/mL) to each well for effector cell density of 125,000/well; re-mix the cells in the reservoir regularly during the process to prevent settling of cells to the bottom. Cover the assay plates with a lid and incubate the plate at 37° C. with 5% CO₂ incubator for 16 hours. Do not stack the plates inside the incubator. Remove the assay plates from the incubator and equilibrate to ambient temperature for 15 minutes. Using a multichannel pipette, add 75 μl of BIO-GLO™ Reagent to the assay plates, taking care not to create bubbles. Incubate the plate 15 minutes at room temperature. Measure luminescence using ENSIGHT® luminescence plate reader. mIgG2a isotype, hIgG1 and ratIgG2b were tested as controls. Human CCR8 (hCCR8) was used to mimic a human clinical setting. As can be seen from the data, each of the anti-hCCR8 mAb tested—L263G8 (BioLegend) and 433H (BD Biosciences)—displayed high induction fold results at antibody concentration levels of about 1 nM—showing induction fold results of more than about 10 and about 12, respectively. The high induction fold for each of these antibodies plateaus at an antibody concentration level of about 40 nM—with induction fold values of about 11 and 13, respectively. The results of these experiments are provided in FIG. 11D.

Activity data from these studies is also provided in the below Table. In summary, each of the antibodies studied, whether having humanized or murine Fc regions, demonstrated ADCC activity in the assay to which the antibody isotype was species-matched to relevant effector reporter cells.

TABLE J
ADCC Activity

| | NK-92 | NK-92 | NK-92 | mFcgRIV |
|---|---|---|---|---|
| | F158 | V158 | F158 | Jurkat |
| | cells | cells | cells | cells with |
| | EC₅₀ | EC₅₀ | EC₅₀ | CHO/hCCR8 |
| | (nM) | (nM) | (nM) | EC₅₀ (nM) |
| Ab tested | FIG. 11A | FIG. 11B | FIG. 11C | FIG. 11D |
| hu.Ab5.H13L1 | 0.14 | 0.094 | | |
| Afuc.hu.Ab5.H13L1 | 0.0047 | 0.0073 | 0.003 | |
| hu.Ab4.H12L3 | 0.27 | 0.21 | | |
| Afuc.hu.Ab4.H12L3 | 0.006 | 0.011 | 0.006 | |
| Yoshida Ab | | | 0.060 | |
| L263G8 (Biolegend) | | | No activity* | 0.28 |
| 433H (BD | | | No activity* | 0.26 |
| Biosciences) | | | | |

*No activity = assay conditions not relevant for specific Ab isotype.

### (d) ADCC Enhancement Against Treg Cells

To induce CCR8 expression on Treg cells from human peripheral blood mononuclear cells (PBMC), 107 human PBMC were intraperitoneally transferred to NOD.Cg-Prkdc^{scid} Il2rg^{tm1Wjl}/SzJ (NSG™) mice (JAX) and spleens collected 2-3 weeks post-transfer. Human T cells were enriched from single cell suspensions of NSG™ splenocytes using the Mouse Lineage Cell Depletion Kit (Miltenyi Biotec), separately primary NK cells were enriched from human PBMC using the Human NK Cell Isolation Kit (Miltenyi Biotec) according to manufacturer's protocol. Human T cells were incubated with 0.001-1 ug/mL CCR8 mAb for 30 minutes at room temperature prior to the addition of primary NK cells at an effector:target ratio of 2:1. After overnight incubation at 37° C., cells were collected, surface stained, and intracellularly stained using the EBIOSCIENCE™ Foxp3/Transcription Factor Staining Buffer Set (ThermoFisher Scientific) according to the manufacturer's protocol. Antibodies used to define T cell populations were CD45 (H130), CD3 (SK7), CD8 (RPA-T8), and CD14 (63D3) from BD Biosciences, CD4 (RPA-T4) from BioLegend, and FOXP3 (236A/E7) from ThermoFisher Scientific. COUNTBRIGHT™ Absolute Counting Beads (ThermoFisher Scientific) was added to each sample prior to acquisition. Flow cytometry was performed on a FORTESSA™ X-20 (BD Biosciences) and analyzed with FLOWJO™ software (BD Biosciences, Version 10.5.3). Absolute cell counts were calculated according to manufacturer's protocol.

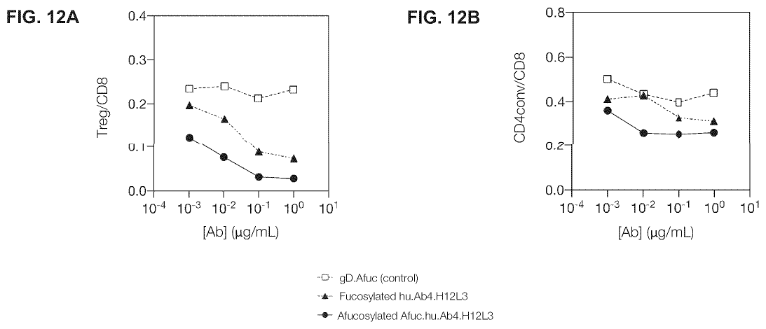

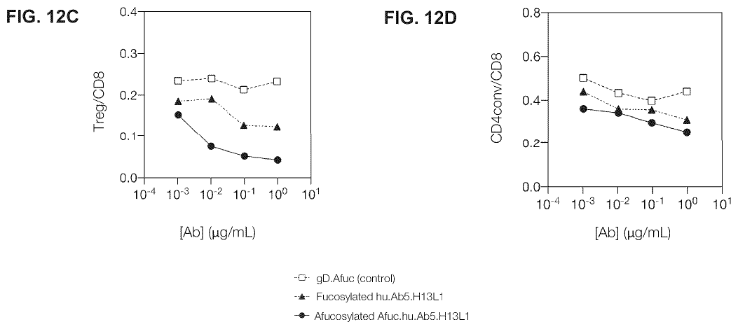

ADCC activity against Treg cells was measured by calculating the ratio of recovered regulatory T cells to recovered CD8 cells (Treg/CD8) or conventional CD4 T cells to recovered CD8 T cells (CD4conv/CD8). The number of CD8 T cells recovered was similar across all concentrations of CCR8 mAbs and isotype control mAb tested (“gD.afuc”). As depicted in FIGS. 12A-12D, afucosylated CCR8 antibodies Afuc.hu.Ab5.H13L1 and Afuc.hu.Ab4.H12L3 and fucosylated CCR8 antibodies hu.Ab5.H13L1 and hu.Ab4.H12L3 selectively mediated ADCC activity with increased depletion of Tregs from in vivo mixed lymphocyte reaction (MLR)-activated human PBMCs (FIGS. 12A and 12C) in comparison to conventional CD4 T cells (FIGS. 12B and 12D), and with the afucosylated variants mediating increased ADCC activity. Low level afucosylated anti-CCR8-mediated ADCC was observed in conventional CD4 T cells, consistent with the moderate upregulation of CCR8 on conventional CD4 T cells upon transfer into NSG™ mice (data not shown).

Additional data demonstrates afucosylated CCR8 mAbs Afuc.hu.Ab5.H13L1 and Afuc.hu.Ab4.H12L3 exhibit selective ADCC against Tregs from RCC tumors. Briefly, human dissociated tumor cells (Renal cell carcinoma, Discovery Life Sciences) were thawed according to the vendor's protocol. Primary NK cells were enriched from human PBMC using the Human NK Cell Isolation Kit (Miltenyi Biotec) according to manufacturer's protocol. Human dissociated tumor cells were incubated with 0.001-1 ug/mL CCR8 mAb for 30 min at room temperature prior to the addition of primary NK cells at an effector:target ratio of 2:1. After overnight incubation at 37° C., cells were processed as above to determine absolute cell counts for CD8, conventional CD4, and regulatory T cells.

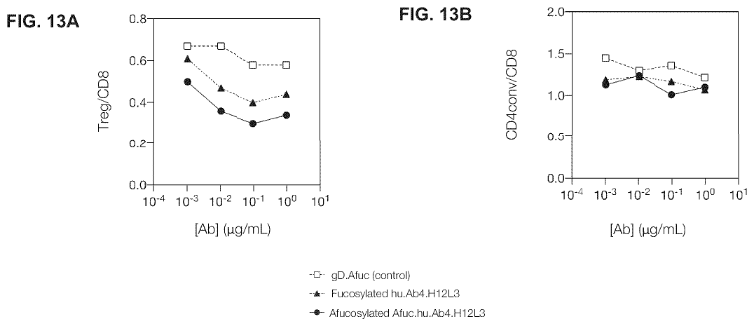

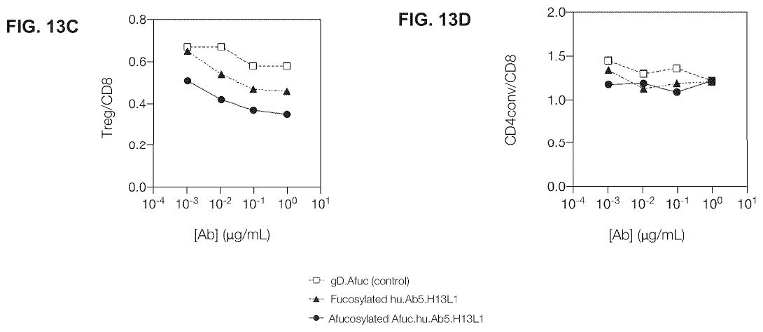

As depicted in FIGS. 13A-13D, afucosylated CCR8 antibodies Afuc.hu.Ab5.H13L1 and Afuc.hu.Ab4.H12L3 and fucosylated CCR8 antibodies hu.Ab5.H13L1 and hu.Ab4.H12L3 mediated selective ADCC activity with increased depletion of Treg cells from human dissociated tumor cells from RCC (FIGS. 13A and 3C) in comparison to conventional CD4 T cells (FIGS. 13B and 13D), and with the afucosylated variants mediating increased ADCC activity. Consistent with the absence of CCR8 staining on intratumoral conventional CD4 T cells, CCR8 mAb-mediated ADCC activity was not observed on conventional CD4 T cells, demonstrating the selectivity of CCR8 mAb-mediated ADCC against intratumoral regulatory T cells.

### (e) ADCP Enhancement

Conflicting reports exist on the impact of afucosylation on ADCP. See, e.g., Herter, et al. J Immunol (2014) 192: 2252-2260; Silence et al., mAbs (2013) 6:523-532; and Kwiatkowski et al., mAbs (2020) 12:e1803645 (9 pages). Furthermore, the G236A.I332E mutant has previously been shown to increase ADCP via enhanced FcgR2a binding. See Richards et al., Molecular Cancer Therapeutics (2008) 7:2517-2527. Therefore, fucosylated and afucosylated hIgG1.G236A.I332E Fc versions of both hu.Ab5.H13L1 and hu.Ab4.H12L3 were prepared to investigate whether ADCP activity would be observed. The G236A.I332E mutant hIgG1 constant domain is provided in the below Table, with mutational differences from the normal hIgG1 constant domain underlined, along with full-length heavy chain sequences of the Ab4 and Ab5 G236A.I332E variants.

TABLE K
hIgG1.G236A.1332E variants

| Description | Sequence |
|---|---|
| hIgG1.G236A. | ASTKGPSVFPLAPSSKSTSGGTAALGCLVKDYFPEPVTVSWNSGALTSGVHTFPAV |
| I332E | LQSSGLYSLSSVVTVPSSSLGTQTYICNVNHKPSNTKVDKKVEPKSCDKTHTCPPC |
| Constant | PAPELLAGPSVFLFPPKPKDTLMISRTPEVTCVVVDVSHEDPEVKFNWYVDGVEVH |
| domain | NAKTKPREEQYNSTYRVVSVLTVLHQDWLNGKEYKCKVSNKALPAPEEKTISKAKG |
| (SEQ ID NO: | QPREPQVYTLPPSREEMTKNQVSLTCLVKGFYPSDIAVEWESNGQPENNYKTTPPV |
| 59) | LDSDGSFFLYSKLTVDKSRWQQGNVFSCSVMHEALHNHYTQKSLSLSPGK |
| Afuc.hu.Ab5. | EVQLLESGGGLVQPGGSLRLSCAASGIDLSTYAMGWVRQAPGKGLEWVGLIHRSGR |
| H13.G236A. | TYYATWAKGRFTISKDSSKNTLYLQMNSLRAEDTAVYYCTRSYPDYSATASIWGQG |
| 1332E | TTVTVSSASTKGPSVFPLAPSSKSTSGGTAALGCLVKDYFPEPVTVSWNSGALTSG |
| Full-length | VHTFPAVLQSSGLYSLSSVVTVPSSSLGTQTYICNVNHKPSNTKVDKKVEPKSCDK |
| Heavy Chain | THTCPPCPAPELLAGPSVFLFPPKPKDTLMISRTPEVTCVVVDVSHEDPEVKFNWY |
| (SEQ ID NO: | VDGVEVHNAKTKPREEQYNSTYRVVSVLTVLHQDWLNGKEYKCKVSNKALPAPEEK |
| 60)^{a} | TISKAKGQPREPQVYTLPPSREEMTKNQVSLTCLVKGFYPSDIAVEWESNGQPENN |
| | YKTTPPVLDSDGSFFLYSKLTVDKSRWQQGNVFSCSVMHEALHNHYTQKSLSLSPG |
| | K |
| Afuc.hu.Ab4. | EVQLLESGGGLVQPGGSLRLSCAASGFSLSNYAMIWVRQAPGKGLEWVSTISLGGY |
| H12.G236A. | TYYANWAKGRFTISRDSSKTTVYLQMNSLRAEDTAVYFCARARWSTDSAIYTYAFD |
| 1332E | PWGPGTLVTVSSASTKGPSVFPLAPSSKSTSGGTAALGCLVKDYFPEPVTVSWNSG |
| Full-length | ALTSGVHTFPAVLQSSGLYSLSSVVTVPSSSLGTQTYICNVNHKPSNTKVDKKVEP |
| Heavy Chain | KSCDKTHTCPPCPAPELLAGPSVFLFPPKPKDTLMISRTPEVTCVVVDVSHEDPEV |
| (SEQ ID NO: | KFNWYVDGVEVHNAKTKPREEQYNSTYRVVSVLTVLHQDWLNGKEYKCKVSNKALP |
| 61)^{b} | APEEKTISKAKGQPREPQVYTLPPSREEMTKNQVSLTCLVKGFYPSDIAVEWESNG |
| | QPENNYKTTPPVLDSDGSFFLYSKLTVDKSRWQQGNVFSCSVMHEALHNHYTQKSL |
| | SLSPGK |

^{a}The light chain full-length sequence for the Ab5 G236A.1332E variant corresponds to hu.Ab5.L1 (SEQ ID NO: 56).
^{b}The light chain full-length sequence for the Ab4 G236A.1332E variant corresponds to hu.Ab4.L3 (SEQ ID NO: 58).

Human CD14⁺ monocytes were first isolated from blood of Genentech donors with known FcgRIIa and FcgRIIIa genotype information, by using EASYSEP™ Human Monocyte Enrichment Kit (Stem Cell Technologies). The purified CD14⁺ monocytes were differentiated into macrophages in RPMI+10% FEBS with 100 ng/mL hM-CSF (PeproTech, Inc) for 5 days. Then 50 ng/mL of hIL-10 ((PeproTech, Inc) were added to polarize the macrophages for 24 hours prior to ADCP assay. NUCLIGHT™ Red transfected CHO/hCCR8.Gna15 target cells were pre-incubated with anti-CCR8 antibodies for 20 minutes in the presence of 20 mg/mL of non-specific human IgG. Then the above cell mixtures were added to the macrophage (effector cell) plate at an E:T ratio of 1:1. After the plate was placed inside the INCUCYTE® Zoom instrument (Essen Biosciences; Ann Harbor, MI), cell images were obtained with bright field and red laser settings every one hour for a period of 6 hours. The red cell count in each well (remaining target cells) was normalized by the macrophage numbers in the same well using the instrument-embedded software. The ADCP activity was calculated as the percentage of decrease of the normalized red cell count in each sample compared to the negative control where isotype control antibody was present. Then the ADCP activity was plotted as a function of antibody concentrations and the data were fitted to an asymmetric sigmoidal four-parameter logistic (4PL) model using PRISM®. The EC₅₀ value for each antibody was determined as the concentration reaching 50% target cell killing.

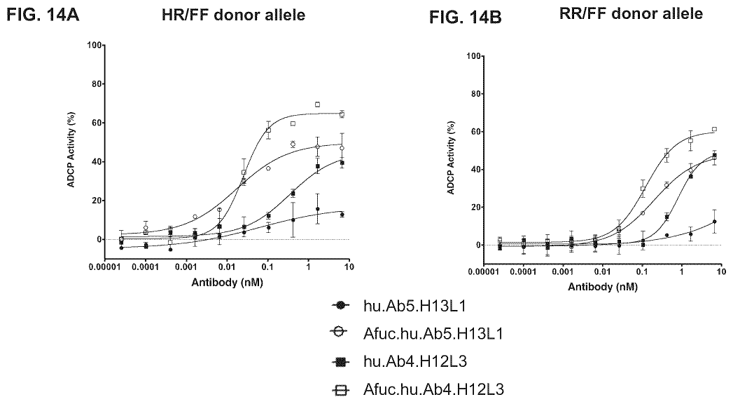

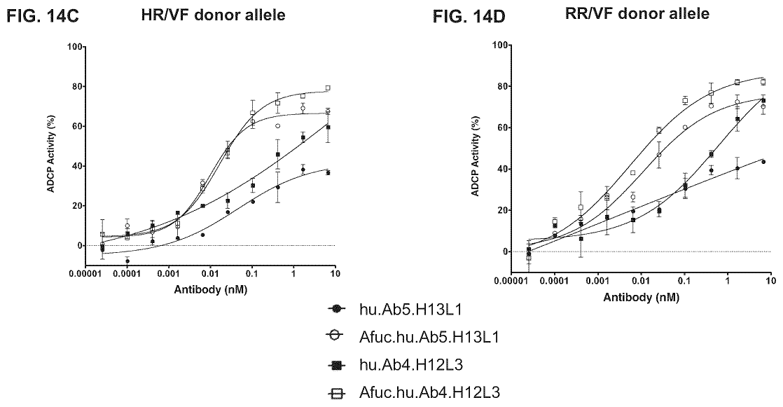

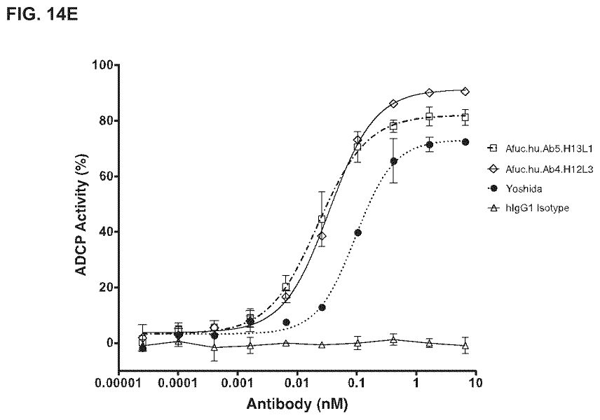

As depicted in FIGS. 14A-14D, afucosylated anti-CCR8 antibodies Afuc.hu.Ab5.H13L1 and Afuc.hu.Ab4.H12L3 exhibited enhanced ADCP activities compared to fucosylated antibodies hu.Ab5.H13L1 and hu.Ab4.H12L3 in CD14⁺ monocytes-derived macrophages from four different donors with FcgRIIa (H131R)/FcgRIIIa (V158F) genotypes of HR/FF (FIG. 14A), RR/FF (FIG. 14B), HR/VF (FIG. 14C), and RR/VF (FIG. 14D). The results indicate that in the context of the antibodies targeting CCR8, afucosylation results in enhanced ADCP.

Afucosylated anti-CCR8 antibodies Afuc.hu.Ab5.H13L1 and Afuc.hu.Ab4.H12L3 also exhibited enhanced ADCP activities compared to the Yoshida humanized anti-human CCR8 antibody (3- to 4-fold improvement) (FIG. 14E).

Activity data from these studies is also provided in the below Table.

TABLE L
ADCP Activity

| | HR/FF | RR/FF | HR/VF | RR/VF | HR/FF |
|---|---|---|---|---|---|
| | EC₅₀ | EC₅₀ | EC₅₀ | EC₅₀ | EC₅₀ |
| | (nM) | (nM) | (nM) | (nM) | (nM) |
| | FIG. | FIG. | FIG. | FIG. | FIG. |
| Antibody | 14A | 14B | 14C | 14D | 14E |
| hu.Ab5.H13L1 | n.d. | n.d. | n.d. | n.d. | |
| Afuc.hu.Ab5.H13L1 | 0.017 | 0.2 | 0.01 | 0.50 | 0.022 |
| hu.Ab4.H12L3 | 0.35 | 0.81 | n.d. | 0.41 | |
| Afuc.hu.Ab4.H12L3 | 0.025 | 0.12 | 0.017 | 0.079 | 0.035 |
| Yoshida Ab | | | | | 0.096 |

n.d. = not determined.

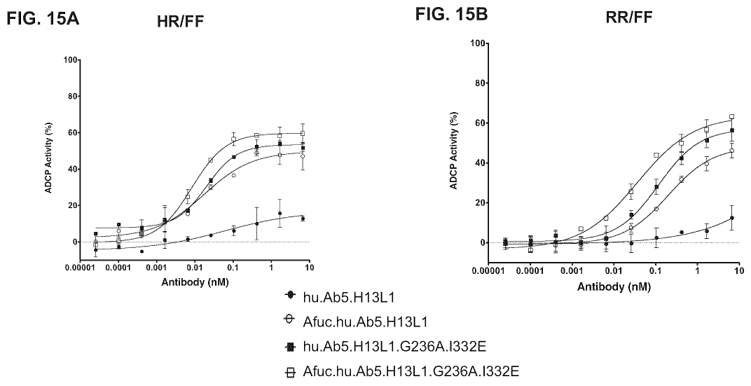

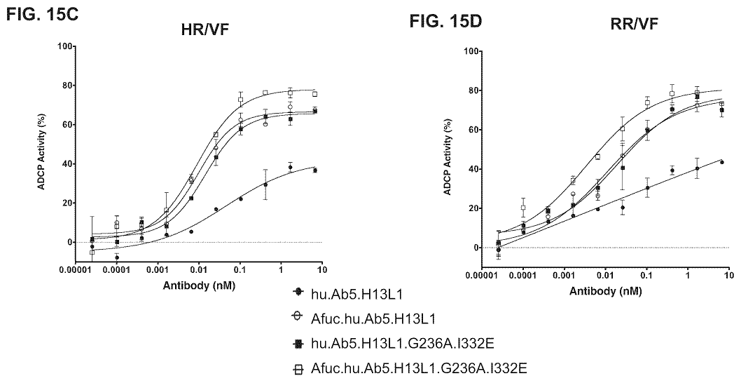

Furthermore, as depicted in FIGS. 15A-15D, the afucosylated anti-CCR8 antibody Afuc.hu.Ab5.H13L1 exhibited similar improved ADCP activities compared to the FcgRIIa-enhanced G236A.I332E variant Afuc.hu.Ab5.H13L1G236A.I332E in CD14⁺ monocytes-derived macrophages from four different donors with FcgRIIa (H131R)/FcgRIIIa (V158F) genotypes of genotypes of HR/FF (FIG. 15A), RR/FF (FIG. 15B), HR/VF (FIG. 15C), and RR/VF (FIG. 15D). The similarity in ADCP activities between the afucosylated hIgG1 variant and the G236A.I332E mutant are surprising given a previous report that incorporating G236A.I332E mediates substantially higher levels of ADCP, albeit with an anti-EPCAM mAb. See Richards et al., Molecular Cancer Therapeutics (2008) 7:2517-2527.

### (f) Physical Characterization of Ab4 and Ab5 Antibodies

The solubility, viscosity, and behavior under thermal stress (shelf life stability), of both Afuc.hu.Ab5.H13L1 and Afuc.hu.Ab4.H12L3 were evaluated at high concentrations. As shown in the below Table, both antibodies showed favorable chemical and physical properties useful in their manufacture and formulation, demonstrating low aggregation, good solubility, low viscosity, and good shelf life stability.

TABLE M
Antibody Physical Characterization

| Assay | Afuc.hu.Ab5.H13L1 | Afuc.hu.Ab4.H12L3 |
|---|---|---|
| Thermal stress | 2.0% SEC Monomer | 1.9% SEC Monomer |
| (150 mg/mL in 200 mM | loss | loss |
| Arginine Succinate, | | |
| pH 5.5) | | |
| Solubility in PBS, | 0.383 | 0.376 |
| pH 7.4 (OD) | | |
| Viscosity (in 200 mM | 9.6 cP | 12.4 cP |
| Arginine Succinate, | (180.3 mg/mL) | (187.1 mg/mL) |
| pH 5.5) | | |

Thermal Stress Conditions: Antibody samples were incubated at 150 mg/mL in 200 mM Arginine Succinate, pH 5.5, for 2 weeks at 40° C. Control samples were stored at −70° C. Size variants were evaluated for the control and stress samples using size exclusion chromatography (SEC). SEC was performed with a Waters Acquity UPLC H-Class (Waters, Milford, MA) with a TSKGEL® UP-SW3000 column, 4.6×300 mm (Tosoh Biosciences, King of Prussia, PA). The mobile phase was 0.2 M potassium phosphate buffer (pH 6.2) containing 0.25 M potassium chloride. The separation was conducted at ambient temperature with a flow rate of 0.3 mL/min and column effluent was monitored at 280 nm UV wavelength.

Solubility in Phosphate Buffered Saline (PBS): The antibodies were formulated at 150 mg/mL in 200 mM arginine succinate, pH 5.5 and dialyzed into PBS, pH 7.4 for 24 hours at 37° C. to determine their solubility. After dialysis, samples were visually inspected for visible particulates and the turbidity was determined by using a SPECTRAMAX® M2/M2e plate reader (Molecular Devices, San Jose, CA) to measure the absorbance at 340, 345, 350, 355, and 360 nanometers. The values at the 5 wavelengths were averaged resulting in the final solubility value.

Viscosity Determination: The viscosity of the sample at 100, 150, and 180 mg/mL in 200 mM Arginine Succinate, pH 5.5 was determined using an AR G2 Rheometer (TA Instruments, New Castle, DE). A 20 mm cone geometry was used, and measurements were conducted over 2.5 minutes at a constant shear rate of 1,000 inverse seconds.

(g) Epitope mapping of hu.Ab5.H13L1

To epitope map fucosylated hu.Ab5.H13L1, the binding to alanine point mutations in human CCR8 was analyzed by flow cytometry.

Constructs encoding for individual alanine point mutations at positions 2-24 in hCCR8 with a C-terminal FLAG® tag were generated. HEK 293 cells were transfected with constructs encoding for mutant hCCR8 or with a mock construct using TRANSIT-X2® (reagent:DNA=3:1) for 24 hrs, and surface stained with the huCCR8 antibody hu.Ab5.H13L1 (hIgG1), then fixed and permeabilized and followed by FITC-anti-FLAG® (Sigma F4049).

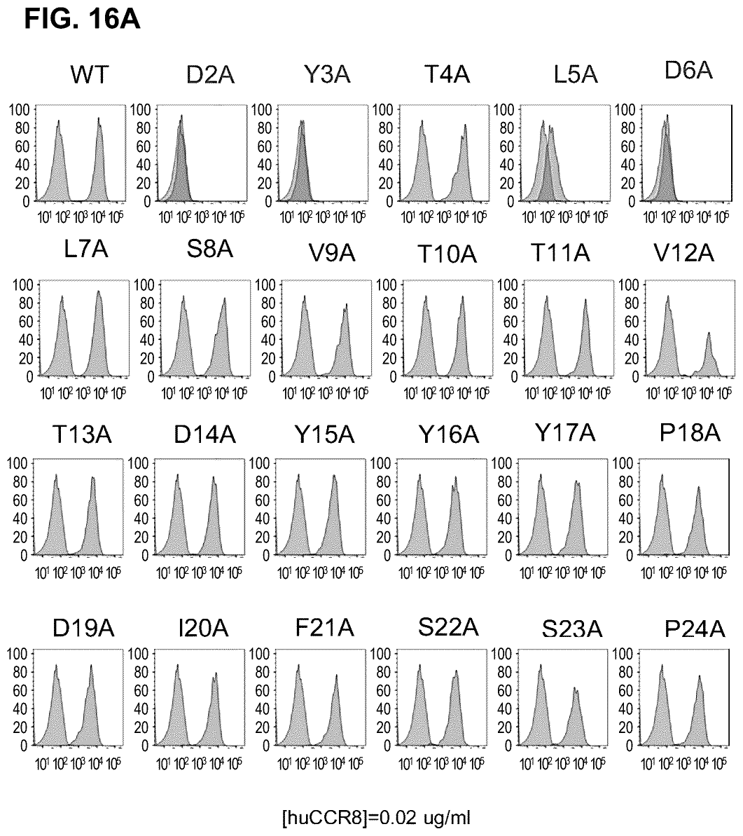

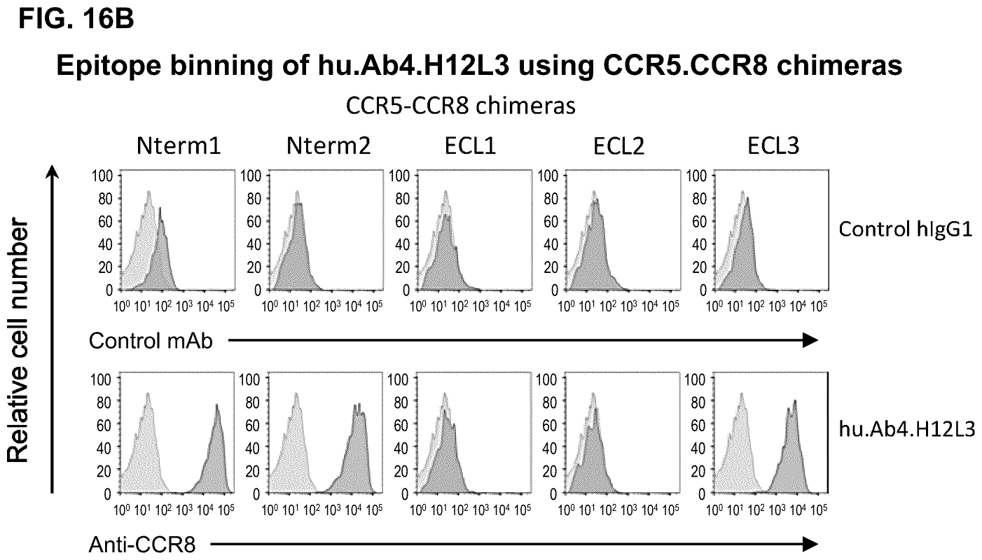

As shown in FIG. 16A, hu.Ab5.H13L1 does not bind D2A, Y3A, L5A, and D6A, indicating that the epitope includes at least one amino acid residue of the DYTLD region of the human CCR8 N-terminus.

### (h) Epitope Mapping of hu.Ab4.H12L3

To epitope map fucosylated hu.Ab4.H12L3, the binding to chimeric forms of human CCR8 was analyzed by flow cytometry.

Constructs encoding for human CCR8.CCR5 chimeras (N-term1 (amino acid residues 1-23 of human CCR8), N-term2 (amino acid residues 1-36 of human CCR8), ECL1 (amino acid residues 91-104 of human CCR8), ECL2 (amino acid residues 172-193 of human CCR8), and ECL3 (amino acid residues 264-271 of human CCR8) in which different extracellular regions of CCR8 were replaced with the corresponding region from CCR5 with a C-terminal FLAG® tag were generated. ECL is defined as an extracellular loop. 293 cells were transfected with constructs encoding for mutant hCCR8 or with a mock construct using TRANSIT-X2® (reagent:DNA=3:1) for 24 hrs, and surface stained with huCCR8-Ab4.H12L3.hIgG1, then fixed and permeabilized and followed by FITC-anti-FLAG® (Sigma F4049).

As shown in FIG. 16B, hu.Ab4.H12L3 does not bind the ECL1 and ECL2 chimeras indicating that the epitope includes at least one amino acid residue of the ECL1 and ECL2 regions of CCR8.

### Example 5. Mouse Surrogate Anti-CCR8 Monoclonal Antibody (mAb) in Murine Colon Cancer Model CT26

### (a) Treg Cell Depletion

To demonstrate the ability of an anti-CCR8 Ab to deplete tumor-infiltrating Treg cells in vivo, BALB/c mice with established CT26 tumors were treated with a mouse surrogate anti-CCR8 mAb and the proportion of Treg cells, conventional CD4 T cells and CD8 T cells among leukocytes in tumors, spleen and tumor-draining lymph nodes was analyzed by flow cytometry.

The light chain and heavy chain CDR regions, light and heavy variable regions, and full-length heavy chain and light chain sequences, of the mouse surrogate anti-CCR8 mAb is provided in the below Tables.

TABLE N1
Light Chain CDR Regions for Mouse Surrogate

| | CDR L1 | CDR L2 | CDR L3 |
|---|---|---|---|
| | (Kabat and | (Kabat and | (Kabat and |
| Description | Chothia) | Chothia) | Chothia) |
| Anti-mCCR8 | RSSKSVYSNYLS | RASTLTP | AGGYSSGSDNT |
| | (SEQ ID NO: | (SEQ ID NO: | (SEQ ID NO: |
| | 62) | 63) | 64) |

TABLE N2
Heavy Chain CDR Regions for Mouse Surrogate

| | | | CDR H2 | CDR H3 |
|---|---|---|---|---|
| Descrip- | CDR H1 | CDR H1 | (Kabat and | (Kabat and |
| tion | (Kabat) | (Chothia) | Chothia) | Chothia) |
| Anti- | EYSMA | RIDLNEY | YIDAGSGSAYYA | DVYPGYTTGT |
| mCCR8 | (SEQ ID | (SEQ ID | SWAKG (SEQ | NLGL (SEQ |
| | NO: 65) | NO: 66) | ID NO: 67) | ID NO: 68) |

TABLE N3
Light Chain and Heavy Chain Variable
Regions for Mouse Surrogate

| Description | Sequence |
|---|---|
| Anti-mCCR8 Variable | AAVLTQTPASVSAAVGGTVSISCRSSKSVYSNYLSWYQQKPGQPPKLL |
| Light Chain | IYRASTLTPGVPSRFKGSGSGTQFSLTIRDVQSADAGSYYCAGGYSSG |
| (SEQ ID NO: 69) | SDNTFGGGTKLEIK |
| Anti-mCCR8 Variable | QSVKESGGRLVTPGGSLTLTCTVSRIDLNEYSMAWVRQAPGKGLEWIG |
| Heavy Chain | YIDAGSGSAYYASWAKGRFTISKTSSTTVDLEMTTLTTEDTATYFCAR |
| (SEQ ID NO: 70) | DVYPGYTTGTNLGLWGPGTLVTVSS |

TABLE N4
Full-length Light and Heavy Chain Mouse Surrogate Sequences

| Description | Sequence |
|---|---|
| Anti-mCCR8 | AAVLTQTPASVSAAVGGTVSISCRSSKSVYSNYLSWYQQKPGQPPKLLIYRA |
| Full-length Light | STLTPGVPSRFKGSGSGTQFSLTIRDVQSADAGSYYCAGGYSSGSDNTFGGG |
| Chain | TKLEIKRADAAPTVSIFPPSSEQLTSGGASVVCFLNNFYPKDINVKWKIDGS |
| (SEQ ID NO: 71) | ERQNGVLNSWTDQDSKDSTYSMSSTLTLTKDEYERHNSYTCEATHKTSTSPI |
| | VKSFNRNEC |
| Anti-mCCR8 | QSVKESGGRLVTPGGSLTLTCTVSRIDLNEYSMAWVRQAPGKGLEWIGYIDA |
| Full-length Heavy | GSGSAYYASWAKGRFTISKTSSTTVDLEMTTLTTEDTATYFCARDVYPGYTT |
| Chain | GTNLGLWGPGTLVTVSSAKTTAPSVYPLAPVCGDTTGSSVTLGCLVKGYFPE |
| (SEQ ID NO: 72) | PVTLTWNSGSLSSGVHTFPAVLQSDLYTLSSSVTVTSSTWPSQSITCNVAHP |
| | ASSTKVDKKIEPRGPTIKPCPPCKCPAPNLLGGPSVFIFPPKIKDVLMISLS |
| | PIVTCVVVDVSEDDPDVQISWFVNNVEVHTAQTQTHREDYNSTLRVVSALPI |
| | QHQDWMSGKEFKCKVNNKDLPAPIERTISKPKGSVRAPQVYVLPPPEEEMTK |
| | KQVTLTCMVTDFMPEDIYVEWTNNGKTELNYKNTEPVLDSDGSYFMYSKLRV |
| | EKKNWVERNSYSCSVVHEGLHNHHTTKSFSRTPGK |

CT26 tumor cells were harvested in log-phase growth and resuspended in HBSS containing MATRIGEL® at a 1:1 ratio. BALB/c mice were inoculated subcutaneously in the flank with 0.1 million CT26 cells in 100 microliters of HBSS+MATRIGEL®. Tumors were monitored until they became established and reached a mean tumor volume 130-230 mm³. Mice were then randomized into treatment groups. Treatment with a mouse surrogate anti-CCR8 (mIgG2a) or an anti-gp120 isotype control Ab was administered intravenously at doses between 0.003 mg/kg and 5 mg/kg anti-CCR8 Ab in Histidine Buffer #08: 20 mM histidine acetate, 240 mM sucrose, 0.02% Polysorbate 20 (Tween-20), pH5.5.

Three days later mice were sacrificed, and tumors, spleens and tumor-draining lymph nodes obtained for analysis. To generate single cell suspensions, tumors were minced and digested in RPMI-1640 media containing 1% FBS, 0.2 U/mL LIBERASE™ DL (Sigma), and 0.2 mg/mL DNaseI (Sigma) for 30 min with agitation at 37° C. Tumor cells were passed through a 100 mm filter and washed with RPMI-1640 media containing 1% FBS. Single cell suspensions were surface stained for 15 min at 4° C. with fluorescently labelled anti-CD45, anti-CD4 and anti-CD8 antibodies and intracellularly stained with fluorescently labelled anti-Foxp3 using the EBIOSCIENCE™ Foxp3/Transcription Factor Staining Buffer Set (Thermo Fisher) according to the manufacturer's protocol. Flow cytometry was performed on a FORTESSA™ X-20 (BD Biosciences) or FACSYMPHONY™ (BD Biosciences) and analyzed with FLOWJO™ software (BD Biosciences).

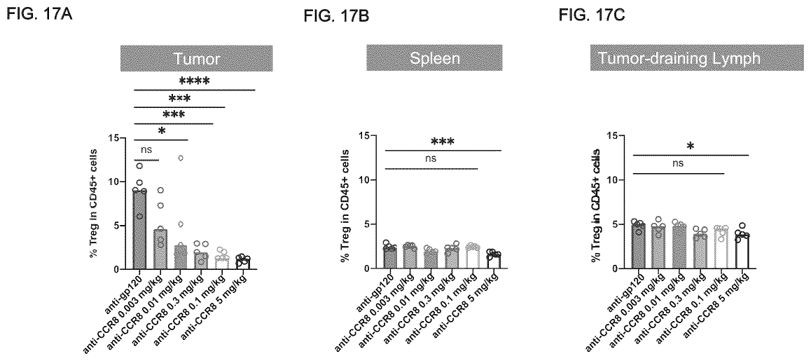

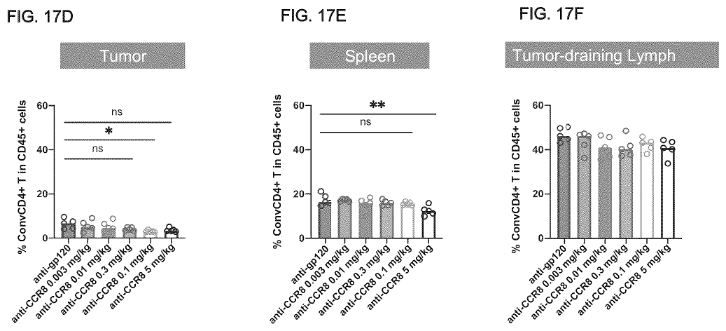

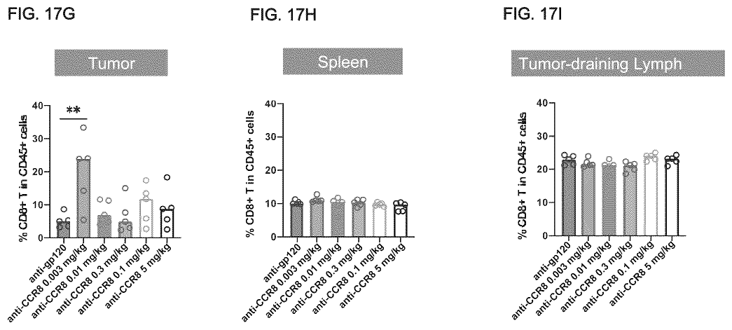

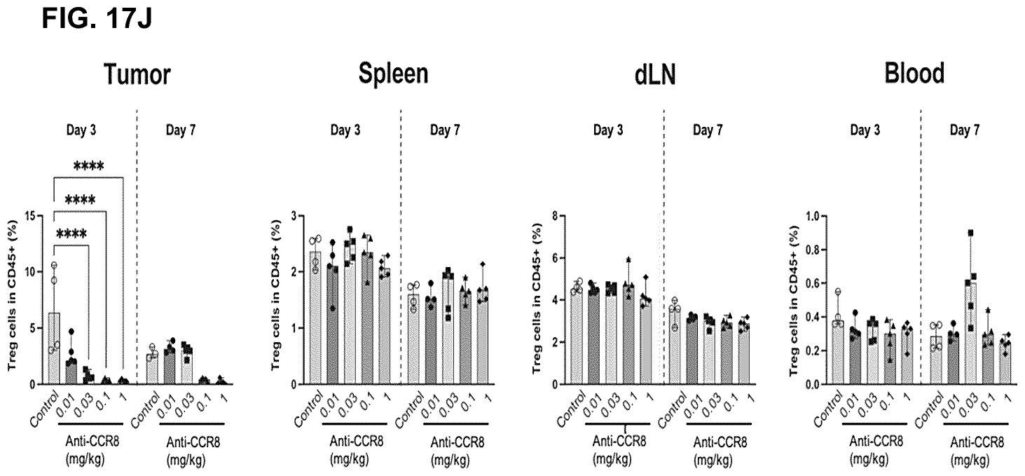

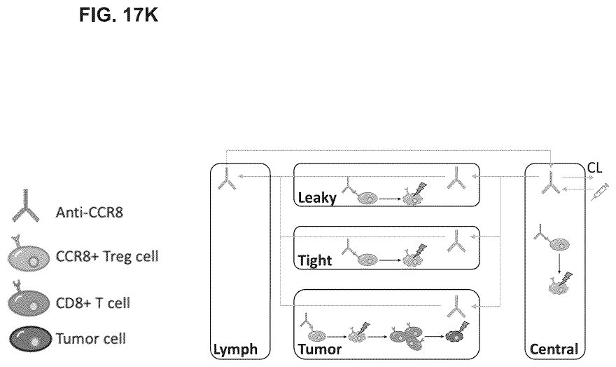

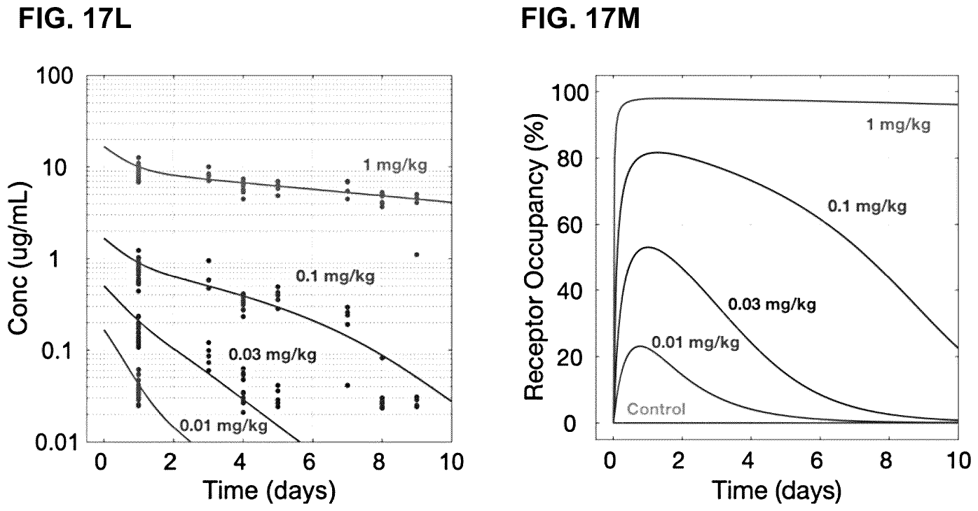

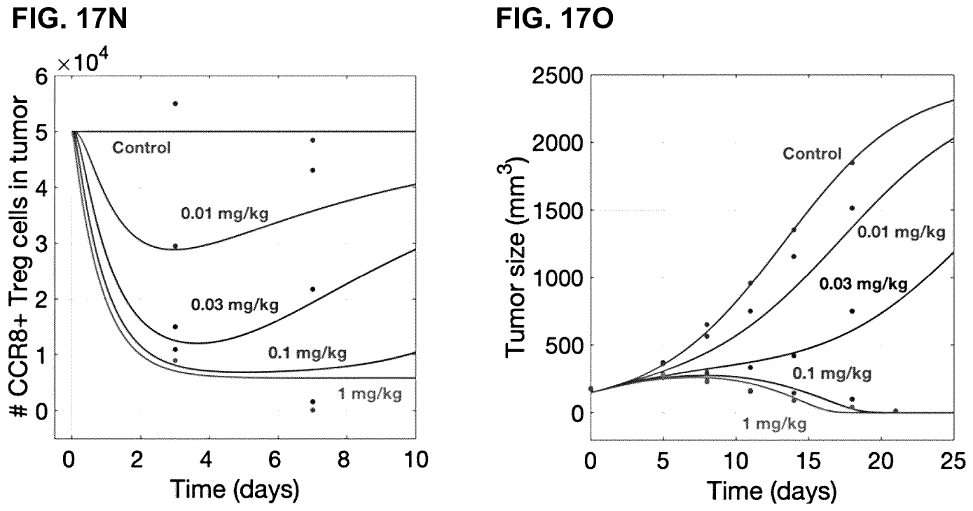

FIGS. 17A-17I depict the dose-dependent depletion of Treg cells (graphed as fraction of Treg cells among CD45+ leukocytes) in tumors, but not in spleens or tumor-draining lymph nodes (FIGS. 17A-17C) of CT26 tumor-bearing mice relative to the isotype-treated group. No reduction in the proportion of conventional CD4 T cells (FIGS. 17D-17F) or CD8 T cells (FIGS. 17G-17I) relative to the isotype control group was observed with anti-CCR8 treatment. These observations demonstrate the specificity of anti-CCR8-mediated depletion of intratumoral Treg cells.

FIG. 17J depicts the dose-dependent depletion of Treg cells (graphed as fraction of Treg cells among CD45+ leukocytes) in tumors but not in the spleen, draining lymph node, or blood in E0771 syngeneic tumor-bearing mice relative to isotype-treated group. Anti-CCR8 antibody leads to selective, dose-dependent CCR8+ Treg depletion in E0771 syngeneic mouse model. Tumor Tregs are depleted by day 3 post-dose while preserving Tregs in the spleen, draining lymph node, and blood. A minimal physiologically based pharmacokinetic-pharmacodynamic (PBPK-PD) model was used to simulate the pharmacokinetic (PK)/receptor occupancy (RO) relationship as well as pharmacodynamic (PD) and efficacy of mouse surrogate anti-CCR8 antibody (FIG. 17K). The minimal PBPK-PD model was built to simulate the PK/RO relationship as well as PD and efficacy of mouse surrogate anti-CCR8 antibody. The model incorporates five key elements: (1) anti-CCR8 antibody PK in blood, tumor, and non-tumor tissue, (2) anti-CCR8 antibody-CCR8 binding, (3) CCR8+ Treg cell depletion in tumor, (4) CD8+ T cell expansion, and (5) tumor cell killing. CCR8+ Treg cell depletion is dependent on amount of antibody-receptor complex. The model incorporates CD8+ T cell expansion as a result of CCR8+ Treg cell depletion, i.e., tumor cell killing is CD8+ T cell dependent.

This model predicts PK (FIG. 17L), RO (FIG. 17M), Treg depletion (FIG. 17N), and anti-tumor efficacy (FIG. 17O) of anti-CCR8 antibody in a dose-dependent fashion. The model captures PK over four dose levels by combining linear and nonlinear clearance terms (FIG. 17L). Since target capacity of CCR8 in mice is low, target-mediated drug disposition (TMDD) may not explain the nonlinear PK. The model predicts that dose levels of 0.01 to 1 mg/kg cover nearly the full range of receptor occupancy (FIG. 17M). The model demonstrates the dynamics of CCR8+ Treg depletion in a dose-dependent fashion (FIG. 17N). The model captures the partial recovery of the average number of Tregs at doses 0.03 mg/kg and below between days 3 and 7. The model captures average tumor killing following Treg depletion and CD8+ T cell expansion (FIG. 17O). The expansion of CD8+ T cells was required to account for the time delay between CCR8-drug binding and tumor killing.

The model outcomes are based on the mechanism of action hypothesis for anti-CCR8 antibody: CCR8+ Treg depletion followed by subsequent CD8+ T cell expansion elicits tumor killing. Dose levels below 1 mg/kg do not fully saturate CCR8 but can drive complete tumor killing in 21 days. The model is equipped to incorporate human data as anti-CCR8 antibody progresses through clinical trials to aid in the clinical study design and active dose range selection.

### (b) Tumor Growth Inhibition

To demonstrate tumor growth inhibition following anti-CCR8-mediated depletion of tumor-infiltrating Treg cells in vivo, BALB/c mice with established CT26 tumors were treated with a mouse surrogate anti-CCR8 mAb and monitored for tumor growth over time.

CT26 tumor cells were harvested in log-phase growth and resuspended in HBSS containing MATRIGEL® at a 1:1 ratio. BALB/c mice were inoculated subcutaneously in the flank with 0.1 million CT26 cells in 100 microliters of HBSS+MATRIGEL®. Tumors were monitored until they became established and reached a mean tumor volume 130-230 mm³. Mice were then randomized into treatment groups. Mice were treated intravenously with a single or twice weekly dose (first dose intravenous, following doses intraperitoneally) of 0.1 mg/kg anti-CCR8 (mIgG2a), 0.1 mg/kg of an anti-CD25 antibody (clone PC61 mIgG2a) or an anti-gp120 isotype control Ab in Histidine Buffer #08: 20 mM histidine acetate, 240 mM sucrose, 0.02% Polysorbate 20 (Tween-20), pH5.5. Tumor volumes were measured in two dimensions (length and width) using Ultra Cal-IV calipers (Fred V. Fowler Co.) and volume was calculated using the formula: Tumor size (mm³)=(length×width²)×0.5.

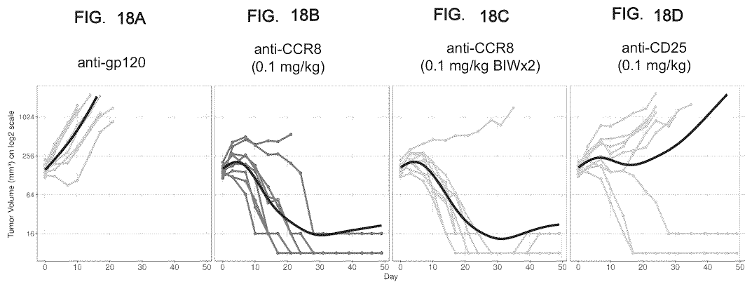

FIGS. 18A-18D depicts the change in tumor volume over time for individual mice (grey lines) and the treatment group (fitted curve, black line). Potent tumor growth inhibition was observed with a mouse surrogate anti-CCR8 mAb administered as single dose (FIG. 18B) or twice weekly (FIG. 18C) in the CT26 colon cancer model. Both treatment regimens resulted in complete tumor regression in 8/9 mice. Treatment with anti-CCR8 mAb was more effective than anti-CD25 Ab treatment (FIG. 18D) which resulted in tumor regression in 3/9 mice. An isotype control mAb (anti-gp120) was used (FIG. 18A).

### Example 6. Comparison of Effector-Competent and Effector-Less Mouse Surrogate Anti-CCR8 Ab

To assess whether anti-CCR8 Ab treatment worked primarily by facilitating ADCC- and ADCP-mediated Treg cell depletion or also by inhibiting ligand-dependent activation of CCR8, we compared a ligand blocking effector-competent mIgG2a mouse surrogate anti-CCR8 Ab to a ligand blocking effector-less mIgG2a.LALAPG mutant of the same anti-CCR8 clone in the CT26 tumor model.

CT26 tumor cells were harvested in log-phase growth and resuspended in HBSS containing MATRIGEL® at a 1:1 ratio. BALB/c mice were inoculated subcutaneously in the flank with 0.1 million CT26 cells in 100 microliters of HBSS+MATRIGEL®. In the first treatment groups, a mouse surrogate anti-CCR8 mAb (mIgG2a) or an effector-less mIgG2a.LALAPG mutant anti-CCR8 or an anti-gp120 isotype control mAb were administered at a dose of 5 mg/kg twice per week starting on the day of tumor inoculation (first dose was given intravenously, all following doses given intraperitoneally) in Histidine Buffer #08: 20 mM histidine acetate, 240 mM sucrose, 0.02% Polysorbate 20 (Tween-20), pH5.5. For the second treatment groups, tumors were monitored until they became established and reached a mean tumor volume 130-230 mm³, mice were then randomized into treatment groups and treated with anti-CCR8 (mIgG2a) or an effector-less mIgG2a.LALAPG mutant anti-CCR8 Ab dosed at 5 mg/kg twice per week (firsts dose intravenously, all following doses intraperitoneally) in Histidine Buffer #08: 20 mM histidine acetate, 240 mM sucrose, 0.02% Polysorbate 20 (Tween-20), pH5.5. Tumor volumes were measured in two dimensions (length and width) using Ultra Cal-IV calipers (Fred V. Fowler Co.) and volume was calculated using the formula: Tumor size (mm³)=(length×width²)×0.5. Mice body weights were measured using an ADVENTURER™ Pro AV812 scale (Ohaus Corporation).

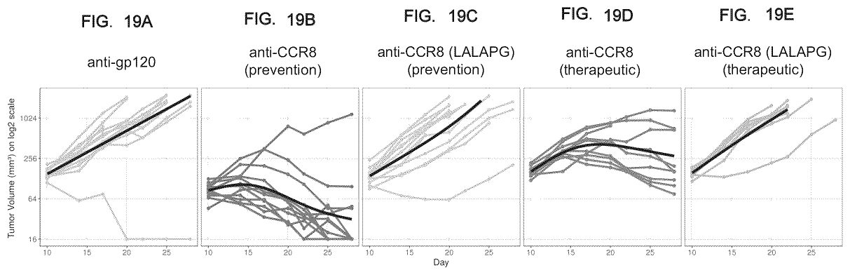

FIGS. 19A-19E depicts the change in tumor volume over time for individual mice (grey lines) and the treatment group (fitted curve, black line). Tumor growth inhibition is observed with an effector-competent mIgG2a mouse surrogate anti-CCR8 mAb (FIGS. 19B and 19D), but not with a ligand-blocking effector-less mIgG2a.LALAPG mutant anti-CCR8 mAb (FIGS. 19C and 19E). The mIgG2a anti-CCR8 Ab is effective when it was given at tumor inoculation (FIG. 19B) or in established tumors (FIG. 19D). These findings demonstrate that blocking of ligand binding to the CCR8 receptor is not sufficient to mediate tumor growth inhibition following anti-CCR8 mAb treatment. An isotype control mAb (anti-gp120) was used (FIG. 19A).

### Example 7. Combination Efficacy of Anti-CCR8 and Anti-PDL1 mAb Treatment

To assess the potential of increased tumor growth inhibition by a combination of anti-CCR8 mAb and checkpoint inhibition, mice with established EMT6 tumors were treated with anti-CCR8 and anti-PDL1 mAb individually or in combination.

EMT6 tumor cells were harvested in log-phase growth and resuspended in HBSS containing MATRIGEL® at a 1:1 ratio. BALB/c mice were inoculated subcutaneously in the 5th mammary fat pad with 0.1 million EMT6 cells in 100 microliters of HBSS+MATRIGEL®. Tumors were monitored until they became established and reached a mean tumor volume of 130-230 mm³. Mice were then randomized into treatment groups. A mouse surrogate anti-CCR8 (mIgG2a) or isotype control Ab were administered as a single dose of 0.1 mg/kg intravenously. An effector-less anti-PDL1 (mIgG2a.LALAPG) Ab was dosed at 10 mg/kg intravenously for the first dose, and 5 mg/kg intraperitoneally for subsequent doses twice a week. Antibodies were diluted in Histidine Buffer #08: 20 mM histidine acetate, 240 mM sucrose, 0.02% Polysorbate 20 (Tween-20), pH5.5. Tumor volumes and body weights were measured twice per week. Tumor volumes were measured in two dimensions (length and width) using Ultra Cal-IV calipers (Fred V. Fowler Co.) and volume was calculated using the formula: Tumor size (mm³)=(length×width²)×0.5. Mice body weights were measured using an ADVENTURER™ Pro AV812 scale (Ohaus Corporation).

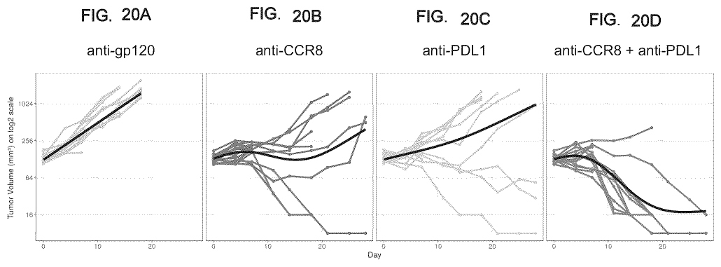

FIGS. 20A-20D depicts the change in tumor volume over time for individual mice (grey lines) and the treatment group (fitted curve, black line). Whereas a mouse surrogate anti-CCR8 and anti-PDL1 mAbs result in partial tumor growth inhibition as single treatments (FIGS. 20B-20C), the combination of both mAbs (FIG. 20D) unexpectedly leads to complete tumor rejection. An isotype control mAb (anti-gp120) was used (FIG. 20A).

### Example 8. Ab1-Ab3 H1L1 Variants and Anti-CCR8 Antibodies Comparators

### (i) Ab1-Ab3 H1L1 Variants

The light chain and heavy chain CDR regions, light and heavy variable regions, and full-length heavy chain and light chain sequences, of the Ab1-Ab3 H1L1 variants is provided in the below Tables.

TABLE O1
Light Chain CDR Regions for Ab1, Ab2, and Ab3 Variants

| | CDR L1 | CDR L2 | CDR L3 |
|---|---|---|---|
| | (Kabat and | (Kabat and | (Kabat and |
| Description | Chothia) | Chothia) | Chothia) |
| hu.Ab1.H1L1 | QASQRIGNALA | RASTLES | LGGYISIGNVYT |
| | (SEQ ID NO: 73) | (SEQ ID NO: 74) | (SEQ ID NO: 75) |
| hu.Ab2.H1L1 | QSSKSVNNNNLA | FASALAS | QGAYSGGSDSYA |
| | (SEQ ID NO: 76) | (SEQ ID NO: 77) | (SEQ ID NO: 78) |
| hu.Ab3.H1L1 | QASQSISNALA | EVSKVAS | QSAYYGNSYVWGA |
| | (SEQ ID NO: 79) | (SEQ ID NO: 80) | (SEQ ID NO: 81) |

TABLE O2
Heavy Chain CDR Regions for
Ab1, Ab2, and Ab3 Variants

| | | | | CDR H3 |
|---|---|---|---|---|
| | | | CDR H2 | (Kabat |
| Descrip- | CDR H1 | CDR H1 | (Kabat and | and |
| tion | (Kabat) | (Chothia) | Chothia) | Chothia) |
| hu.Ab1. | SNYYMC | GFDFSSN | CIHISSVTNTWY | VGNSDYRY |
| H1L1 | (SEQ ID | (SEQ ID | ASWATG (SEQ | FNL (SEQ |
| | NO: 82) | NO: 83) | ID NO: 84) | ID NO: |
| | | | | 85) |
| hu.Ab2. | YGYDMC | GFSFSYG | CISAGSSDNTWY | YLGL |
| H1L1 | (SEQ ID | (SEQ ID | ASWAKG (SEQ | (SEQ ID |
| | NO: 86) | NO: 87) | ID NO: 88) | NO: 89) |
| hu.Ab3. | SYAMS | GFSLSSY | VISASGRIIYAT | GVPSYSLV |
| H1L1 | (SEQ ID | (SEQ ID | WAKG (SEQ ID | MSD (SEQ |
| | NO: 90) | NO: 91) | NO: 92) | ID NO: |
| | | | | 93) |

TABLE O3
Heavy Chain and Light Chain Variable Regions
for Ab1, Ab2, and Ab3 Variants

| Description | Sequence |
|---|---|
| hu.Ab1.H1L1 Light Chain | DIQMTQSPSSLSASVGDRVTITCQASQRIGNALAWYQQKPGKPPKL |
| Variable Region | LIYRASTLESGVPSRFSGSGSGTDFTLTISSLOPEDVATYYCLGGY |
| (SEQ ID NO: 94) | ISIGNVYTFGGGTKVEIK |
| hu.Ab1.H1L1 Heavy Chain | EQQLVESAGGLVQPGGSLRLSCAASGFDFSSNYYMCWVRQAPGKGL |
| Variable Region | EWIGCIHISSVTNTWYASWATGRFTISKDTSSTTVYLQMISLKTED |
| (SEQ ID NO: 95) | TAVYFCTRVGNSDYRYFNLWGPGTLVTVSS |
| hu.Ab2.H1L1 Light Chain | EQVLTQSPGTLSLSPGERATLSCQSSKSVNNNNLAWYQQKPGQPPR |
| Variable Region | LLIYFASALASGVPDRFSGSGSGTDFTLTISRLEPEDFAVYYCQGA |
| (SEQ ID NO: 96) | YSGGSDSYAFGGGTKVEIK |
| hu.Ab2.H1L1 Heavy Chain | EQQLVESGGGVVQPGRSLRLSCAASGFSFSYGYDMCWVRQAPGKGL |
| Variable Region | EWIACISAGSSDNTWYASWAKGRFTISKDTSKTTVYLQMNSLRAED |
| (SEQ ID NO: 97) | TAVYFCSRYLGLWGPGTLVTVSS |
| hu.Ab3.H1L1 Light Chain | EPVMTQSPATLSVSPGERATLSCQASQSISNALAWYQQKPGQPPRL |
| Variable Region | LIYEVSKVASGVPARFSGSGSGTEFTLTISSLQSEDFAVYYCQSAY |
| (SEQ ID NO: 98) | YGNSYVWGAFGQGTKVEIK |
| hu.Ab3.H1L1 Heavy Chain | EQQLVESGGGLVQPGGSLRLSCAVSGFSLSSYAMSWVRQAPGKGLE |
| Variable Region | WIGVISASGRIIYATWAKGRFTISKDSAKTSVYLQMNSLRDEDTAV |
| (SEQ ID NO: 99) | YFCARGVPSYSLVMSDWGPGTTVTVSS |

TABLE O4
Heavy Chain and Light Chain Full-length Sequences
for Ab1, Ab2, and Ab3 Variants

| Description | Sequence |
|---|---|
| hu.Ab1.H1L1 Full- | DIQMTQSPSSLSASVGDRVTITCQASQRIGNALAWYQQKPGKPPKLLIYRAST |
| length | LESGVPSRFSGSGSGTDFTLTISSLQPEDVATYYCLGGYISIGNVYTFGGGTK |
| Light Chain | VEIKRTVAAPSVFIFPPSDEQLKSGTASVVCLLNNFYPREAKVQWKVDNALQS |
| (SEQ ID NO: 100) | GNSQESVTEQDSKDSTYSLSSTLTLSKADYEKHKVYACEVTHQGLSSPVTKSF |
| | NRGEC |
| hu.Ab1.H1L1 Full- | EQQLVESAGGLVQPGGSLRLSCAASGFDFSSNYYMCWVRQAPGKGLEWIGCIH |
| length | ISSVTNTWYASWATGRFTISKDTSSTTVYLQMISLKTEDTAVYFCTRVGNSDY |
| Heavy Chain | RYFNLWGPGTLVTVSSASTKGPSVFPLAPSSKSTSGGTAALGCLVKDYFPEPV |
| (SEQ ID NO: 101) | TVSWNSGALTSGVHTFPAVLQSSGLYSLSSVVTVPSSSLGTQTYICNVNHKPS |
| | NTKVDKKVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLTVLHQDW |
| | LNGKEYKCKVSNKALPAPIEKTISKAKGQPREPQVYTLPPSREEMTKNQVSLT |
| | CLVKGFYPSDIAVEWESNGQPENNYKTTPPVLDSDGSFFLYSKLTVDKSRWQQ |
| | GNVFSCSVMHEALHNHYTQKSLSLSPGK |
| hu.Ab2.H1L1 Full- | EQVLTQSPGTLSLSPGERATLSCQSSKSVNNNNLAWYQQKPGQPPRLLIYFAS |
| length | ALASGVPDRFSGSGSGTDFTLTISRLEPEDFAVYYCQGAYSGGSDSYAFGGGT |
| Light Chain | KVEIKRTVAAPSVFIFPPSDEQLKSGTASVVCLLNNFYPREAKVQWKVDNALQ |
| (SEQ ID NO: 102) | SGNSQESVTEQDSKDSTYSLSSTLTLSKADYEKHKVYACEVTHQGLSSPVTKS |
| | FNRGEC |
| hu.Ab2.H1L1 Full- | EQQLVESGGGVVQPGRSLRLSCAASGFSFSYGYDMCWVRQAPGKGLEWIACIS |
| length | AGSSDNTWYASWAKGRFTISKDTSKTTVYLQMNSLRAEDTAVYFCSRYLGLWG |
| Heavy Chain | PGTLVTVSSASTKGPSVFPLAPSSKSTSGGTAALGCLVKDYFPEPVTVSWNSG |
| (SEQ ID NO: 103) | ALTSGVHTFPAVLQSSGLYSLSSVVTVPSSSLGTQTYICNVNHKPSNTKVDKK |
| | VEPKSCDKTHTCPPCPAPELLGGPSVFLFPPKPKDTLMISRTPEVTCVVVDVS |
| | HEDPEVKFNWYVDGVEVHNAKTKPREEQYNSTYRVVSVLTVLHQDWLNGKEYK |
| | CKVSNKALPAPIEKTISKAKGQPREPQVYTLPPSREEMTKNQVSLTCLVKGFY |
| | PSDIAVEWESNGQPENNYKTTPPVLDSDGSFFLYSKLTVDKSRWQQGNVFSCS |
| | VMHEALHNHYTQKSLSLSPGK |
| hu.Ab3.H1L1 Full- | EPVMTQSPATLSVSPGERATLSCQASQSISNALAWYQQKPGQPPRLLIYEVSK |
| length | VASGVPARFSGSGSGTEFTLTISSLQSEDFAVYYCQSAYYGNSYVWGAFGQGT |
| Light Chain | KVEIKRTVAAPSVFIFPPSDEQLKSGTASVVCLLNNFYPREAKVQWKVDNALQ |
| (SEQ ID NO: 104) | SGNSQESVTEQDSKDSTYSLSSTLTLSKADYEKHKVYACEVTHQGLSSPVTKS |
| | FNRGEC |
| hu.Ab3.H1L1 Full- | EQQLVESGGGLVQPGGSLRLSCAVSGFSLSSYAMSWVRQAPGKGLEWIGVISA |
| length | SGRIIYATWAKGRFTISKDSAKTSVYLQMNSLRDEDTAVYFCARGVPSYSLVM |
| Heavy Chain | SDWGPGTTVTVSSASTKGPSVFPLAPSSKSTSGGTAALGCLVKDYFPEPVTVS |
| (SEQ ID NO: 105) | WNSGALTSGVHTFPAVLQSSGLYSLSSVVTVPSSSLGTQTYICNVNHKPSNTK |
| | VDKKVEPKSCDKTHTCPPCPAPELLGGPSVFLFPPKPKDTLMISRTPEVTCVV |
| | VDVSHEDPEVKFNWYVDGVEVHNAKTKPREEQYNSTYRVVSVLTVLHQDWLNG |
| | KEYKCKVSNKALPAPIEKTISKAKGQPREPQVYTLPPSREEMTKNQVSLTCLV |
| | KGFYPSDIAVEWESNGQPENNYKTTPPVLDSDGSFFLYSKLTVDKSRWQQGNV |
| | FSCSVMHEALHNHYTQKSLSLSPGK |

### (ii) Anti-CCR8 Antibodies Comparators

The full-length heavy chain and full-length light chain sequence of the Yoshida humanized anti-human CCR8 antibody studied herein was disclosed in a U.S. Declaration filed on Oct. 30, 2019, during prosecution of U.S. Ser. No. 16/183,216 (published as US 2019/0071508, and later granted as U.S. Pat. No. 10,550,191). The light chain variable region, light chain constant region, heavy chain variable region, and heavy chain constant region of this same antibody were disclosed in PCT Application Publication No. WO2020138489 as sequences 59, 52, 41, and 53. The Yoshida antibody was expressed as a human hIgG1 antibody (i.e., having a human Fc region). The commercially available murine anti-human CCR8 antibody 433H (BD Biosciences), and murine anti-human CCR8 antibody L263G8 (Biolegend) were purchased for these studies. 433H (BD Biosciences) and L263G8 (Biolegend) are mouse monoclonal antibodies comprising mouse IgG2a isotype Fc regions. See also Mutalithas et al., Clinical & Experimental Allergy (2010) 40:1175 (433H, BD Biosciences), Mitson-Salazar et al., J. Allergy Clin. Immunol. (2016) 907-918 (L263G8, Biolegend) and www.labome.com/review/gene/human/CCR8-antibody.html (L263G8, Biolegend).

### Example 9: Terminal Lysine Variants of Ab1-Ab5

Additional Fc variants of the presently disclosed anti-CCR8 antibodies are contemplated, where the C-terminus of the heavy chain of the parent antibody is a shortened C-terminus in which the C-terminal lysine has been removed, resulting in a shortened C-terminus ending PG. The terminal lysine variants of Ab1-Ab-5 are provided in the below Table P.

The light chain full-length sequence for the Ab5 terminal lysine variant corresponds to hu.Ab5.L1 (SEQ ID NO: 56).

The light chain full-length sequence for the Ab4 terminal lysine variant corresponds to hu.Ab4.L3 (SEQ ID NO: 58).

The light chain full-length sequence for the Ab5 G236A.I332E terminal lysine variant corresponds to hu.Ab5.L1 (SEQ ID NO: 56).

The light chain full-length sequence for the Ab4 G236A.I332E terminal lysine variant corresponds to hu.Ab4.L3 (SEQ ID NO: 58).

The light chain full-length sequence for the Ab1 terminal lysine variant corresponds to hu.Ab1.L1 (SEQ ID NO: 100).

The light chain full-length sequence for the Ab2 terminal lysine variant corresponds to hu.Ab2.L1 (SEQ ID NO: 102).

The light chain full-length sequence for the Ab3 terminal lysine variant corresponds to hu.Ab3.L1 (SEQ ID NO: 104).

TABLE P
Heavy Chain Terminal Lysine Variant Sequences
for Ab1, Ab2, Ab3, Ab4, and Ab5 Variants

| Description | Sequence |
|---|---|
| hu.Ab5.H13 Terminal | EVQLLESGGGLVQPGGSLRLSCAASGIDLSTYAMGWVRQAPGKGLEWVGLI |
| Lysine Variant Heavy | HRSGRTYYATWAKGRFTISKDSSKNTLYLQMNSLRAEDTAVYYCTRSYPDY |
| Chain | SATASIWGQGTTVTVSSASTKGPSVFPLAPSSKSTSGGTAALGCLVKDYFP |
| (SEQ ID NO: 111) | EPVTVSWNSGALTSGVHTFPAVLQSSGLYSLSSVVTVPSSSLGTQTYICNV |
| | NHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFL |
| | YSKLTVDKSRWQQGNVFSCSVMHEALHNHYTQKSLSLSPG |
| hu.Ab4.H12 Terminal | EVQLLESGGGLVQPGGSLRLSCAASGFSLSNYAMIWVRQAPGKGLEWVSTI |
| Lysine Variant Heavy | SLGGYTYYANWAKGRFTISRDSSKTTVYLQMNSLRAEDTAVYFCARARWST |
| Chain | DSAIYTYAFDPWGPGTLVTVSSASTKGPSVFPLAPSSKSTSGGTAALGCLV |
| (SEQ ID NO: 112) | KDYFPEPVTVSWNSGALTSGVHTFPAVLQSSGLYSLSSVVTVPSSSLGTQT |
| | YICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| Afuc.hu.Ab5.H13.G2 | EVQLLESGGGLVQPGGSLRLSCAASGIDLSTYAMGWVRQAPGKGLEWVGLI |
| 36A.I332E Terminal | HRSGRTYYATWAKGRFTISKDSSKNTLYLQMNSLRAEDTAVYYCTRSYPDY |
| Lysine Variant Heavy | SATASIWGQGTTVTVSSASTKGPSVFPLAPSSKSTSGGTAALGCLVKDYFP |
| Chain | EPVTVSWNSGALTSGVHTFPAVLQSSGLYSLSSVVTVPSSSLGTQTYICNV |
| (SEQ ID NO: 113) | NHKPSNTKVDKKVEPKSCDKTHTCPPCPAPELLAGPSVFLFPPKPKDTLMI |
| | SRTPEVTCVVVDVSHEDPEVKFNWYVDGVEVHNAKTKPREEQYNSTYRVVS |
| | VLTVLHQDWLNGKEYKCKVSNKALPAPEEKTISKAKGQPREPQVYTLPPSR |
| | EEMTKNQVSLTCLVKGFYPSDIAVEWESNGQPENNYKTTPPVLDSDGSFFL |
| | YSKLTVDKSRWQQGNVFSCSVMHEALHNHYTQKSLSLSPG |
| Afuc.hu.Ab4.H12.G2 | EVQLLESGGGLVQPGGSLRLSCAASGFSLSNYAMIWVRQAPGKGLEWVSTI |
| 36A.I332E Terminal | SLGGYTYYANWAKGRFTISRDSSKTTVYLQMNSLRAEDTAVYFCARARWST |
| Lysine Variant Heavy | DSAIYTYAFDPWGPGTLVTVSSASTKGPSVFPLAPSSKSTSGGTAALGCLV |
| Chain | KDYFPEPVTVSWNSGALTSGVHTFPAVLQSSGLYSLSSVVTVPSSSLGTQT |
| (SEQ ID NO: 114) | YICNVNHKPSNTKVDKKVEPKSCDKTHTCPPCPAPELLAGPSVFLFPPKPK |
| | DTLMISRTPEVTCVVVDVSHEDPEVKFNWYVDGVEVHNAKTKPREEQYNST |
| | YRVVSVLTVLHQDWLNGKEYKCKVSNKALPAPEEKTISKAKGQPREPQVYT |
| | LPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| hu.Ab1.H1L1 | EQQLVESAGGLVQPGGSLRLSCAASGFDFSSNYYMCWVRQAPGKGLEWIGC |
| Terminal Lysine | IHISSVTNTWYASWATGRFTISKDTSSTTVYLQMISLKTEDTAVYFCTRVG |
| Variant | NSDYRYFNLWGPGTLVTVSSASTKGPSVFPLAPSSKSTSGGTAALGCLVKD |
| Heavy Chain | YFPEPVTVSWNSGALTSGVHTFPAVLQSSGLYSLSSVVTVPSSSLGTQTYI |
| (SEQ ID NO: 115) | CNVNHKPSNTKVDKKVEPKSCDKTHTCPPCPAPELLGGPSVFLFPPKPKDT |
| | LMISRTPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPG |
| hu.Ab2.H1L1 | EQQLVESGGGVVQPGRSLRLSCAASGFSFSYGYDMCWVRQAPGKGLEWIAC |
| Terminal Lysine | ISAGSSDNTWYASWAKGRFTISKDTSKTTVYLQMNSLRAEDTAVYFCSRYL |
| Variant | GLWGPGTLVTVSSASTKGPSVFPLAPSSKSTSGGTAALGCLVKDYFPEPVT |
| Heavy Chain | VSWNSGALTSGVHTFPAVLQSSGLYSLSSVVTVPSSSLGTQTYICNVNHKP |
| (SEQ ID NO: 116) | SNTKVDKKVEPKSCDKTHTCPPCPAPELLGGPSVFLFPPKPKDTLMISRTP |
| | EVTCVVVDVSHEDPEVKFNWYVDGVEVHNAKTKPREEQYNSTYRVVSVLTV |
| | LHQDWLNGKEYKCKVSNKALPAPIEKTISKAKGQPREPQVYTLPPSREEMT |
| | KNQVSLTCLVKGFYPSDIAVEWESNGQPENNYKTTPPVLDSDGSFFLYSKL |
| | TVDKSRWQQGNVFSCSVMHEALHNHYTQKSLSLSPG |
| hu.Ab3.H1L1 | EQQLVESGGGLVQPGGSLRLSCAVSGFSLSSYAMSWVRQAPGKGLEWIGVI |
| Terminal Lysine | SASGRIIYATWAKGRFTISKDSAKTSVYLQMNSLRDEDTAVYFCARGVPSY |
| Variant Heavy Chain | SLVMSDWGPGTTVTVSSASTKGPSVFPLAPSSKSTSGGTAALGCLVKDYFP |
| (SEQ ID NO: 117) | EPVTVSWNSGALTSGVHTFPAVLQSSGLYSLSSVVTVPSSSLGTQTYICNV |
| | NHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFL |
| | YSKLTVDKSRWQQGNVFSCSVMHEALHNHYTQKSLSLSPG |

### Example 10. Serum Concentration and ADA of Test Anti-CCR8 mAbs in Cyno

Anti-CCR8 antibodies Afuc.hu.Ab5.H13L1, Afuc.hu.Ab4.H12L3, and the control anti-gD were used for this study. There were three male cynomolgus monkeys in each of the three dose groups—Control: designated 1001, 1002, 1003; Afuc.hu.Ab5.H13L1: designated 2001, 2002, 2003; and Afuc.hu.Ab4.H12L3: designated 3001, 3002, 3003. Each were given a single 10 mg/kg IV bolus of anti-gD or test anti-CCR8 mAb. Blood samples for analysis were collected at 0.25, 2, and 6 hours; 1, 2, 7, 14, 21, 28, and 35 days post-dose, and serum was assayed for concentrations of anti-gD (control group) and the anti-CCR8 antibodies using a qualified ELISA analytical method. The lower limit of quantitation (LLOQ) of the assay was 0.015625 μg/mL. PK parameters were estimated using Phoenix 1.4 (WINNONLIN™ pharmacokinetic software version 6.4) (Certara, USA) using a non-compartmental analysis consistent with IV bolus administration. Blood samples for anti-drug antibody (ADA) analysis were collected at pre-dose and on Day 1, 8, 15, 22, 29 and 36, and serum was analyzed for antibodies against the test items using a qualified ELISA assay.

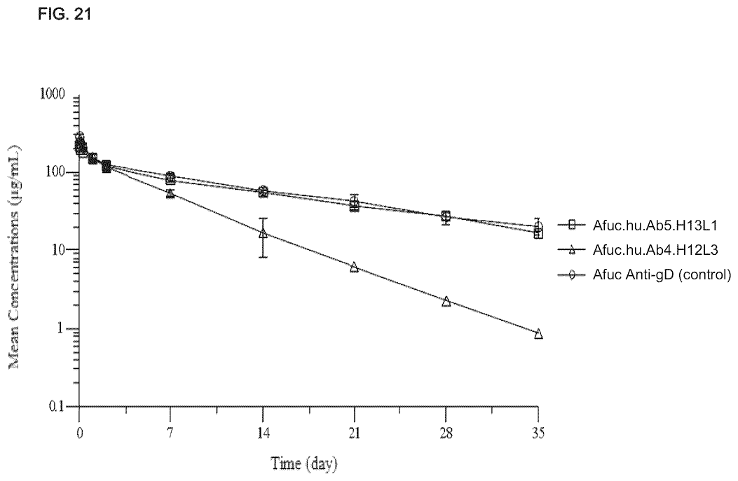

The serum concentration profiles of anti-gD, Afuc.hu.Ab5.H13L1 or Afuc.hu.Ab4.H12L3 in cynomolgus monkeys following administration of single 10 mg/kg IV dose are shown in FIG. 21. Systemic exposures were found to be comparable between the anti-gD and Afuc.hu.Ab5.H13L1 groups, exhibiting sustained serum concentration levels over the 35-day post-dose period, with a respective mean clearance of 3.96±0.412 mL/day/kg and 4.38±0.291 mL/day/kg. In contrast, Afuc.hu.Ab4.H12L3 exhibited lower exposure over that same 35-day post-dose period, with a mean clearance of 9.00±1.01 mL/day/kg. Maintaining serum concentration levels over a longer period of time, with slower clearance, as exhibited by Afuc.hu.Ab5.H13L1, is expected to elicit a more sustained target engagement that may translate to better anti-cancer activity and less frequent dosing.

The difference in systemic exposures observed for Afuc.hu.Ab4.H12L3 compared to anti-gD and Afuc.hu.Ab5.H13L1 groups could be partially explained by the presence of anti-drug antibodies (ADAs) in Afuc.hu.Ab4.H12L3 treated group at later time points. For example, Animals 1001, 1002, and 1003 dosed with anti-gD were negative for the presence of ADAs. Following administration of Afuc.hu.Ab5.H13L1, Animal 2001 was ADA-positive, but the presence of ADAs appeared to have no impact on exposure when compared to the other two Afuc.hu.Ab5.H13L1 dosed animals (Nos. 2002 and 2003) that were ADA-negative. Following administration of Afuc.hu.Ab4.H12L3, Animals 3002 and 3003 were found to be ADA-positive and the presence of ADAs appeared to have an impact on the systemic exposures when compared to ADA-negative Animal 3001.

### Example 11. Monitoring Levels of CCR8+ T-Reg Cells in Cyno

Anti-CCR8 antibodies Afuc.hu.Ab5.H13L1, Afuc.hu.Ab4.H12L3, and the control anti-gD were used for this study. There were three male cynomolgus monkeys in each of the three dose groups—Control Group: designated 1001, 1002, 1003; Afuc.hu.Ab5.H13L1 Group 2: designated 2001, 2002, 2003; and Afuc.hu.Ab4.H12L3 Group 3: designated 3001, 3002, 3003. Blood was collected from each of the animals before dosing on Day 1 (“Pre-study”), as well as on Day 1 at 0 hours (“Pre-dose”). Each of the animals was then dosed with a single dose of 10 mg/kg afucosylated anti-gD (Control Group), Afuc.hu.Ab5.H13L1 (Group 2) or Afuc.hu.Ab4.H12L3 (Group 3) via intravenous injection. Blood containing the initial dose of the test CCR8 mAb was collected at the following time points post-dose starting on Day 1: 6, 24, 48, 168, 336, 504, 668, and 840 hours, and subjected to the following treatment prior to flow cytometry analysis: (i) blood sample not spiked with either test CCR8 mAb (“unspiked”), (ii) blood sample further spiked with a saturating concentration of Afuc.hu.Ab5.H13L1, and (iii) blood sample further spiked with a saturating concentration of Afuc.hu.Ab4.H12L3. Each of the unspiked and spiked samples were then treated with a labeled goat anti-human IgG antibody, which detects for binding of the test anti-CCR8 mAb to cynoCCR8 and analyzed by flow cytometry.

T cell subsets were identified using specific antibodies against phenotypic marker antigens. Specifically, T regulatory (T-reg) cells were identified as CD3+CD4+Foxp3+ cells. Drug bound CCR8+ T-reg cells were identified using unspiked blood samples.

Both test anti-CCR8 mAbs, as observed in the unspiked samples, did not substantially reduce the total T-reg cell absolute counts in whole blood for up to 840 hours post dose. See FIGS. 22A-22C. Both test anti-CCR8 mAbs also did not substantially reduce the total lymphocyte counts in whole blood for up to 840 hours post dose total (data not shown).

As described earlier, both Afuc.hu.Ab5.H13L1 and Afuc.hu.Ab4.H12L3 bind to CCR8, with Afuc.hu.Ab4.H12L3 and Afuc.hu.Ab5.H13L1 both acting as non-competitive CCR8 binders to each other, and with Afuc.hu.Ab4.H12L3 having slightly higher affinity for human and cyno CCR8. See, e.g., FIGS. 16A-16B, and affinity K_{d} (nM) data provided in Table G3. Afuc.hu.Ab4.H12L3 also has an increased propensity for ADA formation at later time points. See Example 10.

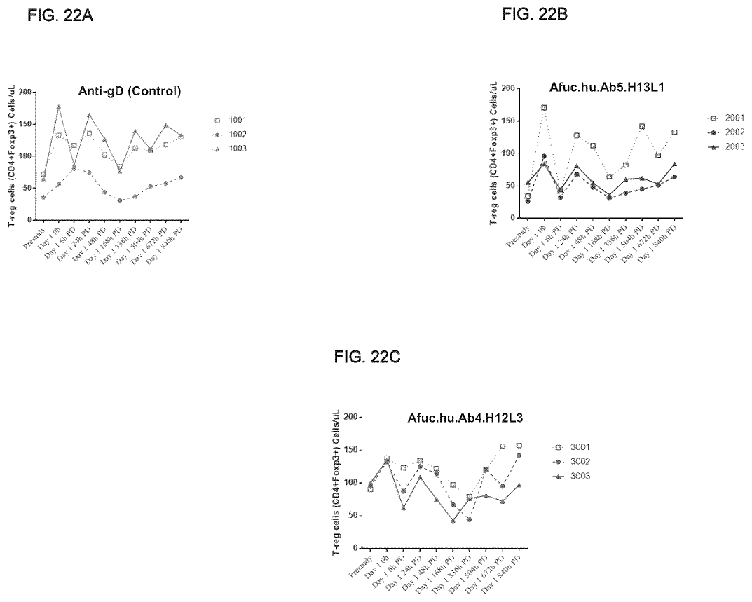

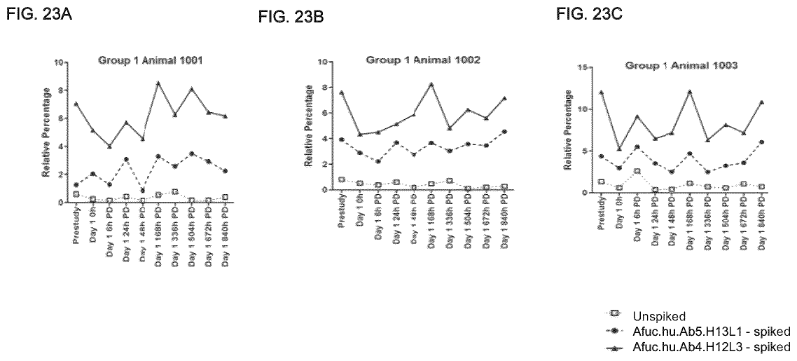

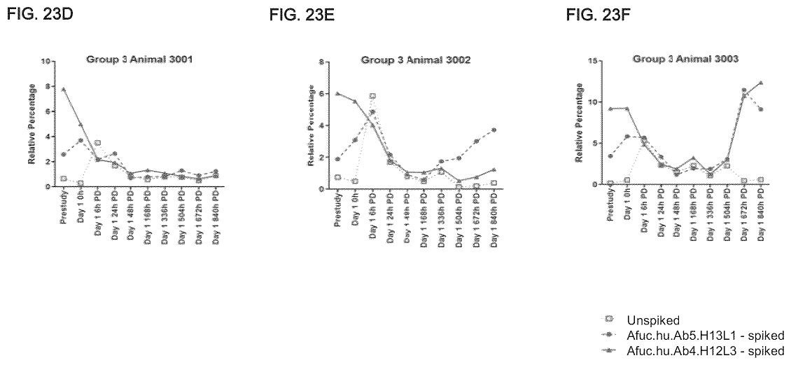

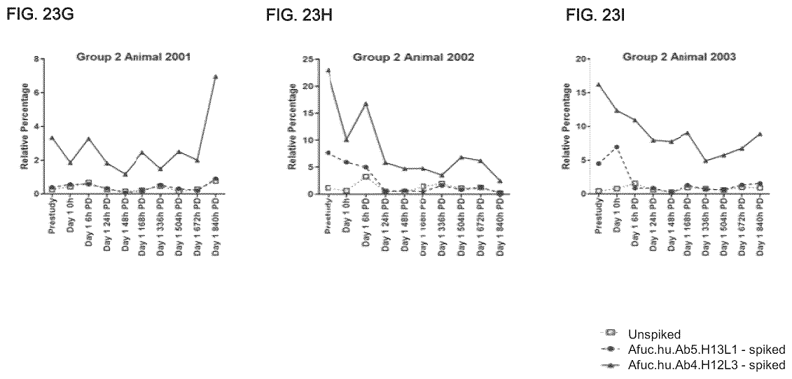

As can be seen in FIGS. 23A-23C, flow cytometry analysis of unspiked blood from cynos initially treated with control (Group 1) demonstrated no modulation of total CCR8+T-reg cells. Furthermore, flow cytometry of spiked blood (i.e., blood initially treated with control (Group 1) then spiked with a saturating concentration of Afuc.hu.Ab5.H13L1 or blood initially treated with control (Group 1) then spiked with a saturating concentration of Afuc.hu.Ab4.H12L3) also had very little effect on the total CCR8+T-reg cell count. Relative percentage refers to the percent of CCR8+T-reg cells as detected by each of the test anti-CCR8 mAbs. Since Afuc.hu.Ab4.H12L3 has a slightly higher affinity compared to Afuc.hu.Ab5.H13L1, the relative percentage of the spiked Afuc.hu.Ab4.H12L3 sample has a higher percentage detected.

With regard to Group 3, as can be seen in FIGS. 23D-23F, flow cytometry of (i) blood initially treated with Afuc.hu.Ab4.H12L3 (“unspiked”), (ii) blood initially treated with Afuc.hu.Ab4.H12L3 then spiked with Afuc.hu.Ab5.H13L1, or (iii) blood initially treated with Afuc.hu.Ab4.H12L3 then spiked with Afuc.hu.Ab4.H12L3, in each of the three animals, demonstrated a decrease in CCR8+T-reg cells up to 168 hours post dose. A partial recovery in the frequency of CCR8+ Treg cells was noticed in two of the animals starting at 336 hours post dose, likely due to the increased presence of ADAs against Afuc.hu.Ab4.H12L3.

With regard to Group 2, as can be seen in FIGS. 23G-231, flow cytometry of (i) blood initially treated with Afuc.hu.Ab5.H13L1 (“unspiked”), (ii) blood initially treated with Afuc.hu.Ab5.H13L1 then spiked with a saturating concentration of Afuc.hu.Ab5.H13L1, or (iii) blood initially treated with Afuc.hu.Ab5.H13L1 then spiked with a saturating concentration of Afuc.hu.Ab4.H12L3, demonstrated a decrease in CCR8+T-reg cells in Animals 2002 and 2003.

Both Group 2 and 3 animals demonstrated little to no effect on the overall Treg cell count (FIGS. 22A-22C) but demonstrated reduced numbers of peripheral blood CCR8+T-reg cells following administration (FIGS. 23D-231), either spiked or unspiked, which is consistent with the proposed mechanism of action (see FIG. 2A).

### Example 12. A Phase Ia/Ib, Open Label, Multicenter, Dose-Escalation Study to Evaluate the Safety, Pharmacokinetics, and Activity of the Anti-CCR8 Antibody RO7502175 as a Single Agent and in Combination with the Anti-PD-L1 Antibody Atezolizumab in Patients with Locally Advanced or Metastatic Solid Tumors

This is a first-in-human study to evaluate the safety, tolerability, pharmacokinetics (PK), and anti-tumor activity of RO7502175 when administered as a single agent and in combination with atezolizumab in adult participants with locally advanced or metastatic solid tumors, including non-small-cell lung cancer (NSCLC), head and neck squamous cell carcinoma (HNSCC), melanoma, triple-negative breast cancer (TNBC), esophageal cancer, gastric cancer, cervical cancer, urothelial carcinoma (UC), clear cell renal cell carcinoma (RCC) and hepatocellular carcinoma (HCC). Participants are enrolled in 2 stages: dose escalation and dose expansion.

### Objectives and Endpoints

This study evaluates the safety, tolerability, pharmacokinetics, pharmacodynamics, immunogenicity, and preliminary anti-tumor activity of RO7502175 as a single agent (Phase Ia) or in combination with the anti-PD-L1 antibody, atezolizumab (Phase Ib), in patients with locally advanced or metastatic solid tumors. Specific objectives and corresponding endpoints for the study are outlined below in Tables 3 and 4.

TABLE 3
Primary outcome measures of the GO43860 study

| Outcome Measure | Time Frame |
|---|---|
| Phase Ia: Number of Participants | From Day 1 to Day 21 of |
| with Dose Limiting Toxicities | Cycle 1 (21 days from date of |
| (DLTs) | first dose of study treatment) |
| Phase Ib: Number of Participants | From Day 1 to Day 21 of |
| with DLTs | Cycle 1 (21 days from date of |
| | first dose of study treatment) |
| Phase Ia: Number of | Up to approximately 5 years |
| Participants with Treatment | |
| Emergent Adverse Events | |
| Phase Ib: Number of | Up to approximately 5 years |
| Participants with Treatment | |
| Emergent Adverse Events | |

TABLE 4
Secondary outcome measures of the GO43860 study

| Outcome Measure | Time Frame |
|---|---|
| Phase Ia and Phase Ib: Maximum Serum | From Cycle 1 (each cycle is 21 days) Day 1 and at |
| Concentration (Cmax) of RO7502175 | multiple timepoints up to each follow-up visits (up to |
| | approximately 5 years) |
| Phase Ia and Phase Ib: Objective | From Cycle 1 (each cycle is 21 days) Day 1, until disease |
| Response Rate (ORR) | progression, death, or end of study (up to approximately |
| | 5 years) |
| Phase Ia and Phase Ib: Duration of | From Cycle 1 (each cycle is 21 days) Day 1, until disease |
| Response (DOR) | progression, death, or end of study (up to approximately |
| | 5 years) |
| Phase Ia and Phase Ib: Progression Free | From Cycle 1 (each cycle is 21 days) Day 1, until disease |
| Survival (PFS) | progression, death, or end of study (up to approximately |
| | 5 years) |
| Phase Ia and Phase Ib: Percentage of | From Cycle 1 (each cycle is 21 days) Day 1 and at |
| Participants With Anti-Drug Antibody | multiple timepoints up to treatment discontinuation (up to |
| (ADA) to RO7502175 | approximately 5 years) |

### Safety Objectives (Primary Study Objective)

The safety objective for this study is to evaluate the safety of RO7502175 when administered as a single agent (Phase Ia) or in combination with atezolizumab (Phase Ib), including characterization of dose-limiting toxicities (DLTs), on the basis of the following endpoints:
Incidence and nature of DLTs
Incidence and severity of adverse events, with severity determined according to National Cancer Institute Common Terminology Criteria for Adverse Events, Version 5.0 (NCI CTCAE v5.0)
Change from baseline in targeted vital signs
Change from baseline in targeted clinical laboratory test results
Change from baseline in electrocardiogram (ECG) parameters

### Pharmacokinetic Objectives

The PK objective for this study is to characterize the RO7502175 PK profile when administered as a single agent (Phase Ia) or in combination with atezolizumab (Phase Ib) on the basis of the following endpoint:
Serum concentration and maximum serum concentration (Cmax) of RO7502175 at specified timepoints

The following exploratory PK objectives may be evaluated for this study:
To evaluate potential relationships between drug exposure and the safety and activity of RO7502175 when administered as a single agent (Phase Ia) or in combination with atezolizumab (Phase Ib) on the basis of the following endpoints:
   Relationship between serum concentration and/or PK parameters for RO7502175 and safety endpoints
   Relationship between serum concentration and/or PK parameters for RO7502175 and activity endpoints
To evaluate potential relationships between selected covariates and exposure to RO7502175 when administered as a single agent (Phase Ia) or in combination with atezolizumab (Phase Ib) using population PK based analysis
To assess potential PK interactions between RO7502175 and atezolizumab on the basis of the following endpoints:
   Serum concentration and/or PK parameters for RO7502175 given in combination with atezolizumab compared with RO7502175 given alone
   Serum concentration and/or PK parameters for atezolizumab given in combination with RO7502175 compared with atezolizumab given alone (based on historical data)

### Activity Objectives

Response is assessed according to Response Evaluation Criteria in Solid Tumors, Version 1.1 (RECIST v1.1) and modified RECIST v1.1 for immune-based therapeutics (iRECIST). Objective response at a single timepoint is determined by the investigator according to RECIST v1.1. Objective response per iRECIST is calculated programmatically by the Sponsor on the basis of investigator assessments of individual lesions at each specified timepoint.

The activity objective for this study is to make a preliminary assessment of the activity of RO7502175 when administered as a single agent (Phase Ia) or in combination with atezolizumab (Phase Ib) on the basis of the following endpoints:
Objective response rate (ORR), defined as the proportion of patients with a complete response (CR) or partial response (PR) on two consecutive occasions ≥4 weeks apart, as determined by the investigator according to RECIST v1.1
Duration of response (DOR), defined as the time from the first occurrence of a documented objective response to disease progression or death from any cause (whichever occurs first), as determined by the investigator according to RECIST v1.1
Progression-free survival (PFS) after enrollment, defined as the time from enrollment to the first occurrence of disease progression or death from any cause (whichever occurs first), as determined by the investigator according to RECIST v1.1

The exploratory activity objective for this study is to make a preliminary assessment of the activity of RO7502175 when administered as a single agent (Phase Ia) or in combination with atezolizumab (Phase Ib) on the basis of the following endpoint:
Overall survival (OS) after enrollment, defined as the time from enrollment to death from any cause
ORR, DOR, and PFS based on radiographic assessment by the investigator according to iRECIST

### Immunogenicity Objectives

The immunogenicity objective for this study is to evaluate the immune response to RO7502175 when administered as a single agent (Phase Ia) or in combination with atezolizumab (Phase Ib) on the basis of the following endpoints:
Prevalence of anti-drug antibodies (ADAs) to RO7502175 at baseline (baseline prevalence) and incidence of ADAs to RO7502175 after initiation of study treatment (post-baseline incidence)

The exploratory immunogenicity objectives for this study are as follows:
To evaluate the prevalence of ADAs to atezolizumab at baseline and incidence of ADAs to atezolizumab after initiation of study treatment (Phase Ib)
To evaluate the relationship between ADA status and PK, activity, safety, or biomarker endpoints as data allow

### Biomarker Objective

The exploratory biomarker objective for this study is to identify and/or evaluate biomarkers that are predictive of response to RO7502175 when administered as a single agent (Phase Ia) or in combination with atezolizumab (Phase Ib) (i.e., predictive biomarkers), can provide evidence of RO7502175 activity (i.e., pharmacodynamic (PD) biomarkers), are associated with progression to a more severe disease state (i.e., prognostic biomarkers), are associated with acquired resistance to RO7502175, are associated with susceptibility to developing adverse events or can lead to improved adverse event monitoring or investigation, or can increase the knowledge and understanding of disease biology and drug safety, on the basis of the following endpoint:
Relationship between biomarkers in blood and tumor tissue and safety, PK, PD, activity, immunogenicity, or other biomarker endpoints

### Additional Objective

An additional objective for this study is to identify a recommended Phase II dose for RO7502175 on the basis of the following endpoint:
Relationship between RO7502175 dose and safety, PK, PD, activity, and immunogenicity endpoints

### Study Design

### Description of Study

This is a first-in-human Phase Ia/Ib, open-label, multicenter, dose-escalation study designed to evaluate the safety, tolerability, pharmacokinetics, pharmacodynamics, immunogenicity, and preliminary anti-tumor activity of RO7502175 as a single agent (Phase Ia) or in combination with atezolizumab (Phase Ib) and to identify a recommended Phase II dose for RO7502175 in patients with locally advanced, recurrent, or metastatic incurable solid tumor malignancies for which standard therapy does not exist, has proven to be ineffective or intolerable, or is considered inappropriate, or for whom a clinical trial of an investigational agent is a recognized standard of care.

Both the Phase Ia and Ib portions of the study consist of a screening period of up to 28 days, a treatment period, a minimum follow-up period of 90 days after treatment, and survival follow-up. In each phase, patients are enrolled in two stages: a dose-escalation stage and an expansion stage.

In the expansion stages, patients are enrolled and treated at or below the maximum tolerated dose (MTD) or maximum administered dose (MAD) of RO7502175 as a single agent (Phase Ia) or in combination with atezolizumab (Phase Ib). Approximately 230-365 patients may be enrolled in this study, at approximately 50 global investigative sites.

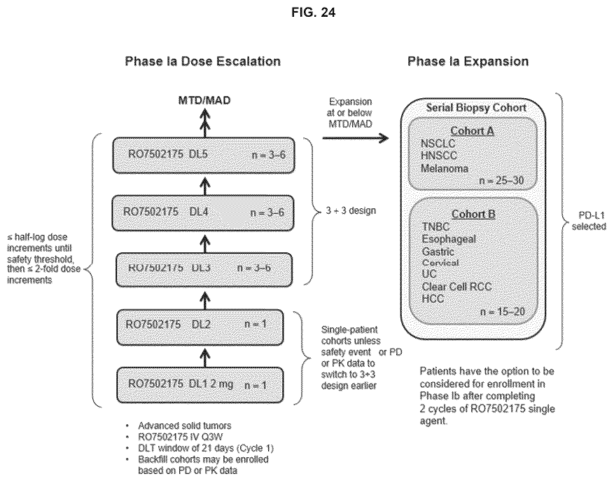

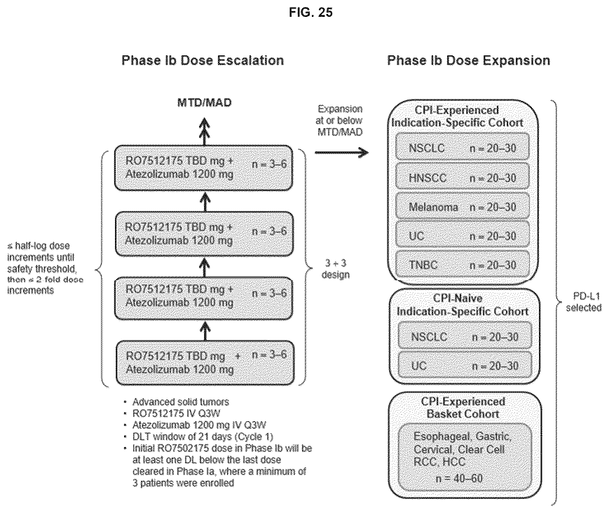

Patients in this study are initially assessed for eligibility during the screening period (lasting ≤28 days). Patients enrolling into Phase Ia and Phase Ib expansion cohorts with PD-L1-selected tumors can have tumor tissue screening for PD-L1 status performed prior to the 28-day screening period. Following confirmation of eligibility, patients receive RO7502175 as a single agent (Phase Ia, FIG. 24) or RO7502175 in combination with atezolizumab (Phase Ib, FIG. 25) by IV infusion on the first day of every 21-day cycle until they experience disease progression or unacceptable toxicity.

The Phase Ib portion of this study is activated only after completion of DLT evaluation in a minimum of 3 patients of at least one dose level of single-agent RO7502175, and all relevant single-agent safety data have been reviewed by investigators and an Internal Monitoring Committee (IMC). The dose-escalation stage of Phase Ib initiates enrollment at a minimum of one dose level below the last RO7502175 dose level cleared as a single agent.

In general, open dose-escalation slots in the Phase Ia portion are enrolled first, followed by open dose-expansion slots in the Phase Ia portion or open dose-escalation slots in the Phase Ib portion, on the basis of investigator discretion and patient eligibility. Thereafter, any available expansion cohort slots in the Phase Ib portion may be enrolled.

Patients undergo tumor assessments at screening (baseline) and at regular intervals during the study, which are measured by RECIST v1.1. Patients may be permitted to continue study treatment even if standard RECIST v1.1 criteria for progressive disease are met, provided that they meet criteria for continued treatment (FIG. 26).

Patients in the Phase Ia portion of the study may be permitted to cross over into the Phase Ib portion and receive treatment with RO7502175 in combination with atezolizumab, provided that they meet the criteria for crossover (FIG. 27).

Patients who discontinue RO7502175 as a single agent (Phase Ia) or RO7502175 in combination with atezolizumab (Phase Ib) for disease progression and are not eligible to continue treatment past disease progression, return to the clinic for a treatment discontinuation visit within 30 days after the final dose of study treatment.

All patients who permanently discontinue RO7502175 as a single agent (Phase Ia) or RO7502175 in combination with atezolizumab (Phase Ib) for reasons other than disease progression (e.g., adverse events or achievement of a confirmed CR) continue tumor assessments per the schedule of activities.

All patients are closely monitored for adverse events throughout the study and for at least 90 days after the final dose of study treatment or until initiation of another systemic anti-cancer therapy, whichever occurs first. After this period, the Sponsor should be notified if the investigator becomes aware of any serious adverse events that are believed to be related to prior study drug treatment. Adverse events are graded according to the NCI CTCAE v5.0. Extended follow-up is required for women of childbearing potential, in which they receive monthly serum or urine pregnancy testing for up to 5 months after treatment discontinuation, or until initiation of new systemic anti-cancer therapy, or withdrawal of consent, whichever occurs first.

All patients in the study are followed for survival and subsequent anti-cancer therapy information approximately every 3 months until death, loss to follow-up, or until Sponsor decision to discontinue survival follow-up because no further clinical development of RO7502175 is planned or the study is terminated by the Sponsor, unless the patient requests to be withdrawn from follow-up.

### Number of Patients

Approximately 230-365 patients with locally advanced, recurrent, or metastatic incurable malignancies that have progressed after available standard therapy; or for whom standard therapy has proven to be ineffective or intolerable, or is considered inappropriate; or for whom a clinical trial of an investigational agent is a recognized standard of care, may be enrolled in this study.

### Target Population

### a) Inclusion Criteria

Patients must meet the following criteria for study entry.

### General Inclusion Criteria

Signed Informed Consent Form
Age ≥18 years at time of signing Informed Consent Form
Ability to comply with the study protocol, in the investigator's judgment
Eastern Cooperative Oncology Group (ECOG) Performance Status of 0 or 1
Life expectancy ≥12 weeks
Adequate hematologic and end-organ function, defined by the following laboratory and diagnostic test results, obtained within 14 days prior to initiation of study treatment:
   Absolute neutrophil count (ANC) ≥1.5×10⁹/L (≥1500/μL) without granulocyte colony-stimulating factor support, with one exception:
   Patients with benign ethnic neutropenia (BEN): ANC >1.3×10⁹/L (1300/μL)
      BEN (also known as constitutional neutropenia) is an inherited cause of mild or moderate neutropenia that is not associated with any increased risk for infections or other clinical manifestations (see, e.g., Atallah-Yunes et al. Blood Rev. 37:100586, 2019). BEN is referred to as ethnic neutropenia because of its increased prevalence in people of African descent and other specific ethnic groups.
   Lymphocyte count ≥0.5×10⁹/L (≥500/μL)
   Platelet count ≥100×10⁹/L (≥100,000/μL) without transfusion within 14 days of Cycle 1, Day 1
   Hemoglobin ≥90 g/L (≥9 g/dL)
      Patients may be transfused or may receive erythropoietic treatment as per local standard of care
   Aspartate transaminase (AST), alanine transaminase (ALT), and alkaline phosphatase (ALP) ≤2.5× upper limit of normal (ULN), with the following exceptions:
      Patients with documented liver metastases: AST and ALT ≤5×ULN
      Patients with documented liver or bone metastases: ALP ≤5×ULN
   Total bilirubin ≤1.5×ULN with the following exception:
      Patients with known Gilbert disease: total bilirubin level ≤3×ULN
   Measured or calculated creatinine clearance ≥50 mL/min on the basis of the Cockcroft-Gault glomerular filtration rate estimation:

$$\frac{( 140 - age ) \times ( weight in kilograms ) \times ( 0.85 if female )}{72 \times ( serum creatinine in mg / dL )}$$

Albumin ≥25 g/L (2.5 g/dL)
For patients not receiving therapeutic anticoagulation: international normalized ratio (INR) and activated partial thromboplastin time (aPTT) ≤1.5×ULN
   Patients receiving therapeutic anticoagulation should be on a stable dose.
Histologically confirmed locally advanced, recurrent, or metastatic incurable solid tumor malignancy
   Additional cohort-specific criteria related to tumor type and prior lines of therapy are detailed below.
Availability of representative tumor specimens in formalin-fixed, paraffin-embedded (FFPE) blocks (preferred) or 215 unstained slides, with an associated pathology report within 3 years of screening
   A patient with insufficient or unavailable archival tissue is eligible, if the patient meets any of the following: can provide at least 10 unstained, serial slides; is willing to consent to and undergo a pretreatment core, punch, or excisional/incisional biopsy; or is enrolled in a dose-escalation cohort.
   If adequate tissue from distinct time points (such as time of initial diagnosis and time of disease recurrence) and/or multiple metastatic tumors is available, priority should be given to the tissue most recently collected (ideally subsequent to the most recent systemic therapy). Multiple samples may be collected for a given patient, on the basis of availability; however, the requirement for a block or ≥15 unstained slides should be satisfied by a single biopsy or resection specimen.
Measurable disease per RECIST v1.1
   Previously irradiated lesions should not be counted as target lesions unless there has been demonstrated progression in the lesion and no other target lesions are available.
   Lesions that are intended to be biopsied must not count as targeted lesions.
   Central nervous system (CNS) lesions should not be counted as target lesions.
For women of childbearing potential: agreement to remain abstinent (refrain from heterosexual intercourse) or use contraception, and agreement to refrain from donating eggs, as defined below:
   Women must remain abstinent or use contraceptive methods with a failure rate of <1% per year during the treatment period and for at least 4 months after the final dose of RO7501275 and/or 5 months after the final dose of atezolizumab (whichever is longer). Women must refrain from donating eggs during this same period.
   A woman is considered to be of childbearing potential if she is postmenarchal, has not reached a postmenopausal state (>12 continuous months of amenorrhea with no identified cause other than menopause), and is not permanently infertile due to surgery (i.e., removal of ovaries, fallopian tubes, and/or uterus) or another cause as determined by the investigator (e.g., Müllerian agenesis). The definition of childbearing potential may be adapted for alignment with local guidelines or regulations.
   Examples of contraceptive methods with a failure rate of <1% per year include bilateral tubal ligation, male sterilization, hormonal contraceptives that inhibit ovulation, hormone-releasing intrauterine devices, and copper intrauterine devices.
   Hormonal contraceptive methods must be supplemented by a barrier method.
   The reliability of sexual abstinence should be evaluated in relation to the duration of the clinical trial and the preferred and usual lifestyle of the patient. Periodic abstinence (e.g., calendar, ovulation, symptothermal, or postovulation methods) and withdrawal are not adequate methods of contraception. If required per local guidelines or regulations, locally recognized adequate methods of contraception and information about the reliability of abstinence are described in the local Informed Consent Form.
For men: agreement to remain abstinent (refrain from heterosexual intercourse) or use a condom, and agreement to refrain from donating sperm, as defined below:
   With a female partner of childbearing potential or pregnant female partner, men must remain abstinent or use a condom during the treatment period and for 4 months after the final dose of RO7502175 to avoid exposing the embryo. Men must refrain from donating sperm during this same period.
   The reliability of sexual abstinence should be evaluated in relation to the duration of the clinical trial and the preferred and usual lifestyle of the patient. Periodic abstinence (e.g., calendar, ovulation, symptothermal, or postovulation methods) and withdrawal are not adequate methods of preventing drug exposure. If required per local guidelines or regulations, information about the reliability of abstinence is described in the local Informed Consent Form.

### Additional Inclusion Criteria for Specific Cohorts

### Phase Ia and Ib Dose-Escalation Cohorts

Patients enrolling in the Phase Ia dose-escalation cohorts must meet the following additional criterion:
Disease for which all available standard therapy has proven to be ineffective or intolerable or is contraindicated

Patients enrolling in the Phase Ib dose-escalation cohorts must meet the following additional criterion:
Disease that has progressed after at least one available standard therapy, and for which standard therapy has proven to be ineffective or intolerable or is considered inappropriate, or for which a clinical trial of an investigational agent is a recognized standard of care
   If a patient who has progressed after at least one available standard therapy has additional approved standard treatment options available, the study doctor must discuss the risks and benefits of those treatments before informed consent to participate in this study is obtained. This discussion must be documented in patient records.

### Phase Ia Expansion, Serial Biopsy Cohorts

Disease that has progressed after at least one available standard therapy, and for which standard therapy has proven to be ineffective or intolerable or is considered inappropriate, or for which a clinical trial of an investigational agent is a recognized standard of care
   If a patient who has progressed after at least one available standard therapy has additional approved standard treatment options available, the study doctor must discuss the risks and benefits of those treatments before informed consent to participate in this study is obtained. This discussion must be documented in patient records.
One of the following tumor types:
   Cohort A: NSCLC, HNSCC, cutaneous melanoma
   Cohort B: TNBC, UC, esophageal cancer, gastric cancer, cervical cancer, clear cell RCC, or HCC
Accessible lesion that permits a pretreatment and on-treatment biopsy (i.e., serial biopsies) without unacceptable risk of a significant procedural complication
PD-L1 selected based on evaluation of tumor as outlined below:
   Archival tumor tissue must be submitted and evaluated for PD-L1 expression through central testing prior to enrollment, unless PD-L1 expression has been determined by a Clinical Laboratory Improvement Amendments (CLIA)-certified local laboratory or equivalent laboratory using a Health Authority-cleared device for that indication. Patients whose tumor tissue is not evaluable for PD-L1 expression are not eligible.
   If no archival tissue is available, fresh tumor tissue may be submitted and evaluated for PD-L1 expression through central testing prior to enrollment. Patients whose tumor tissue is not evaluable for PD-L1 expression are not eligible.
   Prior to signing the main study Informed Consent Form, patients may sign a Pre-Screening Informed Consent Form to specifically allow the collection and PD-L1 testing of archival or fresh tumor specimens.
   PD-L1 expression may be evaluated in either immune-infiltrating cells or tumor cells. If multiple tumor specimens are submitted (e.g., an archival specimen and tissue from relapsed disease), patients may be eligible if at least one specimen is evaluable for PD-L1. The PD-L1 score of the patient is the maximum PD-L1 score among the samples. The initial target level for PD-L1 expression is Tumor Cell (TC), Immune Cell (IC), Cell Proportion Score (CPS), or Tumor Proportion Score (TPS) >1%, which may be modified if emerging PD/PK/efficacy data suggest that a higher target level may be required for mechanistic activity of RO7502175 and patient benefit.
Patients must have derived clinical benefit from anti-PD-L1/PD-1 containing therapy (treatment duration for 26 months and/or PR/CR as best objective response) prior to disease progression.

### Phase Ib Expansion Cohorts (All)

Evaluation of tumor PD-L1 expression as outlined below:
   Archival tumor tissue must be submitted and evaluated for PD-L1 expression through central testing prior to enrollment, unless PD-L1 expression has been determined by a CLIA-certified local laboratory or equivalent laboratory using a Health Authority-cleared device for that indication. Patients whose tumor tissue is not evaluable for PD-L1 expression are not eligible.
   If no archival tissue is available, fresh tumor tissue may be submitted and evaluated for PD-L1 expression through central testing prior to enrollment. Patients whose tumor tissue is not evaluable for PD-L1 expression are not eligible.
   Prior to signing the main study Informed Consent Form, patients may sign a Pre-Screening Informed Consent Form to specifically allow the collection and testing of archival or fresh tumor specimens.
   PD-L1 expression may be evaluated in either immune-infiltrating cells or tumor cells. If multiple tumor specimens are submitted (e.g., an archival specimen and tissue from relapsed disease), patients may be eligible if at least one specimen is evaluable for PD-L1. The PD-L1 score of the patient is the maximum PD-L1 score among the sample. The initial target level for PD-L1 expression is TC, IC, CPS, or TPS ≥1%, which may be modified if emerging PD/PK/efficacy data suggest that a higher target level may be required for mechanistic activity of RO7502175 and patient benefit.

### Phase Ib Expansion, Checkpoint Inhibitor (CPI)-Experienced Cohorts (Indication-Specific and Basket)

Disease that has progressed after at least one available standard therapy, and for which standard therapy has proven to be ineffective or intolerable or is considered inappropriate, or for which a clinical trial of an investigational agent is a recognized standard of care
   If a patient who has progressed after at least one available standard therapy has additional approved standard treatment options available, the study doctor must discuss the risks and benefits of those treatments before informed consent to participate in this study is obtained. This discussion must be documented in patient records.
Patients must have derived clinical benefit from anti-PD-L1/PD-1 containing therapy (treatment duration for ≥6 months and/or PR/CR as best objective response) prior to disease progression.
Enrollment may be limited to specific indications based on real-time review of data and enrollment demographics. Additional cohort-specific criteria are detailed below.

### Phase Ib Expansion, CPI-Experienced, Indication-Specific Cohorts

One of the following tumor types: HNSCC, NSCLC, cutaneous melanoma, UC, or TNBC
For all patients with HNSCC:
   HNSCC of the oral cavity, oropharynx, hypopharynx, or larynx
   Patients with HNSCC of any other primary anatomic location in the head and neck, patients with HNSCC of unknown primary, or patients with tumors of non-squamous histologies are not eligible.
For all patients with NSCLC:
   Patients whose tumors have a targetable somatic alteration, including those involving EGFR, ALK, ROS1, BRAFV600E, NTRK, MET, RET and KRAS must have experienced disease progression (during or after treatment) or intolerance to treatment with a targeted agent, if available and approved by local regulatory authorities.
For all patients with cutaneous melanoma:
   Patients whose tumors have a known BRAFV600 mutation must also have experienced disease progression (during or after treatment) or intolerance with BRAF inhibitor(s) and/or MEK inhibitor(s).
For all patients with UC:
   Patients with histologically confirmed incurable advanced transitional cell carcinoma of the urothelium (including renal pelvis, ureters, urinary bladder, and urethra)
   Patients with mixed histologies are required to have a dominant transitional cell pattern.
For all patients with TNBC:
   Triple-negative status must be documented as defined by the American Society of Clinical Oncology-College of American Pathologists guidelines:
   <1% of tumor-cell nuclei immunoreactive for estrogen receptor and <1% of tumor-cell nuclei immunoreactive for progesterone receptor
   HER2 negative based on IHC and/or in situ hybridization
For at least 10 (out of 30) patients enrolling in indication-specific CPI-experienced cohorts: an accessible lesion that permits a pretreatment and on-treatment biopsy without unacceptable risk of a significant procedural complication

### Phase Ib Expansion, CPI-Experienced, Basket Cohort

One of the following tumor types: esophageal cancer, gastric cancer, cervical cancer, clear cell RCC, or HCC
For at least 20 patients enrolling in CPI-experienced basket cohort: an accessible lesion that permits a pretreatment and on-treatment biopsy without unacceptable risk of a significant procedural complication

### Phase Ib Expansion, CPI-Naïve Cohorts

Patients for whom a clinical trial of an investigational agent in combination with an anti-PD-L1 antibody is considered an acceptable treatment option may be enrolled if CPI (including anti-PD-L1/PD-1 agents) is approved as treatment by local regulatory authorities.
One of the following tumor types: NSCLC, UC
No prior treatment with a CPI (investigational or approved, including anti-PD-L1/PD-1 and/or anti-CTLA4) with the following exceptions for adjuvant therapies:
   Adjuvant treatment with anti-PD1/PD-L1 or anti-CTLA-4, if discontinued at least 6 months prior to Cycle 1, Day 1 is permitted.
Eligible for treatment with a CPI
For all patients with NSCLC:
   Patients whose tumors have a targetable somatic alteration, including those involving EGFR, ALK, ROS1, BRAFV600E, NTRK, MET, RET and KRAS must have experienced disease progression (during or after treatment) or intolerance to treatment with a targeted agent, if available and approved by local regulatory authorities.
For all patients with UC:
   Patients with histologically confirmed incurable advanced transitional cell carcinoma of the urothelium (including renal pelvis, ureters, urinary bladder, and urethra) Patients with mixed histologies are required to have a dominant transitional cell pattern.
   Patients who are eligible for cisplatin chemotherapy must have experienced disease progression (during or after treatment) or intolerance to treatment with a cisplatin-containing chemotherapy, if available and approved by local regulatory authorities.
For at least 10 (out of 30) patients enrolling in indication specific CPI-naïve cohorts: an accessible lesion that permits a pretreatment and on-treatment biopsy without unacceptable risk of a significant procedural complication

### a) Exclusion Criteria

Patients who meet any of the following criteria are excluded from study entry.

### General Exclusion Criteria

Pregnant or breastfeeding, or intending to become pregnant during the study or within 5 months after the final dose of study treatment
   Women of childbearing potential must have a negative serum pregnancy test result within 14 days prior to initiation of study drug.
Any anti-cancer therapy, whether investigational or approved, including chemotherapy, hormonal therapy, and/or radiotherapy, within 3 weeks prior to initiation of study treatment, with the following exceptions:
   Hormone-replacement therapy or oral contraceptives
   Tyrosine kinase inhibitor(s) (TKIs) approved by local regulatory authorities for treatment of cancer that have been discontinued >7 days prior to Day 1 of Cycle 1
      Baseline scans must be obtained after discontinuation of prior TKIs, and criteria pertaining to adverse events attributed to prior cancer therapies must be met.
   Herbal therapy intended for the treatment of cancer >7 days before Day 1 of Cycle 1
   Palliative radiotherapy for painful metastases or metastases in potentially sensitive locations (e.g., epidural space) >2 weeks prior to Day 1 of Cycle 1
Treatment with cancer vaccines within 6 weeks or 5 drug elimination half-lives (whichever is shorter) prior to initiation of study treatment
Systemic immunostimulatory agents (including, but not limited to, IFNs and IL-2) within 4 weeks or 5 drug-elimination half-lives (whichever is longer) prior to initiation of study treatment and during study treatment
Prior treatment with regulatory T-cell depleting agents including, but not limited to CD25-targeting agent (e.g., RO7296682, basiliximab), CC chemokine receptor 4 (e.g., mogamulizumab), CCR8-targeting agents (e.g., BMS-986340, GS-1811), with the exception of RO7502175 treatment during the Phase Ia portion of the study (i.e., for patients crossing over to the Phase Ib portion of the study)
Symptomatic, untreated, or actively progressing CNS metastases
   Asymptomatic patients with treated CNS lesions are eligible, provided that all of the following criteria are met:
Measurable disease, per RECIST v1.1, must be present outside the CNS.
No evidence of acute or subacute brain metastasis—related hemorrhage was seen on CNS imaging at screening.
The patient has not undergone stereotactic radiotherapy within 7 days prior to initiation of study treatment, whole-brain radiotherapy within 14 days prior to initiation of study treatment, or neurosurgical resection within 28 days prior to initiation of study treatment.
The patient has no ongoing requirement for corticosteroids as therapy for CNS disease, with corticosteroids discontinued for ≥2 weeks prior to enrollment.
Anticonvulsant therapy at a stable dose is permitted.
There is no evidence of interim progression between completion of CNS-directed therapy and initiation of study treatment.
Metastases are limited to the cerebellum or the supratentorial region (i.e., no metastases to the midbrain, pons, medulla, or spinal cord)
Note: Asymptomatic patients with CNS metastases newly detected at screening are eligible for the study after receiving radiotherapy or surgery, with no need to repeat the screening brain scan.
History of leptomeningeal disease
Spinal cord compression not definitively treated with surgery and/or radiation or previously diagnosed and treated spinal cord compression without evidence that disease has been clinically stable for ≥2 weeks prior to screening
Significant cardiovascular disease (such as New York Heart Association Class II or greater cardiac disease, myocardial infarction, or cerebrovascular accident) within 3 months prior to initiation of study treatment, unstable arrhythmia, or unstable angina
Mean (average of triplicate measurements) QT interval corrected through use of Fridericia's formula (QTcF) >470 ms
Any diseases, metabolic dysfunction, physical examination finding, or clinical laboratory finding that contraindicates the use of an investigational drug, may affect the interpretation of the results, or may render the patient at high risk from treatment complications, including, but not limited to:
   Known clinically significant liver disease, including active viral, alcoholic, or other hepatitis, cirrhosis, and inherited liver disease or current alcohol abuse
   Poorly controlled Type 2 diabetes mellitus, defined as a hemoglobin A1C ≥8% or a fasting plasma glucose ≥160 mg/dL (or 8.8 mmol/L)
   Severe dyspnea or requiring supplemental oxygen at rest
   History of Stevens-Johnson syndrome, toxic epidermal necrolysis, or drug rash with eosinophilia and systemic symptoms
   Participants with current or history of wound healing complications and/or participants with open wounds until resolution
Uncontrolled tumor-related pain
   Patients requiring pain medication must be on a stable regimen at study entry.
   Symptomatic lesions (e.g., bone metastases or metastases causing nerve impingement) amenable to palliative radiotherapy should be treated prior to enrollment. Patients should be recovered from the effects of radiation.
   Asymptomatic metastatic lesions that would likely cause functional deficits or intractable pain with further growth (e.g., epidural metastasis that is not currently associated with spinal cord compression) should be considered for loco-regional therapy if appropriate prior to enrollment.
Uncontrolled pleural effusion, pericardial effusion, or ascites requiring recurrent drainage procedures (once monthly or more frequently)
   Patients with indwelling catheters (e.g., PLEURX®) are allowed.
Uncontrolled or symptomatic hypercalcemia (ionized calcium >1.5 mmol/L, calcium >12 mg/dL, or corrected calcium >ULN)
History of malignancy other than disease under study within 3 years prior to screening, with the exception of malignancies with a negligible risk of metastasis or death (e.g., 5-year OS rate >90%), such as adequately treated carcinoma in situ of the cervix, non-melanoma skin carcinoma, localized prostate cancer, ductal carcinoma in situ, or Stage I uterine cancer
Adverse events from prior anti-cancer therapy (with the exception of immune-related adverse events attributed to cancer immunotherapy; see below) that have not resolved to Grade ≤1 except for alopecia, vitiligo, or endocrinopathy managed with replacement therapy
Any history of an immune-mediated Grade 4 adverse event attributed to prior cancer immunotherapy (other than endocrinopathy managed with replacement therapy or asymptomatic elevation of serum lipase)
Any history of an immune-mediated Grade 3 adverse event attributed to prior cancer immunotherapy (other than endocrinopathy managed with replacement therapy or asymptomatic elevation of serum lipase) that resulted in permanent discontinuation of the prior immunotherapeutic agent and/or occurred ≤6 months prior to planned Day 1 of Cycle 1
Any immune-mediated adverse events related to prior cancer immunotherapy (other than endocrinopathy managed with replacement therapy or stable vitiligo) that have not resolved completely to baseline
   Patients treated with corticosteroids for immune-mediated adverse events must demonstrate absence of related symptoms or signs for >4 weeks following discontinuation of corticosteroids.
Active or history of autoimmune disease or immune deficiency, including, but not limited to, myasthenia gravis, myositis, autoimmune hepatitis, systemic lupus erythematosus, rheumatoid arthritis, inflammatory bowel disease, antiphospholipid antibody syndrome, Wegener granulomatosis, Sjögren syndrome, Guillain-Barré syndrome, or multiple sclerosis, with the following exceptions:
   Patients with a history of autoimmune-mediated hypothyroidism who are on thyroid-replacement hormone are eligible for the study.
   Patients with controlled Type 1 diabetes mellitus who are on an insulin regimen are eligible for the study.
   Patients with eczema, psoriasis, lichen simplex chronicus, or vitiligo with dermatologic manifestations only (e.g., patients with psoriatic arthritis are excluded) are eligible for the study, provided all of following conditions are met:
Rash must cover <10% of body surface area.
Disease is well controlled at baseline and requires only low-potency topical corticosteroids.
No occurrence of acute exacerbations of the underlying condition requiring psoralen plus ultraviolet A radiation, methotrexate, retinoids, biologic agents, oral calcineurin inhibitors, or high-potency or oral corticosteroids within the previous 12 months.
History of idiopathic pulmonary fibrosis, organizing pneumonia (e.g., bronchiolitis obliterans), drug-induced pneumonitis, or idiopathic pneumonitis, or evidence of active pneumonitis on screening chest CT scan
   History of radiation pneumonitis in the radiation field (fibrosis) is permitted.
Active tuberculosis
Severe infection within 4 weeks prior to initiation of study treatment, including, but not limited to, hospitalization for complications of infection, bacteremia, or severe pneumonia
Treatment with therapeutic oral or IV antibiotics within 2 weeks prior to initiation of study treatment
   Patients receiving prophylactic antibiotics (e.g., to prevent a urinary tract infection or chronic obstructive pulmonary disease (COPD) exacerbation) are eligible for the study.
Positive test for human immunodeficiency virus (HIV) infection
Positive hepatitis B surface antigen (HbsAg) test, and/or positive total hepatitis B core antibody (HbcAb) test at screening.
   NOTE: Participants with positive total HbcAb test followed by a negative hepatitis B virus (HBV) DNA test at screening can be enrolled.
   Antiviral prophylaxis for patients at risk for HBV reactivation is permitted.
Positive hepatitis C virus (HCV) antibody test at screening
   Patients positive for HCV antibody are eligible only if polymerase chain reaction (PCR) is negative for HCV RNA.
Acute or chronic active Epstein-Barr virus (EBV) infection at screening
   EBV status should be assessed by EBV serology (e.g., anti-VCA IgM and IgG, anti-EA IgG, anti-EBNA IgG) and/or EBV PCR (plasma or serum).
   If EBV serology results indicate prior EBV infection, patients must have a negative EBV PCR (plasma or serum) to be eligible for the study.
Known infection with SARS-CoV-2 (the virus that causes coronavirus disease 2019 (COVID-19)), persistent symptoms of known prior SARS-CoV-2 infection, and/or known positive COVID-19 test within 4 weeks prior to screening
Administration of a live, attenuated vaccine (e.g., FLUMIST®) within 4 weeks before first RO7502175 infusion or anticipation that such a live, attenuated vaccine is required during the study or within 5 months after the final dose of study treatment
Treatment with systemic immunosuppressive medication (including, but not limited to, corticosteroids, cyclophosphamide, azathioprine, methotrexate, thalidomide, and anti-TNF-α agents) within 2 weeks prior to initiation of study treatment, or anticipation of need for systemic immunosuppressive medication during study treatment, with the following exceptions:
   Patients who received acute, low-dose systemic immunosuppressant medication or a one-time pulse dose of systemic immunosuppressant medication (e.g., 48 hours of corticosteroids for a contrast allergy) are eligible for the study.
   Patients who received mineralocorticoids (e.g., fludrocortisone), corticosteroids for COPD or asthma, or low-dose corticosteroids for orthostatic hypotension or adrenal insufficiency are eligible for the study.
Major surgical procedure or significant traumatic injury within 28 days prior to first RO7502175 and atezolizumab infusion, or anticipation of the need for major surgery before end of treatment period. After major surgery, participant must wait until surgical wounds are fully healed before initiating treatment.
Prior allogeneic stem cell or organ transplantation
History of severe allergic anaphylactic reactions to chimeric or humanized antibodies or fusion proteins
Known hypersensitivity to Chinese hamster ovary cell products or to any component of the atezolizumab formulation
Known allergy or hypersensitivity to any component of the RO7502175 formulation (refer to the RO7502175 Investigator's Brochure for details on the RO7502175 formulation)

### Additional Exclusion Criteria for Specific Cohorts

### Phase Ia Dose Escalation and Expansion Cohorts

Treatment with CPIs, immunomodulatory monoclonal antibodies or immunomodulatory monoclonal antibody-derived therapies within 6 weeks prior to initiation of study treatment

### Phase Ib Dose Escalation and Expansion, CPI-Experienced Cohorts

Treatment with CPIs, immunomodulatory monoclonal antibodies, or immunomodulatory monoclonal antibody-derived therapies within 6 weeks prior to initiation of study treatment
Prior anti-PD-L1/PD-1 is subject to a minimum 3-week washout period.

### Phase Ib Expansion, CPI-Naïve Cohorts

Treatment with non-CPI immunomodulatory monoclonal antibodies or immunomodulatory monoclonal antibody-derived therapies within 6 weeks prior to initiation of study treatment

### End of Study

The end of this study is defined as the date at which the last data point required for all study analyses is collected. The end of the study is expected to occur approximately 12 months after the last patient is enrolled. In addition, the Sponsor may decide to terminate the study at any time.

### Length of Study

The total length of the study, from screening of the first patient to the end of the study, is expected to be approximately 4-5 years.

### Study Treatment

The investigational medicinal products for this study are RO7502175 (Phase Ia and Ib) and atezolizumab (Phase Ib only). RO7502175 is administered on Day 1 of every 21-day cycle by intravenous (IV) infusion in the Phase Ia and Phase Ib portions of this study. The starting dose of RO7502175 is 2 mg. Atezolizumab (Phase Ib) is administered by IV infusion at a fixed dose of 1200 mg on Day 1 of each 21-day cycle in combination with RO7502175. Atezolizumab is administered after RO7502175 and the subsequent observation period.

### Statistical Methods

### Primary Analysis

The safety analysis population consists of all patients who received any amount of study drug. Safety is assessed through summaries of DLTs, adverse events, changes in laboratory test results, changes in ECG parameters, and changes in vital signs. Summaries are presented overall, by cohort, and by cancer type.

All verbatim descriptions of adverse events are mapped to MedDRA thesaurus terms. All collected adverse event data are listed by study site, assigned dose level, cohort, and patient number. All adverse events occurring on or after treatment on Day 1 of Cycle 1 are summarized by mapped term, appropriate thesaurus levels, and NCI CTCAE toxicity grade. In addition, all serious adverse events, including deaths, are listed separately. DLTs, adverse events leading to treatment discontinuation, and adverse events of special interest are also listed separately.

Relevant laboratory, ECG, and vital sign data are displayed by time.

### Determination of Sample Size

The sample sizes for the dose-escalation stages of this trial are based on the dose-escalation rules described in the protocol. Any patient who does not complete the DLT assessment window for any reason other than a DLT is considered non-evaluable for dose-escalation decisions and MTD assessment and is replaced by an additional patient at the same dose level.

### Interim Analyses

Interim analyses are conducted by the IMC for each expansion cohort in Phase Ib to guide potential early stopping of enrollment when there is no evidence of activity.

The IMC conducts periodic interim efficacy analyses after approximately 15 patients have completed at least one tumor assessment in a given indication-specific expansion cohort, or basket cohort in Phase Ib in order to inform potential early stopping of enrollment. For cohorts restricted to CPI-experienced patients, the following rule applies: if no anti-tumor activity and/or clinical benefit per investigator is observed from the first 15 patients, enrollment is suspended for that cohort. For cohorts that enroll CPI-naïve patients, the IMC makes a recommendation on whether enrollment should continue based on an assessment of benefit and risk relative to the expected performance of anti-PD-1/PD-L1 monotherapy and/or other available therapies for the individual diseases under evaluation. For example, enrollment may be stopped if there is a greater than 60% chance, using a posterior probability approach, that the true ORR is less than or equal to a pertinent disease-specific benchmark, which may evolve as the trial progresses. The IMC may also recommend that enrollment continue in a given exploration/expansion cohort if there is sufficient evidence of activity or if the enrolled patients are judged to be uninformative (e.g., insufficient patients with specific tumor PD-L1 expression status).

In all cases, decisions to stop enrollment into any expansion cohort based on futility are made following the IMC recommendation by the Medical Monitor in consultation with the study investigators. The Medical Monitor may also request additional ad-hoc meetings of the IMC to review ongoing data in each expansion cohort in Phase Ia or Phase Ib.

### Example 13. Preclinical and Translational Pharmacology of Afucosylated Anti-CCR8 Antibody for Depletion of Tumor-Infiltrating Regulatory T Cells

Treg cells, a subset of CD4+ T cells, are highly immunosuppressive cells and play a key role in regulating immune system activity and preventing autoimmunity. Presence of Treg cells is associated with poor clinical outcomes and prognosis in patients with certain cancers including melanoma, non-small-cell lung cancer (NSCLC), and gastric cancer. C-C motif chemokine receptor 8 (CCR8) is a seven transmembrane G-protein coupled receptor (GPCR) selectively expressed on activated and proliferating intratumoral Treg cells, which constitute the majority of Treg cells in tumors. RO7502175 (also referred to as anti-CCR8) is a humanized immunoglobulin G1 (IgG1) antibody that binds to human and cynomolgus monkey CCR8.

Herein, we describe an approach used to inform clinical translation based on available nonclinical data for RO7502175. The results from in vitro and in vivo studies including the pharmacokinetics (PK), pharmacodynamics (PD), and safety profile of anti-CCR8 are discussed. We utilized a minimal physiologically-based PK-PD (mPBPK-PD) model to support clinical translation and enable projection of clinical PK and receptor occupancy (RO) in patients. For the first-in-human (FiH) dose selection, we used an integrated approach based on the totality of preclinical data and modeling insights that would allow us to start at a higher dose in a safe manner while minimizing administration of sub-therapeutic dose levels to patients. Traditional approaches for FiH dose selection (e.g., as described in Saber et al. Regul. Toxicol. Pharmacol. 2016; 81: 448-456) including in vitro minimum anticipated biological effect level (MABEL) and minimal pharmacologically active dose (mPAD) approaches will be discussed, however, they were not considered appropriate for this molecule due to its superior preclinical safety profile, and the restricted target expression in the tumor microenvironment. Additionally, the antibody is not a direct immune activator and therefore the mechanism-of-action (MOA) does not necessitate a conservative approach. The preclinical data and translational work described in this Example informed the FiH dose selection and the design of the Phase 1 clinical study with RO7502175. The proposed FiH dose selection strategy has been accepted by health authorities in the US (clinicaltrials.gov identifier: NCT05581004) and across the globe.

### Methods

### Anti-CCR8 Antibodies

RO7502175 is a humanized rabbit-derived full-length IgG1 monoclonal antibody that binds to human CCR8. The antibody was expressed via transient transfection of CHO FUT8KO cells and purified via affinity chromatography followed by size exclusion chromatography (SEC) using standard methods to generate material with an afucosylated Fc region (see, e.g., Louie et al. Biotechnol. Bioeng. 2017; 114: 632-644). Anti-murine CCR8 antibody is a chimeric rabbit-derived full-length murine IgG2a (mIgG2a) antibody that binds to mouse CCR8 and serves as a murine surrogate for RO7502175. The antibody was expressed via transient transfection of CHO cells and purified via affinity chromatography followed by SEC using standard methods.

### In Vitro Assays

We extensively characterized RO7502175 by determining the binding specificity to CCR8, binding activity to human Fc gamma receptors (FcγRs), ADCC activity, and cytokine release potential in in vitro studies. The methods for each of these assays are detailed below.

Binding Specificity to CCR8 from Various Species

To test the binding of RO7502175 to human, cyno, and murine CCR8, human CCR8-expressing (hCCR8.GNA15 CHO), cyno CCR8-expressing (cynoCCR8.GNA15 CHO), and mouse CCR8-expressing (mCCR8.GNA15 CHO) Chinese hamster ovary (CHO) stable cell lines generated at Genentech were used. To test the binding of RO7502175 to dog, rabbit, pig, and rat CCR8, human embryonic kidney (HEK) 293 cells (ATCC CRL-1573) were separately transfected with Genentech-generated C-terminal FLAG®-tagged DNA constructs for dog, rabbit, pig, and rat CCR8. Cells were stained with 5 μg/mL of RO7502175 washed twice with FACS buffer and stained with ALEXA FLUOR® 647 AffiniPure F(ab′)₂ Fragment Goat Anti-Human IgG, Fc fragment specific at 4° C. for 15 minutes. Transfected cells were washed twice, fixed and permeabilized with the BD CYTOFIX/CYTOPERM™ Fixation/Permeabilization Kit and stained with a mouse monoclonal ANTI-FLAG® M2-FITC antibody at 4° C. for 30 minutes. The cells were analyzed on a BD FACSCELESTA™ instrument. Data were analyzed using FLOWJO™ software (Version 10.6.1; FlowJo LLC; Ashland, OR). Additional details related to this in vitro assay can be found in the Additional Methods below.

### RO7502175 Binding Activity to Human Fc Gamma Receptors (FcγRs)

The binding interactions of test antibodies with human FcγRs (IIIA-F158 and IIIA-V158) were assessed in a panel of ELISA-based ligand binding assays (see, e.g., Shields et al. J. Biol. Chem. 2001; 276: 6591-6604). Each human FcγR was expressed as a fusion protein containing the extracellular domain of the receptor linked to a Gly-6× His-glutathione S-transferase (GST) polypeptide tag at the C-terminus. The test antibodies were analyzed as multimers by cross-linking with F(ab′)₂ fragments of polyclonal goat anti-human kappa light chain. Dose-response binding curves were generated by plotting the mean absorbance values (measured using a SPECTRAMAX® i3 microplate reader (Molecular Devices; Sunnyvale, CA) at 450 nm) from duplicates of sample dilutions against the sample concentrations. The 50% effective concentration (EC50) values of the antibody, at which 50% of the maximum response from binding to the FcγR was detected, were calculated by fitting the data to a 4-parameter model using SOFTMAX® Pro 6.5.1 software (Molecular Devices). Additional details related to this assay are described in the Additional Methods below.

### In Vitro ADCC Assays

The in vitro ADCC activity of RO7502175 was evaluated in three separate assays that used either pre-activated PBMCs, dissociated tumor cells or CCR8-expressing CHO cells as target cells. The relevant methods are described below in brief and additional details are provided in the Additional Methods below.

ADCC Assay with PBMC-Derived Treg Cells with Induced CCR8 Expression:

Human peripheral blood mononuclear cells (PBMCs) were transferred into immune deficient NSG™ mice, and T cells were recovered from the spleens 19 days later. NK cells isolated from PBMCs of a healthy human donor were then co-cultured with recovered human T cells at a 2:1 effector-to-target cell (E:T) ratio overnight in the presence of increasing concentrations of RO7502175 or fucosylated anti-CCR8 control or afucosylated anti-glycoprotein D (anti-gD) isotype control. After incubation, the frequencies of Treg cells (forkhead box P3 (FoxP3)+), conventional CD4+ T cells (FoxP3−), and CD8+ T cells were quantified by flow cytometry using a FORTESSA™ X-20 apparatus and FLOWJO™ software (Version 10.5.3; BD Biosciences; Franklin Lakes, NJ).

ADCC Assay with Dissociated Tumor Cells:

NK cells isolated from healthy donor PBMCs were co-cultured with dissociated renal cell carcinoma (RCC) tumor cells at a 2:1 or 3:1 E:T ratio overnight in the presence of increasing concentrations of RO7502175 or fucosylated anti-CCR8 control or afucosylated anti-gD isotype control. The recovery of Treg cells (FoxP3+), conventional CD4+ T cells (FoxP3−), and CD8+ T cells was quantified by flow cytometry using a FORTESSA™ X-20 apparatus and FLOWJO™ software (Version 10.5.3; BD Biosciences; Franklin Lakes, NJ).

### ADCC Activity Against CCR8-Expressing CHO Cells

RO7502175 was serially diluted in assay media containing 10 mg/mL of human IgG to mimic clinical setting. Serial dilutions of RO7502175 (0.004-1000 ng/mL) were added to hCCR8.GNA15 CHO (target cells), followed by incubation for 20-30 minutes. Then, freshly isolated healthy donor PBMCs (effector cells) were added at 25:1 E:T ratio, followed by 3-hour incubation. After centrifugation, fluorescent signals in supernatants were measured using the SPECTRAMAX® i3 microplate reader with excitation at 485 nm and emission at 520 nm. ADCC values were plotted against antibody concentrations, and the dose-response curve was fitted with a 4-parameter model using SOFTMAX® Pro 6.5.1 software.

### In Vitro Cytokine Release Assay

PBMCs were isolated from heparinized whole blood of 8 healthy donors via the ficoll method. 200,000 PBMCs were cultured in 96-well U-bottom plates for 18 hours in the presence of test articles in RPMI-1640 supplemented with 10% FBS, GLUTAMAX™, 2-[4-(2-hydroxyethyl)piperazin-1-yl]ethanesulfonic acid (HEPES), sodium pyruvate, non-essential amino acids, and penicillin/streptomycin. The positive controls for cytokine release from PBMCs were lipopolysaccharide (LPS, 300 ng/mL) and anti-CD3 (clone OKT3, 50 ng/mL)+anti-CD28 (clone 28.2, 50 ng/mL). RO7502175 or the isotype control (afucosylated anti-gD) were tested at concentrations of 0.0192, 0.096, 0.48, 2.4, 12, 60, 300, and 1500 μg/mL. The soluble assay format added test articles at the same time as adding the PBMCs to the culture. The immobilized assay format incubated test articles overnight at 4° C. and the wells were subsequently washed 3 times with PBS prior to adding PBMCs. Following 18 hours incubation, the plates were centrifuged at 2000×g for 5 minutes and the supernatants were harvested. Samples were stored at −80° C. until analyzed. Four pro-inflammatory cytokines, IL-2, IL-6, interferon gamma (IFNγ), and tumor necrosis factor alpha (TNFα), were chosen for analyses in this assay because of their known roles in cytokine release syndrome, consistent with industry-wide cytokine release assays (see, e.g., Finco et al. Cytokine. 2014; 66: 143-155; Riegler et al. Ther. Clin. Risk. Manag. 2019; 15: 323-335; Vessillier et al. Cytokine X. 2020; 2:100042; and Vidal et al. Cytokine. 2010; 51: 213-215). Each sample was assayed in triplicate for IFNγ, IL-2, IL-6, and TNFα using a custom Ella SIMPLE PLEX™ cartridge (ProteinSimple; San Jose, CA).

### Pharmacodynamic and Anti-Tumor Efficacy in Syngeneic Mouse Tumor Models

Murine medullary breast adenocarcinoma E0771 cells were implanted interstitially in the left fifth mammary fat pads of female C57BL/6 mice in both the PD and efficacy studies. Each mouse was injected with 1×10⁵ cells in a volume of 100 μL. At mean tumor volume of 130 mm³ in the PD study and 179 mm³ in the efficacy study, mice were distributed to treatment groups (n=10 mice per group) and a single dose of mIgG2a anti-gp120 (control, 1 mg/kg in PD study and 0.1 mg/kg in efficacy study) or anti-murine CCR8 antibody at 0.01, 0.03, 0.1, or 1 mg/kg IV was administered.

In the PD study, animals were 6-7 weeks old (average weight of 19.1 g). Blood samples for PK and PD analyses were collected on Day 1 (PK), 3 (PK, PD), and 7 (PK, PD). Blood was collected by retro orbital sampling (125 μL) from each animal on Day 1 for PK analysis. On Days 3 and 7, 4-5 mice per group were euthanized, and whole blood collected via terminal cardiac puncture for PK and PD analyses. In the efficacy study, animals were 10-11 weeks old (average weight of 20.5 g) and blood samples for PK analyses were collected on Day 1, 4, and 8 by retro orbital sampling (125 μL) from 5 animals per group (serial sampling). Blood samples from both studies were processed for serum and analyzed by a murine IgG2a (Allotype a) ELISA to determine the anti-murine CCR8 antibody concentrations (details in the Additional Methods below). The minimum quantifiable concentration (MQC) was 0.01563 μg/mL. Tumor sizes and mouse body weights were recorded twice weekly over the course of the studies. Tumors were measured in two dimensions (length and width) using calipers, and tumor volume was calculated using the formula (length×width^{A}2)/2. Mice were euthanized, per IACUC guidelines, when tumor volumes exceeded 2000 mm³ or if body weight loss was 220% of initial weights.

Sample collected for PD measurements were analyzed using flow cytometry on a FORTESSA™ X-20 (BD Biosciences) or FACSYMPHONY™ (BD Biosciences) instrument and FLOWJO™ software (BD Biosciences, Version 10.5.3). Frequencies of Treg cells, conventional CD4+ T cells, and CD8+ T cells were quantified in tumors, tumor-draining lymph nodes, spleens, and blood samples. The data was analyzed using EXCEL® (Microsoft) and PRISM® 8 (GraphPad; San Diego, CA) software, and analysis of variance was used for statistical testing. Additional details related to the PD sample processing and flow cytometry analysis are described in the Additional Methods below.

### Pharmacokinetic-Pharmacodynamic Evaluation in Cynomolgus Monkeys

Cynomolgus monkeys were selected to investigate the PK/toxicokinetics (TK), PD profiles, cytokine modulation and safety of RO7502175 as RO7502175 binds to human and cynomolgus monkey CCR8 with comparable binding affinities, and cynomolgus monkey is an accepted nonrodent species for nonclinical toxicity testing by regulatory agencies.

### Single-Dose Cynomolgus Monkey Study

In the single-dose study, each group consisted of 3 healthy male cynomolgus monkeys (2.4-3.0 kg) and were 2.7 to 3.5 years old. Animals were randomly assigned to receive afucosylated anti-gD (control) or RO7502175 as a single 10 mg/kg intravenous (IV) bolus. Approximately 0.25-0.5 mL of blood was collected by venipuncture from individual animals for assessing RO7502175 concentration and anti-drug antibodies (ADA). Blood was sampled at 0.25, 2, and 6 hours; 1, 2, 7, 14, 21, 28, and 35 days post-dose, and serum was assayed for concentrations of anti-gD and RO7502175 using a qualified generic total human IgG ELISA (GRIP) analytical method (details in the Additional Methods below). The lower limit of quantitation (LLOQ) of the assay was 0.015625 μg/mL. Blood samples for ADA analysis were collected at predose and <24 hours, 7, 14, 21, 28 and 35 post-dose, and serum was analyzed for antibodies against RO7502175 using a qualified sandwich ELISA assay (details in the Additional Methods below). All samples were stored at −70° C. until analysis.

Blood samples were collected at baseline, 6 hours, 1, 2, 14, 21, and 35 days post-dose for cytokine analysis. Each sample was assayed in duplicate using validated Meso Scale Discovery (MSD) multi-spot assay system using three different kits; proinflammatory panel 1 (NHP) kit (catalog #K15056G lot #K00818829) including analytes IFNγ, IL-1β, IL-2, IL-6, IL-10, IL-8; cytokine panel 1(NHP) kit (catalog #K15057D lot #K0081762) including analytes macrophage inflammatory protein 1 beta (MIP-1p), Eotaxin-3, thymus and activation regulated chemokine (TARC), Interferon gamma-induced protein 10 (IP-10), monocyte chemoattractant protein 1 (MCP-1), macrophage derived chemokine (MDC), monocyte chemoattractant protein 4 (MCP-4), macrophage inflammatory protein 1 alpha (MIP-1a); and chemokine panel 1 (NHP) kit (catalog #K15055D lot #K0081788) including analytes granulocyte-macrophage colony-stimulating factor (GM-CSF), IL-5, IL-7, IL-12/IL-13p40, IL-15, IL-16, IL-17A, tumor necrosis factor beta (TNFβ) and vascular endothelial growth factor A (VEGF-A). Manufacturer's protocols were followed for all the kits.

### Repeat-Dose Cynomolgus Monkey Study

The repeat-dose study included healthy cynomolgus monkeys weighing 2.0-2.4 kg and 2-3 years old. RO7502175 was administered at 30 or 100 mg/kg IV bolus to 4 animals/sex/group, and placebo to 3 animals/sex, once weekly for 7 injections, with terminal necropsy at 44 days post-dose. Approximately 0.3 mL of blood was collected by venipuncture at each timepoint to assess RO7502175 concentration and ADA. Blood sampling was performed at 0.25 and 6 hours, 2 and 7 days post 1st dose; 0.25 hours and 7 days post 2nd dose; 0.25 hours and 7 days post 3rd dose; 0.25 hours and 7 days post 4th dose; 0.25 hours and 7 days post 5th dose; 0.25 and 6 hours, 2 and 7 days post 6th dose; 0.25 hours and 2 days post 7th dose, and serum was assayed for quantification of RO7502175 concentrations using a LC-MS/MS method with a LLOQ of 50 μg/mL, which was validated at Pharmaceutical Product Development (PPD), LLC. Blood samples for ADA analysis were collected on Days −12 (pretreatment), and prior to dosing on Days 14, 21, 28, 35 and 42, and serum was analyzed for antibodies against RO7502175 using a bridging ELISA method, which was validated at Syneos Health. All animals were euthanized via sedation by intramuscular injection of ketamine hydrochloride, followed by an IV sodium pentobarbital prior to exsanguination. All collected samples were stored at −70° C. until analysis.

### Blood Immunophenotyping in Cynomolgus Monkey Studies

In the single-dose study, blood was collected for PD analysis on day −14 (pretreatment), predose, 6 and 24 hours, 2, 7, 14, 21, 28, and 35 days post-dose. In the repeat-dose study, blood was collected on days −4 (pretreatment), 3, 8, 22, 36 and 45 post-dose. In both studies, 100 μL of blood was added into v-bottom deep-well 96 well plates and 2 wells per condition (200 μL total blood) were used to acquire enough cells. Samples were spiked with drug tolerant 10 μg/ml anti-CCR8-1912 (hu.CCR8-1912.H12L3.hIgG1) and were incubated for 30 minutes at RT. After washing 3 times with 1 mL of stain buffer, samples were incubated with secondary antibody goat F(ab′)₂ anti-human IgG-AF647 (Jackson ImmunoResearch 109-606-170) for 10 minutes at RT. After secondary staining, samples were incubated with the following surface antibodies: mouse IgG1κ anti-human CD3-AF488 (BD Biosciences 557705), mouse IgG1κ anti-human CD4-BV605 (BD Biosciences 562843), mouse IgG1κ anti-human CD8-AF700 (BioLegend 344724), mouse IgG1κ anti-human CD25-BV786 (BD Biosciences 563701), armenian hamster IgG anti-human/mouse/rat CD278 (ICOS)-BV421 (BioLegend 313524) and mouse IgG1κ anti-NHP CD45RA-PE-Cy7 (BD Biosciences 561216). Samples were then lysed with 1×RBC lysis buffer for 15 minutes at room temperature (RT) protected from light. After washing 3 times, cells were put in Fixation/Permeabilization Buffer (Thermo Fisher 00-5523-00) for overnight incubation. On the next day, cells were washed twice with permeabilization buffer and were incubated with 10 μL of mouse IgG1κ anti-human FoxP3-PE (Biolegend 320108) diluted in permeabilization buffer for 60 minutes at RT. After washing twice with the stain buffer, cells were acquired on the LSRFORTESSA™ (BD Biosciences) and analyzed using BD FACSDIVA™ software (Version 8.0.1 and 9.0.1; BD Biosciences).

### Data and Statistical Analysis

All graphs were plotted using PRISM® (GraphPad Inc., CA). The data from in vitro binding specificity assay was analyzed using FLOWJO™ software (Version 10.6.1; FlowJo LLC; Ashland, OR). The data from in vitro ADCC assays and mouse PD studies were analyzed using FLOWJO™ software (Version 10.5.3; BD Biosciences; Franklin Lakes, NJ). The in vitro FcγR binding assay data and dose-response curves from CHO-cell ADCC assays were fitted to a 4-parameter model using SOFTMAX® Pro 6.5.1 software (Molecular Devices). The immunophenotyping data from cynomolgus monkey studies was analyzed using BD FACSDIVA™ software (Version 8.0.1 and 9.0.1; BD Biosciences).

For PK data analysis, nominal sample collection times and nominal dose solution concentrations were used. PK analyses from cynomolgus monkey studies were based on individual animal data. Data from both ADA-positive and -negative animals were included in PK parameter estimation. PK analyses from mouse studies were either based on individual animal data or composite (naïve-pooled) animal data due to sparse sampling. The PK parameters were calculated with PHOENIX® WINNONLIN® version 6.4 (Certara USA, Inc., NJ) software using a non-compartmental analysis approach consistent with IV bolus administration.

For the in vitro cytokine release assay, two-way analysis of variance with repeated measures was used to account for data points derived from the same donor. For each combination of drug/dose/analyte/assay format, a test was performed to assess whether the cytokine response was statistically significantly different from that in the vehicle control condition. For each combination of dose/analyte/assay combination, a test was also performed to assess whether RO7502175 was statistically significantly different from isotype control. Hypothesis tests were performed using Dunnett's testing procedure to control for multiple comparisons across dose levels within each combination of drug/analyte/assay format. For each combination, the family-wise error rate was controlled at 0.05 across dose levels, and adjusted p values >0.05 were considered not significant. If the estimated cytokine level was lower after application of RO7502175 than after application of isotype control, the result was considered non-significant. Analysis of variance was used for statistical testing of results from mouse PD studies.

mPBPK-PD Model Development

The model incorporates five key mechanisms to capture pharmacologic activity of anti-CCR8 antibodies: (a) antibody PK in blood, tumor (mouse only), and non-tumor tissue, (b) bivalent binding of antibody to CCR8, (c) CCR8+ Treg cell depletion in tumor as a function of number of CCR8 receptors occupied by antibody, (d) CD8+ T cell increases in tumor as a function inversely related to number of CCR8+ Treg cells in tumor, and (e) tumor cell depletion as a function of the ratio of tumor CD8+ T cells to tumor cells.

Anti-CCR8 antibody PK was modeled according to Cao et al. J. Pharmacokinet. Pharmacodyn. 2013; 40: 597-607. Mouse, cynomolgus monkey and human antibody binding affinities were quantified using in vitro assays. In mice, PK, tumor CCR8+ Treg cell depletion, tumor CD8+ T cell increase, and tumor killing parameters were calibrated from in vivo mouse studies (0.01, 0.03, 0.1, and 1 mg/kg IV single dose). The mouse model incorporated both linear and nonlinear clearance terms to account for nonlinear PK observed at dose levels 0.1 mg/kg and below. In cynomolgus monkeys, PK and circulating CCR8+ Treg cell depletion parameters were calibrated to 10 mg/kg single-dose study. Human clearance was scaled from cynomolgus monkey clearance (see, e.g., Deng et al. MAbs. 2011; 3: 61-66). A linear PK behavior is expected for RO7502175 in patients due to the low target expression levels. All model simulations were run using gQSPSim v1.1, a MATLAB® toolbox for running Simbiology models (MATLAB 2022a) (see, e.g., Hosseini et al. CPT Pharmacometrics Syst. Pharmacol. 2020; 9:165-176). Additional details related to the model including assumptions, equations, and parameters can be found in the Additional Methods below.

### Additional Methods

### In Vitro Assays

Various in vitro studies were performed to characterize RO7502175. A detailed description of the methods for these assays are provided below.

Binding Specificity to CCR8 from Various Species

To test the binding of RO7502175 to human, cyno, and murine CCR8, human CCR8-expressing (hCCR8.GNA15 CHO), cyno CCR8-expressing (cynoCCR8.GNA15 CHO), and mouse CCR8-expressing (mCCR8.GNA15 CHO) CHO stable cell lines generated at Genentech were used. CHO-K1 (ATCC CCL-61 Manassas, VI) cell line was used as a negative control. hCCR8.GNA15 CHO, cynoCCR8.GNA15 CHO, and mCCR8.GNA15 CHO stable cell lines were cultured in F-12K Medium (Kaighn's Modification of Ham's F-12 Medium; ATCC; Catalog No. 30 2004) supplemented with 10% fetal bovine serum and harvested by digestion with 0.5% (weight to volume) trypsin-ethylenediaminetetraacetic acid (EDTA) solution. To test the binding of RO7502175 to dog, rabbit, pig, and rat CCR8, human embryonic kidney (HEK) 293 cells (ATCC CRL-1573) were separately transfected with the following Genentech-generated C-terminal FLAG®-tagged DNA constructs for dog, rabbit, pig, and rat CCR8. To transfect HEK293 cells, 600,000 cells in 1 mL of complete Dulbecco's Modified Eagle Medium were seeded per well of a 12-well cell culture plate and cultured at 37° C. in a 5% CO₂ incubator overnight. Cells were then transfected with each DNA construct by using the TRANSIT-X2® Dynamic Delivery System (Mirus Bio LLC; Madison, WI; Catalog No. MIR6000), with a reagent:DNA ratio of 3:1 and incubated for 24 hours. Cells were stained with 5 μg/mL of RO7502175 in fluorescence-activated cell sorting (FACS) buffer (phosphate-buffered saline with 0.5% bovine serum albumin and 2 mM EDTA) at 4° C. for 30 minutes, washed twice with FACS buffer and stained with ALEXA FLUOR® 647 AffiniPure F(ab′)₂ Fragment Goat Anti-Human IgG, Fc fragment specific (Jackson ImmunoResearch Laboratories; West Grove, PA; Catalog No. 109-606-170; 1:500 dilution) at 4° C. for 15 minutes. Transfected cells were washed twice, fixed and permeabilized with the BD CYTOFIX/CYTOPERM™ Fixation/Permeabilization Kit (BD Biosciences; Catalog No. 554714), and stained with a mouse monoclonal ANTI-FLAG® M2-FITC antibody (Sigma-Aldrich; St. Louis, MO; Catalog No. F4049; 1:100 dilution) at 4° C. for 30 minutes. Then, the cells were washed twice with FACS buffer and resuspended in FACS buffer containing propidium iodide (BD Biosciences; Catalog No. 556463; 0.5 μg/mL) for analysis on a BD FACSCELESTA™ instrument. Data were analyzed using FLOWJO™ software (Version 10.6.1; FlowJo LLC; Ashland, OR).

### RO7502175 Binding Activity to Human Fc Gamma Receptors (FcγRs)

The binding interactions of test antibodies with human FcγRs (IIIA-F158 and IIIA-V158) were assessed in a panel of ELISA-based ligand binding assays (see, e.g., Shields et al. J. Biol. Chem. 2001; 276: 6591-6604). Each human FcγR was expressed as a fusion protein containing the extracellular domain of the receptor linked to a Gly-6× His-glutathione S-transferase (GST) polypeptide tag at the C-terminus. The test antibodies were analyzed as multimers by cross-linking with F(ab′)₂ fragments of polyclonal goat anti-human kappa light chain (MP Biomedicals; Solon, OH) at an approximate molar ratio of 1:3, and the mixtures were incubated at RT for 1 hour before use in the assays. Plates were coated with 2.0 μg/mL anti-GST antibody (Genentech) in a 0.05 M sodium carbonate buffer (pH 9.6) overnight at 28° C. After blocking with the assay buffer, the plates were incubated with 0.5 μg/mL FcγRs at RT for 2 hours. Serial dilutions of test antibodies were added as multimeric complexes and the plates were incubated at RT for 2 additional hours. Plates were washed 5 times with phosphate buffered saline (PBS) supplemented with 0.05% TWEEN® 20 using an ELx405™ plate washer (BioTek Instruments; Winooski, VT) after each incubation step. Antibodies bound to the FcγRs were detected with horseradish peroxidase (HRP)-conjugated F(ab′)₂ fragments of polyclonal goat anti-human F(ab′)₂ antibodies (Jackson ImmunoResearch Laboratories; West Grove, PA), and substrate tetramethylbenzidine (Kirkegaard & Perry Laboratories; Gaithersburg, MD) was added. The plates were incubated at RT for 5-20 minutes (depending on the FcγR tested) to allow color development. The reaction was terminated with 1 M phosphoric acid, and absorbance at 450 nm (with the background at 630 nm subtracted) was measured using a SPECTRAMAX® i3 microplate reader (Molecular Devices; Sunnyvale, CA). Dose-response binding curves were generated by plotting the mean absorbance values from duplicates of sample dilutions against the sample concentrations. The 50% effective concentration (EC50) values of the antibody, at which 50% of the maximum response from binding to the FcγR was detected, were calculated by fitting the data to a 4-parameter model using SOFTMAX® Pro 6.5.1 software (Molecular Devices).

In Vitro ADCC Assays with PBMC-Derived Treg Cells and Dissociated Tumor Cells
Isolation of NK Cells from Human Peripheral Blood Mononuclear Cells (PBMCs):

Buffy coats from healthy human volunteers were obtained from the Vitalant Community Blood Center (Brisbane, CA). Buffy coat was diluted 1:1 with phosphate-buffered saline and carefully overlayed onto 15 mL of FICOLL-PAQUE™ PLUS (Catalog No. 17144003; Cytiva; Marlborough, MA), and centrifuged at 800×g at RT without the stopping brake for 20 minutes. PBMCs were collected at the interface, washed with cold magnetic-activated cell sorting (MACS) buffer and centrifuged at 300×g at RT for 5 minutes. NK cells were isolated by magnetic separation using the Human NK Cell Isolation Kit (Catalog No. 130-092-657; Miltenyi Biotec; North Rhine-Westphalia, Germany) according to the manufacturer's protocol.

ADCC Assay with PBMC-Derived Treg Cells with Induced CCR8 Expression:

Human PBMCs (10×10⁶ cells diluted in sterile 200 μL of 1×PBS) were injected into female NSG™ mice of 8-10 weeks of age, intraperitoneally (IP). On Day 19 after cell transfer, mice were euthanized, and spleens were collected for cell isolation. Spleens were minced through a 100-μm cell strainer into cold MACS buffer and centrifuged at 350×g at 4° C. for 5 minutes. Spleen samples were incubated in EBIOSCIENCE™ 1×RBC Lysis Buffer (Catalog No. 00-4333-57; Thermo Fisher Scientific; San Diego, CA). Lysis was stopped after 1 minute with cold MACS buffer. Cells were filtered through a 40-μm cell strainer to remove any clumps and centrifuged again. Spleen samples were resuspended in MACS buffer. Human T cells were enriched from mouse spleen samples using the mouse Direct Lineage Cell Depletion Kit (Catalog No. 130-110-470; Miltenyi Biotec; Bergisch Gladbach, Germany) according to the manufacturer's protocol. Cells were resuspended in culture media (RPMI 1640 (Catalog No. A0806; Genentech), 10% fetal bovine serum (Catalog No. SH30071.03 Lot AZA180864; Hyclone; Logan, UT), GIBCO™ 2 mM GLUTAMAX™ (Catalog No. 35050-061; Thermo Fisher Scientific), 1 mM GIBCO™ Sodium Pyruvate (Catalog No. 11360-070; Thermo Fisher Scientific), 0.1 mM GIBCO™ MEM Non-Essential Amino Acids (Catalog No. 11140-050; Thermo Fisher Scientific), 55 μM GIBCO™ β-mercaptoethanol (Catalog No. 21985023; Thermo Fisher Scientific), 100 U/mL GIBCO™ penicillin and 100 μg/mL GIBCO™ streptomycin (Catalog No. 15140-122; Thermo Fisher Scientific), 10 mM HEPES) at concentration 1 million cells/mL.

Human T cells (100,000 cells) recovered from spleens of NSG™ mice, as described above, were incubated for 30 minutes at RT with 50 μL of RO7502175 or fucosylated anti-CCR8 control or afucosylated anti-glycoprotein D (anti-gD) isotype control. Antibodies were 10-fold serial diluted through 4 steps, with the top concentration at 1 μg/mL, in 96-well U-bottom plates. Thereafter, 50 μL of human NK cells in culture media (200,000 cells, for a 2:1 NK:T-cell ratio) containing IL-7 (final concentration 25 ng/mL; Catalog No. 130-095-367; Miltenyi Biotec) and IL-15 (final concentration 50 ng/mL; Catalog No. 130-095-760; Miltenyi Biotec) were added. Cultures were incubated overnight at 37° C.

Samples stained as indicated were analyzed by flow cytometry using a FORTESSA™ X-20 apparatus and FLOWJO™ software (Version 10.5.3; BD Biosciences; Franklin Lakes, NJ). Cell numbers were calculated using the following formula:

$$Absolute count ( \frac{cells}{[ μ L ]} ) = \frac{( Cell count \times Counting beads volume )}{( Counting beads count \times Cell volume )} \times Counting beads concentration ( \frac{beads}{[ μ L ]} )$$

ADCC Assay with Dissociated Tumor Cells:

Dissociated tumor cells (100,000 cells in 100 μL of culture medium) were incubated with 50 μL of RO7502175 or fucosylated anti-CCR8 control or afucosylated anti-gD isotype control in culture medium at RT for 30 minutes. Antibodies were added in a 4-step 10-fold serial dilution, with a top final concentration of 1 μg/mL, in 96-well U-bottom plates. Next, 50 μL of NK cells (200,000 cells for a 2:1 effector-to-target cell [E:T] ratio or 300,000 cells for a 3:1 E:T ratio) resuspended in culture medium containing (IL-7; final concentration 25 ng/mL; Catalog No. 130-095-367; Miltenyi Biotec) and IL-15 (final concentration 50 ng/mL; Catalog No. 130-095-760; Miltenyi Biotec) were added. Cultures were incubated at 37° C., with 5% CO₂ overnight.

Following overnight incubation, cells were transferred to a 96-well V-bottom plate, centrifuged at 800×g at 4° C. for 2 minutes, and washed with fluorescence-activated cell sorting (FACS) buffer (PBS, 0.5% BSA, 0.05% sodium azide; Catalog No. A4439; Genentech). Samples were then stained with Fixable Viability Dye (1:2000 dilution) at 4° C. for 15 minutes. Cells were washed with cold PBS, resuspended in 200 μL of fixation/permeabilization buffer (Fixation/Permeabilization Concentrate (Catalog No. 00-5123-43; Thermo Fisher Scientific) diluted in Fixation/Permeabilization diluent (Catalog No. 00-5223-56; Thermo Fisher Scientific)), and incubated at RT in the dark for 30 minutes. Cells were then washed with the fixation/permeabilization buffer and intracellularly stained with an intracellular antibody mix at 4° C. for 30 minutes. Cells were centrifuged, washed with FACS buffer, centrifuged again, and resuspended in 125 μL of FACS buffer. Before acquisition, 12.5 μL of COUNTBRIGHT™ Absolute Counting Beads (Catalog No. C36950; Thermo Fisher Scientific) was added in each sample for cell enumeration. Samples stained as indicated were analyzed by flow cytometry using a FORTESSA™ X-20 apparatus and FLOWJO™ software (Version 10.5.3; BD Biosciences; Franklin Lakes, NJ). Cell numbers were calculated using the following formula:

$$Absolute count ( \frac{cells}{[ μ L ]} ) = \frac{( Cell count \times Counting beads volume )}{( Counting beads count \times Cell volume )} \times Counting beads concentration ( \frac{beads}{[ μ L ]} )$$

### In Vitro ADCC Activity Against CCR8-Expressing CHO Cells

ADCC assays were carried out using freshly isolated PBMCs from healthy donors as effector cells and hCCR8.GNA15 CHO as target cells. Briefly, PBMCs were isolated by density gradient centrifugation using a Uni-Sep blood separation tube (Accurate Chemical & Scientific Corporation; Carle Place, NY). Target cells were pre-labeled with 1.4 mM solution of calcein AM (Molecular Probes; Eugene, OR) and seeded in a 96-well, round-bottom plate (BD Biosciences; Mississauga, Canada) at 2×10⁴ cells/well. Serial dilutions of test antibodies were added to the plate containing the target cells, followed by incubation at 37° C. with 5% carbon dioxide for 20-30 minutes to allow binding of the antibody to its target. RO7502175 was serially diluted in assay media containing 10 mg/mL of human IgG to mimic the clinical in vivo setting. The final concentrations of antibodies ranged 0.004-1000 ng/mL following a 4-fold serial dilution for a total of 10 samples per test antibody.

After the incubation, 5×10⁵ PBMC effector cells in 100 μL assay medium were added to each well to give an effector:target cell ratio of 25:1. The assay plate was centrifuged at 700 rpm for 1 minute to concentrate the cells at the bottom of the well, and the plate was incubated for an additional 3 hours. The plate was centrifuged at the end of incubation, and fluorescent signals in supernatants were measured using the SPECTRAMAX® i3 microplate reader, with excitation at 485 nm and emission at 520 nm. Signal of wells containing only the target cells represented spontaneous release of green-fluorescent calcein from labeled cells, whereas wells containing target cells lysed with TRITON™ X-100 (Genentech) provided the maximum signal available (maximum lysis). Spontaneous lysis control, antibody-independent cellular cytotoxicity (AICC), was measured in wells containing target and effector cells without the addition of antibody. The extent of specific ADCC was calculated as follows:

$$\% ADCC = 100 \times \frac{( Sample signal - AICC signal )}{( Maximum lysis signal - Spontaneous release signal )}$$

The ADCC values of sample dilutions were plotted against the antibody concentration, and the dose-response curve was fitted with a 4-parameter model using SOFTMAX® Pro 6.5.1 software.

### Syngeneic Mouse Tumor Studies—PD Sample Processing and Flow Cytometry Analysis

Tumor samples were minced, and pieces were washed with 2 mL RPMI medium containing 1% FBS. Thereafter, 0.5 mL of RPMI medium, containing 1% FBS, 0.2 U/mL LIBERASE™ DL (Catalog No. 5466202001; Sigma Aldrich; St. Louis, MO), and 0.2 mg/mL DNase I (final concentration, 0.2 mg/mL; Catalog No. 10104159001; Sigma Aldrich), were added. The tumor samples were incubated at 37° C., shaking at approximately 200 rpm at an angle, for 30 minutes. Tissue digestion was stopped by adding approximately 30 mL of cold RPMI medium containing 10% FBS and immediately placing the tubes on ice. Samples were transferred to new tubes through 100 μm filters. The remaining pieces of tumor were passed through the filter using the plunger end of a syringe. The filter was washed with an additional approximately 10 mL of cold RPMI containing 10% FBS. The samples were centrifuged at 350×g at 4° C. for 5 minutes, and the supernatant was removed. The cells were washed with fluorescence activated cell sorting (FACS) buffer (1×PBS containing 0.5% bovine serum albumin and 0.1% sodium azide; Genentech) and filtered through a 40 μm cell strainer. The cells from each tumor were centrifuged again and resuspended in 0.1-0.8 mL of cold FACS buffer.

The lymph nodes and spleens were minced through 100 μm cell strainers into a cold FACS buffer and centrifuged at 350×g at 4° C. for 5 minutes. The spleen samples were then suspended in 5 mL of red blood cell lysis buffer (Catalog No. 00-4333-57; Thermo Fisher Scientific; San Diego, CA). Lysis was stopped after 1 minute with cold FACS buffer. The cells were filtered through a 40 μm cell strainer to remove any clumps and centrifuged again. Lymph node and spleen samples were resuspended in 0.1 mL or 3 mL, respectively, of cold FACS buffer. Blood samples were transferred to 15 mL conical tubes. Red blood cell lysis buffer (5 mL) was added, and cells were incubated for 5 minutes at RT. The reaction was stopped by adding 10 mL of cold FACS buffer. After centrifugation at 350×g at 4° C. for 5 minutes, the cells were resuspended in 130 μL of cold FACS buffer.

Cell suspension samples (50 μL) were transferred to a 96-well V-bottom plate, and 2× antibody surface stain mix (Fixable Viability Dye, Catalog No. 423106, BioLegend; Anti-mouse CD62L, Catalog No. 563252, BD Biosciences) was added directly to the cells. The cells were stained at 4° C. for 30 minutes, and an aliquot was taken for counting. Tumor cells (20 μL) and other tissue cells (3 μL) were placed into wells of a 96-well plate pre-filled with 215 μL of 1:40 counting bead dilution (ACBP-100-10; Spherotech; Lake Forest, IL) and anti-CD45.2 BV786 (1:200 dilution) and then incubated on ice for 30 minutes. The remaining cells were washed twice with cold FACS buffer before being resuspended in 200 μL of fixation/permeabilization buffer (Fixation/Permeabilization Concentrate (Catalog No. 00-5123-43; Thermo Fisher Scientific; Waltham, MA) diluted in Fixation/Permeabilization diluent (Catalog No. 00-5223-56; Thermo Fisher Scientific)). The cells were then fixed for 30 minutes on ice in the dark. Thereafter, the cells were washed with fixation/permeabilization buffer and blocked with mouse and rat serum (Catalog No. 015-000-120 and 012-000-120, respectively; Jackson ImmunoResearch; West Grove, PA) at 1:20 dilution at RT in the dark for 30 minutes. An intracellular antibody mix (Anti-mouse CD3e, Catalog No. 563565, BD Biosciences; Anti-mouse CD4, Catalog No. 564933, BD Biosciences; Anti-mouse FoxP3, Catalog No. 48-5773-82, Thermo Fisher Scientific; Anti-mouse CD45.2, Catalog No. 563686, BD Biosciences; Anti-mouse CD44, Catalog No. 553133, BD Biosciences; Anti-mouse CD8a, Catalog No. 564933, BD Biosciences; Anti-mouse CCR8, Catalog No. 150304, BioLegend) was then added, and the cells were stained at 4° C. overnight. The cells were centrifuged and washed twice with FACS buffer, centrifuged again, and resuspended in 60 μL of FACS buffer for analysis.

PK and ADA assays

### Mouse IgG2a (Allotype a) ELISA

For analysis of anti-murine CCR8 antibody concentrations in C57BL/6 mouse serum, NUNC® MAXISORP™ 384-well plates (Thermo, cat #464718) were coated with 3 μg/mL mouse anti-mouse IgG2a[a] (BD Biosciences, Cat #553501) diluted in PBS pH 7.4 and incubated overnight at 4° C. The plates were washed 3 times with a wash buffer (0.05% TWEEN®-20 in PBS buffer, pH 7.4) and treated with block buffer (PBS/0.5% BSA/15 ppm PROCLIN™, pH 7.4) for 1 to 2 hours at room temperature (RT). The plates were then washed 3 times with wash buffer and samples diluted in sample diluent (PBS/0.5% BSA/0.05% TWEEN® 20/5 mM EDTA/0.25% CHAPS/0.35M NaCl/0.2% BgG/15 ppm PROCLIN™, pH 7.4) were added to the wells, and incubated for 2 hours at RT with gentle agitation. After washing the plates 6 times with wash buffer, a detection antibody, horseradish peroxidase (HRP) conjugated rat anti-mouse IgG2a (GeneTex Inc, Cat #GTX11571), diluted to 250 ng/mL in an assay buffer (PBS/0.5% BSA/15 ppm PROCLIN™/0.05% TWEEN® 20, pH7.4) was added to the wells and incubated on a shaker for 1 hour at RT. The plates were washed 6 times with wash buffer and developed using 3,3′,5,5′-tetramethylbenzidine (TMB) peroxidase substrate (KPL, Cat #5120-0047) for 20 minutes followed by 1 M phosphoric acid to stop the reaction. Absorbance was measured at 450 nm against a reference wavelength of 620 nm. The concentration of anti-murine CCR8 antibody in the samples was extrapolated from a 4-parameter fit of the standard curve.

### Generic Total Human IgG ELISA (GRIP)

For analysis of RO7502175 in cynomolgus monkey serum samples from single-dose study, NUNC® MAXISORP™ 384-well plates (Thermo, cat #464718) were coated with 0.5 μg/mL monkey adsorbed sheep anti-human IgG (Binding Site, cat #AU003.M) diluted in 0.05 M carbonate/bicarbonate buffer pH 9.6 and incubated overnight at 4° C. The plates were washed 3 times with a wash buffer (0.05% TWEEN®-20 in PBS buffer, pH 7.4) and treated with block buffer (PBS/0.5% BSA/15 ppm PROCLIN™, pH 7.4) for 1 to 2 hours at RT. The plates were then washed 3 times with wash buffer and samples diluted in sample diluent (PBS/0.5% BSA/0.05% TWEEN® 20/5 mM EDTA/0.25% CHAPS/0.35M NaCl/15 ppm PROCLIN™, pH 7.4) were added to the wells, and incubated for 2 hours at RT with gentle agitation. After washing the plates 6 times with wash buffer, a detection antibody, HRP conjugated-monkey absorbed goat anti-Human IgG (Bethyl Laboratories, Inc., cat #A80-319P-12), diluted to 100 ng/mL in assay buffer (PBS/0.5% BSA/15 ppm PROCLIN™/0.05% TWEEN® 20, pH7.4) was added to the wells and incubated on a shaker for 1 hour at RT. The plates were washed 6 times with wash buffer and developed using TMB peroxidase substrate (KPL, Catalog #5120-0077) for 20 minutes followed by 1 M Phosphoric acid to stop the reaction. Absorbance was measured at 450 nm against a reference wavelength of 620 nm. The concentration of RO7502175 in the samples was extrapolated from a 4-parameter fit of the standard curve.

### ADA Assay

The ADA titers against RO7502175 in cynomolgus monkey serum samples from single-dose study were measured using a sandwich ELISA assay. Briefly, serum samples, negative control, and positive control antibody (monkey anti-huIgG, Genentech, Inc) were combined with a mixture of 0.25 μg/mL of biotin- and digoxigenin (DIG)-conjugated drug diluted in 2% naive monkey serum. ADA complexes were captured with neutravidin coated plates and detected with HRP-conjugated chicken anti-DIG polyclonal antibody (Abcam, cat #ab51949).

mPBPK-PD Model Structure

The model includes five compartments for the anti-CCR8 antibody to distribute through: (a) central (blood), (b) leaky tissue, (c) tight tissue, (d) tumor, and (e) lymph compartments. In each compartment except lymph, the model includes a CCR8 target capacity. The antibody distributes through the five compartments following intravenous dosing, is nonspecifically cleared from the blood, binds to CCR8 in compartments containing CCR8, and undergoes receptor-mediated internalization. Following binding, CCR8+ Treg cells in the compartment are depleted as a function of CCR8 receptor occupancy. In the tumor, as CCR8+ Treg cells are depleted, CD8+ T cells expand and drive tumor cell killing. Equations and parameters describing each of these mechanisms are highlighted below.

### Anti-CCR8 Antibody Transport:

Free anti-CCR8 antibody (D) is transported out of the i^{th} compartment according to Cao et al. J. Pharmacokinet. Pharmacodyn. 2013; 40: 597-607 (Eq. 1). We estimated tumor anti-CCR8 antibody concentration by fitting σₜᵤₘₒᵣ such that the ratio of AUCs of tumor to blood compartments over 21 days was 10%.

$${r}_{free antibody 𝔱rans} = {L}_{i} ( 1 - {σ}_{i} ) {D}_{i} \qquad Eq . 1$$

### Anti-CCR8 Antibody Binding to CCR8:

Anti-CCR8 antibody binds reversibly to free CCR8 (R) to form double-bound CCR8-antibody-CCR8 complex (RDR) according to a second order bivalent binding reaction (Eq. 2). Due to the high affinity of anti-CCR8 antibody, monovalent binding is ignored such that the antibody exists in only two states: unbound or bound to two CCR8 molecules.

$${r}_{bind} = {k}_{on} ( 2 [ R ] [ D ] - {K}_{D} [ RDR ] ) \qquad Eq . 2$$

The number of CCR8 receptors (Eq. 3) occupied is used to determine the rate at which CCR8+ Treg cells die.

$$\# {R}_{Bound} = \frac{2 * [ RDR ]}{[ Treg ]} \frac{{N}_{AV} V}{1 e 12} \qquad Eq . 3$$

### CCR8+ Treg Cells and Total CCR8 Level Balance:

Both CCR8+ Treg cells as well as the overall levels of CCR8 receptor are balanced in the model. In general, CCR8+ Treg cells are produced, die naturally by apoptosis, or die due to anti-CCR8 antibody-mediated ADCC.

CCR8+ Treg cells production and death are modeled assuming 0^{th} order kinetics (Eq. 4) and 1^{st} order kinetics (Eq. 5), respectively, in each compartment. The pre-dose amount of CCR8+ Treg cells in mice was set by the experimental pre-dose observation in the in vivo E0771 mouse tumor study. In cynomolgus monkeys and humans, the number of CCR8+ Treg cells was determined based on CCR8+ Treg cell counts from the GLP Toxicity study.

$${k}_{syn𝔱h CCR 8 + Treg} = {[ {T}_{reg} ]}_{ss} {k}_{death , Treg} \qquad Eq . 4$$

$${r}_{death CCR 8 + Treg} = {k}_{death , Treg} [ {T}_{reg} ] \qquad Eq . 5$$

To maintain a fixed receptor hypothesis, CCR8 synthesized is modeled with two mechanisms. The first synthesis reaction occurs as a result of new CCR8+ Treg cell production (Eq. 6). The second synthesis reaction balances the intracellular degradation of CCR8 (Eq. 7).

$${r}_{CCR 8} formation from Treg birth = {k}_{synth CCR 8 + Treg} rpc \frac{1 e 12}{{N}_{AV} V} \qquad Eq . 6$$

$${r}_{CCR 8} formation from Tregs = 2 {k}_{in𝔱} [ {T}_{reg} ] rpc \frac{1 e 12}{{N}_{AV} V} \qquad Eq . 7$$

CCR8 degradation is also represented by two mechanisms. First, free CCR8 can be degraded via receptor internalization (Eq. 8). Since each antibody-CCR8 complex contains two CCR8 molecules due to bivalent binding, the rate of internalization for the bound complex is twice that of the unbound receptor (Eq. 9). These assumptions result in a fixed receptor level, where the total amount of CCR8 in the system remains constant regardless of the presence of anti-CCR8 antibody. Second, CCR8 is degraded during CCR8+ Treg cell apoptosis (Eq. 10).

$${r}_{unbound receptor deg} = {k}_{deg} [ R ] \qquad Eq . 8$$

$${r}_{bound antibody in𝔱} = {k}_{in𝔱} [ RDR ] = 2 {k}_{deg} [ RDR ] \qquad Eq . 9$$

$${r}_{CCR 8 degradation , Treg death} = {k}_{dea𝔱h , Treg} [ R ] \qquad Eq . 10$$

### CCR8+ Treg Cell Anti-CCR8 Antibody-Induced Depletion:

CCR8+ Treg cells are depleted by assuming that the rate of depletion is a function of the number of CCR8 receptors bound by anti-CCR8 antibody (Eq. 11).

$${r}_{Treg depletion} = \frac{{{k}_{deple𝔱e} [ \# {R}_{bound} ]}^{{h}_{deplete}}}{{K}_{M , deple𝔱e}^{{h}_{deplete}} + {[ \# {R}_{bound} ]}^{{h}_{deplete}}} [ {T}_{reg} ] \qquad Eq . 11$$

Anti-CCR8 antibody-induced CCR8 depletion in cynomolgus monkey and clinical simulations was turned off in order to examine the theoretical RO of CCR8+ Treg cells over the entire dosing regimen.

### CD8+ T Cell Expansion:

In the tumor only, depletion of CCR8+ Treg cells directly drives CD8+ T cell expansion (Eq. 12).

$${r}_{CD 8 + T cell expansion} = \frac{{{ν}_{max , expansion} [ \% {T}_{reg} depleted ]}^{{h}_{expansion}}}{{K}_{M , CD 8 + T cell expansion}^{{h}_{expansion}} + {[ \% {T}_{reg} depleted ]}^{{h}_{expansion}}} \qquad Eq . 12$$

### CD8+ T Cell Synthesis and Death:

To capture CD8+ T cells in mouse tumors, we assumed a 0^{th} order production (Eq. 13) and 1^{st} order death rate (Eq. 14). The pre-dose CD8+ T cell count in tumor was set equivalent to experimental pre-dose values in the in vivo E0771 mouse tumor study.

$${r}_{CD 8 + T cell synthests} = {k}_{syn𝔱h , CD 8 + T cell} \qquad Eq . 13$$

$${r}_{CD 8 + T cell death} = {k}_{CD 8 + T cell death} [ CD 8 T cell ) \qquad Eq . 14$$

### Tumor Cell Killing:

Tumor cells are killed according to an effector:tumor cell (E:T) ratio (Eq. 15-16).

$$[ E : T ] = \frac{[ CD 8 T cell ]}{[ Tumor cell ]} \qquad Eq . 15$$

$${r}_{tumor killing} = \frac{{{k}_{kill 𝔱umor} [ E : T ]}^{{h}_{tumor killing}}}{{K}_{M , kill 𝔱umor}^{{h}_{tumor killing}} + {[ E : T ]}^{{h}_{tumor killing}}} [ Tumor cell ] \qquad Eq . 16$$

### Results

RO7502175 binds to human and cynomolgus monkey CCR8, exhibits potent ADCC activity, and causes minimal in vitro cytokine release

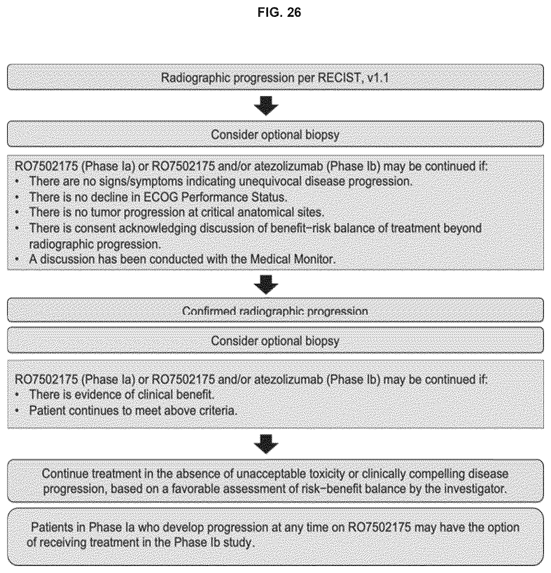

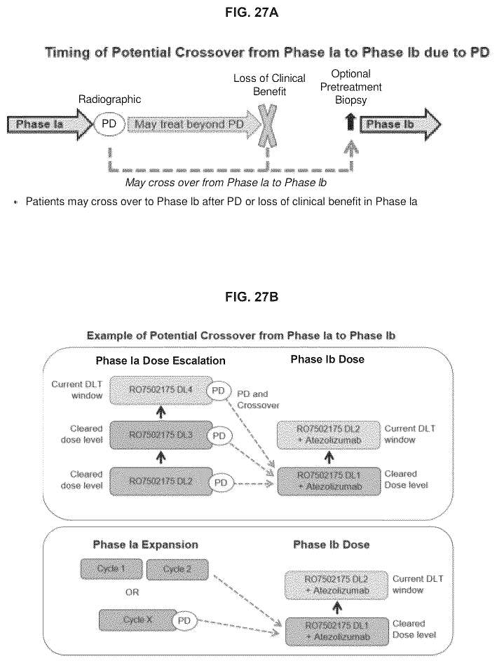

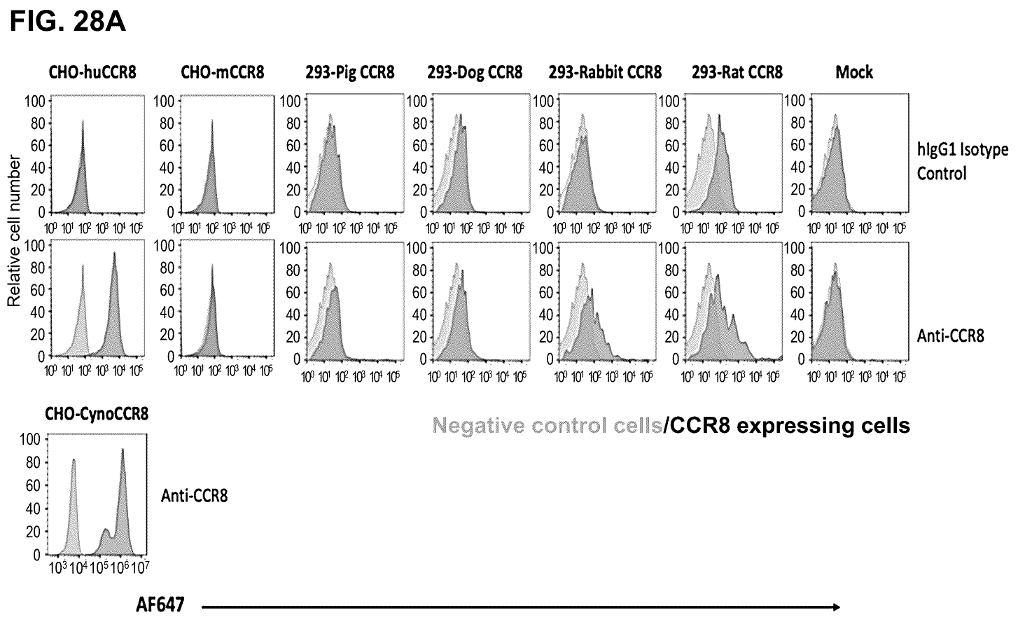

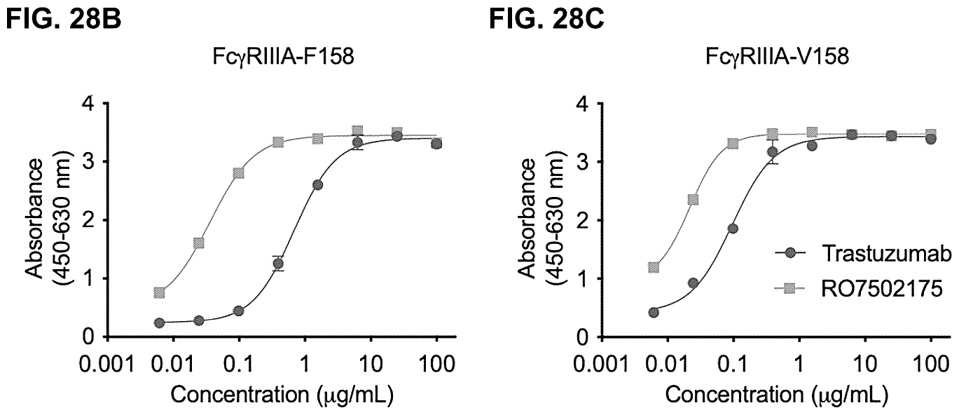

RO7502175 binds to human and cynomolgus monkey CCR8 but does not cross-react with mouse, pig, dog, rabbit, or rat CCR8 (FIG. 28A). RO7502175 demonstrated comparable low picomolar binding affinities to human and cynomolgus monkey CCR8 (K_{D} values of 32 μM and 27 μM, respectively), justifying the pharmacological relevance of cynomolgus monkeys as the most appropriate test species for toxicity studies. Anti-murine CCR8 antibody binds to murine CCR8 with an affinity of 218 μM, slightly weaker compared to the binding of RO7502175 to human and cynomolgus monkey CCR8. The affinity values were utilized to calculate the empirical RO to inform the MABEL-derived FiH dose and will be discussed subsequently.

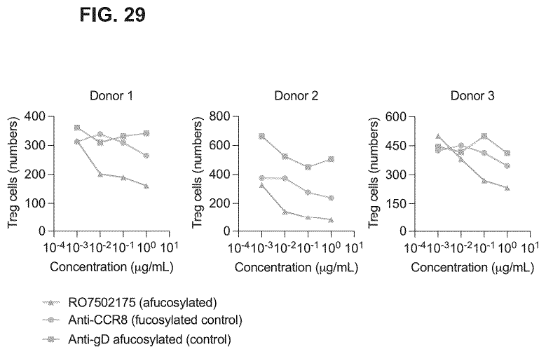

RO7502175 showed enhanced binding to both allotypes of FcγRIIIA, F158 and V158, compared with a control molecule containing a wild type, fucosylated Fc region (FIGS. 28B and 28C). RO7502175 depleted human Treg cells from PBMCs that had been previously activated to induce CCR8 expression across three donors and showed enhanced ADCC activity relative to the isotype and wild type, fucosylated anti-CCR8 controls (FIG. 29). Additionally, RO7502175 showed selective and concentration-dependent ADCC-mediated depletion of intratumoral Treg cells but not other CD4+ or CD8+ effector T cells from dissociated renal cell carcinoma tumors (FIG. 30A). RO7502175 demonstrated enhanced ADCC activity as compared to the anti-CCR8 antibody with a wild type Fc and the afucosylated isotype control in these assays (FIGS. 29 and 30A). Potent ADCC activity was observed in human CCR8-expressing CHO cell-based assays with geometric mean EC50 of 0.003 μg/mL using 8 different donors (FIG. 30B). The ADCC data from CHO-cell based assays informed the in vitro MABEL-derived FiH dose.

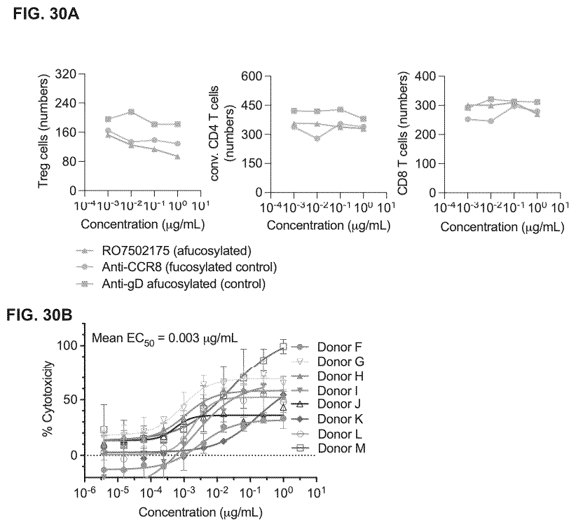

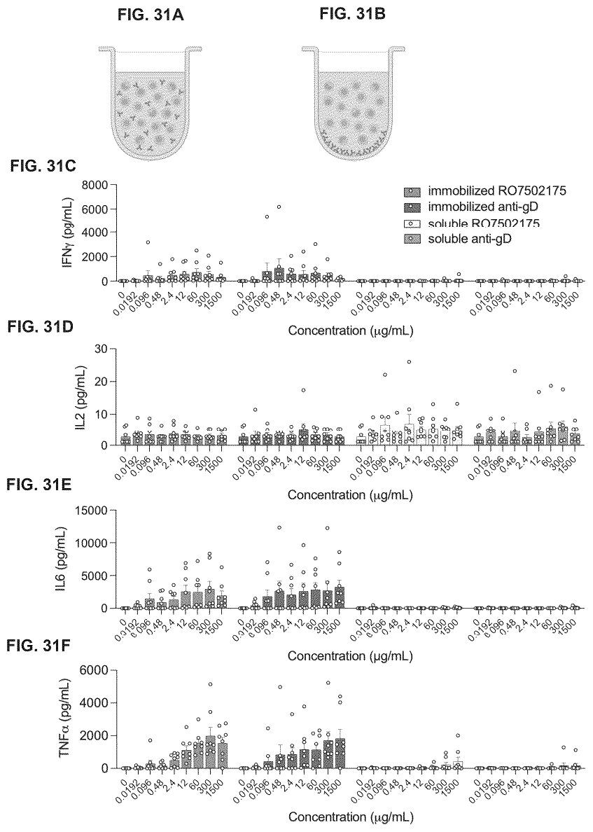

An in vitro assay assessing cytokine release from human PBMCs incubated with RO7502175 was used to evaluate the potential risk of acute cytokine release in patients (FIGS. 31A and 31B). In the immobilized assay format, incubation with either RO7502175 or afucosylated anti-gD (isotype control) resulted in no elevation of IL-2 and comparable increases in TNFα, IL-6, and IFNγ relative to the observed levels with vehicle control (FIGS. 31C-31F). These results suggest a target-independent mechanism of cytokine release and were not considered attributable to binding of RO7502175 to CCR8 on the surface of cells. Given that the immobilization of antibodies leads to a planar, high-density deposition, which may lead to altered antibody presentation and conformation, the immobilized assay format is considered less physiologically relevant than the soluble format. In the soluble format, incubation with RO7502175 up to 1500 μg/mL did not alter cytokine secretion relative to that observed with anti-gD and did not result in significant increases in cytokine levels as compared with the control group (FIGS. 31C-31F), suggesting that RO7502175 poses a low risk of cytokine release in the clinic. The in vitro cytokine assay results supported the integrated approach used for RO7502175 FiH dose selection.

### Anti-Murine CCR8 Antibody Demonstrated Preferential Depletion of Treg Cells in Tumor and Potent Anti-Tumor Efficacy in a Syngeneic Mouse Model

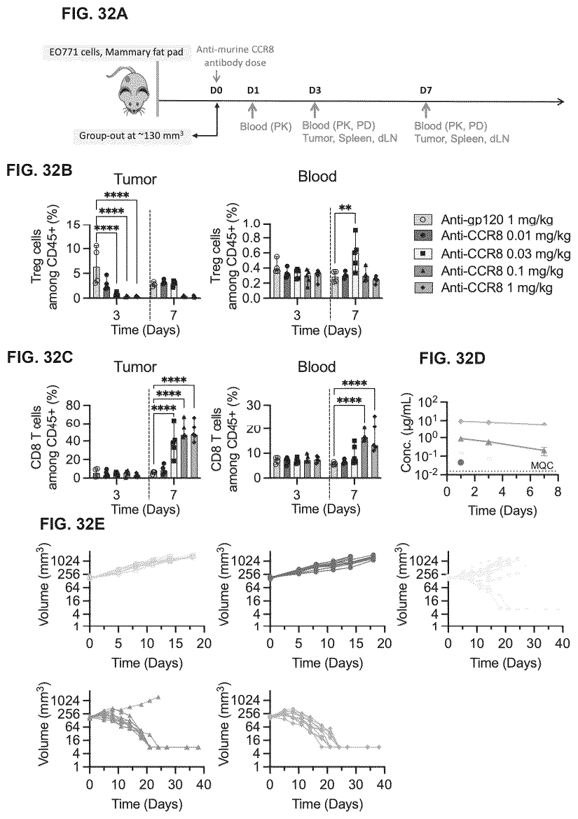

In a PD study in the syngeneic mouse E0771 breast tumor model (FIG. 32A), treatment with anti-murine CCR8 antibody led to a dose-dependent depletion of total Treg cells in tumors but no changes in other tissues, including tumor-draining lymph nodes, spleens, and blood by Day 3 post-dose (FIGS. 32B and 33A). The results showed ˜50% Treg cell depletion by Day 3 at 0.01 mg/kg, and ˜100% depletion at 0.1 mg/kg and 1 mg/kg. Full Treg cell depletion was maintained at Day 7 for the 0.1 mg/kg and 1 mg/kg dose levels. The frequencies of CD8+ T cells in tumors, tumor-draining lymph nodes, spleens, and blood were not substantially altered by Day 3 across dose levels (FIGS. 32C and 33B). However, doses of 0.03 mg/kg or higher resulted in significant increases in CD8+ T cells in tumors on Day 7. Also, doses of 0.1 mg/kg or higher resulted in an increase in CD8+ T cells in blood on Day 7. Limited PK data from this study suggests a trend of greater-than-dose-proportional increase in systemic exposures of anti-murine CCR8 antibody (based on C_{Day}1 and AUC parameters) (FIG. 32D and Table 5).

TABLE 5
Mean (±SD) of pharmacokinetic parameter estimates from non-compartmental analysis following
single IV administration of anti-murine CCR8 antibody in C57BL/6 mice bearing E0771 tumors.

| PK Parameter | Group 2 | Group 3 | Group 4 | Group 5 |
|---|---|---|---|---|
| Nominal Dose (mg/kg) | 0.01 | 0.03 | 0.1 | 1 |
| C_{Day 1} (μg/mL) | 0.0469 ± 0.00317 | 0.178 ± 0.0115 | 0.899 ± 0.0518 | 9.41 ± 0.503 |
| DN C_{Day 1} (μg/mL/(mg/kg)) | 4.69 | 5.92 | 8.99 | 9.41 |
| AUC₁₋₇ (day · μg/mL) | ND | ND | 3.12 | 46.6 |
| DN AUC₁₋₇ (day · μg/mL/(mg/kg)) | ND | ND | 31.2 | 46.6 |

C_{Day 1} = serum concentration on Day 1 post-dose; DN = dose-normalized; AUC₁₋₇ = area under the serum concentration-time curve from Days 1 to 7 post-dose; ND = not determined due to insufficient data points.

In an efficacy study using the same tumor model, administration of a single dose of anti-murine CCR8 antibody resulted in a potent dose-dependent tumor growth inhibition and showed similar effects at 0.1 mg/kg and 1.0 mg/kg with >80% of mice experiencing a partial or complete response to treatment (FIG. 32E). The efficacy results correlated with Treg cell depletion and increased CD8+ T cells in the tumor. The serum PK in the efficacy study (FIG. 34 and Table 6) was consistent with the PK data obtained from the PD study and exhibited a trend of greater-than-dose-proportional increase in systemic exposures. The in vivo mouse PK, PD and efficacy results informed the mPAD-based FiH dose determination.

TABLE 6
Mean (±SD) of pharmacokinetic parameter estimates from non-compartmental analysis
following single IV administration of anti-murine CCR8 antibody in C57BL/6 mice.

| PK Parameter | Group 2 | Group 3 | Group 4 | Group 5 |
|---|---|---|---|---|
| Nominal Dose (mg/kg) | 0.01 | 0.03 | 0.1 | 1 |
| C_{Day 1} (μg/mL) | 0.0337 ± 0.00262 | 0.122 ± 0.0128 | 0.688 ± 0.130 | 8.31 ± 0.726 |
| DN C_{Day 1} ((μg/mL)/(mg/kg)) | 3.37 ± 0.262 | 4.06 ± 0.427 | 6.88 ± 1.30 | 8.31 ± 0.726 |
| AUC₁₋₈ (day · μg/mL) | ND | ND | 2.74 ± 0.380 | 44.1 ± 4.60 |
| DN AUC₁₋₈ (day · μg/mL/(mg/kg)) | ND | ND | 27.4 ± 3.80 | 44.1 ± 4.60 |

C_{Day 1} = serum concentration on Day 1 post-dose; DN = dose-normalized; AUC₁₋₈ = area under the serum concentration-time curve from Days 1 to 8 post-dose; ND = not determined due to insufficient data points.

### Ro7502175 Exhibited Dose-Dependent Exposures, Elicited Minimal and Transient Cytokine Secretion, and Reduced CCR8+ Treg Cells in Cynomolgus Monkeys

Cynomolgus monkeys were selected as the appropriate species to investigate the PK/toxicokinetics (TK), PD profiles, cytokine modulation and safety of RO7502175 because RO7502175 binds to human and cynomolgus monkey CCR8 with comparable binding affinities.

### Single-Dose Study in Cynomolgus Monkeys

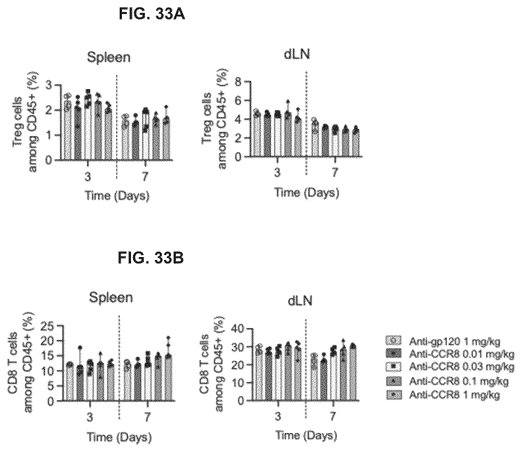

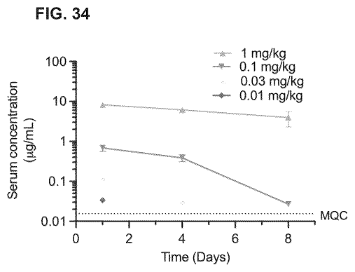

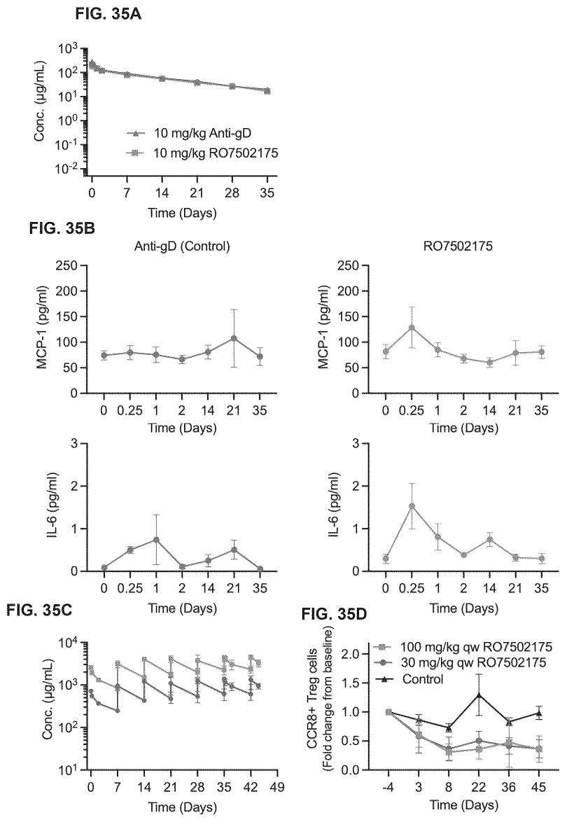

The PK of RO7502175 was evaluated following administration of a single IV dose of 10 mg/kg to cynomolgus monkeys (FIG. 35A, Table 7). RO7502175 exhibited a biphasic concentration-time profile, as expected for typical human IgG1 antibodies, characterized by a rapid initial distribution phase followed by a slower elimination phase. Systemic exposures for afucosylated anti-gD (control) and RO7502175 were comparable with respective mean clearance of 3.96±0.412 and 4.38±0.291 mL/day/kg. The clearance was consistent with that for typical human IgG1 monoclonal antibodies (see, e.g., Deng et al. MAbs. 2011; 3: 61-66). Anti-drug antibodies (ADA) were observed in 1 out of 3 animals (33%) dosed with RO7502175, but no ADA-related impact on systemic exposure was observed.

TABLE 7
Mean (±SD) of pharmacokinetic parameter estimates from non-compartmental analyses
following single IV administration of RO7502175 at 10 mg/kg in cynomolgus monkeys.

| | | Cₘₐₓ | AUCₜₗₐₛₜ | AUC_{0-inf} | CL | Vss |
|---|---|---|---|---|---|---|
| Grp | Treatment | (μg/mL) | (day · μg/mL) | (day · μg/mL) | (mL/day/kg) | (mL/kg) |
| 1 | Anti-gD | 287 ± 16.3 | 2170 ± 146 | 2540 ± 252 | 3.96 ± 0.41 | 68.4 ± 8.20 |
| 2 | RO7502175 | 222 ± 26.6 | 1980 ± 127 | 2290 ± 147 | 4.38 ± 0.291 | 74.5 ± 4.18 |

SD, standard deviation; Cₘₐₓ, maximum observed concentration; AUCₜₗₐₛₜ, area under the serum concentration time curve from time 0 to last observed quantifiable concentration; AUC_{0-inf}, area under the serum concentration time curve from time 0 extrapolated to infinity; CL, clearance; Vss, volume of distribution at steady-state.

The single IV administration of RO7502175 at 10 mg/kg was well-tolerated in cynomolgus monkeys, with no mortality, or RO7502175-related findings in clinical observations, body weights, food evaluation, or changes in clinical pathology parameters. No effects on the total lymphocyte, total T cell, helper T cell (Th), cytotoxic T lymphocytes, Treg cell, B lymphocytes, or NK-cell counts were observed in either whole blood or the inguinal lymph nodes. In some animals, CCR8+ Treg cells (CCR8+FoxP3+CD4+) were detected at a decreased frequency starting as early as 6-24 hours, and this decrease was generally maintained until the end of the study (FIG. 36B). In addition, cytokine modulation was limited to a minimal and transient increase of monocyte chemoattractant protein-1 (MCP-1) and interleukin-6 (IL-6) in a subset of cynomolgus monkeys (FIG. 35B).

### Repeat-Dose Study in Cynomolgus Monkeys

A 6-week repeat-dose study was conducted to determine the potential toxicity, characterize TK, and measure PD effect of RO7502175 at 30 or 100 mg/kg by weekly IV administration to cynomolgus monkeys for 45 days. The concentrations of RO7502175 in serum were examined over the duration of the study. Systemic exposure of RO7502175 was demonstrated in both dose groups, and the exposures were maintained for the duration of the study in all animals (FIG. 35C, Table 8). There was a dose-proportional increase in systemic exposures (based on Cmax and AUC parameters) following the first dose. No marked sex-related differences in TK were observed. Accumulation of systemic exposure (Cmax and AUC) was observed on repeat administration at both dose levels, with mean accumulation ratios of 2.42 and 2.39 in the 30 mg/kg and 100 mg/kg groups, respectively. ADAs were observed in 8 out of 16 animals (50%) across the two dose groups, with ADA first detected on Day 14. In most animals, ADA titers peaked on Day 21 or 28 and decreased thereafter. ADA occurrence was associated with no to minimal impact on systemic exposure.

TABLE 8
Mean (±SD) of pharmacokinetic parameter estimates
from non-compartmental analyses following repeat IV
administration of RO7502175 to cynomolgus monkeys.

| | Group 2 | Group 3 |
|---|---|---|
| Dose | 30 | 100 |
| (mg/kg) | | |

| | | |
|---|---|---|
| Cₘₐₓ | 687 ± 113 | 2400 ± 361 |
| (μg/mL) | | |
| Cₘₐₓ/Dose (μg*kg/mL/mg) | 22.9 ± 3.77 | 24 ± 3.61 |
| AUC₀₋₇ | 2480 ± 128 | 8730 ± 740 |
| (μg*day/mL) | | |
| AUC₀₋₇/Dose | 82.8 ± 4.26 | 87.3 ± 7.4 |
| (μg*day*kg/mL/mg) | | |
| AUC₃₅₋₄₂ | 5980 ± 1190 | 20800 ± 4020 |
| (μg*day/mL) | | |
| AUC₃₅₋₄₂/Dose | 199 ± 39.5 | 208 ± 40.2 |
| (μg*day*kg/mL/mg) | | |
| AUC₀₋₂₁ | 13200 ± 810 | 45200 ± 3380 |
| (μg*day/mL) | | |
| R_{AUC} | 2.42 ± 0.53 | 2.39 ± 0.47 |
| (AUC₃₅₋₄₂/AUC₀₋₇) | | |

First dose clearance (CL) were 6.49 ± 0.28 and 6.32 ± 1.18 mL/day/kg at 30 and 100 mg/kg doses, respectively; SD, standard deviation; Cₘₐₓ, maximum observed concentration; AUC₀₋ₜ, area under the serum concentration time curve from time 0 to t days post-dose; R_{AUC}, Accumulation ratio or AUC ratio.

In RO7502175-treated animals, the relative percentage of CCR8+ Treg cells (CCR8+FoxP3+CD4+) was reduced compared to pretreatment baseline levels, and this reduction was sustained from Day 3 until the end of the study (FIG. 35D). The PD effect was most evident on the CD45RA− ICOS+ effector Treg cell subset, which showed the highest baseline expression of the CCR8 receptor. No RO7502175-related changes were observed in the total lymphocyte counts or in the total T cell, CD4+Th, CD8+ cytotoxic T cell (Tc cell), Treg cell, B-cell, or NK-cell populations.

No RO7502175-related mortality was observed in this study. Hematology, clinical chemistry, and bone marrow effects were observed in a subset of animals treated with RO7502175 at 30 mg/kg or 100 mg/kg. Findings were first evident on Day 21 and consisted of decreases in circulating neutrophils and platelets, increases in globulin, and decreases in the albumin:globulin ratio. All impacted animals were positive for ADAs on Day 21. Despite continued dose administration with RO7502175, the neutrophil and platelet counts were reversed, and the measurements were in the normal range at the time of the terminal necropsy. A retrospective analysis demonstrated an association between transient neutropenia and ADA-induced inflammation in cynomolgus monkeys treated with afucosylated humanized monoclonal antibodies. In addition, assessment of bone marrow smears in these animals indicated a relative increase in the myeloid:erythroid (M:E) ratio, consistent with an appropriate bone marrow response to replenish the reduced neutrophils and platelets in the periphery.

Overall, weekly IV administration of RO7502175 for 45 days was well-tolerated in cynomolgus monkeys at 30 mg/kg and 100 mg/kg. The findings observed in hematology, clinical chemistry, and bone marrow cytology were considered potentially attributable to the emergence of ADAs and reversed during the course of the study, despite continued administration of RO7502175. Given the absence of RO7502175-related adverse findings, the no-observed-adverse-effect level (NOAEL) was established to be 100 mg/kg. The cynomolgus monkey PK data, safety data and cytokine secretion results supported the integrated approach used for FiH dose selection of RO7502175.

mPBPK-PD Model Captures Preclinical PK, PD, and Efficacy and Predicts Clinical PK and RO for Anti-CCR8 Antibodies

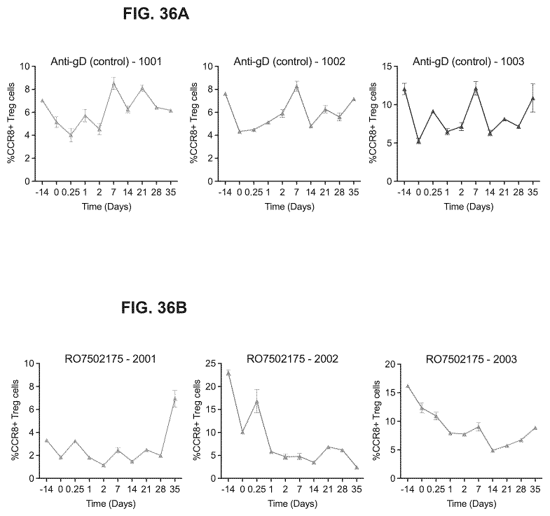

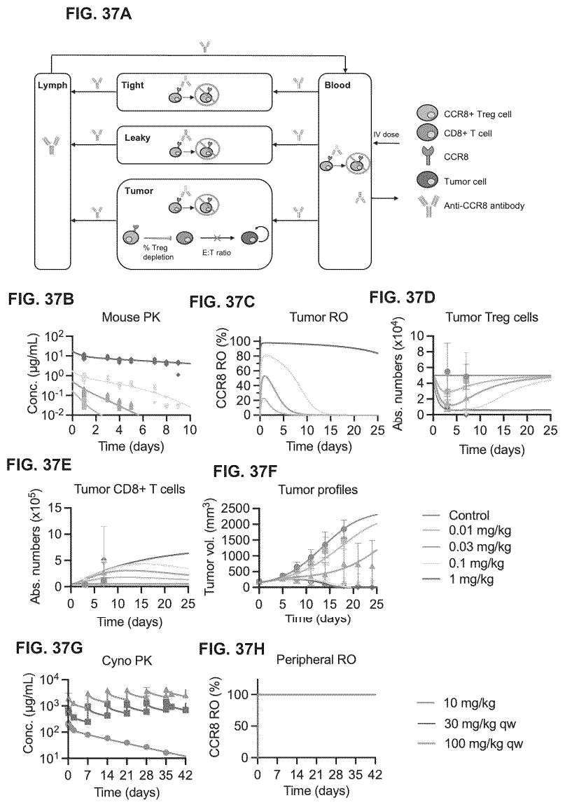

We developed a mPBPK-PD model to elucidate the PK-PD-efficacy relationship and capture the PK, RO, and PD of RO7502175 in mice, cynomolgus monkeys and humans (FIG. 37A). Briefly, the model simulates IV dosing in the central compartment, drug transport through leaky, tight, tumor (mouse and human), and lymphatic tissue, and drug clearance from the blood. The drug can bind to CCR8 receptors on CCR8+ Treg cells in relevant compartments. Following binding of the drug to CCR8 in the tumor, CCR8+ Treg cells are depleted, leading to removal of Treg cell suppression, an increase in CD8+ T cells and subsequent tumor cell killing.

The model captures the PK profiles observed in the E0771 tumor-bearing mice after single IV administration of 0.01, 0.03, 0.1, and 1 mg/kg (FIG. 37B). Upon dosing, the model predicts the CCR8 RO in the tumor over 25 days (FIG. 37C). The model uses RO-mediated cell killing to capture the in vivo mouse CCR8+ tumor Treg cell counts measured on Days 3 and 7 (FIG. 37D). Following CCR8+ tumor Treg cell depletion, the model simulates CD8+ T cell increases in the tumor (FIG. 37E) and tumor cell killing (FIG. 37F), thus providing a mathematical representation of the minimum set of biological mechanisms required to describe PK, Treg cell depletion and CD8+ T cell increase in the tumor, and anti-tumor efficacy of RO7502175 in blood and key tissue compartments in the mouse models.

The model was further developed to capture cynomolgus monkey PK data to support clinical translation, project clinical PK and inform clinical RO estimates in the tumor. The cynomolgus monkey PK model parameters were calibrated to PK data at 10 mg/kg (FIG. 37G) and then validated against 30 and 100 mg/kg IV weekly repeat dose PK data. The model predicts that ˜100% CCR8 RO is maintained in blood over all three of these dosing regimens for at least 40 days (FIG. 37H).

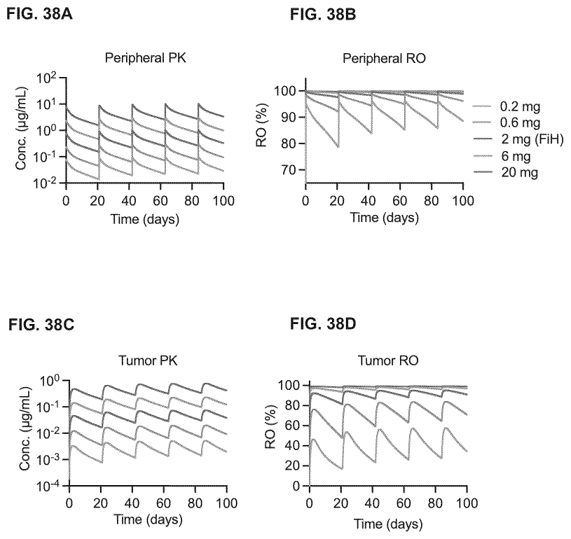

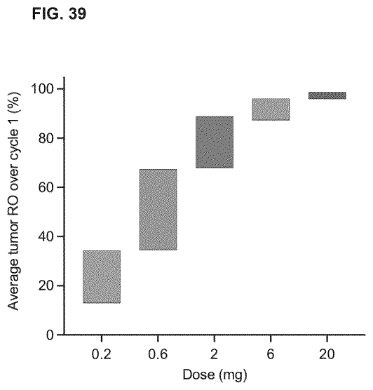

The mPBPK-PD model predicts the clinical PK/RO relationship to support FiH dose selection using expected human target expression levels. Clinical PK was simulated at dose levels of 0.2, 0.6, 2, 6, and 20 mg IV q3w to examine the PK and RO in the blood and tumor (FIGS. 38A-38D). Human PK clearance for RO7502175 was scaled from the cynomolgus monkey clearance using allometric scaling with an exponent of 0.85 (see, e.g., Deng et al. MAbs. 2011; 3: 61-66). The scaled human clearance of 2.8 mL/day/kg and elimination half-life of approximately 21 days support a q3w clinical dosing frequency. At 2 mg, the model predicts an average RO in the tumor in cycle 1 of approximately 80% (assumes ˜5-10% drug-partitioning to tumor) (FIG. 38D). A sensitivity analysis to assess uncertainty in parameter values (plasma volume, nonspecific clearance, tumor partitioning, and antibody binding affinity) identified that a dose of 2 mg would result in an average RO of 80% (68-89%) in the tumor in cycle 1 (FIG. 39).

### RO7502175 FiH Dose Selection was Based on an Integrated Approach Utilizing the Comprehensive Nonclinical Data

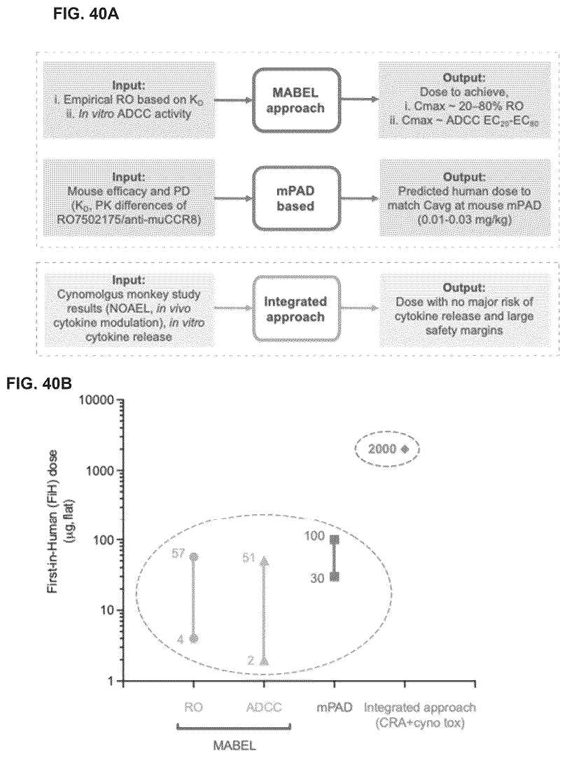

A starting dose of 2 mg IV was selected for the FiH clinical study in patients with advanced solid tumors (Table 9) using an integrated approach that was based on the totality of nonclinical data for RO7502175 (FIGS. 40A and 40B), was supported by clinical experience with other Treg-cell-targeting afucosylated molecules and was guided by insights from the mPBPK-PD model. There were no RO7502175-related toxicity findings in the single-dose (10 mg/kg IV) and repeat-dose (30 and 100 mg/kg IV qw) studies in cynomolgus monkeys, while pharmacologic activity (reflected by reduction of CCR8+ Treg cells in peripheral blood) was observed in both studies. The highest dose of RO7502175 tested (100 mg/kg) in the repeat-dose toxicology study was identified as the NOAEL and this provides a safety margin of greater than 3000-fold for the proposed starting dose of 2 mg in humans (Table 9). In the single-dose study, treatment with 10 mg/kg IV RO7502175 resulted in minimal and transient cytokine secretion in a subset of cynomolgus monkeys. RO7502175 did not induce cytokine release in an in vitro human PBMC assay at concentrations up to 1500 μg/mL, the highest concentration assessed, in the soluble assay format. This provides approximately 1900-fold safety margin compared to the predicted Cmax (0.77 μg/mL) at the proposed FiH dose. These observations suggest that RO7502175 poses a low risk of inducing cytokine release syndrome (CRS) in the clinic. Moreover, molecules with afucosylated Fc have been safely administered in the clinic and suggest low risk for exaggerated infusion related reactions (IRRs) and CRS. For instance, administration of mogamulizumab (anti-CCR4) at 0.1-1 mg/kg qw in the clinic was reported to be well-tolerated with a manageable safety profile. Clinical administration of anti-CD25 (RO7296682, afucosylated Fc) as a monotherapy in a Phase I dose-escalation study to 29 patients with advanced solid tumors at 0.3-35 mg IV q3w was well-tolerated. CCR8 has a more restricted expression profile (lower copy number per cell and more selective expression in tumor) than CCR4 and CD25, both of which have been used as targets for Treg cell depletion. Furthermore, the average RO over the first dosing interval of 21 days in the tumor (target site) is predicted to be about 80% based on the mPBPK-PD model simulations, at the proposed starting dose. This metric was used as an anchor point to select 2 mg as the FiH dose.

TABLE 9
Proposed First-in-Human dose for RO7502175 is supported by in
vitro cytokine release and cynomolgus monkey toxicology data.

| Proposed | Predicted | | |
|---|---|---|---|
| FiH dose | Cmax^{a} | Cytokine assay | Safety Factor |
| 2 mg | 0.77 μg/mL | covered up to 1500 μg/mL | AUC - 7386^{b} |
| (0.03 mg/kg) | | | Cmax - 3117^{c} |
| | | | Dose - 3333 |

Cₘₐₓ, maximum observed concentration; AUC, area under the serum concentration time curve; ^{a}Predicted Cmax in humans after first dose of 2 mg IV, assumes 70 kg body weight; ^{b}Predicted human AUC₍₀₋₂₁₎ following 2 mg dose compared to observed AUC₍₀₋₂₁₎ at NOAEL of 100 mg/kg IV qw in repeat-dose toxicology study; ^{c}Predicted first dose Cmax in humans after 2 mg dose compared with observed Cmax in monkeys following 100 mg/kg.

Other approaches were also considered for the selection of FiH dose including a MABEL dose based on in vitro activity readouts and empirical RO in circulation, and a mPAD derived from in vivo mouse studies (FIG. 40A). Using a MABEL approach based on EC20-EC80 from in vitro ADCC or 20-80% empirical RO in peripheral blood will result in a FiH dose range of 2-51 μg or 4-57 μg, respectively (FIG. 40B). Additionally, a mPAD approach was used based on mouse studies assessing in vivo efficacy and PD activity in tumor models and resulted in a FiH dose range of 30-100 μg (FIG. 40B). The in vivo mPAD approach accounted for differences in target binding affinity as well as PK properties between the anti-murine CCR8 antibody and RO7502175. However, these methods have limitations that are discussed in the next section and also resulted in a very low starting dose that would expose patients to sub-therapeutic doses, and hence were not deemed appropriate for RO7502175 with a superior preclinical safety profile.

### DISCUSSION AND CONCLUSIONS

In the current work, we studied an afucosylated antibody, RO7502175, designed to eliminate CCR8+ Treg cells, which are enriched in tumors. RO7502175 exhibited enhanced binding to FcγRIIIA and potent ADCC activity in in vitro assays. In mouse studies, a surrogate anti-murine CCR8 antibody demonstrated efficient dose-dependent tumor-specific Treg cell depletion and led to an increase in CD8+ T cells in the tumor, and potent anti-tumor efficacy in the E0771 breast carcinoma model. Efficacy correlated with Treg cell depletion and increases in CD8+ T cells in the tumor. In the cynomolgus monkey studies, RO7502175 exhibited a biphasic systemic concentration-time profile as expected for typical human IgG1 antibodies and the PK exposures were dose proportional for the evaluated dose levels. RO7502175 caused a decrease in CCR8+ Treg cells in the blood of cynomolgus monkeys, demonstrating evidence of PD activity. RO7502175 was well-tolerated in cynomolgus monkeys with no treatment-related adverse findings and the NOAEL was established as 100 mg/kg. RO7502175 treatment resulted in only minimal and transient cytokine secretion in the single-dose study. Furthermore, RO7502175 elicited minimal cytokine release in an in vitro PBMC assay with soluble format, suggesting a low risk for excessive cytokine release in patients. While the in vitro ADCC and mouse studies established the proof-of-concept for targeted depletion of CCR8+ Treg cells to enhance anti-tumor immune responses, the results from cynomolgus monkey studies and in vitro cytokine assays support the safety profile of RO7502175.

While a trend of greater-than-dose-proportional increase in systemic exposures of anti-murine CCR8 antibody was observed in mouse studies, the expected CCR8 target capacity is too low to explain that the higher clearance at low dose levels was due to target-mediated drug disposition (TMDD). Furthermore, the PD results showed CCR8+ Treg cell depletion in tumor by Day 3 following 0.1 and 1 mg/kg doses and was sustained at Day 7 in mouse studies, suggesting that a significant level of TMDD is unlikely at later time points due to extremely low target levels. Also, ADA could have potentially impacted the PK at later time points, however, ADA measurements were not available from mouse studies. Taken together, the non-linearity in PK could be specific to the anti-murine CCR8 antibody and the findings are not expected to translate to RO7502175 in patients.

We developed a mPBPK-PD model that captures the anti-murine CCR8 antibody PK, PD, and tumor growth inhibition in mice, and RO7502175 PK in cynomolgus monkeys. The modeling mechanistically represents anti-CCR8 antibody PK, binding to CCR8 receptors, CCR8+ Treg cell depletion, CD8+ T cell expansion in the tumor, and tumor cell killing. CD8+ T cells in the tumor were included in the model to account for the observed time delay between the observed tumor Treg cell depletion and anti-tumor efficacy in mice. Our mechanistic model aimed to provide a mathematical representation of the minimum set of biological mechanisms required to support the MOA hypothesis for anti-CCR8 and reproduce the observed PK, PD, and anti-tumor efficacy in the mouse models. This model was used to project RO7502175 clinical PK and RO profiles in plasma and tumors of patients after translating relevant PK parameters and expected target expression levels. We expect linear PK behavior for RO7502175 in patients due to the low target expression levels. The human PK and RO projections informed the Phase I clinical study design for RO7502175 in cancer patients. Moreover, the model will be used to capture clinical data (PK, PD, and efficacy) as it becomes available and will be refined to describe emerging clinical data and to inform future clinical decisions.

The FiH dose of 2 mg IV for RO7502175 in patients with advanced solid tumors was selected using an integrated approach that was based on the totality of nonclinical data for RO7502175 (safety in cynomolgus monkey single- and repeat-dose studies, in vivo cytokine modulation in single-dose cynomolgus monkey study, and in vitro cytokine release in PBMC assay), was guided by insights from the mPBPK-PD model, and was supported by clinical experience with other Treg-cell-depleting afucosylated antibodies. The preclinical data for RO7502175 supports 2 mg as a safe starting dose in the clinic. At the proposed FiH dose, the average RO over the first dosing interval of 21 days in the tumor is predicted to be about 80% based on mPBPK-PD model simulations. Given the large safety margins based on results from cynomolgus monkey studies, we relied on clinical RO predictions at the site of action to allow us to determine a suitable starting dose. Although the predicted RO in circulation is ˜99% at Cmax following 2 mg dose in the clinic, the RO at the site of action, i.e., tumor, is more relevant. The mPBPK-PD model framework provided a quantitative metric as an anchor point to select the proposed FiH dose of 2 mg. Overall, the proposed FiH dose of 2 mg IV is expected to be safe, provide some level of pharmacological activity and minimize administration of sub-therapeutic dose levels to patients.

Although we considered multiple methods for FiH dose selection including MABEL-based and mPAD-based approaches, we believe that these approaches are not appropriate for this antibody with a superior preclinical safety profile. CCR8 is predominantly expressed on tumor-resident Treg cells with minimal off-tumor-site expression. The antibody is not a direct immune activator but rather binds a target with low expression in the tumor, to enable ADCC-mediated depletion of CCR8+ Treg cells enriched in tumors. Therefore, FiH dose selection based on the MABEL approach using RO in circulation is not relevant. Moreover, a conservative MABEL approach based on ADCC would result in a much lower starting dose for this antibody, which has a superior safety profile demonstrated by minimal cytokine modulation and a high NOAEL of 100 mg/kg in cynomolgus monkey. Consequently, more dose levels would potentially be needed to escalate to a therapeutic dose range in the clinic, exposing a larger number of patients to sub-therapeutic dose levels. For these reasons, MABEL based FiH dose selection was not used for RO7502175. On the other hand, results from mouse studies assessing in vivo efficacy and PD activity in tumor models were used for identifying mPAD, and FiH dose selection using mPAD based approach was also evaluated but not considered. These studies utilized a murine surrogate clone since RO7502175 does not cross-react with rodent CCR8. Some differences such as target binding affinity as well as PK properties between the murine surrogate clone and RO7502175 were factored in the determination of mPAD based starting dose.

Overall, based on the aforementioned rationale and considerations, we believe that the integrated approach and the modeling analysis used for FiH dose selection is more appropriate for RO7502175. Our approach would allow about three to four fewer dose escalations in patients, assuming half-log increments in dose between cohorts in the clinic. The selected starting dose of 2 mg would allow a safe entry in the clinic with the potential to reach the therapeutic dose range in a reasonable timeframe in a safe manner. The proposed FiH dose selection strategy for the Phase 1 clinical study has been accepted by health authorities in the US (clinicaltrials.gov identifier: NCT05581004) and across the globe.

### OTHER EMBODIMENTS

Although the foregoing has been described in some detail by way of illustration and example for purposes of clarity of understanding, the descriptions and examples should not be construed as limiting the scope of the present disclosure. All patents and scientific literature cited herein are expressly incorporated in their entirety by reference.

1. A method of treating a locally advanced, recurrent, or metastatic solid tumor malignancy in a subject in need thereof, the method comprising administering to the subject a monoclonal antibody that binds to C-C motif chemokine receptor 8 (CCR8).

2. The method of claim 1, wherein:
(i) the subject has progressed after at least one available standard therapy; or
(ii) the subject is one for whom all available standard therapy has been proven to be ineffective or intolerable or is contraindicated;
(iii) the locally advanced, recurrent, or metastatic solid tumor is incurable;
(iv) the subject's age is 18 years or older;
(v) the locally advanced, recurrent, or metastatic solid tumor malignancy is non-small cell lung cancer (NSCLC), head and neck squamous cell carcinoma (HNSCC), melanoma, triple-negative breast cancer (TNBC), urothelial carcinoma (UC), esophageal cancer, gastric cancer, cervical cancer, renal cell carcinoma (RCC), or hepatocellular carcinoma (HCC);
(vi) a tumor sample from the subject has been determined to have a detectable level of PD-L1 expression; and/or
(vii) the monoclonal antibody that binds to CCR8 is administered to the subject as a monotherapy.

3-5. (canceled)

6. The method of claim 2, wherein:
(a)
(i) the locally advanced, recurrent, or metastatic solid tumor malignancy is RCC, and the RCC is clear cell RCC;
(ii) the locally advanced, recurrent, or metastatic solid tumor malignancy is HNSCC, and the HNSCC is HNSCC of the oral cavity, oropharynx, hypopharynx, or larynx;
(iii) the locally advanced, recurrent, or metastatic solid tumor malignancy is NSCLC, wherein the subject's tumor comprises a targetable somatic alteration, and the subject has experienced disease progression during or after treatment, or intolerance to treatment, with a targeted agent;
(iv) the locally advanced, recurrent, or metastatic solid tumor malignancy is melanoma, and the melanoma is cutaneous melanoma;
(v) the locally advanced, recurrent, or metastatic solid tumor malignancy is UC, and wherein the subject has (1) histologically confirmed incurable advanced transitional cell carcinoma of the urothelium; and/or (2) a mixed histology, wherein the subject's tumor has a dominant transitional cell pattern; or
(vi) the locally advanced, recurrent, or metastatic solid tumor malignancy is TNBC, and the TNBC is defined by the American Society of Clinical Oncology-College of American Pathologists guidelines for (1) having <1% of tumor-cell nuclei being immunoreactive for estrogen receptor and <1% of tumor-cell nuclei being immunoreactive for progesterone receptor; and/or (2) being HER2-negative based on immunohistochemistry (IHC) and/or in situ hybridization; and/or
(b) the tumor sample from the subject has a Tumor Cell (TC) Score, an Immune Cell (IC) Score, a Combined Positive Score (CPS), or a Tumor Proportion Score (TPS) of PD-L1 expression level greater than or equal to 1%.

7. (canceled)

8. The method of claim 2, wherein the locally advanced, recurrent, or metastatic solid tumor malignancy is NSCLC, wherein the subject's tumor comprises a targetable somatic alteration, and the subject has experienced disease progression during or after treatment, or intolerance to treatment, with a targeted agent.

9. (canceled)

10. The method of claim 6, wherein:
(i) the locally advanced, recurrent, or metastatic solid tumor malignancy is NSCLC, and wherein the targetable somatic alteration comprises a somatic alteration involving epidermal growth factor receptor (EGFR), anaplastic lymphoma kinase (ALK), ROS proto-oncogene 1 (ROS1), proto-oncogene B-Raf (BRAF) V600E, neurotrophic tyrosine receptor kinase (NTRK), MET proto-oncogene (MET), RET proto-oncogene (RET), or Kirsten rat sarcoma virus (KRAS);
(ii) the locally advanced, recurrent, or metastatic solid tumor malignancy is cutaneous melanoma, and wherein the subject's tumor comprises a BRAFV600 mutation, and the subject has experienced disease progression during or after treatment, or intolerance to treatment, with one or more serine/threonine-protein kinase B-Raf (BRAF) inhibitors and/or one or more mitogen-activated protein kinase kinase (MEK) inhibitors; or
(iii) the locally advanced, recurrent, or metastatic solid tumor malignancy is UC, and wherein the urothelium comprises one or more of renal pelvis, ureters, urinary bladder, and urethra.

11-16. (canceled)

17. The method of claim 1, wherein the subject is checkpoint inhibitor (CPI)-naïve or CPI-experienced.

18. The method of claim 17, wherein:
(a) the subject is CPI-naïve, and:
(i) the subject's locally advanced, recurrent, or metastatic solid tumor malignancy is NSCLC or UC,
(ii) the subject has had no prior treatment with a CPI, or the subject has had adjuvant treatment with a CPI that was discontinued at least six months prior to first administration of the monoclonal antibody that binds to CCR8 to the subject; and/or
(iii) the CPI is a PD-1 axis binding antagonist or a CTLA4 antagonist; or
(b) the subject is CPI-experienced, and:
(i) the subject's locally advanced, recurrent, or metastatic solid tumor malignancy is NSCLC, HNSCC, melanoma, UC, TNBC, esophageal cancer, gastric cancer, cervical cancer, clear cell RCC, or HCC;
(ii) the subject derived clinical benefit from treatment comprising a PD-1 axis binding antagonist prior to disease progression;
(iii) (1) the subject has not received treatment with a CPI, an immunomodulatory monoclonal antibody, or an immunomodulatory monoclonal antibody-derived therapy within 6 weeks prior to first administration of the monoclonal antibody that binds to CCR8 to the subject; or (2) the subject was previously treated with a PD-1 axis binding antagonist, and the last administration of the PD-1 axis binding antagonist to the subject was at least 3 weeks prior to first administration of the monoclonal antibody that binds to CCR8 to the subject; and/or
(iv) the CPI is a PD-1 axis binding antagonist or a CTLA4 antagonist.

19. The method of claim 18, wherein:
(a) the subject is CPI-naïve, and:
(i) the subject's locally advanced, recurrent, or metastatic solid tumor malignancy is UC, wherein the subject is eligible for treatment with cisplatin, and the subject has experienced disease progression during or after treatment, or intolerance to treatment, with cisplatin; and/or
(ii) the PD-1 axis binding antagonist is an anti-PD-L1 antibody or an anti-PD-1 antibody; or
(b) the subject is CPI-experienced, and:
(i) the PD-1 axis binding antagonist is an anti-PD-L1 antibody or an anti-PD-1 antibody; and/or
(ii) the subject had a treatment duration with the treatment comprising the PD-1 axis binding antagonist of greater than or equal to 6 months and/or had a partial response or complete response as best objective response.

20-28. (canceled)

29. The method of claim 1, wherein:
(i) the monoclonal antibody that binds to CCR8 is administered to the subject in a dosing regimen comprising one or more dosing cycles;
(ii) the monoclonal antibody that binds to CCR8 is administered to the subject at a dose of 2 mg;
(iii) the monoclonal antibody that binds to CCR8 is administered to the subject intravenously; and/or
(iv) the monoclonal antibody that binds to CCR8 is administered to the subject in combination with one or more additional therapeutic agents.

30. The method of claim 29, wherein:
(i) the one or more dosing cycles of the monoclonal antibody that binds to CCR8 comprise 21-day dosing cycles;
(ii) the monoclonal antibody that binds to CCR8 is administered to the subject until disease progression or unacceptable toxicity;
(iii) the monoclonal antibody that binds to CCR8 is administered to the subject intravenously by infusion; and/or
(iv) the monoclonal antibody that binds to CCR8 is administered to the subject in combination with one or more additional therapeutic agents, and the one or more additional therapeutic agents comprises atezolizumab.

31. The method of claim 30, wherein:
(i) the monoclonal antibody that binds to CCR8 is administered to the subject on Day 1 of each 21-day dosing cycle;
(ii) the atezolizumab is administered to the subject in a dosing regimen comprising one or more dosing cycles;
(iii) the atezolizumab is administered to the subject at a dose of 1200 mg;
(iv) the atezolizumab is administered to the subject intravenously; and/or
(v) the subject has received at least two cycles of the monoclonal antibody that binds to CCR8 prior to administration of atezolizumab to the subject.

32-39. (canceled)

40. The method of claim 31, wherein:
(i) the one or more dosing cycles of atezolizumab comprise 21-day dosing cycles; and/or
(ii) the atezolizumab is administered to the subject intravenously by infusion.

41. The method of claim 40, wherein the atezolizumab is administered to the subject on Day 1 of each 21-day dosing cycle.

42-47. (canceled)

48. The method of claim 1, wherein the monoclonal antibody that binds to CCR8 comprises a heavy chain variable domain (VH) comprising (a) a CDR-H1 comprising the amino acid sequence of SEQ ID NO: 29 or SEQ ID NO: 30, (b) a CDR-H2 comprising the amino acid sequence of SEQ ID NO: 31, and (c) a CDR-H3 comprising the amino acid sequence of SEQ ID NO: 32, and a light chain variable domain (VL) comprising (d) a CDR-L1 comprising the amino acid sequence of SEQ ID NO: 26, (e) a CDR-L2 comprising the amino acid sequence of SEQ ID NO: 27, and (f) a CDR-L3 comprising the amino acid sequence of SEQ ID NO: 28.

49. The method of claim 48, wherein:
(i) the monoclonal antibody that binds to CCR8 binds to CCR8 independent of sulfation of CCR8;
(ii) the monoclonal antibody that binds to CCR8 binds to an epitope comprising one or more of amino acid residues 2-6 of SEQ ID NO: 106;
(iii) the monoclonal antibody that binds to CCR8 comprises a sequence selected from the group consisting of (1) a VH sequence having at least about 95%, at least about 96%, at least about 97%, at least about 98%, or at least about 99% sequence identity to an amino acid sequence selected from the group consisting of SEQ ID NOs: 35-47: (2) a VL sequence having at least about 95%, at least about 96%, at least about 97%, at least about 98%, or at least about 99% sequence identity to an amino acid sequence selected from the group consisting of SEQ ID NOs: 48-52; and (3) a VH sequence as defined in (1) and a VL sequence as defined in (2);
(iv) the VL comprises (1) a V4M mutation, a P43A mutation, a F46L mutation, a C90Q mutation, or a combination thereof (numbering according to Kabat); or (2) a C90Q mutation and a Y21 mutation (numbering according to Kabat);
(v) the VH comprises (1) a G49S mutation, a K71R mutation, a S73N mutation, or a combination thereof (numbering according to Kabat); or (2) a V78L mutation, a T76N mutation, a F91Y mutation, and a P105Q mutation (numbering according to Kabat); and/or
(vi) the VH comprises (1) a CDR-H1 comprising the amino acid sequence of SEQ ID NO: 29, (2) a CDR-H2 comprising the amino acid sequence of SEQ ID NO: 31, and (3) a CDR-H3 comprising the amino acid sequence of SEQ ID NO: 32, and the VL comprises (4) a CDR-L1 comprising the amino acid sequence of SEQ ID NO: 26, (5) a CDR-L2 comprising the amino acid sequence of SEQ ID NO: 27, and (6) a CDR-L3 comprising the amino acid sequence of SEQ ID NO: 28.

50-51. (canceled)

52. The method of claim 49, wherein:
(a) the monoclonal antibody that binds to CCR8 comprises:
(i) a VH sequence selected from the group consisting of SEQ ID NOs: 35-47 and a VL sequence selected from the group consisting of SEQ ID NOs: 48-52;
(ii) a sequence selected from the group consisting of (1) a VH sequence having at least about 95%, at least about 96%, at least about 97%, at least about 98%, or at least about 99% sequence identity to an amino acid sequence of SEQ ID NO: 47: (2) a VL sequence having at least about 95%, at least about 96%, at least about 97%, at least about 98%, or at least about 99% sequence identity to an amino acid sequence of SEQ ID NO: 48; and (3) a VH sequence as defined in (1) and a VL sequence as defined in (2); or
(iii) a VH sequence having at least about 95%, at least about 96%, at least about 97%, at least about 98%, or at least about 99% sequence identity to the amino acid sequence of SEQ ID NO: 47 and a VL sequence having at least about 95%, at least about 96%, at least about 97%, at least about 98%, or at least about 99% sequence identity to the amino acid sequence of SEQ ID NO: 48;
(b) the monoclonal antibody that binds to CCR8 comprises the heavy chain amino acid sequence of SEQ ID NO: 55, 60, 111, or 113, and the light chain amino acid sequence of SEQ ID NO: 56; and/or
(c) the VH comprises a V78L mutation, a T76N mutation, a F91Y mutation, and a P105Q mutation, and the VL comprises a C90Q mutation and a Y21 mutation (numbering according to Kabat).

53-60. (canceled)

61. The method of claim 1, wherein the monoclonal antibody that binds to CCR8 comprises:
(a) the VH sequence of SEQ ID NO: 47 and the VL sequence of SEQ ID NO: 48;
(b) a VH comprising (i) a CDR-H1 comprising the amino acid sequence of SEQ ID NO: 4 or SEQ ID NO: 5, (ii) a CDR-H2 comprising the amino acid sequence of SEQ ID NO: 6, and (iii) a CDR-H3 comprising the amino acid sequence of SEQ ID NO: 7, and a VL comprising (iv) a CDR-L1 comprising the amino acid sequence of SEQ ID NO: 1, (v) a CDR-L2 comprising the amino acid sequence of SEQ ID NO: 2, and (vi) a CDR-L3 comprising the amino acid sequence of SEQ ID NO: 3;
(c) the VH sequence of SEQ ID NO: 21 and the VL sequence of SEQ ID NO: 24;
(d) a VH comprising (i) a CDR-H1 comprising the amino acid sequence of SEQ ID NO: 82 or SEQ ID NO: 83, (ii) a CDR-H2 comprising the amino acid sequence of SEQ ID NO: 84, and (iii) a CDR-H3 comprising the amino acid sequence of SEQ ID NO: 85, and a VL comprising (iv) a CDR-L1 comprising the amino acid sequence of SEQ ID NO: 73, (v) a CDR-L2 comprising the amino acid sequence of SEQ ID NO: 74, and (vi) a CDR-L3 comprising the amino acid sequence of SEQ ID NO: 75;
(e) a VH comprising (i) a CDR-H1 comprising the amino acid sequence of SEQ ID NO: 86 or SEQ ID NO: 87, (ii) a CDR-H2 comprising the amino acid sequence of SEQ ID NO: 88, and (iii) a CDR-H3 comprising the amino acid sequence of SEQ ID NO: 89, and a VL comprising (iv) a CDR-L1 comprising the amino acid sequence of SEQ ID NO: 76, (v) a CDR-L2 comprising the amino acid sequence of SEQ ID NO: 77, and (vi) a CDR-L3 comprising the amino acid sequence of SEQ ID NO: 78;
(f) a VH comprising (i) a CDR-H1 comprising the amino acid sequence of SEQ ID NO: 90 or SEQ ID NO: 91, (ii) a CDR-H2 comprising the amino acid sequence of SEQ ID NO: 92, and (iii) a CDR-H3 comprising the amino acid sequence of SEQ ID NO: 93, and a VL comprising (iv) a CDR-L1 comprising the amino acid sequence of SEQ ID NO: 79, (v) a CDR-L2 comprising the amino acid sequence of SEQ ID NO: 80, and (vi) a CDR-L3 comprising the amino acid sequence of SEQ ID NO: 81; or
(q) a VH comprising (i) a CDR-H1 comprising the amino acid sequence of SEQ ID NO: 65 or SEQ ID NO: 66, (ii) a CDR-H2 comprising the amino acid sequence of SEQ ID NO: 67 and (iii) a CDR-H3 comprising the amino acid sequence of SEQ ID NO: 68, and a VL comprising (iv) a CDR-L1 comprising the amino acid sequence of SEQ ID NO: 62, (v) a CDR-L2 comprising the amino acid sequence of SEQ ID NO: 63, and (vi) a CDR-L3 comprising the amino acid sequence of SEQ ID NO: 64.

62-63. (canceled)

64. The method of claim 61, wherein:
(a) the monoclonal antibody that binds to CCR8 comprises a VH comprising (i) a CDR-H1 comprising the amino acid sequence of SEQ ID NO: 4 or SEQ ID NO: 5, (ii) a CDR-H2 comprising the amino acid sequence of SEQ ID NO: 6, and (iii) a CDR-H3 comprising the amino acid sequence of SEQ ID NO: 7, and a VL comprising (iv) a CDR-L1 comprising the amino acid sequence of SEQ ID NO: 1, (v) a CDR-L2 comprising the amino acid sequence of SEQ ID NO: 2, and (vi) a CDR-L3 comprising the amino acid sequence of SEQ ID NO: 3; and wherein:
(i) the monoclonal antibody binds to CCR8 independent of sulfation of CCR8;
(ii) the monoclonal antibody binds to an epitope comprising one or more of amino acid residues 91-104 and 172-193 of SEQ ID NO: 106; and/or
(iii) the monoclonal antibody comprises a sequence selected from the group consisting of (1) a VH sequence having at least about 95%, at least about 96%, at least about 97%, at least about 98%, or at least about 99% sequence identity to an amino acid sequence selected from the group consisting of SEQ ID NOs: 10-21: (2) a VL sequence having at least about 95%, at least about 96%, at least about 97%, at least about 98%, or at least about 99% sequence identity to an amino acid sequence selected from the group consisting of SEQ ID NOs: 22-25; and (3) a VH sequence as defined in (1) and a VL sequence as defined in (2);
(iv) the VL comprises a Y21 mutation (numbering according to Kabat);
(v) the VH comprises a S73N mutation, a V78L mutation, a T76N mutation, a F91Y mutation, and a P105Q mutation, or a combination thereof (numbering according to Kabat); and/or
(vi) the monoclonal antibody comprises the heavy chain amino acid sequence of SEQ ID NO: 57, 61, 112, or 114, and the light chain amino acid sequence of SEQ ID NO: 58;
(b) the monoclonal antibody that binds to CCR8 comprises a VH comprising (i) a CDR-H1 comprising the amino acid sequence of SEQ ID NO: 82 or SEQ ID NO: 83, (ii) a CDR-H2 comprising the amino acid sequence of SEQ ID NO: 84, and (iii) a CDR-H3 comprising the amino acid sequence of SEQ ID NO: 85, and a VL comprising (iv) a CDR-L1 comprising the amino acid sequence of SEQ ID NO: 73, (v) a CDR-L2 comprising the amino acid sequence of SEQ ID NO: 74, and (vi) a CDR-L3 comprising the amino acid sequence of SEQ ID NO: 75; and wherein:
(i) the monoclonal antibody comprises a sequence selected from the group consisting of (1) a VH sequence having at least about 95%, at least about 96%, at least about 97%, at least about 98%, or at least about 99% sequence identity to an amino acid sequence of SEQ ID NO: 95: (2) a VL sequence having at least about 95%, at least about 96%, at least about 97%, at least about 98%, or at least about 99% sequence identity to an amino acid sequence of SEQ ID NO: 94; and (3) a VH sequence as defined in (1) and a VL sequence as defined in (2); and/or
(ii) the monoclonal antibody comprises the heavy chain amino acid sequence of SEQ ID NO: 101 or 115, and the light chain amino acid sequence of SEQ ID NO: 100:
(c) the monoclonal antibody that binds to CCR8 comprises a VH comprising (i) a CDR-H1 comprising the amino acid sequence of SEQ ID NO: 86 or SEQ ID NO: 87, (ii) a CDR-H2 comprising the amino acid sequence of SEQ ID NO: 88, and (iii) a CDR-H3 comprising the amino acid sequence of SEQ ID NO: 89, and a VL comprising (iv) a CDR-L1 comprising the amino acid sequence of SEQ ID NO: 76, (v) a CDR-L2 comprising the amino acid sequence of SEQ ID NO: 77, and (vi) a CDR-L3 comprising the amino acid sequence of SEQ ID NO: 78; and wherein:
(i) the monoclonal antibody comprises a sequence selected from the group consisting of (1) a VH sequence having at least about 95%, at least about 96%, at least about 97%, at least about 98%, or at least about 99% sequence identity to an amino acid sequence of SEQ ID NO: 97: (2) a VL sequence having at least about 95%, at least about 96%, at least about 97%, at least about 98%, or at least about 99% sequence identity to an amino acid sequence of SEQ ID NO: 96; and (3) a VH sequence as defined in (1) and a VL sequence as defined in (2); and/or
(ii) the monoclonal antibody that binds to CCR8 comprises the heavy chain amino acid sequence of SEQ ID NO: 103 or 116, and the light chain amino acid sequence of SEQ ID NO: 102;
(d) the monoclonal antibody that binds to CCR8 comprises a VH comprising (i) a CDR-H1 comprising the amino acid sequence of SEQ ID NO: 90 or SEQ ID NO: 91, (ii) a CDR-H2 comprising the amino acid sequence of SEQ ID NO: 92, and (iii) a CDR-H3 comprising the amino acid sequence of SEQ ID NO: 93, and a VL comprising (iv) a CDR-L1 comprising the amino acid sequence of SEQ ID NO: 79, (v) a CDR-L2 comprising the amino acid sequence of SEQ ID NO: 80, and (vi) a CDR-L3 comprising the amino acid sequence of SEQ ID NO: 81; and wherein:
(i) the monoclonal antibody comprises a sequence selected from the group consisting of (1) a VH sequence having at least about 95%, at least about 96%, at least about 97%, at least about 98%, or at least about 99% sequence identity to an amino acid sequence of SEQ ID NO: 99: (2) a VL sequence having at least about 95%, at least about 96%, at least about 97%, at least about 98%, or at least about 99% sequence identity to an amino acid sequence of SEQ ID NO: 98; and (3) a VH sequence as defined in (1) and a VL sequence as defined in (2); and/or
(ii) the monoclonal antibody comprises the heavy chain amino acid sequence of SEQ ID NO: 105 or 117, and the light chain amino acid sequence of SEQ ID NO: 104; or
(e) the monoclonal antibody that binds to CCR8 comprises a VH comprising (i) a CDR-H1 comprising the amino acid sequence of SEQ ID NO: 65 or SEQ ID NO: 66, (ii) a CDR-H2 comprising the amino acid sequence of SEQ ID NO: 67 and (iii) a CDR-H3 comprising the amino acid sequence of SEQ ID NO: 68, and a VL comprising (iv) a CDR-L1 comprising the amino acid sequence of SEQ ID NO: 62, (v) a CDR-L2 comprising the amino acid sequence of SEQ ID NO: 63, and (vi) a CDR-L3 comprising the amino acid sequence of SEQ ID NO: 64; and wherein:
(i) the monoclonal antibody comprises a sequence selected from the group consisting of (1) a VH sequence having at least about 95%, at least about 96%, at least about 97%, at least about 98%, or at least about 99% sequence identity to an amino acid sequence of SEQ ID NO: 70: (2) a VL sequence having at least about 95%, at least about 96%, at least about 97%, at least about 98%, or at least about 99% sequence identity to an amino acid sequence of SEQ ID NO: 69; and (3) a VH sequence as defined in (1) and a VL sequence as defined in (2); and/or
(ii) the monoclonal antibody comprises the VH sequence of SEQ ID NO: 70 and the VL sequence of SEQ ID NO: 69.

65-66. (canceled)

67. The method of claim 64, wherein:
(a) the monoclonal antibody that binds to CCR8 comprises a VH comprising (i) a CDR-H1 comprising the amino acid sequence of SEQ ID NO: 4 or SEQ ID NO: 5, (ii) a CDR-H2 comprising the amino acid sequence of SEQ ID NO: 6, and (iii) a CDR-H3 comprising the amino acid sequence of SEQ ID NO: 7, and a VL comprising (iv) a CDR-L1 comprising the amino acid sequence of SEQ ID NO: 1, (v) a CDR-L2 comprising the amino acid sequence of SEQ ID NO: 2, and (vi) a CDR-L3 comprising the amino acid sequence of SEQ ID NO: 3; and wherein:
(i) the monoclonal antibody comprises a VH sequence selected from the group consisting of SEQ ID NOs: 10-21 and a VL sequence selected from the group consisting of SEQ ID NOs: 22-25;
(ii) the monoclonal antibody comprises a sequence selected from the group consisting of (1) a VH sequence having at least about 95%, at least about 96%, at least about 97%, at least about 98%, or at least about 99% sequence identity to an amino acid sequence of SEQ ID NO: 21: (2) a VL sequence having at least about 95%, at least about 96%, at least about 97%, at least about 98%, or at least about 99% sequence identity to an amino acid sequence of SEQ ID NO: 24; and (3) a VH sequence as defined in (1) and a VL sequence as defined in (2); or
(iii) the monoclonal antibody comprises a VH sequence having at least about 95%, at least about 96%, at least about 97%, at least about 98%, or at least about 99% sequence identity to an amino acid sequence of SEQ ID NO: 21 and a VL sequence having at least about 95%, at least about 96%, at least about 97%, at least about 98%, or at least about 99% sequence identity to an amino acid sequence of SEQ ID NO: 24;
(b) the monoclonal antibody that binds to CCR8 comprises a VH comprising (i) a CDR-H1 comprising the amino acid sequence of SEQ ID NO: 82 or SEQ ID NO: 83, (ii) a CDR-H2 comprising the amino acid sequence of SEQ ID NO: 84, and (iii) a CDR-H3 comprising the amino acid sequence of SEQ ID NO: 85, and a VL comprising (iv) a CDR-L1 comprising the amino acid sequence of SEQ ID NO: 73, (v) a CDR-L2 comprising the amino acid sequence of SEQ ID NO: 74, and (vi) a CDR-L3 comprising the amino acid sequence of SEQ ID NO: 75; and wherein the monoclonal antibody comprises the VH sequence of SEQ ID NO: 95 and the VL sequence of SEQ ID NO: 94;
(c) the monoclonal antibody that binds to CCR8 comprises a VH comprising (i) a CDR-H1 comprising the amino acid sequence of SEQ ID NO: 86 or SEQ ID NO: 87, (ii) a CDR-H2 comprising the amino acid sequence of SEQ ID NO: 88, and (iii) a CDR-H3 comprising the amino acid sequence of SEQ ID NO: 89, and a VL comprising (iv) a CDR-L1 comprising the amino acid sequence of SEQ ID NO: 76, (v) a CDR-L2 comprising the amino acid sequence of SEQ ID NO: 77, and (vi) a CDR-L3 comprising the amino acid sequence of SEQ ID NO: 78; and wherein the monoclonal antibody comprises the VH sequence of SEQ ID NO: 97 and the VL sequence of SEQ ID NO: 96;
(d) the monoclonal antibody that binds to CCR8 comprises a VH comprising (i) a CDR-H1 comprising the amino acid sequence of SEQ ID NO: 90 or SEQ ID NO: 91, (ii) a CDR-H2 comprising the amino acid sequence of SEQ ID NO: 92, and (iii) a CDR-H3 comprising the amino acid sequence of SEQ ID NO: 93, and a VL comprising (iv) a CDR-L1 comprising the amino acid sequence of SEQ ID NO: 79, (v) a CDR-L2 comprising the amino acid sequence of SEQ ID NO: 80, and (vi) a CDR-L3 comprising the amino acid sequence of SEQ ID NO: 81; and wherein the monoclonal antibody comprises the VH sequence of SEQ ID NO: 99 and the VL sequence of SEQ ID NO: 98; or
(e) the monoclonal antibody that binds to CCR8 comprises a VH comprising (i) a CDR-H1 comprising the amino acid sequence of SEQ ID NO: 65 or SEQ ID NO: 66, (ii) a CDR-H2 comprising the amino acid sequence of SEQ ID NO: 67 and (iii) a CDR-H3 comprising the amino acid sequence of SEQ ID NO: 68, and a VL comprising (iv) a CDR-L1 comprising the amino acid sequence of SEQ ID NO: 62, (v) a CDR-L2 comprising the amino acid sequence of SEQ ID NO: 63, and (vi) a CDR-L3 comprising the amino acid sequence of SEQ ID NO: 64; and wherein the monoclonal antibody comprises the VH sequence of SEQ ID NO: 70 and the VL sequence of SEQ ID NO: 69.

68-91. (canceled)

92. The method of claim 1, wherein:
(i) the monoclonal antibody that binds to CCR8 binds to CCR8 independent of sulfation of CCR8;
(ii) the monoclonal antibody that binds to CCR8 is a human antibody, a humanized antibody, or a chimeric antibody;
(iii) the monoclonal antibody that binds to CCR8 is an antibody fragment that binds to CCR8 or a full-length antibody;
(iv) the monoclonal antibody that binds to CCR8 comprises an IgG1 constant domain comprising the amino acid sequence of SEQ ID NO: 53 or SEQ ID NO: 59;
(v) the monoclonal antibody that binds to CCR8 comprises a kappa constant domain comprising the amino acid sequence of SEQ ID NO: 54;
(vi) the monoclonal antibody that binds to CCR8 binds to CCR8 with a binding affinity (K_{d}) of from about 1×10⁻¹² M to about 1×10⁻¹¹ M;
(vii) the CCR8 is a human CCR8;
(viii) the monoclonal antibody that binds to CCR8 is afucosylated;
(ix) the regulatory T cells present in the tumor microenvironment, or outside of the tumor microenvironment, of the locally advanced, recurrent, or metastatic solid tumor malignancy are depleted; and/or
(x) the subject is a human.

93. The method of claim 92, wherein:
(i) the monoclonal antibody binds to an epitope comprising (1) one or more of amino acid residues 2-6 of SEQ ID NO: 106; or (2) one or more of amino acid residues 91-104 and 172-193 of SEQ ID NO: 106;
(ii) the monoclonal antibody is a humanized, full-length IgG1 antibody;
(iii) the proportion of afucosylation of the monoclonal antibody is between about 80% to about 95%; and/or
(iv) the monoclonal antibody comprises an IgG1 constant domain comprising the amino acid sequence of SEQ ID NO: 53 and a kappa constant domain comprising the amino acid sequence of SEQ ID NO: 54.

94-114. (canceled)
